(19) United States
(12) Patent Application Publication
(10) Pub. No.: US 2009/0035215 A1
(43) Pub. Date: Feb. 5, 2009

(54) RADIOFLUORINATION

(76) Inventors: Ananth Srinivasan, Berlin (DE); Ulrich Klar, Berlin (DE); Lutz Lehmann, Berlin (DE); Ulrike Voigtmann, Berlin (DE); Timo Stellfeld, Berlin (DE); Aileen Hohne, Zurich (CH); Linjing Mu, Allschwil (CH); Simon Ametamey, Zurich (CH)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/851,936

(22) Filed: Sep. 7, 2007

Related U.S. Application Data
(60) Provisional application No. 60/851,286, filed on Oct. 13, 2006.
(60) Provisional application No. 60/914,886, filed on Apr. 30, 2007.

(30) Foreign Application Priority Data
Oct. 2, 2006 (EP) 06076823.1
Oct. 11, 2006 (EP) 06076869.4
Mar. 12, 2007 (EP) 07090043.6
Apr. 23, 2007 (EP) 07090079.0

(51) Int. Cl. A61K 51/08; C07F 7/10; C07F 7/08; C07K 5/06; C07K 5/08; C07K 5/10; C07K 7/06; C07K 7/08; C07K 14/00; A61K 31/695; A61P 35/00; A61K 51/04; A61K 38/05; A61K 38/06; A61K 38/07; A61K 38/08; A61K 38/10; A61K 38/16; A61K 49/00

(52) U.S. Cl. 424/1.69; 556/419; 556/438; 530/331; 530/330; 530/329; 530/328; 530/327; 530/326; 530/350; 514/63; 514/19; 514/18; 514/17; 514/16; 514/15; 514/14; 514/13; 514/12; 424/9.1; 424/1.89

(57) ABSTRACT

The present invention relates to novel compounds suitable for or already radiolabeled with ¹⁸F, methods of making such compounds and use of such compounds for diagnostic imaging. Such labeled compounds are characerized by Formula II, wherein the moieties F, R¹, R², B^{1,2}, Y^{1,2}, Z^{1,2} and E have the meaning as defined in the specification and claims.

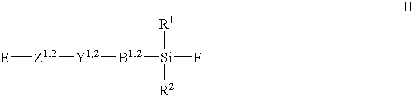

### FIELD OF INVENTION

This invention relates to novel compounds suitable for labelling with fluorine isotope, preferably ¹⁸F, or which are already labelled with fluorine isotope, preferably ¹⁸F, methods of preparing such compounds, compositions comprising such compounds, kits comprising such compounds or compositions and uses of such compounds, compositions or kits for diagnostic imaging, preferably positron emission tomography (PET).

### BACKGROUND ART

Molecular imaging has the potential to detect disease progression or therapeutic effectiveness earlier than most conventional methods in the fields of oncology, neurology and cardiology. Of the several promising molecular imaging technologies having been developed as optical imaging and MRI, Positron Emission Tomography (PET) is of particular interest for drug development because of its high sensitivity and ability to provide quantitative and kinetic data.

Over the last few years, in vivo scanning using PET has increased. PET is both a medical and research tool. It is used heavily in clinical oncology for medical imaging of tumors and the search for metastases, and for clinical diagnosis of certain diffuse brain diseases such as those causing various types of dementias. Radiotracers consisting of a radionuclide stably bound to a biomolecule is used for in vivo imaging of disorders.

In designing an effective radiopharmaceutical tracer for use as a diagnostic agent, it is imperative that the drugs have appropriate in vivo targeting and pharmacokinetic properties. Fritzberg et al., J. Nucl. Med., 1992, 33:394, further state that radionuclide chemistry and associated linkages underscore the need to optimize attachment and labelling chemical modifications of the biomolecule carrier. Hence the type of radionuclide, the type of biomolecule and the method used for linking them to one another may have a crucial effect onto the radiotracer properties.

The radionuclides used in PET scanning are typically isotopes with short half lives such as ¹¹C (˜20 min), ¹³N (˜10 min), ¹⁵O (˜2 min), ⁶⁸Ga (˜68 min) or ¹⁸F (˜110 min). Due to their short half lives, the radionuclides must be produced in a cyclotron which is not too far away in delivery-time from the PET scanner. These radionuclides are incorporated into biologically active compounds or biomolecules that have the function to vehicle the radionuclide into the body to the targeted site, e.g., to the tumor.

Positron emitting isotopes include carbon, nitrogen, and oxygen. These isotopes can replace their non-radioactive counterparts in target compounds to produce tracers that function biologically and are chemically identical to the original molecules for PET imaging. On the other hand, ¹⁸F is the most convenient labelling isotope due to its relatively long half life (109.6 min) which permits the preparation of diagnostic tracers and subsequent study of biochemical processes. In addition, its low β+ energy (635 keV) is also advantageous.

PET tracers are or often include a molecule of biological interest. Biomolecules developed for use in PET have been numerously intended for specific targeting in the patient as, e.g., FDG, FLT, L-DOPA, methionine and deoxythymidine. Due to their specific use, such biomolecules are often designated as “targeting agents”.

Peptides are biomolecules that play a crucial role in many physiological processes including actions as neurotransmitters, hormones and antibiotics. Research has shown their importance in such fields as neuroscience, immunology, pharmacology, and cell biology. Some peptides can act as chemical messenger. They bind to receptor on the target cell surface and the biological effect of the ligand is transmitted to the target tissue. Hence the specific receptor binding property of the ligand can be exploited by labelling the ligand with a radionuclide. Theoretically, the high affinity of the ligand for the receptor facilitates retention of the radiolabelled ligand in receptor expressing tissues. However, it is still under investigation which peptides can be efficiently labelled and under which conditions the labelling shall occur. It is well known that the receptor specificity of a ligand peptide may be altered during chemical reaction. Therefore an optimal peptidic construct has to be determined.

Tumors overexpress various receptor types to which peptides bound specifically. Boerman et al., Seminar in Nuclear Medicine, July, 2000, 30, (3); 195-208, provide a non exhaustive list of peptides binding to receptors involved in tumors, i.e., somatostatin, vasoactive intestinal peptide (VIP), bombesin binding to gastrin-releasing peptide (GRP) receptor, gastrin, cholecystokinin (CCK), and calcitonin.

The linkage of the radionuclide to the biomolecule is done by various methods resulting to the presence or not of a linker between the radionuclide and the biomolecule. Hence, various linkers are known. C. J. Smith et al., “Radiochemical investigations of ¹⁷⁷Lu-DOTA-8-Aoc-BBN[7-14]NH2: an in vitro/in vivo assessment of the targeting ability of this new radiopharmaceutical for PC-3 human prostate cancer cells.” Nucl. Med. Bio., 2003, 30(2):101-9, disclose radiolabeled bombesin wherein the linker is DOTA-X where X is a carbon tether. However, the radiolabel ¹⁷⁷Lu (half life 6.5 days) does not match the biological half-life of the native bombesin what makes the ¹⁷⁷Lu-DOTA-X-bombesin a non-appropriate radiotracer for imaging tumors.

E. Garcia Garayoa et al., “Chemical and biological characterization of new Re(CO)₃/[^{99m}Tc](CO)3 bombesin analogues.” Nucl. Med. Biol., 2007:17-28, disclose a spacer between the radionuclide [^{99m}Tc] and the bombesin wherein the spacer is

-β-Ala-β-Ala- and 3,6-dioxa-8-aminooctanoic acid. E. Garcia Garayoa et al. conclude that the different spacer did not have a significant effect on stability or on receptor affinity.

Listed above linkers have been specifically designed for a specific type of radionuclide and determine the type and chemical conditions of the radiobinding method.

More recently, peptides have been conjugated to macrocyclic chelators for labelling of ⁶⁴Cu, ⁸⁶Y, and ⁶⁸Ga for PET application. However, such radionuclides interact with the in vivo catabolism resulting in unwanted physiologic effects and chelate attachment.

Various methods of radiofluorination have been published using different precursor or starting materials for obtaining ¹⁸F-labelled peptides. Due to the smaller size of peptides, both higher target-to-background ratios and rapid blood clearance can often be achieved with radiolabeled peptides. Hence, short-lived positron emission tomography (PET) isotopes are potential candidates for labelling peptides. Among a number of positron-emitting nuclides, fluorine-18 appears to be the best candidate for labelling bioactive peptides by virtue of its favourable physical and nuclear characteristics. The major disadvantage of labelling peptides with ¹⁸F is the laborious and time-consuming preparation of the ¹⁸F labelling agents. Due to the complex nature of peptides and several functional groups associated with the primary structure, ¹⁸F-labelled peptides are not prepared by direct fluorination. Hence, difficulties associated with the preparation of ¹⁸F-labeled peptides were alleviated with the employment of prosthetic groups as shown below. Several such prosthetic groups have been proposed in the literature, including N-succinimidyl-4-[¹⁸F]fluorobenzoate, m-maleimido-N-(p-[¹⁸F]fluorobenzyl)-benzamide, N-(p-[¹⁸F]fluorophenyl) maleimide, and 4-[¹⁸F]fluorophenacylbromide. Almost all of the methodologies currently used today for the labeling of peptides and proteins with ¹⁸F utilize active esters of the fluorine labeled synthon.

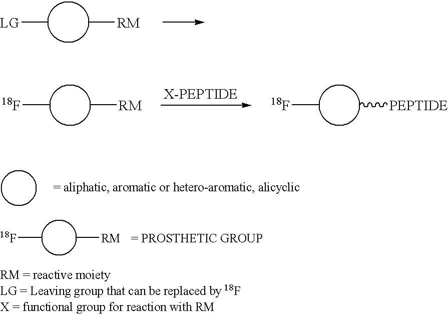

Okarvi et al., “Recent progress in fluorine-18 labelled peptide radiopharmaceuticals.” Eur. J. Nucl. Med., July 2001, 28(7):929-38, present a review of the recent developments in ¹⁸F-labelled biologically active peptides used in PET.

Zhang Xianzhong et al., “¹⁸F-labeled bombesin analogs for targeting GRP receptor-expressing prostate cancer.” J. Nucl. Med., 2006, 47(3):492-501, relate to the 2-step method detailed above. [Lys3]Bombesin ([Lys3]BBN) and aminocaproic acid-bombesin(7-14) (Aca-BBN(7-14)) were labeled with ¹⁸F by coupling the Lys3 amino group and Aca amino group, respectively, with N-succinimidyl-4-¹⁸F-fluorobenzoate (18F-SFB) under slightly basic condition (pH 8.5). Unfortunately, the obtained ¹⁸F-FB-[Lys3]BBN is relatively metabolically unstable having for result to reduce the extent of use of the ¹⁸F-FB-[Lys3]BBN for reliable imaging of tumors.

Poethko Thorsten et al., “Two-step methodology for high-yield routine radiohalogenation of peptides: ¹⁸F-labeled RGD and octreotide analogs.” J. Nucl Med., May 2004, 45(5):892-902, relate to a 2-step method for labelling RGD and octreotide analogs. The method discloses the steps of radiosynthesis of the ¹⁸F-labeled aldehyde or ketone and the chemoselective ligation of the ¹⁸F-labeled aldehyde or ketone to the aminooxy functionalized peptide.

Poethko Thorsten et al., “First ¹⁸F-labeled tracer suitable for routine clinical imaging of somatostatin receptor-expressing tumors using positron emission tomography.” Clin. Cancer Res., Jun. 2004 1, 10(11):3593-606, apply the 2-step method for the synthesis of ¹⁸F-labeled carbohydrated Tyr(3)-octreotate (TOCA) analogs with optimized pharmacokinetics suitable for clinical routine somatostatin-receptor (sst) imaging.

WO 2003/080544 A1 and WO 2004/080492 A1 relate to radiofluorination methods of bioactive peptides for diagnostics imaging using the 2-step method shown above.

¹⁸F-labeled compounds are gaining importance due to their availability as well as due to the development of methods for labeling biomolecules. It has been shown that some compounds labeled with ¹⁸F, produce images of high quality. Additionally, the longer lifetime of ¹⁸F would permit longer imaging times and allows preparation of radiotracer batches for multiple patients and delivery of the tracer to other facilities, making the technique more widely available to clinical investigators. Additionally, it has been observed the development of PET cameras and availability of the instrumentation in many PET centers is increasing. Hence, it is increasingly important to develop new tracers labeled with ¹⁸F.

Several approaches for incorporating ¹⁸F into more complex biomolecules as, e.g., peptides are described in the following references: European J. Nucl. Med. Mol. Imaging, 2001, 28:929-938; European J. Nucl. Med. Mol. Imaging, 2004, 31:1182-1206; Bioconjugate Chem., 1991, 2:44-49; Bioconjugate Chem., 2003, 14:1253-1259.

These methods are indirect. They demand at least a two step procedure for tracer synthesis. Therefore they are time consuming thereby reducing PET image resolution as a result of nuclear decay.

The most crucial aspect in the successful treatment of any cancer is early detection. Likewise, it is crucial to properly diagnose the tumors and metastases.

Routine application of ¹⁸F-labeled peptides for quantitative in vivo receptor imaging of receptor-expressing tissues and quantification of receptor status using PET is limited by the lack of appropriate radiofluorination methods for routine large-scale synthesis of ¹⁸F-labeled peptides. There is a clear need for radiofluorination method that can be conducted rapidly without loss of receptor affinity by the peptide and leading to a positive imaging (with reduced background), wherein the radiotracer is stable and shows enhanced clearance properties.

In general, the preparation of silyl fluorides is well understood in the art especially in the cleavage of trialkyl silyl hydroxy protecting groups by TBAF, HF or KF which results in trialkyl silyl fluorides. (Greene et al., “Protective Groups in Organic Synthesis”, Third Edition, 1999, Wiley VCH.)

S. McN. Sieburth et al., J. Org. Chem., 2005, 70, 15:5781-5789, describe the conversion of silicon-oxygen bonds in peptides like ACE inhibitors to silicon fluoride bonds by using HF followed by the hydrolysis using sodium hydroxide to avoid any oligomer formation during direct hydrolysis.

Very few publications are known which describe the preparation of silicon based ¹⁸F-labelled tracers:

Rosenthal et al., Int. J. Appl. Radiat. Isot, 1985, 36 (4):318-319, have published the first report on ¹⁸F labelling of silyl derivatives. ¹⁸F-Fluorotrimethylsilane was prepared by reaction of TMAF with chlorotrimethylsilane in aqueous acetonitrile. Rats which were allowed to inhale the gas showed an extensive uptake of ¹⁸F in the bone, demonstrating a fast release of fluoride in vivo.

J. C. Walsh et al., “Application of silicon-fluoride chemistry to fluorine-18 labeling agents for biomolecules: a preliminary note”, J. Labelled Cpd. Radiopharm., 1999, 42 Suppl. 1:S1-S3; J. Nuclear Medicine, Supp. S., 2000, 41:1098, describe a PET model compound containing one ¹⁸F, two phenyl groups and a tertiary butyl group attached to silicon with sufficient hydrolytic stability. Based on this model compound, the inclusion of a thio-reactive or amine-reactive group for subsequent attachment to a targeting agent was planned according to the abstract, but to our knowledge not published yet. Nevertheless, such an indirect approach needs at least two synthetic steps, labelling and binding to a targeting agent to get a useful PET probe.

Choudhry et al. reported in their poster abstract on a one-step F-18 labelling towards Si-18F derivatives (European Journal of Nuclear Medicine and Molecular Imaging, September 2006, Vol. 33, Supplement (EANM Athene 2006), P394, p. 306).

It turned out that diphenyl-tert-butyl-SiF-derivatives are superior to triphenyl-Si—F, phenyl-dimethyl-Si—F and tert-butyl-dimethyl-SiF regarding hydrolytic stability. The title of the abstract suggests an instant labelling of biomolecules by this methods, but examples are not presented or given elsewhere.

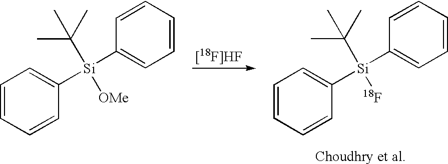

In the corresponding poster only methoxy was presented as leaving group regarding F-18 labelling of silicon-containing molecules. The disadvantage of methoxy is that the precursor (starting material) cannot be separated easily from the F-18 labeled molecule or targeting agent: the retention time of both are too similar to achieve a convenient purification of the desired F-18 product. It can be shown that those leaving groups presented by alkoxy, comprising more than one carbon atom, hydrogen, hydroxyl and aralkoxy are more suited to achieve a F-18 labeling and a subsequent successful separation of the desired F-18 labelled product from the precursor.

Perrin et al., J. Am. Chem. Soc., 2005, 127:13094-13095 and WO 2005/077967 A, describe that multiple F atoms may be joined to silicon to incorporate a greater number of ¹⁸F atoms into a single tracer. This enhances the density of positron emitters in the resulting product and stabilizes the silicon moiety under physiological conditions with a rate that is on par with that of ¹⁸F decay. But, depending on the number of fluorine atoms incorporated, compounds of this invention may be charged or uncharged which is not predictable leading to mixtures of products.

Very recently, R. Schirrmacher et al., “¹⁸F-Markierung von Peptiden mihilfe eines Organosilicium-Fluoridacceptors”, Angew. Chem., 2006, 118:6193-6197, have described the preparation of ¹⁸F-organofluorsilanes via ¹⁸F-¹⁹F isotope exchange reactions. A ¹⁸F-Tyr3-octreotate derivative used for diagnosis of neuroendocrine tumors was synthesized. Nevertheless, this tracer prepared via isotope exchange reactions contains predominantly the corresponding non-radioactive ¹⁹F-compound which lead to products with relatively low specific activity. The use of other leaving groups followed by an efficient separation of the non-radioactive precursor would therefore be preferred.

Further, M. Ushioda et al., “Unique participation of unprotected internucleotidic phosphodiester residues on unexpected cleavage reaction of the Si—O Bond of the diisopropylsilandiyl group used as a linker for the solid-phase synthesis of 5′-terminal guanylated oligodeoxynucleotides”, Helv. Chim. Acta, 2002, 85:2930-45, disclose Ethoxy-di-iso-propyl-silanol derivatives of desoxynucleotides.

Recently, Coenen, “Fluorine-18 Labelling Methods: Features and Possibilities of Basic Reactions; PET chemistry—the driving force in molecular imaging”, Springer, 2006, in press, has published a review article describing chemical and technical aspects of ¹⁸F-labeling: “[¹⁸F]fluoride is obtained as an aqueous solution [ . . . ]. Due to the high charge density of the anion it is strongly hydrated (ΔHhydr=506 kJ/mol) and inactivated for nucleophilic reactions. [ . . . ]. It is very easily protonated, forming hydrogen fluoride (EB=565 kJ/mol), which then makes it unavailable for further reactions. Labelling, therefore, has to take place under aprotic but polar conditions. [ . . . ] However, routinely the nucleophilic radiofluorination with appropriate precursors is performed in dipolar aprotic solvents using fluoride salts with a soft cation (Cs⁺, Rb⁺) thus rendering weak—easy to separate—ion pairs and ‘naked’ [¹⁸F]fluoride of high nucleophilicity. For further anion activation phase transfer catalysts (PTC) like tetraalkylammonium carbonates (hydrogen carbonates) [ . . . ] or mainly the aminopolyether Kryptofix® 2.2.2 in combination with potassium carbonate or oxalate [ . . . ] are optimal. [ . . . ] Since fluoride in dipolar aprotic solvents exhibits also a strong basic character and generally the reaction medium is basic (CO₃²⁻, HCO₃⁻), elimination reactions can compete with nucleophilic substitution. [ . . . ] A problem is the basicity of the reaction solution. Thus, base labile compounds such as butyrophenone neuroleptics, for example, could only be ¹⁸F-labelled directly when the [K⊂2.2.2]₂CO₃/C₂O₄ buffer system was used [ . . . ]”

For these reasons it would be desirable to have a method which allows the ¹⁸F-radiolabelling, when acid is added; in particular the ¹⁸F-radiolabeling of base-labile compounds which would profit from such a method.

Therefore it is an object of the present invention, to develop a practical and mild technique for ¹⁸F labelling, of targeting agents like peptides in only one rather than two or more chemical steps in order to save time, costs and additional purification steps of radioactive compounds and to provide radiofluorination methods for obtaining radiotracer based on receptor specific peptides for the detection of tumors.

### SUMMARY OF THE INVENTION

In a first aspect, the present invention provides silicon substituted target compounds having general chemical Formula I, which can be labelled with ¹⁸F, in a one step radiolabelling procedure. These compounds are precursors for single step radiolabeling, i.e., radiofluorination.

In a second aspect, the present invention provides fluorinated silicon substituted target compounds, more preferably compounds being labelled with fluorine isotope, having general chemical Formula II, which are suitable as radiotracers.

In a third aspect, the present invention provides silicon substituted building blocks having general chemical Formula III, which are suitable for preparing compounds having general chemical Formula I.

In a fourth aspect, the present invention is related to a method for producing a compound having general chemical Formula I, as defined herein above, more preferably a method of radiofluorination of such compound, wherein a compound having general chemical formula III, as also defined herein above, is reacted with a compound having general chemical Formula IV. Such method yields a compound having general chemical Formula II.

In a fifth aspect, the present invention is directed to a method of radiolabeling, of compounds having any one of general chemical Formulae I and III with ¹⁸F, under appropriate reaction conditions to yield compounds having general chemical Formula II. Such method comprises the step of reacting a compound having any one of general chemical Formulae I and III with a fluorinating agent.

In a sixth aspect, the present invention relates to a composition comprising a compound having general chemical Formula I or a compound being prepared with the method of the fifth aspect.

In a seventh aspect, the present invention relates to a method of imaging diseases, comprising introducing into a patient a detectable quantity of a labelled compound having general chemical Formula II.

In an eighth aspect, the present invention relates to a kit comprising a vial containing a predetermined quantity of a compound having any one of general chemical Formulae I, II and III, including a compound which is prepared with the method of the fifth aspect, or a composition of the sixth aspect, along with an acceptable carrier, diluent, excipient or adjuvant for the manufacture of ¹⁸F radiolabeled compounds. According to this aspect of the present invention, the kit comprises any of the ¹⁸F radiolabeled compounds as defined hereinabove or a composition comprising the same, e.g., in powder form, and a container containing an appropriate solvent for preparing a physiologically acceptable solution of the compound or composition for administration to an animal, including a human. Further, according to this aspect of the present invention the kit comprises a compound having general chemical Formula I as disclosed above along with an acceptable carrier, diluent, excipient or adjuvant supplied as a mixture with the compound having general chemical Formula I or independently for the manufacture of a compound having general chemical Formula II.

In a ninth aspect, the present invention is directed to a labelled compound with ¹⁸F isotope, having general chemical Formula II for use as medicament, more preferably for use as diagnostic imaging agent and more preferably for use as imaging agent for positron emission tomography. In another variation of this aspect, the present invention also relates to fluorinated compounds, which are more preferably labelled with ¹⁹F isotope and which have general chemical Formula II, for use in biological assays and chromatographic identification.

In a tenth aspect, the present invention relates to the use of any fluorinated compound, as defined hereinabove, or of the precursor thereof for diagnostic imaging, in particular with positron emission tomography or for the manufacture of a medicament, more preferably for the manufacture of a diagnostic imaging agent, most preferably for imaging tissue at a target site using the imaging agent.

In an eleventh aspect, the present invention relates to the use of the compounds having general chemical Formulae I or II, including compounds being prepared with the method of the fifth aspect, or of the composition of the sixth aspect or of the kit of the eighth aspect for diagnostic imaging, in particular for positron emission tomography and most preferably for imaging of tumors, of inflammatory and/or neurodegenerative diseases, such as multiple sclerosis or Alzheimer's disease, or for imaging of angiogenesis-associated diseases, such as growth of solid tumors, and of rheumatoid arthritis.

### DETAILED DESCRIPTION OF THE INVENTION

As used hereinafter in the description of the invention and in the claims, the term “alkyl”, by itself or as part of another group, refers to a straight chain or branched chain alkyl group with 1 to 20 carbon atoms, such as, for example, methyl, ethyl, propyl, iso-propyl, butyl, iso-butyl, tert-butyl, pentyl, iso-pentyl, neo-pentyl, heptyl, hexyl, decyl. Alkyl groups can also be substituted, such as by halogen atoms, hydroxyl groups, C₁-C₄-alkoxy groups or C₆-C₁₂-aryl groups (which, intern, can also be substituted, such as by 1 to 3 halogen atoms). More preferably alkyl is C₁-C₁₀ alkyl, C₁-C₆ alkyl or C₁-C₄ alkyl.

As used hereinafter in the description of the invention and in the claims, the term “cycloalkyl” by itself or as part of another group, refers to mono- or bicyclic chain of alkyl group with 3 to 20 carbon atoms such as, for example cyclopropyl, cyclobutyl, cyclopentyl, cyclohexyl or cycloheptyl. More preferably cycloalkyl is C₃-C₁₀ cycloalkyl or C₅-C₈ cycloalkyl, most preferably C₆ cycloalkyl.

As used hereinafter in the description of the invention and in the claims, the term “heterocycloalkyl”, by itself or as part of another group, refers to groups having 5 to 14 mon- or bi-ring atoms of a cycloalkyl; and containing carbon atoms and 1, 2, 3 or 4 oxygen, nitrogen or sulfur heteroatoms. More preferably heterocycloalkyl is C₃-C₁₀ heterocycloalkyl, C₅-C₈ heterocycloalkyl or C₅-C₁₄ heterocycloalkyl, most preferably C₆ heterocycloalkyl.

As used hereinafter in the description of the invention and in the claims, the term “aralkyl” refers to aryl-substituted alkyl radicals such as benzyl, diphenylmethyl, triphenylmethyl, phenylethyl, phenylbutyl and diphenylethyl.

As used hereinafter in the description of the invention and in the claims, the terms “aryloxy” refers to aryl groups having an oxygen through which the radical is attached to a nucleus, examples of which are phenoxy.

As used hereinafter in the description of the invention and in the claims, the terms “alkenyl” and “alkynyl” are similarly defined as for alkyl, but contain at least one carbon-carbon double or triple bond, respectively. More preferably C₂-C₆ alkenyl and C₂-C₆ alkynyl.

As used hereinafter in the description of the invention and in the claims, the term “unbranched or branched lower alkyl” shall have the following meaning: a substituted or unsubstituted, straight or branched chain monovalent or divalent radical consisting substantially of carbon and hydrogen, containing no unsaturation and having from one to eight carbon atoms, e.g., but not limited to methyl, ethyl, n-propyl, n-pentyl, 1,1-dimethylethyl (t-butyl), n-heptyl and the like.

As used hereinafter in the description of the invention and in the claims, the terms “aralkenyl” refers to aromatic structure (aryl) coupled to alkenyl as defined above.

As used hereinafter in the description of the invention and in the claims, the terms “alkoxy (or alkyloxy), aryloxy, and aralkenyloxy” refer to alkyl, aryl, and aralkenyl groups respectively linked by an oxygen atom, with the alkyl, aryl, and aralkenyl portion being as defined above.

As used hereinafter in the description of the invention and in the claims, the term “aryl”, by itself or as part of another group, refers to monocyclic or bicyclic aromatic groups containing from 6 to 12 carbon atoms in the ring portion, preferably 6-10 carbons in the ring portion, such as phenyl, naphthyl or tetrahydronaphthyl.

As used hereinafter in the description of the invention and in the claims, the term “heteroaryl”, by itself or as part of another group, refers to groups having 5 to 14 ring atoms; 6, 10 or 14 π electrons shared in a cyclic array; and containing carbon atoms and 1, 2, 3 or 4 oxygen, nitrogen or sulfur heteroatoms. Examples of heteroaryl groups are: thienyl, benzo[b]thienyl, naphtho[2,3-b]thienyl, thianthrenyl, furyl, pyranyl, isobenzofuranyl, benzoxazolyl, chromenyl, xanthenyl, phenoxythiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinazolinyl, cinnolinyl, pteridinyl, 4H-carbazolyl, carbazolyl, carbolinyl, phenanthridinyl, acridinyl, perimidinyl, phenanthrolinyl, phenazinyl, isothiazolyl, phenothiazinyl, isoxazolyl, furazanyl und phenoxazinyl.

Whenever the term substituted is used, it is meant to indicate that one or more hydrogens on the atom indicated in the expression using “substituted” is replaced with a selection from the indicated group, provided that the indicated atom's normal valency is not exceeded, and that the substitution results in a chemically stable compound, i.e. a compound that is sufficiently robust to survive isolation to a useful degree of purity from a reaction mixture, and formulation into a pharmaceutical composition. The substituent groups may be selected from halogen atoms, hydroxyl groups, C₁-C₄ alkoxy groups or C₆-C₁₂ aryl groups (which, intern, can also be substituted, such as by 1 to 3 halogen atoms).

As used hereinafter in the description of the invention and in the claims, the term “fluorine isotope” (F) refers to all isotopes of the fluorine atomic element. Fluorine isotope (F) is selected from radioactive or non-radioactive isotope. The radioactive fluorine isotope is selected from ¹⁸F. The non-radioactive “cold” fluorine isotope is selected from ¹⁹F.

As used hereinafter in the description of the invention and in the claims, the term “prodrug” means any covalently bonded compound, which releases the active parent pharmaceutical. The term “prodrug” as used throughout this text means the pharmacologically acceptable derivatives such as esters, amides and phosphates such that the resulting in vivo biotransformation product of the derivative is the active drug as defined in the compounds of formula (I). The reference by Goodman and Gilman (The Pharmaco-logical Basis of Therapeutics, 8 ed, McGraw-HiM. Int. Ed. 1992, “Biotransformation of Drugs”, p 13-15) describing prodrugs generally is hereby incorporated. Prodrugs of a compound of the present invention are prepared by modifying functional groups present in the compound in such a way that the modifications are cleaved, either in routine manipulation or in vivo, to the parent compound. Prodrugs of the compounds of the present invention include those compounds wherein for instance a hydroxy group, such as the hydroxy group on the asymmetric carbon atom, or an amino group is bonded to any group that, when the prodrug is administered to a patient, cleaves to form a free hydroxyl or free amino, respectively.

Typical examples of prodrugs are described for instance in WO 99/33795, WO 99/33815, WO 99/33793 and WO 99/33792 all incorporated herein by reference.

Prodrugs are characterized by excellent aqueous solubility, increased bioavailability and are readily metabolized into the active inhibitors in vivo.

As used hereinafter in the description of the invention and in the claims, the terms “amino acid sequence” and “peptide” are defined herein as a polyamide obtainable by (poly) condensation of at least two amino acids.

As used hereinafter in the description of the invention and in the claims, the term “amino acid” means any molecule comprising at least one amino group and at least one carboxyl group, but no peptide bond within the molecule. In other words, an amino acid is a molecule that has a carboxylic acid functionality and an amine nitrogen having at least one free hydrogen, preferably in alpha position thereto, but no amide bond in the molecule structure. Thus, a dipeptide having a free amino group at the N-terminus and a free carboxyl group at the C-terminus is not to be considered as a single “amino acid” within the above definition. The amide bond between two adjacent amino acid residues which is obtained from such a condensation is defined as “peptide bond”.

An amide bond as used herein means any covalent bond having the structure

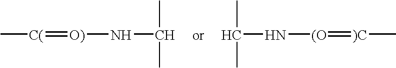

wherein the carbonyl group is provided by one molecule and the NH-group is provided by the other molecule to be joined. The amide bonds between two adjacent amino acid residues which are obtained from such a polycondensation are defined as “peptide bonds”. Optionally, the nitrogen atoms of the polyamide backbone (indicated as NH above) may be independently alkylated, e.g., with —C₁-C₆-alkyl, preferably —CH₃.

As used hereinafter in the description of the invention and in the claims, an amino acid residue is derived from the corresponding amino acid by forming a peptide bond with another amino acid.

As used hereinafter in the description of the invention and in the claims, an amino acid sequence may comprise naturally occurring and/or synthetic/artificial amino acid residues, proteinogenic and/or non-proteinogenic amino acid residues. The non-proteinogenic amino acid residues may be further classified as (a) homo analogues of proteinogenic amino acids, (b) β-homo analogues of proteinogenic amino acid residues and (c) further non-proteinogenic amino acid residues.

Accordingly, the amino acid residues are derived from the corresponding amino acids, e.g., from
proteinogenic amino acids, namely Ala, Arg, Asn, Asp, Cys, Gln, Glu, Gly, His, Ile, Leu, Lys, Met, Phe, Pro, Ser, Thr, Trp, Tyr and Val; or
non-proteinogenic amino acids, such as
   homo analogues of proteinogenic amino acids wherein the sidechain has been extended by a methylene group, e.g., homoalanine (Hal), homoarginine (Har), homocysteine (Hcy), homoglutamine (Hgl), homohistidine (Hhi), homoisoleucine (Hil), homoleucine (Hle), homolysine (Hly), homomethionine (Hme), homophenylalanine (Hph), homoproline (Hpr), homoserine (Hse), homothreonine (Hth), homotryptophane (Htr), homotyrosine (Hty) and homovaline (Hva);
   β-homoanalogues of proteinogenic amino acids wherein a methylene group has been inserted between the α-carbon and the carboxyl group yielding β-amino acids, e.g., β-homoalanine (βHal), β-homoarginine (βHar), β-homoasparagine (βHas), β-homocysteine (βHcy), β-homoglutamine (βHgl), β-homohistidine (βHhi), β-homoisoleucine (βHil), β-homoleucine (βHle), β-homolysine (βHly), β-homomethionine (βHme), β-homophenylalanine (βHph), β-homoproline (βHpr), β-homoserine (βHse), β-homothreonine (βHth), β-homotryptophane (βHtr), β-homotyrosine (βHty) and β-homovaline (βHva);
   further non-proteinogenic amino acids, e.g., α-aminoadipic acid (Aad), β-aminoadipic acid (βAad), α-aminobutyric acid (Abu), α-aminoisobutyric acid (Aib), P alanine (βAla), 4-aminobutyric acid (4-Abu), 5-aminovaleric acid (5-Ava), 6-aminohexanoic acid (6-Ahx), 8-aminooctanoic acid (8-Aoc), 9-aminononanoic acid (9-Anc), 10-aminodecanoic acid (10-Adc), 12-aminododecanoic acid (12-Ado), α-aminosuberic acid (Asu), azetidine-2-carboxylic acid (Aze), β-cyclohexylalanine (Cha), citrulline (Cit), dehydroalanine (Dha), γ-carboxyglutamic acid (Gla), α-cyclohexylglycine (Chg), propargylglycine (Pra), pyroglutamic acid (Glp), α-tert-butylglycine (Tle), 4-benzoylphenylalanine (Bpa), δ-hydroxylysine (Hyl), 4-hydroxyproline (Hyp), allo-isoleucine (alle), lanthionine (Lan), (1-naphthyl)alanine (1-Nal), (2-naphthyl)alanine (2-Nal), norleucine (Nle), norvaline (Nva), ornithine (Orn), phenylglycin (Phg), pipecolic acid (Pip), sarcosine (Sar), selenocysteine (Sec), statine (Sta), β-thienylalanine (Thi), 1,2,3,4-tetrahydroisochinoline-3-carboxylic acid (Tic), allo-threonine (aThr), thiazolidine-4-carboxylic acid (Thz), γ-aminobutyric acid (GABA), iso-cysteine (iso-Cys), diaminopropionic acid (Dpr), 2,4-diaminobutyric acid (Dab), 3,4-diaminobutyric acid (γβDab), biphenylalanine (Bip), phenylalanine substituted in para-position with —C₁-C₆ alkyl, -halide, —NH₂, —CO₂H or Phe(4-R) (wherein R═C₁-C₆ alkyl, -halide, —NH₂, or —CO₂H); peptide nucleic acids (PNA, cf., P. E. Nielsen, Acc. Chem. Res., 32, 624-30);
   or their N-alkylated analogues, such as their N-methylated analogues.

Cyclic amino acids may be proteinogenic or non-proteinogenic, such as Pro, Aze, Glp, Hyp, Pip, Tic and Thz.

For further examples and details reference can be made to, e.g., J. H. Jones, J. Peptide Sci., 2003, 9, 1-8 which is herein incorporated by reference.

As used hereinafter in the description of the invention and in the claims, the terms “non-proteinogenic amino acid” and “non-proteinogenic amino acid residue” also encompass derivatives of proteinogenic amino acids. For example, the side chain of a proteinogenic amino acid residue may be derivatized thereby rendering the proteinogenic amino acid residue “non-proteinogenic”. The same applies to derivatives of the C-terminus and/or the N-terminus of a proteinogenic amino acid residue terminating the amino acid sequence.

As used hereinafter in the description of the invention and in the claims, a proteinogenic amino acid residue is derived from a proteinogenic amino acid selected from the group consisting of Ala, Arg, Asn, Asp, Cys, Gln, Glu, Gly, His, Ile, Leu, Lys, Met, Phe, Pro, Ser, Thr, Trp, Tyr and Val either in L- or D-configuration; the second chiral center in Thr and Ile may have either R- or S-configuration. Therefore, for example, any posttranslational modification of an amino acid sequence, such as N-alkylation, which might naturally occur renders the corresponding modified amino acid residue “non-proteinogenic”, although in nature said amino acid residue is incorporated in a protein. Preferably modified amino acids are selected from N-alkylated amino acids, β-amino acids, γ-amino acids, lanthionines, dehydro amino acids, and amino acids with alkylated guanidine moieties.

As used hereinafter in the description of the invention and in the claims, the term “peptidomimetic” relates to molecules which are related to peptides, but with different properties. A peptidomimetic is a small protein-like chain designed to mimic a peptide. They typically arise from modification of an existing peptide in order to alter the molecule's properties. For example, they may arise from modifications to change the molecule's stability or biological activity. This can have a role in the development of drug-like compounds from existing peptides. These modifications involve changes to the peptide that will not occur naturally.

As used hereinafter in the description of the invention and in the claims, the term “peptide analogs”, by itself refers to synthetic or natural compounds which resemble naturally occurring peptides in structure and/or function.

As used hereinafter in the description of the invention and in the claims, the terms “salts of inorganic or organic acids”, “inorganic acid” and “organic acid” refer to mineral acids, including, but not being limited to: acids such as carbonic, nitric, phosphoric, hydrochloric, perchloric or sulphuric acid or the acidic salts thereof such as potassium hydrogen sulphate, or to appropriate organic acids which include, but are not limited to: acids such as aliphatic, cycloaliphatic, aromatic, araliphatic, heterocyclic, carboxylic and sulphonic acids, examples of which are formic, acetic, trifluoracetic, propionic, succinic, glycolic, gluconic, lactic, malic, fumaric, pyruvic, benzoic, anthranilic, mesylic, fumaric, salicylic, phenylacetic, mandelic, embonic, methansulfonic, ethanesulfonic, benzenesulfonic, phantothenic, toluenesulfonic, trifluormethansulfonic and sulfanilic acid, respectively.

As used hereinafter in the description of the invention and in the claims, the term “pharmaceutically acceptable salt” relates to salts of inorganic and organic acids, such as mineral acids, including, but not limited to, acids such as carbonic, nitric or sulfuric acid, or organic acids, including, but not limited to, acids such as aliphatic, cycloaliphatic, aromatic, araliphatic, heterocyclic, carboxylic and sulphonic acids, examples of which are formic, acetic, trifluoroacetic, propionic, succinic, glycolic, gluconic, lactic, malic, fumaric, pyruvic, benzoic, anthranilic, mesylic, salicylic, phenylacetic, mandelic, embonic, methansulfonic, ethanesulfonic, benzenesulfonic, phantothenic, toluenesulfonic and sulfanilic acid.

As used hereinafter in the description of the invention and in the claims, the term “oligonucleotide” shall have the following meaning: short sequences of nucleotides, typically with twenty or fewer bases. Examples are, but are not limited to, molecules named and cited in the book: “The aptamers handbook. Functional oligonuclides and their application” by Svenn Klussmann, Wiley-VCH, 2006. An example for such an oligonucleotide is TTA1 (J. Nucl Med., 2006, April, 47(4):668-78).

As used hereinafter in the description of the invention and in the claims, the term “aptamer” refers to an oligonucleotide, comprising from 4 to 100 nucleotides, wherein at least two single nucleotides are connected to each other via a phosphodiester linkage. Said aptamers have the ability to bind specifically to a target molecule (see, e.g., M Famulok, G Mayer, “Aptamers as Tools in Molecular Biology and Immunology”, in: “Combinatorial Chemistry in Biology, Current Topics in Microbiology and Immunology” (M Famulok, C H Wong, E L Winnacker, Eds.), Springer Verlag Heidelberg, 1999, Vol. 243, 123-136). There are many ways known to the skilled person of how to generate such aptamers that have specificity for a certain target molecule. An example is given in WO 2001/09390 A, the disclosure of which is hereby incorporated by reference. Said aptamers may comprise substituted or non-substituted natural and non-natural nucleotides. Aptamers can be synthesized in vitro using, e.g., an automated synthesizer. Aptamers according to the present invention can be stabilized against nuclease degradation, e.g., by the substitution of the 2′-OH group versus a 2′-fluoro substituent of the ribose backbone of pyrimidine and versus 2′-O-methyl substituents in the purine nucleic acids. In addition, the 3′ end of an aptamer can be protected against exonuclease degradation by inverting the 3′ nucleotide to form a new 5′-OH group, with a 3′ to 3′ linkage to a penultimate base.

For the purpose of this invention, the term “nucleotide” refers to molecules comprising a nitrogen-containing base, a 5-carbon sugar, and one or more phosphate groups. Examples of said base comprise, but are not limited to, adenine, guanine, cytosine, uracil, and thymine. Also non-natural, substituted or non-substituted bases are included. Examples of 5-carbon sugar comprise, but are not limited to, D-ribose, and D-2-desoxyribose. Also other natural and non-natural, substituted or non-substituted 5-carbon sugars are included. Nucleotides as used in this invention may comprise from one to three phosphates.

If a chiral center or another form of an isomeric center is present in a compound having general chemical Formula I, II, III or IV of the present invention, as given hereinafter, all forms of such isomers, including enantiomers and diastereoisomers, are intended to be covered herein. Compounds containing a chiral center may be used as a racemic mixture or as an enantiomerically enriched mixture, or the racemic mixture may be separated using well-known techniques and an individual enantiomer maybe used alone. In cases in which compounds have unsaturated carbon-carbon double bonds, both the cis-isomer and trans-isomers are within the scope of this invention. In cases in which compounds may exist in tautomeric forms, such as keto-enol tautomers, each tautomeric form is contemplated as being included within the scope of the present invention whether existing in equilibrium or predominantly in one form.

As used hereinafter in the description of the invention and in the claims, the term “halogen” refers to F, Cl, Br and I.

According to the first aspect, the present invention relates to novel compounds having general chemical Formula I:

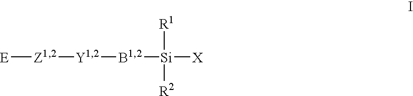

wherein
X represents a leaving group suitable for fluorination wherein X is a group of atoms or a reactive moiety attached to Si that can be displaced by fluorine isotope, to provide a chemically and biologically stable bond, selected from the group comprising hydrogen and OR³,
wherein R³ represents hydrogen, C₁-C₁₀ alkyl, C₂-C₁₀ alkenyl, C₂-C₁₀ alkynyl, aryl, heteroaryl or aralkyl;
R¹ and R², independently, are selected from the group comprising hydrogen, linear or branched C₁-C₁₀ alkyl, aryl, heteroaryl and aralkyl;
wherein further either:
   —B¹— is selected from the group comprising —[CH₂]ₘ-D-[CH₂]ₙ-A-,
   wherein n and m, independently, are any integer from 0 to 5,
   -D- represents a bond, —S—, —O— or —NR⁴—,
   wherein R⁴ represents hydrogen, C₁-C₁₀ alkyl, aryl, heteroaryl or aralkyl, and
   A represents alkyl, unsubstituted or substituted aryl, or unsubstituted or substituted heteroaryl, and
   E-Z¹-Y¹— represents a moiety selected from the group comprising
   EO—C(═O)—, ENR⁵—C(═O)—, EC(═O)—O—, EC(═O)—NR⁵—, ENR⁵—SO₂—, ESO₂—NR⁵—, E-O—, E-(S)ₚ—, E-NR⁵—, ENR⁶C(═O)—NR⁷—,
   ENR⁶—C(═O)NR⁷—, ENR⁶C(═S)—NR⁷—, ENR⁶—C(═S)NR⁷—_EO—C(═O)O—, EOC(═O)—O—, EOC(═S)—O—, EO-C(═S)O—, wherein the long single bond explicitly shown in the Formulae herein above is the bond between Z¹ and Y¹, and

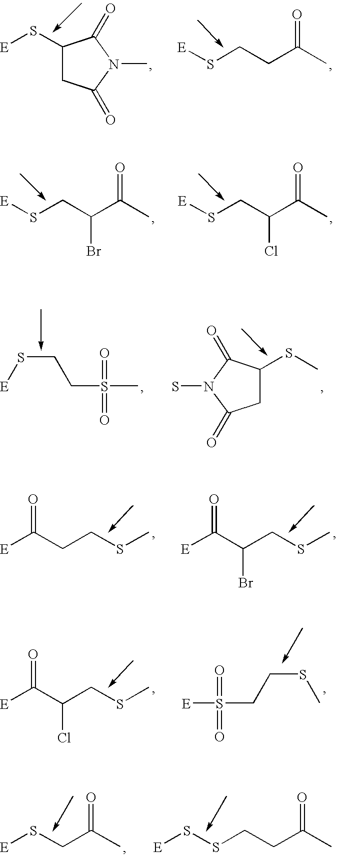

wherein the arrows shown in the Formulae herein above indicate the bond between Z¹ and Y¹,
wherein
R⁵, R⁶ and R⁷, independently, represent hydrogen, linear or branched C₁-C₁₀ alkyl, aryl, heteroaryl or aralkyl and
p is any integer from 1 to 3, and
wherein E-Z¹- is a targeting agent radical and E- is a biomolecule; or
—B²— represents a C₁-C₁₀ alkyl-, unsubstituted or substituted -aryl- or unsubstituted or substituted -heteroaryl-,
—Y²— is selected from the group comprising a bond, —C(═O)—, —SO₂—,
—C(═O)—(CH₂)_{d}—, —S(═O)—, —C(═O)—C≡C—, —C(═O)-[CH₂]ₘ-D-[CH₂]ₙ—, —SO₂-[CH₂]ₘ-D-[CH₂]ₙ—, O—C(═O)—, —NR¹⁰—, —O—, —(S)ₚ—, —NR¹²—C(═O)—, —NR¹²—C(═S)—, —O—C(═S)—, —C₁-C₆-cycloalkyl-, -alkenyl-, -heterocycloalkyl-, unsubstituted or substituted -aryl-, unsubstituted or substituted -heteroaryl-, -aralkyl-, -heteroaralkyl-, -alkyloxy-, -aryloxy-, aralkoxy —NR¹³—SO₂—, —SO₂—NR¹³—, —O—C(═O)—NR¹³—, —NR¹²—C(═O)—NR¹³—, —NH—NH— or —O—NH—,
wherein d is an integer from 1 to 6,
m and n, independently, are any integer from 0 to 5,
-D- represents a bond, —S—, —O— or —NR⁹—,
wherein R⁹ represents hydrogen, C₁-C₁₀ alkyl, aryl, heteroaryl or aralkyl,
p is an integer from 1 to 3,
R¹⁰ and R¹², independently, are selected from the group comprising hydrogen, unsubstituted or substituted linear or branched C₁-C₁₀ alkyl, aryl, heteroaryl and aralkyl and
R¹³ represents hydrogen, unsubstituted or substituted linear or branched C₁-C₁₀ alkyl, aryl, cycloalkyl, heterocycloalkyl, heteroaryl, aralkyl and heteroaralkyl
wherein E-Z²- is a targeting agent radical, wherein E is a biomolecule and Z² represents a moiety selected from the group comprising a bond and a spacer,
wherein the spacer is a natural or un-natural amino acid sequence or a non-amino acid group; Z² serves the linking of the biomolecule to the rest of the compound of the invention.

The invention further refers to pharmaceutically acceptable salts of an inorganic or organic acid thereof, hydrates, complexes, esters, amides, solvates and prodrugs having general chemical Formula I.

More specifically, in a first alternative of the invention according to the first aspect thereof, the general chemical Formula I representing the compound according to this first aspect of the present invention:

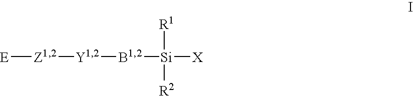

has the following meaning:

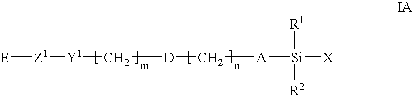

wherein:
(in general chemical Formula IA: E corresponds to E, Z¹ corresponds to Z¹², Y¹ corresponds to Y^{1,2}, [CH₂]ₘ-D-[CH₂]ₙ-A corresponds to B^{1,2}, R¹ corresponds to R¹, R² corresponds to R² and X corresponds to X)
X, R¹, R², A, n, m, D and E-Z¹-Y¹ have the same meanings as in Formula I above, and
E-Z¹ is a targeting agent radical and E is a biomolecule.

Accordingly, the invention also refers to the respective pharmaceutically acceptable salts of an inorganic or organic acid thereof, hydrates, complexes, esters, amides, solvates and prodrugs of compounds having general chemical Formula IA.

In a further preferred embodiment of the present invention of the first alternative:

E-Z¹-Y¹— represents a moiety selected from the group comprising ENR⁵—C(═O)—, EC(═O)—NR⁵—, ENR⁵—SO₂—, ESO₂—NR⁵—, ENR⁶C(═O)—NR⁷—, ENR⁶C(═S)—NR⁷—, EO-C(═O)O—, EOC(═S)—O—,

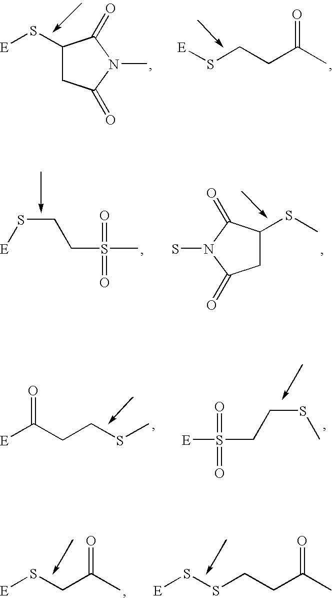

wherein the arrows shown herein above indicate the bond between Z¹ and Y¹,
wherein R⁵, R⁶ and R⁷, independently, represent hydrogen, linear or branched C₁-C₁₀ alkyl, and p can be any integer from 1 to 3.

More preferably, E-Z¹-Y¹ represents a moiety selected from the group comprising

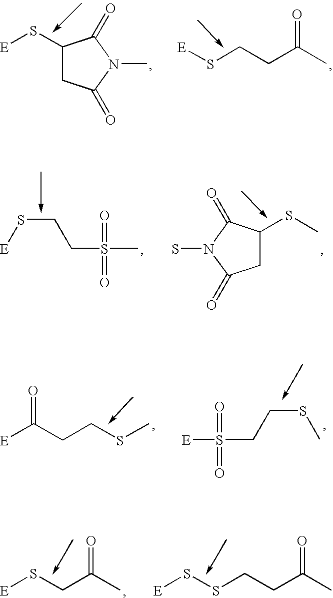

or from the group comprising
ENR⁵—C(═O)—, EC(═O)—NR⁵—, ENR⁵—SO₂—, ESO₂—NR⁵—, ENR⁶C(═O)—NR⁷—, ENR⁶C(═S)—NR⁷—, EO—C(═O)O—, EOC(═S)—O— wherein R⁵, R⁶ and R⁷, independently, represent hydrogen, linear or branched C₁-C₁₀ alkyl, and p can be any integer from 1 to 3.
—B¹— is selected from the group comprising —[CH₂]ₘ-D-[CH₂]ₙ-A-,
wherein independently
n and m, independently, are any integer from 0 to 5, more preferably 0 to 3,
-D- represents a bond, —S—, —O— or —NR⁴—, wherein R⁴ represents hydrogen,
more preferably -D- is bond or —O—
-A- is unsubstituted or substituted aryl.
E is a biomolecule. Preferably E is a biomolecule selected from peptide, peptidomimetic, oligonucleotide or small molecule. More preferably, E is a peptide.

In a further preferred embodiment of the present invention, the biomolecule is selected from the group comprising peptides, peptidomimetics, small molecules and oligonucleotides. The biomolecule E being optionally linked to a reacting moiety Z¹ which serves the linking between the biomolecule and the rest of the compound and which may be, e.g., —NR′, —NR′—(CH₂)ₙ—, —O—(CH₂)ₙ— or —S—(CH₂)ₙ—, wherein R′ is hydrogen or alkyl and n is an integer from 1 to 6.

In a more preferred embodiment of the present invention, the targeting agent radical E-Z¹- is —NR′— biomolecule or —NR′—(CH₂)ₙ— biomolecule, wherein R′ being selected from the group comprising hydrogen and alkyl, wherein n is from 1 to 6.

In an even more preferred embodiment of the present invention, the targeting agent radical E-Z¹- is —NR′— peptide or —NR′—(CH₂)ₙ— peptide, —NR′— small molecules or —NR′—(CH₂)ₙ— small molecules —NR′— oligonucleotide or —NR′—(CH₂)ₙ— oligonucleotide wherein R′ being selected from the group comprising hydrogen and alkyl, wherein n is from 1 to 6.

See table 2.

Further, in a second alternative of the invention according to the first aspect thereof, the general chemical Formula I representing the compound according to this first aspect of the present invention:

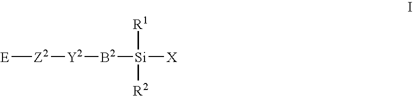

has the following meaning:

E-Z²-Y²-L-X IB

(which is E-Y—B₁-L₁-RG, wherein E corresponds to E, Y corresponds to Z², RG corresponds to X and B₁-L₁ corresponds to Y²-L, and wherein, in general chemical Formula IB, E corresponds to E, Z² corresponds to Z², Y²-L corresponds to Y²—B²—Si(R¹)(R²) and X corresponds to X)
X, Z², Y², A, n, m, and D have the same meanings as in Formula I above, and
wherein:

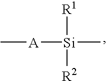

### -L- is

wherein R¹ and R², independently, are selected from the group comprising hydrogen, branched or linear C₁-C₁₀ alkyl, aryl, heteroaryl or aralkyl and
A represents a C₁-C₁₀ alkyl, unsubstituted or substituted aryl or unsubstituted or substituted heteroaryl,

In a further preferred embodiment of the present invention of the second alternative:

Y²— is a functional group or a chain containing functional group connecting -L- to -Z²- and which is selected from the group comprising a bond, —C(═O)—, —SO₂—, —C(═O)—(CH₂)_{d}—,
—SO—, —C(═O)—C≡C—, C(═O)-[CH₂]ₘ-D-[CH₂]ₙ—, SO₂—[CH₂]ₘ-D-[CH₂]ₙ—, —O—C(═O)—,
—NR¹⁰—, —O—, —(S)ₚ—, —NR¹²—C(═O)—, —NR¹²—C(═S)—, —O—C(═S)—, —C₁-C₆ cycloalkyl-, —NR¹³SO₂—, —SO₂NR¹³—, OC(═O)—NR¹³—, —NR¹²C(═O)NR¹³

### —NH—NH—, and —O—NH—,

wherein d is an integer from 1 to 6,
m and n, independently, are any integer from 0 to 5;
-D- represents a bond, —S—, —O— or —NR⁹—,
wherein R⁹ represents hydrogen, C₁-C₁₀ alkyl, aryl, heteroaryl or aralkyl,
p is any integer from 1 to 3;
R¹⁰ and R¹², independently, are selected from the group comprising hydrogen, unsubstituted or substituted or branched or linear C₁-C₁₀ alkyl, aryl, heteroaryl and aralkyl, and
R¹³ represents hydrogen, unsubstituted or substituted linear or branched C₁-C₆ alkyl, aryl, cycloalkyl, heterocycloalkyl, heteroaryl, aralkyl or heteroaralkyl.

More preferably, —Y²— is C(═O)—[CH₂]ₘ-D-[CH₂]ₙ—, —SO₂—[CH₂]ₘ-D-[CH₂]ₙ—,
wherein m and n, independently, are any integer from 0 to 5,
-D- represents a bond, —S—, —O— or —NR⁹—,
wherein R⁹ represents hydrogen, C₁-C₁₀ alkyl, aryl, heteroaryl or aralkyl
   Most preferably, —Y²— is selected from the group comprising —C(═O)—, —SO₂— and —C(═O)—C≡C—.

More preferably, Y² is selected from the group comprising —C(═O)—, and —SO₂—.

—B²— represents a C₁-C₁₀ alkyl-, unsubstituted or substituted -aryl- or unsubstituted or substituted -heteroaryl-.
More preferably, —B²— represents a C₁-C₁₀ alkyl- or unsubstituted or substituted -aryl-.

In a preferred embodiment of the present invention, -Z²- is an amino acid sequence comprising two (2) to twenty (20) amino acid residues.

In a more preferred embodiment of the present invention, -Z²- is Arg-Ser, Arg-Ava, Lys(Me)-2-β-ala, Lys(Me)2-ser, Arg-β-ala, Ser-Ser, Ser-Thr, Arg-Thr, S-alkylcysteine, Cysteic acid, thioalkylcysteine (S—S-Alkyl) or

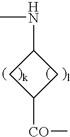

wherein k and l is 0-4.

In an even more preferred embodiment of the present invention, -Z²- is a non-amino acid moiety selected from the group comprising

—C(═O)—(CH₂)ₚ—NH—, with p being an integer from 2 to 10,
—C(═O)—(CH₂—CH₂—O)_{q}—CH₂—CH₂—NH—, with q being an integer from 0 to 5
—NH-cycloalkyl-CO— wherein cycloalkyl is selected from C₅-C₈ cycloalkyl, more preferably C₆ atom cycloalkyl, and
—NH-heterocycloalkyl-(CH₂)ᵥ—CO— wherein heterocycloalkyl is selected from C₅-C₈ heterocycloalkyl containing carbon atoms and 1, 2, 3 or 4 oxygen, nitrogen or sulfur heteroatoms more preferably 1 to 2 heteroatom even more preferably 1 heteroatom and v is an integer of from 1 to 4, more preferably v is an integer of from 1 to 2.
E is a biomolecule. Preferably E is a biomolecule selected from peptide, peptidomimetic, oligonucleotide or small molecule. More preferably, E is a peptide.

Further embodiments apply to both alternatives:

Accordingly, the invention in this specific embodiment also refers to the respective pharmaceutically acceptable salts of an inorganic or organic acid thereof, hydrates, complexes, esters, amides, solvates and prodrugs of compounds having general chemical Formula IA or IB.

In a preferred embodiment of the present invention, the leaving group X is selected from the group consisting of hydrogen or OR³

wherein R³ hydrogen, (C₁-C₁₀)alkyl, C₁-C₁₀ alkenyl or C₁-C₁₀ alkynyl.

More preferably R³ is hydrogen, C₁-C₆ alkyl, C₁-C₆ alkenyl or C₁-C₆ alkynyl.

More preferably R³ is hydrogen, C₇-C₁₀ alkyl, C₇-C₁₀ alkenyl or C₇-C₁₀ alkynyl.

Even more preferably R³ is hydrogen or C₁-C₆ alkyl.

Most preferably when R³ is C₁-C₆ alkyl then C₁-C₆ alkyl is preferably methyl or ethyl.

Even most preferably R³ is hydrogen.

Further, advantageously, R¹ and R², independently, are branched C₂-C₅ alkyl groups.

Most preferably, R¹ and R² are iso-propyl, tert-butyl or iso-butyl.

In a preferred embodiment of the present invention, A represents unsubstituted or substituted aryl.

In a further preferred embodiment of the present invention, m and n, independently, can be any integer from 0 to 3.

In a further preferred embodiment of the present invention, D represents a bond or —O—.

In a further preferred embodiment of the present invention, —Y^{1,2}— is selected from the group comprising —C(═O)—, and —SO₂—.

E is a biomolecule. The biomolecule E is preferably selected from the group comprising peptides, peptidomimetics, small molecules and oligonucleotides.

As used hereinafter in the description of the invention and in the claims, the terms “targeting agent” and “biomolecules” are directed to compounds or moieties that target or direct the radionuclide attached to them to a specific site in a biological system. A targeting agent or biomolecule can be any compound or chemical entity that binds to or accumulates at a target site in a mammalian body, i.e., the compound localizes to a greater extent at the target site than to surrounding tissue.

The compounds of this invention are useful for the imaging of a variety of cancers including but not limited to: carcinoma such as bladder, breast, colon, kidney, liver, lung, including small cell lung cancer, esophagus, gall-bladder, ovary, pancreas, stomach, cervix, thyroid, prostate and skin, hematopoetic tumors of lymphoid and myeloid lineage, tumors of mesenchymal origin, tumors of central peripheral nervous systems, other tumors, including melanoma, seminoma, teratocarcinoma, osteosarcoma, xeroderma pigmentosum, keratoxanthoma, thyroid follicular cancer and Karposi's sarcoma.

Most preferably, the use is not only for imaging of tumors, but also for imaging of inflammatory and/or neurodegenerative diseases, such as multiple sclerosis or Alzheimer's disease, or imaging of angiogenesis-associated diseases, such as growth of solid tumors, and rheumatoid arthritis.

Preferably the targeting agent is a peptide or a peptidomimetic or an oligonucleotide, particularly one which has specificity to target the complex to a specific site in a biological system. Smaller organic molecules effective for targeting certain sites in a biological system can also be used as the targeting agent.

Small molecules effective for targeting certain sites in a biological system can be used as the biomolecule E. Small molecules may be “small chemical entities”. As used in this application, the term “small chemical entity” shall have the following meaning: a small chemical entity is a compound that has a molecular mass of from 200 to 800 or of from 150 to 700, more preferably of from 200 to 700, more preferably of from 250 to 700, even more preferably of from 300 to 700, even more preferably of from 350 to 700 and most preferably of from 400 to 700. A small chemical entity as used herein may further contain at least one aromatic or heteroaromatic ring and/or may also have a primary and/or secondary amine, a thiol or hydroxyl group coupled via which the moiety containing the silyl residue in the compounds of general chemical Formulae I and II is coupled. Such targeting moieties are known in the art, so are methods for preparing them.

The small molecule may preferably be selected from those described in the following references: P. L. Jager, M. A. Korte, M. N. Lub-de Hooge, A. van Waarde, K. P. Koopmans, P. J. Perik and E. G. E. de Vries, Cancer Imaging, (2005) 5, 27-32; W. D. Heiss and K. Herholz, J. Nucl. Med., (2006) 47(2), 302-312; and T. Higuchi and M. Schwaiger, Curr. Cardiol. Rep., (2006) 8(2), 131-138. More specifically examples of small molecule are listed hereinafter:

| Name | Abbr. | target |
|---|---|---|
| 18F-2b-Carbomethoxy-3b-(4- | CFT | DAT (dopamine transporter) |
| fluorophenyl)tropane | | |
| 18F-Fluoroethylspiperone | FESP | D2 (dopamine 2 receptor), 5- |
| | | HT₂ |
| | | (5-hydroxytryptamine receptor) |
| 18F-Fallypride | | D2 (dopamine 2 receptor) |
| 18F-Altanserin | | 5-HT2A receptor |
| 18F-Cyclofoxy | | Opioid receptors |
| 18F-CPFPX | | Adenosine A1 receptor |
| Batimastat | | MMP |
| Fatty acids and analogues | | |
| Choline analogues | | |
| (metabolism) | | |
| Flumazenil | | Benzodiazepine receptors |
| Raclopride | | D2 receptors |
| Dihydrotestosteron and | | AR |
| analogues | | |
| Tamoxifen and analogues | | |
| Deoxyglucose | | |
| Thymidine | | Proliferation marker-thymidine |
| | | kinase |
| DOPA | | |
| Benzazepines | | D₁ antagonists |
| N-methyl spiperone and | | dopamine receptors |
| derivatives thereof | | |
| Benzamide raclopride; | | D₂ receptors |
| benzamide derivatives, e.g., | | |
| fallopride, iodo benzamide; | | |
| clozapine, quietapine | | |
| Nomifensine, substituted | | DAT |
| analogs of cocaine, e.g., | | |
| tropane type derivatives of | | |
| cocaine, methyl phenidate | | |
| 2β-Carboxymethoxy-3β-(4- | CIT | DAT |
| iodophenyl)tropane | | |
| | CIT-FE, CIT-FM | DAT |
| Altanserin, setoperon, | | 5-HT_{2A} |
| ketanserin | | |
| | McN5652, 403U76 derivative | 5-HTT |
| | ADAM, DASP, MADAM | |
| Acetylcholine analogues | MP3A, MP4A, PMP; QNB, | acetylcholine receptors |
| | TKB, NMPB, | |
| Scopolamine, benztropine | | acetylcholine receptors |
| Flumazenil | | GABA receptor |
| | RO-15-4513, FDG | GABA receptor |
| | PK-11195 | benzodiazepine receptor |
| Xanthine analogues | CPFPX, MPDX | adenosine receptor |
| Carfentanyl, diprenorphine | | opoid receptor |

Further various small molecules are given in Table 1 in W. D. Heiss and K. Herholz, ibid. and in FIG. 1 in T. Higuchi, M. Schwaiger, ibid.

Further preferred biomolecules are sugars, oligosaccharides, polysaccharides, aminoacids, nucleic acids, nucleotides, nucleosides, oligonucleotides, proteins, peptides, peptidomimetics, antibodies, aptamers, lipids, hormones (steroid and nonsteroid), neurotransmitters, drugs (synthetic or natural), receptor agonists and antagonists, dendrimers, fullerenes, virus particles and other targeting molecules/biomolecules (e.g., cancer targeting molecules).

Further, the biomolecule E may be a peptide. E may be a peptide comprising from 4 to 100 amino acids.

In a preferred embodiment of the present invention, the peptide is never a Tyr3-octreotate derivative used for diagnosis of neuroendocrine tumors.

In a preferred embodiment of the present invention, the biomolecule may be a peptide which is selected from the group comprising somatostatin and derivatives thereof and related peptides, somatostatin receptor specific peptides, neuropeptide Y and derivatives thereof and related peptides, neuropeptide Y₁ and the analogs thereof, bombesin and derivatives thereof and related peptides, gastrin, gastrin releasing peptide and the derivatives thereof and related peptides, epidermal growth factor (EGF of various origin), insulin growth factor (IGF) and IGF-1, integrins (α₃β₁, αᵥβ₃, αᵥβ5, allb₃), LHRH agonists and antagonists, transforming growth factors, particularly TGF-α; angiotensin; cholecystokinin receptor peptides, cholecystokinin (CCK) and the analogs thereof; neurotensin and the analogs thereof, thyrotropin releasing hormone, pituitary adenylate cyclase activating peptide (PACAP) and the related peptides thereof, chemokines, substrates and inhibitors for cell surface matrix metalloproteinase, prolactin and the analogs thereof, tumor necrosis factor, interleukins (IL-1, IL-2, IL-4 or IL-6), interferons, vasoactive intestinal peptide (VIP) and the related peptides thereof.

In a more preferred embodiment of the present invention, the biomolecule may be selected from the group comprising bombesin and bombesin analogs, preferably those having the sequences listed herein below, somatostatin and somatostatin analogs, preferably those having the sequences listed herein below, neuropeptide Y₁ and the analogs thereof, preferably those having the sequences listed herein below, vasoactive intestinal peptide (VIP) and the analogs thereof.

In a more preferred embodiment of the present invention, the biomolecule may be selected from the group comprising bombesin, somatostatin, neuropeptide Y₁ Vasoactive intestinal peptide (VIP) and the analogs thereof.

In an even more preferred embodiment of the present invention, the biomolcule E is bombesin, somatostatin or neuropeptide Y₁ or the analog thereof.

In an even more preferred embodiment of the present invention, the biomolecule is bombesin or the analog thereof.

Bombesin is a fourteen amino acid peptide that is an analog of human gastrin releasing peptide (GRP) that binds with high specificity to human GRP receptors present in prostate tumor, breast tumor and metastasis. In an even more preferred embodiment of the present invention, the biomolecule E comprises bombesin analogs having sequence III or IV:
AA₁-AA₂-AA₃-AA₄-AA₅-AA₆-AA₇-AA₈-NT₁T₂ (type A) III, with
   T₁=T₂=H, T₁=H, T₂=OH, T₁=CH₃, T₂=OH
   AA₁=Gln, Asn, Phe(4-CO—NH₂)
   AA₂=Trp, D-Trp
   AA₃=Ala, Ser, Val
   AA₄=Val, Ser. Thr
   AA₅=Gly, (N-Me)Gly
   AA₆=His, His(3-Me), (N-Me)His, (N-Me)His(3-Me)
   AA₇=Sta, Statine analogs and isomers, 4-Am,5-MeHpA, 4-Am,5-MeHxA and γ-substituted aminoacids
   AA₈=Leu, Cpa, Cba, CpnA, Cha, t-buGly, tBuAla, Met, Nle, iso-Bu-Gly
AA₁-AA₂-AA₃-AA₄-AA₅-AA₆-AA₇-AA₈-NT₁T₂ (type B) IV, with:
   T₁=T₂=H, T₁=H, T₂=OH, T₁=CH₃, T₂=OH
   AA₁=Gln, Asn, Phe(4-CO—NH₂)
   AA₂=Trp, D-Trp
   AA₃=Ala, Ser, Val
   AA₄=Val, Ser. Thr
   AA₅=βAla, β² and β³-amino acids as shown herein after

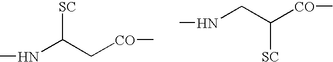

wherein SC represents side chain found in proteinogenic amino acids and homologs of proteinogenic amino acids,
AA₆=His, His(3-Me), (N-Me)His, (N-Me)His(3-Me)
AA₇=Phe, Tha, NaI,
AA₈=Leu, Cpa, Cba, CpnA, Cha, t-buGly, tBuAla, Met, Nle, iso-Bu-Gly.

Therefore, in an even more preferred embodiment of the present invention the biomolecule is selected from the group comprising bombesin analogs having sequence III or IV.

In an even more preferred embodiment, bombesin analogs have the following sequences:

| | |
|---|---|
| Seq ID | E |

| | |
|---|---|
| Seq ID 1 | Gln-Trp-Ala-Val-NMeGly-His-Sta-Leu-NH₂ |
| Seq ID 2 | Gln-Trp-Ala-Val-Gly-His(Me)-Sta-Leu-NH₂ |
| Seq ID 3 | Gln-Trp-Ala-Val-NMeGly-His(3Me)-Sta- |
| | Leu-NH₂ |
| Seq ID 4 | Gln-Trp-Ala-Val-Gly-His(3Me)-Sta-Leu- |
| | NH₂ |
| Seq ID 7 | Gln-Trp-Ala-Val-NMeGly-His(3Me)-Sta- |
| | Cpa-NH₂ |
| Seq ID 8 | Gln-Trp-Ala-Val-Gly-His(3Me)-4-Am,5- |
| | MeHpA-Leu-NH₂ |
| Seq ID 12 | Gln-Trp-Ala-Val-Gly-His(3Me)-4-Am,5- |
| | MeHpA-Leu-NH₂ |
| Seq ID 17 | Gln-Trp-Ala-Val-Gly-His-4-Am,5-MeHpA-- |
| | Leu-NH₂ |
| Seq ID 23 | Gln-Trp-Ala-Val-NMeGly-His(3Me)-4-Am,5- |
| | MeHpA-Cpa-NH₂ |
| Seq ID 27 | Gln-Trp-Ala-Val-NMeGly-His-FA02010-Cpa- |
| | NH₂ |
| Seq ID 28 | Gln-Trp-Ala-Val-NMeGly-His-4-Am,5- |
| | MeHpA-tbuGly-NH₂ |
| Seq ID 30 | Gln-Trp-Ala-Val-NMeGly-His(3Me)-Sta- |
| | tBuGly-NH₂ |
| Seq ID 32 | Gln-Trp-Ala-Val-NMeGly-His(3Me)-4-Am,5- |
| | MeHpA-Leu-NH₂ |
| Seq ID 33 | Gln-DTrp-Ala-Val-Gly-His-4-Am,5-MeHpA- |
| | tbuGly-NH₂ |
| Seq ID 34 | Gln-DTrp-Ala-Val-Gly-His-4-Am-5-MeHxA- |
| | Cpa-NH₂ |
| Seq ID 35 | Gln-Trp-Ala-Val-NMeGly-His(3Me)-Sta- |
| | Cpa-NH₂ |
| Seq ID 36 | Gln-DTrp-Ala-Val-Gly-His-Sta-tbuAla-NH₂ |
| Seq ID 42 | Gln-Trp-Ala-Val-Gly-His(3Me)-Sta-Cpa- |
| | NH₂ |
| Seq ID 43 | Gln-Trp-Ala-Val-Gly-His(3Me)-Sta- |
| | tBuGly-NH₂ |
| Seq ID 46 | Gln-Trp-Ala-Val-Gly-His(3Me)-4-Am,5- |
| | MeHpA-Leu-NH₂ |
| Seq ID 48 | Gln-Trp-Ala-Val-Gly-His(3Me)-4-Am,5- |
| | MeHpA-Leu-NH₂ |
| Seq ID 49 | Gln-Trp-Ala-Val-Gly-NMeHis-4-Am,5- |
| | MeHpA-Cpa-NH₂ |
| Seq ID 49 | Gln-Trp-Ala-Val-Gly-NMeHis(3Me)-4-Am,5- |
| | MeHpA-Leu-NH₂ |
| Seq ID 50 | Gln-Trp-Ala-Val-Gly-NMeHis-4-Am,5- |
| | MeHpA-Leu-NH₂ |
| Seq ID 51 | Gln-Trp-Ala-Val-NMeGly-Hls-AHMHxA -Leu- |
| | NH₂ |
| Seq ID 52 | Gln-Trp-Ala-Val-βAla-NMeHis-Tha-Cpa-NH₂ |
| Seq ID 53 | Gln-Trp-Ala-Val-βAla-NMeHis-Phe-Cpa-NH₂ |
| Seq ID 54 | Gln-Trp-Ala-Val-βAla-NMeHis-Phe-Leu-NH₂ |
| Seq ID 55 | Gln-Trp-Ala-Val-βAla-DHis-Phe-Leu-NH₂ |
| Seq ID 56 | Gln-Trp-Ala-Val-βAla-His-βhLeu-Leu-NH₂ |
| Seq ID 57 | Gln-Trp-Ala-Val-βAla-His-βhIle-Leu-NH₂ |
| Seq ID 58 | Gln-Trp-Ala-Val-βAla-His-βhLeu-tbuGly- |
| | NH₂ |
| Seq ID 59 | Gln-Trp-Ala-Val-βAla-His(3Me)-Phe-Tha- |
| | NH₂ |
| Seq ID 60 | Gln-Trp-Ala-Val-βAla-His(3Me)-Phe-Nle- |
| | NH₂ |
| Seq ID 61 | Gln-Trp-Ala-Val-βAla-NMeHis-Phe-tbuGly- |
| | NH₂ |
| Seq ID 62 | Gln-Trp-Ala-Val-βAla-NMeHis-Tha-tbuGly- |
| | NH₂ |
| Seq ID 63 | Gln-Trp-Ala-Val-βAla-His(3Me)-Tha- |
| | tbuGly-NH₂ |
| Seq ID 64 | Gln-Trp-Ala-Val-βAla-His(3Me)-Phe-Cpa- |
| | NH₂ |
| Seq ID 65 | Gln-Trp-Ala-NMeVal-βAla-His-Phe-Leu- |
| | NH₂ |
| Seq ID 66 | Gln-Trp-Ala-Val-βAla-His-NMePhe-Leu- |
| | NH₂ |
| Seq ID 67 | Gln-DTrp-Ala-Val-βAla-His-Phe-Leu-NH₂ |
| Seq ID 68 | Gln-Trp-DAla-Val-βAla-His-Phe-Leu-NH₂ |
| Seq ID 69 | Gln-Trp-Ala-DVal-βAla-His-Phe-Leu-NH₂ |
| Seq ID 70 | Gln-Trp-Ala-Val-βAla-His-DPhe-Leu-NH₂ |
| Seq ID 71 | Gln-Trp-Ala-Val-βAla-His-βhIle-tbuGly- |
| | NH₂ |
| Seq ID 72 | Gln-Trp-Ala-Val-NMeGly-His-4-Am,5- |
| | MeHpA-Cpa-NH₂ |
| Seq ID 73 | Gln-Trp-Ala-Val-NMeGly-His-Sta-Cpa-NH₂ |
| Seq ID 74 | Gln-Trp-Ala-Val-NMeGly-His-Sta-tbuAla- |
| | NH₂ |
| Seq ID 75 | Gln-Trp-Ala-Val-NMeGly-His-4-Am,5- |
| | MeHpA-tbuAla-NH₂ |
| Seq ID 82 | Gln-Trp-Ala-Val-Gly-His(3Me)-FA4-Am,5- |
| | MeHpA-Leu-NH₂ |
| Seq ID 90 | Gln-Trp-Ala-Val-Gly-His(3Me)-4-Am,5- |
| | MeHpA-Leu-NH₂ |
| Seq ID 91 | Gln-Trp-Ala-Val-Gly-His-4-Am,5-MeHpA- |
| | Leu-NH₂ |
| Seq ID 101 | Gln-Trp-Ala-Val-Gly-His(3Me)-4-Am-5- |
| | MeHpA-4-amino-5-methylheptanoic acid - |
| | Leu-NH₂ |
| Seq ID 102 | Gln-Trp-Ala-Val-NMeGly-His(3Me)-4-Am-5- |
| | MeHpA-4-amino-5-methyiheptanoic acid - |
| | Cpa-NH₂ |

More preferably the bombesin analog may additionally be labelled more preferably radiolabeled with a fluorine isotope (F) wherein F is ¹⁸F or ¹⁹F. More preferably the bombesin analog is radiolabeled using the radiofluorination method of the present invention.

The above bombesin analogs that bind specifically to human GRP receptors present in prostate tumor, breast tumor and metastasis, may be part of the compound having general chemical Formula I, in that they form the biomolecule, wherein the biomolecule may optionally be linked to a reacting moiety Z which serves the linking between the biomolecule and the rest of the compound of the invention (Formulae I, II), e.g., —NR′, —NR′-(CH₂)ₙ—, —O—(CH₂)ₙ— or —S—(CH₂)ₙ—, wherein R′ is hydrogen or alkyl and n is an integer from 1 to 6. The bombesin analogs may be peptides having sequences from Seq ID 1 to Seq ID 102 and preferably may have one of them.

In a more preferred embodiment, somatostatin analogs have the following sequences:

Seq ID 104----c[Lys-(NMe)Phe-1Nal-D-Trp-Lys-Thr]
Seq ID 105----c[Dpr-Met-(NMe)Phe-Tyr-D-Trp-Lys]

In a more preferred embodiment, neuropeptide Y₁ analogs have the following sequences:

| | |
|---|---|
| Seq ID 106 | -DCys-Leu-Ile-Thr-Arg-Cys-Arg-Tyr-NH₂ |
| Seq ID 107 | -DCys-Leu-Ile-Val-Arg-Cys-Arg-Tyr-NH₂ |

(_indicates disulfide bridge)

In other preferred embodiments E is selected to be an oligonucleotide. In a further preferred embodiment E may be selected from the group comprising oligonucleotides comprising from 4 to 100 nucleotides.

Preferred oligonucleotide is TTA1 (see experimental part).

In a further preferred embodiment of the present invention, the biomolecule E may comprise a combination of any of the aforementioned bioactive molecules suitable to bind to a target site together with a reacting moiety which serves the linking between the bioactive molecule and the rest of the compound of the invention (Formulae I, II), e.g., —NR′, —NR′—(CH₂)ₙ—, —O—(CH₂)ₙ— or —S—(CH₂)ₙ—, wherein R′ is hydrogen or alkyl and n is an integer from 1 to 6.

In a more preferred embodiment, the compound of formula I is selected from the following list wherein E is a bombesin analog:

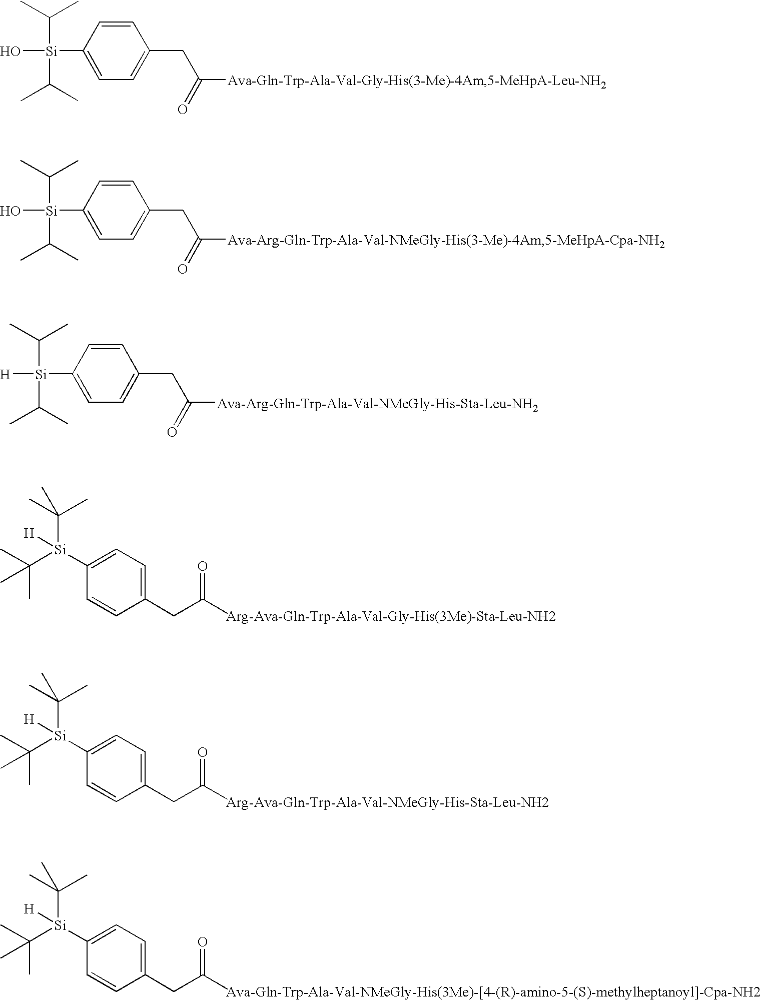

In a second aspect, the present invention refers to novel compounds having general chemical Formula II:

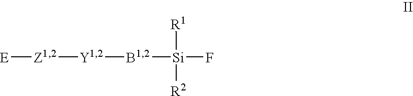

wherein B^{1,2}, Y^{1,2}, Z^{1,2}, E, R¹ and R² have the same meanings as in Formula I above, including all meanings which are assigned to these parameters according to preferred and alternative embodiments of the present invention, and
wherein F is fluorine isotope wherein F is selected from radioactive or non-radioactive isotope.

The radioactive fluorine isotope is preferably selected from ¹⁸F. The non-radioactive “cold” fluorine isotope is preferably selected from ¹⁹F.

Accordingly, the invention referring to this second aspect also refers to the respective pharmaceutically acceptable salts of an inorganic or organic acid thereof, hydrates, complexes, esters, amides, solvates and prodrugs having general chemical Formula II.

In a first alternative of the invention according to the second aspect thereof, the general chemical formula of the fluorinated compound of the invention:

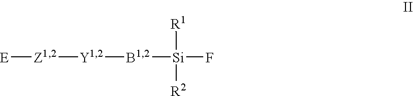

has the following meaning:

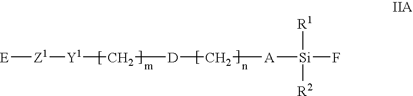

wherein:
   F is fluorine isotope wherein F is selected from radioactive or non-radioactive isotope,
   R¹, R², A, n, m, D and E-Z¹-Y¹ have the same meanings as in Formula I above, including all meanings which are assigned to these parameters according to preferred and alternative embodiments of the present invention, and
   wherein E-Z¹- is a targeting agent radical, wherein E is a biomolecule.

Accordingly, the invention in this specific embodiment also refers to the respective pharmaceutically acceptable salts of an inorganic or organic acid thereof, hydrates, complexes, esters, amides, solvates and prodrugs having general chemical Formula IIA.

In a second alternative of the invention according to the second aspect thereof, the general chemical formula of the pharmaceutical labelled compound of the invention:

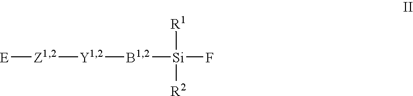

has the following meaning:

E-Z²-Y²-L-F IIB
(which is E-Y—B₂-L₂-F or, in the most preferred embodiment, E-Y—B₂-L₂-[18]F or E-Y—B₂-L₂-[1,9]F, wherein E corresponds to E, Y corresponds to Z², F is fluorine isotope, more preferably ¹⁸F or ¹⁹F, and B₂-L₂ corresponds to Y²-L)
wherein:
   F is fluorine isotope wherein F is selected from radioactive or non-radioactive isotope more preferably a radioactive fluorine is ¹⁸F and non-radioactive (“cold”) fluorine ¹⁹F.

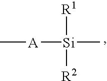

-L- is
wherein R¹ and R², independently, are selected from the group comprising hydrogen, linear or branched C₁-C₁₀ alkyl, aryl, heteroaryl and aralkyl and
A represents alkyl, unsubstituted or substituted aryl, or unsubstituted or substituted heteroaryl,
—Y²— is a functional group or a chain containing functional group connecting -L- to -Z²- and which is selected from the group comprising a bond, —C(═O)—, —SO₂—, —C(═O)—(CH₂)_{d}—, —SO—, —C(═O)—C≡C—, —C(═O)-[CH₂]ₘ-D-[CH₂]ₙ—, SO₂-[CH₂]ₘ-D-[CH₂]n, —O—C(═O)—, —NR¹⁰—, —O—, —(S)ₚ—, —NR¹²C(═O)—, —NR¹²—C(═S)—, —O—C(═S)—,
—C₁-C₆ cycloalkyl-, -alkenyl-, -heterocycloalkyl-, unsubstituted or substituted aryl-, unsubstituted or substituted -heteroaryl, -aralkyl-, -heteroaralkyl, -alkyloxy-, aryloxy-, -aralkyloxy-, -aryl-, —NR¹³SO₂—, —SO₂NR¹³—, OC(═O)—NR¹³—, —NR¹²C(═O)NR¹³—, —NH—NH—, and —O—NH—,
wherein d is an integer from 1 to 6,
m and n, independently, are any integer from 0 to 5,
-D- represents a bond, —S—, —O— or —NR⁹—,
Wherein R⁹ represents hydrogen, C₁-C₁₀ alkyl, aryl, heteroaryl or aralkyl,
p is any integer from 1 to 3,
R¹⁰ and R¹², independently, are selected from the group comprising hydrogen, unsubstituted or substituted or linear or branched C₁-C₁₀ alkyl, aryl, heteroaryl and aralkyl, and
R¹³ represents hydrogen, substituted or unsubstituted, linear or branched C₁-C₁₀ alkyl, aryl, cycloalkyl, heterocycloalkyl, heteroaryl, aralkyl or heteroaralkyl.
E-Z²- is a targeting agent radical, wherein E is a targeting agent and Z² represents a moiety selected from the group comprising a bond and a spacer, wherein the spacer is a natural or un-natural amino acid sequence or a non-amino acid group and
E is a biomolecule. Preferably E is a biomolecule selected from peptide, peptidomimetic, oligonucleotide or small molecule. More preferably, E is a peptide.

Accordingly, the invention in this specific embodiment also refers to the respective pharmaceutically acceptable salts of an inorganic or organic acid thereof, hydrates, complexes, esters, amides, solvates and prodrugs having general chemical Formula IIB.

F is fluorine isotope and preferably ¹⁸F or ¹⁹F. Thus, the compound having general chemical Formula II may have the following general chemical Formula II-18F:

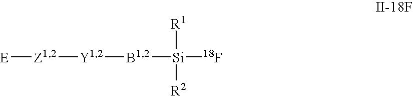

See table 4
or general chemical Formula II-19F:

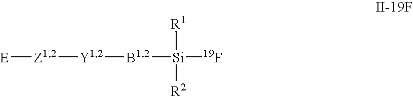

See table 3

More specifically, E in the compound having general chemical Formulae II, IIA, IIB, II-18F and II-1 gF is identical to E in the compound having general chemical Formulae I, IA and IB, respectively, and preferred embodiment. More preferably, E is bombesin or an bombesin analog and any having the sequences listed above. More preferably, E is somatostatin or a somatostatin analog and any having the sequences listed above. More preferably, E is neuropeptide Y₁ or a neuropeptide Y₁ analog and any having the sequences listed above.

In a preferred embodiment, the pharmaceutical labelled with fluorine is selected from the following list wherein E is a bombesin analog:

### IIA-c-2: ¹⁸F—Si(tBu)₂-C₆H₄—CH₂—CO-Ava-Gln-Trp-Ala-Val-NMeGly-His(3Me)-4-Am,5-MeHpA-Cpa-NH₂,

### IB-c-1: ¹⁹F—Si(iPr)₂-C₆H₄—CH₂—CO-Ava-Gln-Trp-Ala-Val-Gly-His(3Me)-4-Am,5-MeHpA-Leu-NH₂,

### IIB-c-2: ¹⁹F—Si(tBu)₂-C₆H₄—CH₂—CO-Ava-Gln-Trp-Ala-Val-NMeGly-His(3Me)-4-Am,5-MeHpA-Cpa-NH₂.

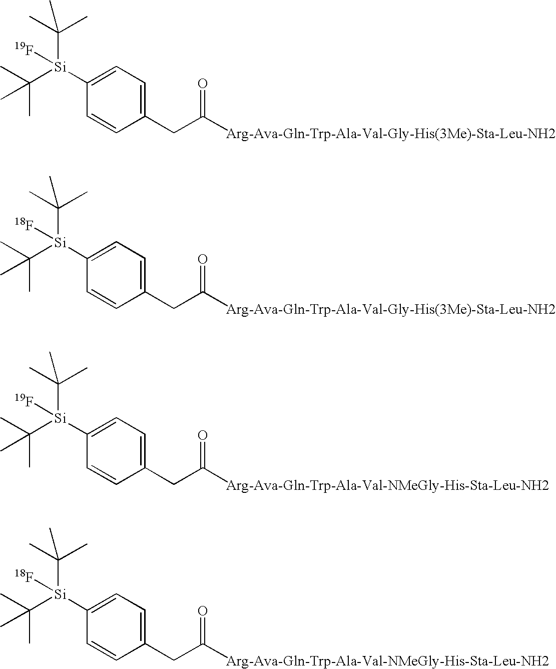

Further, compounds having general chemical Formula II, which are obtainable by a method as given herein below, are for example:
[4-(Fluoro-di-iso-propyl-silanyl)-phenyl]-acetic acid
2-[4-(Fluoro-di-iso-propyl-silanyl)-phenyl]-ethanol
4-(Fluoro-di-iso-propyl-silanyl)-benzoic acid
[4-(Fluoro-di-iso-propyl-silanyl)-phenyl]-methanol
3-[4-(Fluoro-di-iso-propyl-silanyl)-phenyl]-propan-1-ol
3-[4-(Fluoro-di-iso-propyl-silanyl)-phenyl]-propionic acid
3-[3-(Fluoro-di-iso-propyl-silanyl)-phenyl]-propan-1-ol
3-[3-(Fluoro-di-iso-propyl-silanyl)-phenyl]-propionic acid
2-[3-(Fluoro-di-iso-propyl-silanyl)-phenyl]-ethanol
[3-(Fluoro-di-iso-propyl-silanyl)-phenyl]-acetic acid
[4-(Fluoro-di-iso-butyl-silanyl)-phenyl]-acetic acid and
4-(Fluoro-di-iso-butyl-silanyl)-benzoic acid
   wherein fluoro means ¹⁸F or ¹⁹F.

In a third aspect, the present invention refers to novel compounds represented by formula III:

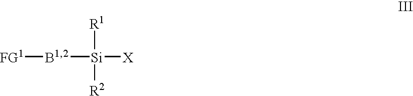

wherein
FG¹- represents —OH, -Hal, —N₃, —CO₂R⁸, —NHR⁵, —N═C═O, —O—C≡N, —S—C≡N, —N═C═S, —O—SO₂-Aryl, —O—SO₂-Alkyl, —SO₂-Hal, —S₃H, —SH, —O—C(═O)—Hal, —O—C(═S)-Hal,

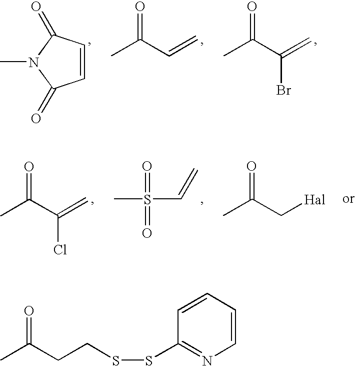

wherein Hal represents a halogen atom, and
R⁵ represents hydrogen, linear or branched C₁-C₁₀ alkyl, aryl, heteroaryl or aralkyl; and
R⁸ represents hydrogen, C₁-C₁₀ alkyl, C₂-C₁₀ alkenyl, aralkyl or

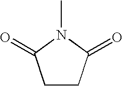

and wherein X, R¹, R² and B^{1,2} have the same meanings as in Formula I, including all meanings which are assigned to these parameters according to preferred and alternative embodiments of the present invention.

In a preferred embodiment of the present invention, Hal is halogen selected from Cl, Br or I.

In a preferred embodiment of the present invention, the general chemical Formula III of the compound:

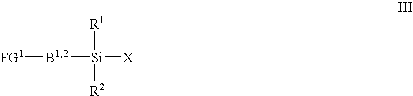

has the following meaning:

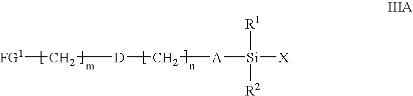

wherein X, R¹, R², A, D, m and n have the same meanings as in Formula IA, including all meanings which are assigned to these parameters according to preferred and alternative embodiments of the present invention, and FG¹ has the same meaning as in Formula III above.

Preferred compounds of formula IIIA are:
(Di- tert-butyl-hydroxy-silanyl)-acetic acid
[4-(Hydroxy-di-iso-propyl-silanyl)-phenyl]-acetic acid
[4-(Hydroxy-di-iso-propyl-silanyl)-phenyl]-acetic acid
(4-Di-iso-propylsilanyl-phenyl)-acetic acid
(4-Di-iso-propylsilanyl-phenyl)-acetic acid 2,5-dioxo-pyrrolidin-1-yl ester
[4-(Hydroxy-di-iso-propyl-silanyl)-phenyl]-acetic acid 2,5-dioxo-pyrrolidin-1-yl ester
4-Di-iso-propylsilanyl-benzoic acid
4-(Hydroxy-di-iso-propyl-silanyl)-benzoic acid
4-Di-iso-propylsilanyl-benzoic acid 2,5-dioxo-pyrrolidin-1-yl ester
4-(Hydroxy-di-iso-propyl-silanyl)-benzoic acid 2,5-dioxo-pyrrolidin-1-yl ester
3-(4-Di-iso-propylsilanyl-phenyl)-propionic acid
3-(4-Di-iso-propylsilanyl-phenyl)-propionic acid 2,5-dioxo-pyrrolidin-1-yl ester
3-(3-Di-iso-propylsilanyl-phenyl)-propionic acid
3-(3-Di-iso-propylsilanyl-phenyl)-propionic acid 2,5-dioxo-pyrrolidin-1-yl ester
(3-Di-iso-propylsilanyl-phenyl)-acetic acid
(3-Di-iso-propylsilanyl-phenyl)-acetic acid 2,5-dioxo-pyrrolidin-1-yl ester
(4-Di-iso-butylsilanyl-phenyl)-acetic acid
(4-Di-iso-butylsilanyl-phenyl)-acetic acid 2,5-dioxo-pyrrolidin-1-yl ester
[4-(Hydroxy-di-iso-butyl-silanyl)-phenyl]-acetic acid 2,5-dioxo-pyrrolidin-1-yl ester
[4-(Hydroxy-di-iso-butyl-silanyl)-phenyl]-acetic acid
4-Di-iso-butylsilanyl-benzoic acid
4-(Hydroxy-di-iso-butyl-silanyl)-benzoic acid
4-Di-iso-butylsilanyl-benzoic acid 2,5-dioxo-pyrrolidin-1-yl ester
4-(Hydroxy-di-iso-butyl-silanyl)-benzoic acid 2,5-dioxo-pyrrolidin-1-yl ester
4-[3-(Ethoxy-di-iso-propyl-silanyl)-propylcarbamoyl]-butyric acid
4-[3-(Ethoxy-di-iso-propyl-silanyl)-propylcarbamoyl]-butyric acid 2,5-dioxo-pyrrolidin-1-yl ester
5-[Di-iso-butyl-(4-phenyl-butoxy)-silanyl]-pentanoic acid 2,5-dioxo-pyrrolidin-1-yl ester
5-[(4-Polystyrene-methoxy-benzyloxy)-di-iso-butyl-silanyl]-pentanoic acid 2,5-dioxo-pyrrolidin-1-yl ester
5-(Polystyrene-methoxy-di-iso-butyl-silanyl)-pentanoic acid 2,5-dioxo-pyrrolidin-1-yl ester and
5-(Polystyrene-ethoxy-di-iso-butyl-silanyl)-pentanoic acid 2,5-dioxo-pyrrolidin-1-yl ester.

In a fourth aspect, the present invention also relates to a method for producing a compound having general chemical Formula I, as defined herein above, wherein a compound having general chemical Formula III, as also defined herein above, is reacted with a compound having general chemical Formula IV:

E-FG² IV

wherein
FG² has the same meanings as listed for FG¹, and E is a biomolecule and has the same meaning as defined herein above, and wherein FG¹ and FG² are selected to establish Z^{1,2}Y^{1,2} as defined herein above, wherein Z^{1,2} and Y^{1,2} are as defined herein above.

In a sixth aspect, the present invention furthermore relates to a composition, which comprises a compound having general chemical Formula I or a pharmaceutically acceptable salt of an inorganic or organic acid thereof, a hydrate, complex, ester, amide, solvate and prodrug thereof and further preferably comprises a physiologically acceptable carrier, diluent, adjuvant or excipient.

In a seventh aspect, the present invention furthermore relates to a method of imaging diseases, said method comprising introducing into a patient a detectable quantity of a labelled compound having general chemical Formula II as defined herein above or of a pharmaceutically acceptable salt of an inorganic or organic acid thereof, a hydrate, complex, ester, amide, solvate and prodrug thereof.

In an eighth aspect, the present invention refers to a kit comprising a sealed vial containing a predetermined quantity of a compound or composition, as defined herein above, in powder form, and a container containing an appropriate solvent for preparing a solution of the compound or composition for administration to an mammalian, including a human.

In a ninth aspect, the present invention furthermore relates to a compound having general chemical Formula II as defined herein above or a pharmaceutically acceptable salt of an inorganic or organic acid thereof, a hydrate, complex, ester, amide, solvate and prodrug thereof for use as medicament

The present invention furthermore relates to a compound having general chemical Formula II as defined herein above or a pharmaceutically acceptable salt of an inorganic or organic acid thereof, a hydrate, complex, ester, amide, solvate and prodrug thereof for use as diagnostic imaging agent and preferably for use as imaging agent for positron emission tomography (PET).

In another variation of this aspect, the present invention also relates to fluorinated compounds, more preferably labelled with ¹⁹F isotope, having general chemical Formula II for use in biological assays and chromatographic identification.

More preferably, the invention relates to the use of compounds having general chemical Formula I for the manufacture of compounds having general chemical Formula II, in which F=¹⁹F as a measurement agent. More preferably, the invention relates to the use of a compound having general chemical Formula I for the manufacture of a compound having general chemical Formula II as a measurement agent.

In a tenth aspect, the present invention furthermore relates to the use of a compound having general chemical Formula I as defined herein above or of a compound having general chemical Formula II as defined herein above, including a compound being prepared with the method as defined herein above, or of a pharmaceutically acceptable salt of an inorganic or organic acid thereof, a hydrate, complex, ester, amide, solvate and prodrug thereof for the manufacture of a medicament

The present invention furthermore relates to the use of a compound having general chemical Formula I as defined herein above or of a compound having general chemical Formula II as defined herein above, including a compound being prepared with the method as defined herein above, or of a pharmaceutically acceptable salt of an inorganic or organic acid thereof, a hydrate, complex, ester, amide, solvate and prodrug thereof for the manufacture of a diagnostic imaging agent and most preferably for the manufacture of a diagnostic imaging agent for imaging tissue at a target site using the imaging agent more preferably for imaging agent for positron emission tomography (PET).

In an eleventh aspect, the present invention relates to the use of a compound having general chemical Formula I or the use of a compound having general chemical Formula II, including a compound being prepared with the method as defined herein above, or of a composition as defined herein above or of a kit as defined herein above, for diagnostic imaging, in particular for positron emission tomography and most preferably for imaging of tumors, of inflammatory and/or neurodegenerative diseases, such as multiple sclerosis or Alzheimer's disease, or for imaging of angiogenesis-associated diseases, such as growth of solid tumors, and of rheumatoid arthritis.

The compounds of the invention are useful for the imaging of a variety of cancers including but not limited to: carcinoma such as bladder, breast, colon, kidney, liver, lung, including small cell lung cancer, esophagus, gall-bladder, ovary, pancreas, stomach, cervix, thyroid, prostate and skin, hematopoetic tumors of lymphoid and myeloid lineage, tumors of mesenchymal origin, tumors of central peripheral nervous systems, other tumors, including melanoma, seminoma, teratocarcinoma, osteosarcoma, xeroderma pigmentosum, keratoxanthoma, thyroid follicular cancer and Karposi's sarcoma.

Most preferably, the use is for only for imaging of tumors, but also for imaging of inflammatory and/or neurodegenerative diseases, such as multiple sclerosis or Alzheimer's disease, or imaging of angiogenesis-associated diseases, such as growth of solid tumors, and rheumatoid arthritis.

The radioactively labeled compounds according to Formula II provided by the invention may be administered intravenously in any pharmaceutically acceptable carrier, e.g., conventional medium such as an aqueous saline medium, or in blood plasma medium, as a pharmaceutical composition for intravenous injection. Such medium may also contain conventional pharmaceutical materials such as, for example, pharmaceutically acceptable salts to adjust the osmotic pressure, buffers, preservatives and the like. Among the preferred media are normal saline and plasma. Suitable pharmaceutical acceptable carriers are known to the person skilled in the art. In this regard reference can be made to e.g., Remington's Practice of Pharmacy, 11^{th} ed. and in J. of. Pharmaceutical Science & Technology, Vol. 52, No. 5, September-October, p. 238-311 see table page 240 to 311, both publications include herein by reference.

The concentration of the compound having general chemical Formula II and the pharmaceutically acceptable carrier, for example, in an aqueous medium, varies with the particular field of use. A sufficient amount is present in the pharmaceutically acceptable carrier when satisfactory visualization of the imaging target (e.g., a tumor) is achievable.

In accordance with the invention, the radiolabeled compounds having general chemical Formula II either as a neutral composition or as a salt with a pharmaceutically acceptable counter-ion are administered in a single unit injectable dose. Any of the common carriers known to those with skill in the art, such as sterile saline solution or plasma, can be utilized after radiolabelling for preparing the injectable solution to diagnostically image various organs, tumors and the like in accordance with the invention. Generally, the unit dose to be administered for a diagnostic agent has a radioactivity of about 0.1 mCi to about 100 mCi, preferably 1 mCi to 20 mCi. For a radiotherapeutic agent, the radioactivity of the therapeutic unit dose is about 10 mCi to 700 mCi, preferably 50 mCi to 400 mCi. The solution to be injected at unit dosage is from about 0.01 ml to about 30 ml. For diagnostic purposes after intravenous administration, imaging of the organ or tumor in vivo can take place in a matter of a few minutes. However, imaging takes place, if desired, in hours or even longer, after injecting into patients. In most instances, a sufficient amount of the administered dose will accumulate in the area to be imaged within about 0.1 of an hour to permit the taking of scintigraphic images. Any conventional method of scintigraphic imaging for diagnostic purposes can be utilized in accordance with this invention.

The use of silicon derivatives described herein facilitates the process. Thus, a desired PET imaging agent may be proposed starting from a silicon derivative which is then subjected to ¹⁸F fluorination.

Substituents on such silicon derivatives include linking groups or reactive groups designed for subsequent addition of a targeting agent. Linking groups may include aliphatic or aromatic molecules and readily form a bond to a selected, appropriate functionalized targeting agent. A variety of such groups is known in the art. These include carboxylic acids, carboxylic acid chlorides and active esters, sulfonic acids, sulfonylchlorides amines, hydroxides, thiols etc. on either side.

Contemplated herein are also groups which provide for ionic, hydrophobic and other non-convalent bonds between silicon derivative and targeting agent.

In a fifth aspect, the present invention furthermore relates to a method for producing a compound having general chemical Formula II, as defined herein above, said method comprising reacting a compound having general chemical Formula I or III, respectively, with a fluorinating agent.

The X— group attached to the silyl moiety in the compound having general chemical Formula I or in the compound having general chemical Formula III can be displaced with fluorine isotope, to provide a chemically and biologically stable bond.

The radiofluorination reactions can be carried out in dimethylformamide with potassium carbonate as base and “kryptofix” as crown-ether. But also other solvents can be used which are well known to experts. In a preferred embodiment, the fluorination agent is 4,7,13,16,21,24-Hexaoxa-1,10-diazabicyclo[8.8.8]-hexacosane K18F (crownether salt Kryptofix K18F), K¹⁸F, H¹⁸F, KH¹⁸F₂ or tetraalkylammonium salt of ¹⁸F. More preferably, the fluorination agent is K¹⁸F, H¹⁸F, or KH¹⁸F₂.

The conditions include, but are not limited to: dimethylsulfoxid and acetonitrile as solvent and tetraalkyl ammonium and tetraalkyl phosphonium carbonate as base. Water and/or alcohol can be involved in such a reaction as co-solvent. The radiofluorination reactions are conducted for 1 to 45 minutes. Preferred reaction times are 3 to 40 minutes. Further preferred reaction times are 5 to 30 min.

Preferably organic acids are used in the ¹⁸F radiolabeling, reaction. More preferably aliphatic, cycloaliphatic, aromatic, araliphatic, heterocyclic carboxylic and sulphonic acids are used in the ¹⁸F radiolabeling, reaction. Most preferably aliphatic carboxylic acids are used, including but not limited to propionic acid, acetic acid and formic acid.

In a preferred method of preparing a compound having general chemical Formula II, the step of flurination more preferably radiofluorination of a compound having general chemical Formula I is carried out at a temperature at or below 90° C., more preferably at a temperature in a range of from 10° C. to 90° C., even more preferably at a reaction temperature from room temperature to 80° C., even more preferably at a temperature in a range of from 10° C. to 70° C., even more preferably at a temperature in a range of from 30° C. to 60° C., even more preferably at a temperature in a range of from 45 to 55° C. and most preferably at a temperature at 50° C.

A new method is warranted in which the final product is prepared in a single step from the precursor. Only a single purification step is optionally carried out, thereby the preparation can be accomplished in a short time (considering the half-life of ¹⁸F). In a typical prosthetic group preparation, very often temperatures of 100° C. and above are employed. The invention provides methods to accomplish the preparation at temperatures (80° C. or below) that preserve the biological properties of the final product.

### Example for Labeling

¹⁸F-fluoride (up to 40 GBq) was azeotropically dried in the presence of Kryptofix 222 (5 mg in 1.5 ml MeCN) and cesium carbonate (2.3 mg in 0.5 ml water) by heating under a stream of nitrogen at 110-120° C. for 20-30 minutes. During this time 3×1 ml MeCN were added and evaporated. After drying, a solution of the precursor (2 mg) in 150 μl DMSO was added. The reaction vessel was sealed and heated at 50-70° C. for 5-15 mins to effect labeling. The reaction was cooled to room temperature and diluted with water (2.7 ml). The crude reaction mixture was analyzed using an analytical HPLC. The product was obtained by preparative radio HPLC to give the desired ¹⁸F labeled peptide.

Thus, the embodiments of the present invention include methods involving the ¹⁸F fluorination, of compounds ready for use as imaging agents and the ¹⁸F containing compounds derived from them. The compounds subjected to fluorination, may already include a targeting agent for imaging purposes. Preferred embodiments of this invention involve the formation of a precursor molecule, which may include a targeting agent, prior to fluorinate with ¹⁸F, being the last step in the process prior to preparation of the compound for administration to an animal, in particular a human.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

The entire disclosure[s] of all applications, patents and publications, cited herein are incorporated by reference herein.

The following examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

### General Method for the Preparation of Compounds

The targeting agent radical portion, preferably peptide portion, of the molecule part E-Z-Y— can be conveniently prepared according generally established techniques known in the art of peptide synthesis, such as solid-phase peptide synthesis. They are amenable Fmoc-solid phase peptide synthesis, employing alternate protection and deprotection. These methods are well documented in peptide literature. (Reference: “Fmoc Solid Phase Peptide Synthesis” A practical approach”, Edited by W. C. Chan and P. D. White, Oxford University Press 2000) (For Abbreviations see Descriptions).

### EXAMPLES

Examples of synthesis of compounds having general chemical Formulae I and III are shown below. Model fluoro silanes using non-radioactive fluoride (19F) were synthesized in order to test the procedures and used to confirm the preparation of the corresponding labelled derivatives.

### I. Synthesis of Precursors (Silanes) Via Nucleophilic Substitution:

The following Scheme A describes the general synthetic route of suitable silyl building blocks which can be coupled to biomolecules followed by fluorination or subsequent direct radiolabelling towards the corresponding ¹⁸F labelled compounds having general chemical Formula I. Compound 7 was prepared by nucleophilic substitution of silyl chlorides with aryl metal complexes followed by acidic deprotection (4) and Jones oxidation (5) to generate functionalities like carbonic acids 5 or active esters 6 for coupling to biomolecules (7). The coupling can be performed using well known coupling reagents like EDCI or DCC. Silane 7 was then fluorinated using potassium fluoride and acetic acid to yield the tracer 8. It turned out that the use of acid guarantees improved yields for this reaction and also for the reactions described in following sections II, III, IV and V. This result is surprising due to the fact that the expected or potential formation of hydrogen fluoride would rather stop the reaction than accelerating it.

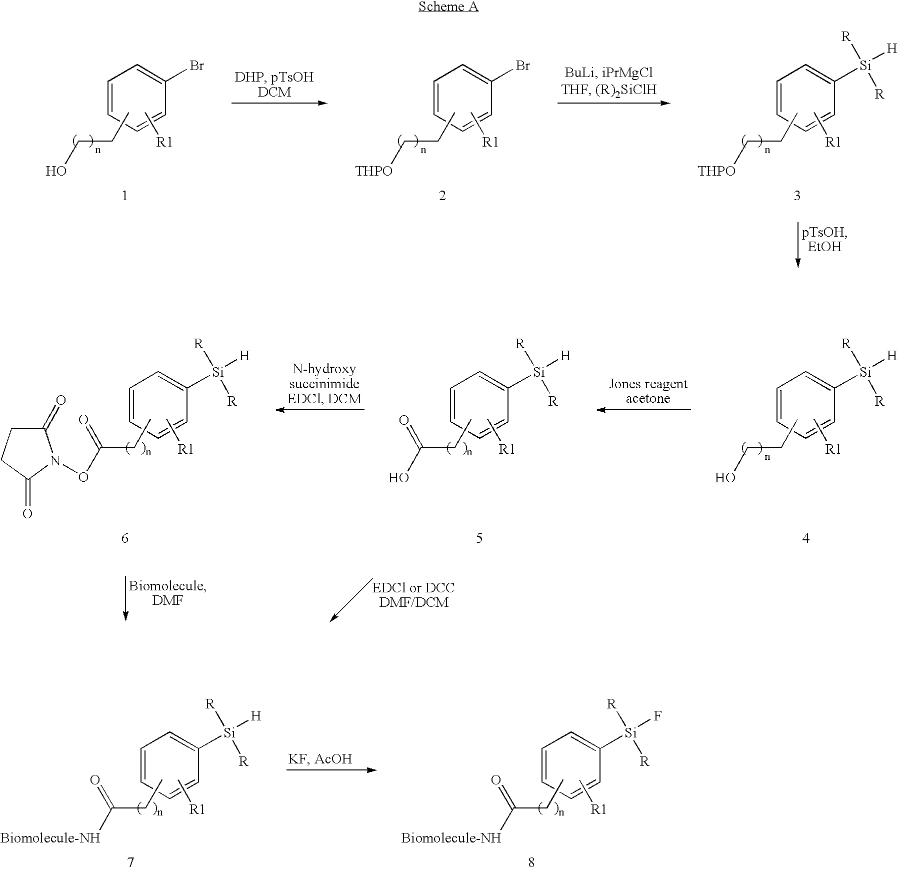

### II. Synthesis of Precursors (Silanoles) Via Nucleophilic Substitution:

Alternatively, silanoles were applied as labelling precursors. Here silane 6 was subjected to an oxidation to silanole 9 prior coupling to the biomolecule to yield precursors of type 10 which were readily fluorinated with potassium fluoride, potassium carbonate, kryptofix and acetic acid to give tracers of type 8 (Scheme B).

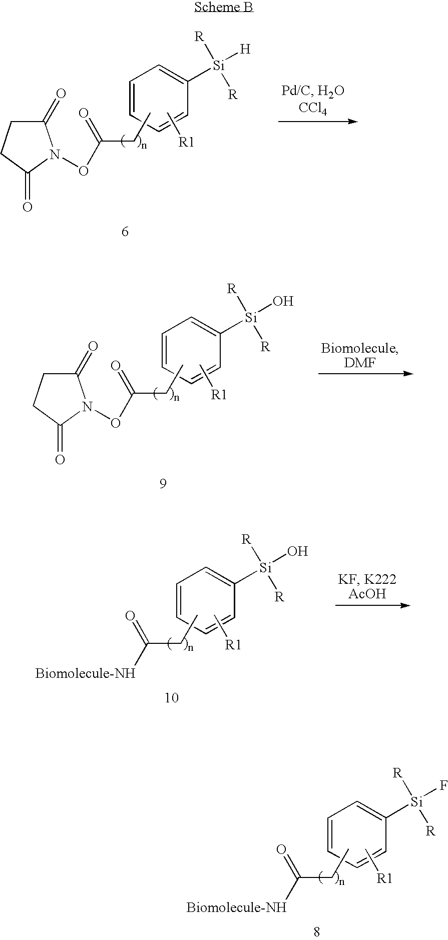

### III. Synthesis of Precursors Via Carbene Insertion:

Benzyl diazoacetate 12 was prepared by diazotization of benzyl glycine by the method described by N. E. Searle, Org. Synth., Coll., 1963, Vol. 4:p. 424, 1956, Vol. 36:p. 25, for the synthesis of ethyl diazoacetate (Scheme C). Addition of benzyl diazoacetate 12 to a mixture of di-alkyl chlorosilane and a catalytic amount of Rh₂(OAc)₄ in anhydrous dichloromethane yields a chlorosilane intermediate which can either be added to a mixture of alcohol, imidazole and 4-DMAP in dry DMF to give alkoxysilanes 13. Or, respectively, the chlorosilane intermediate was treated with NEt₃ and H₂O to give the silanol 13 (R¹═OH). The benzyl ester group of the silanol 13 can then be cleaved off by hydrogenation in presence of 10% Pd/C catalyst to give 14 which can be coupled to biomolecules 15 followed by fluorination with potassium fluoride, potassium carbonate, kryptofix, and acetic acid to 16.

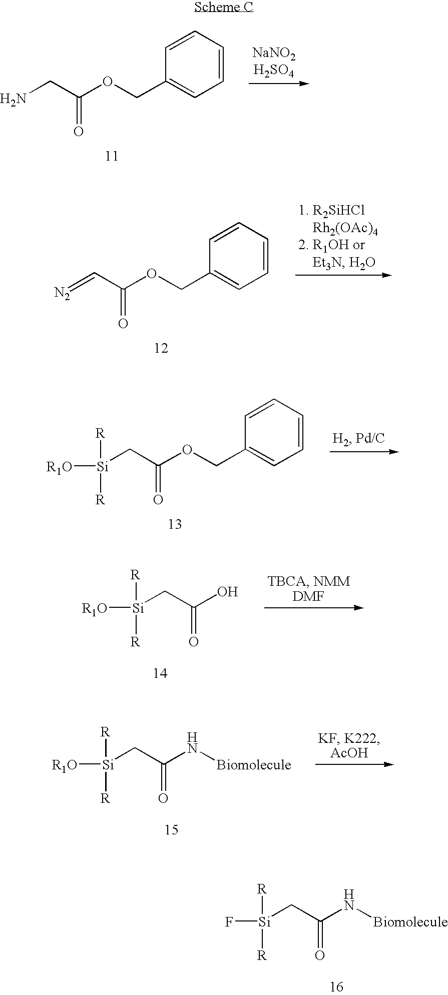

IV. Synthesis of Precursors and Fluorinated Targeting Agents Starting from Commercially Available Si-Derivatives:

The commercially available di-iso-propyl silicon amine 17 reflects an example of silicon derivatives which can be used as described in the following Scheme in the synthesis of a precursor 19 that can be coupled to a targeting agent towards 20 followed by fluorination with potassium fluoride, potassium carbonate, kryptofix, and acetic acid to 21 (Scheme D).

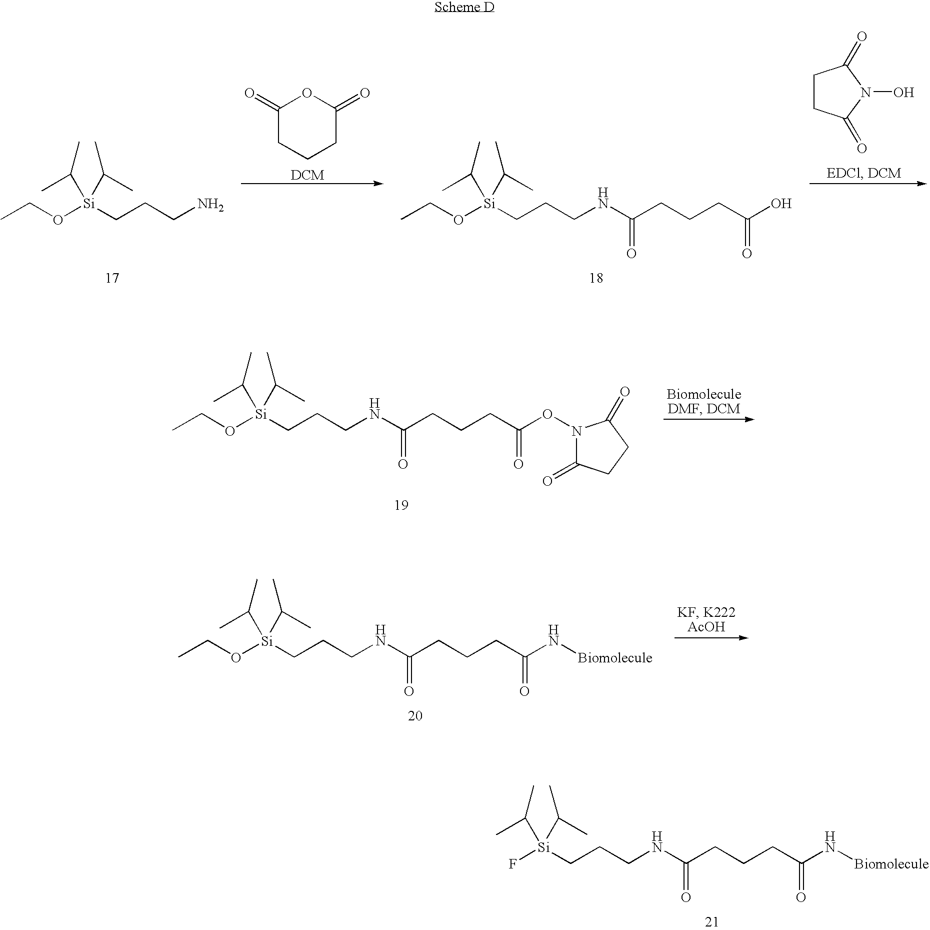

### V. Synthesis of Precursors Via Hydrosilylation:

Suitable silicon building blocks for labelling can also be prepared via hydrosilylation as shown in the following Scheme E. The functionalized alkene 26 can be transformed to chlorosilane 27 using the Karstedt Catalyst Pt₂{[(CH₂═CH)Me₂Si]₂O}₃. Treatment with alcohols or water yields silanols or alkoxysilanes 28 which can be coupled to biomolecules to receive labeling precursors of type 29 applicable for subsequent fluorination with potassium fluoride, potassium carbonate, kryptofix and acetic acid to 30.

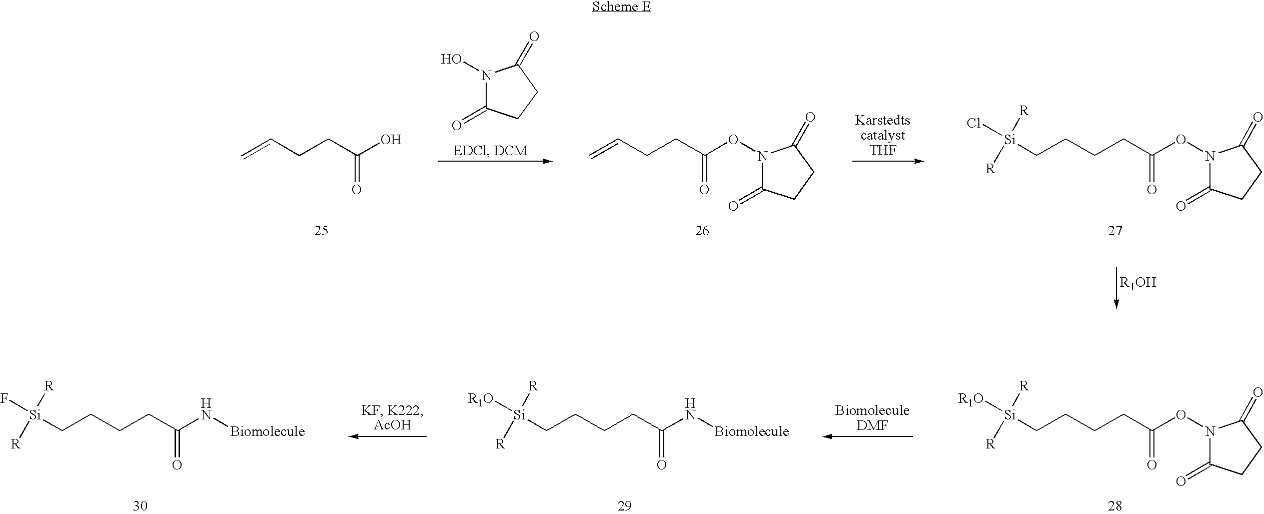

### Experimental Part

### Example I

### Part A

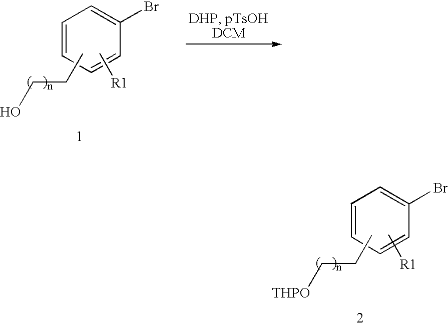

### General Method

To a solution of 53.5 mmol of 1 in 220 ml dichloro-methane 36 ml 3,4-dihydro-2H-pyran and 202 mg pyridinium toluene-4-sulfonate was added. After the reaction mixture was stirred for 16 hours at 23° C. it was added to a solution of sodium bicarbonate. The organic extract was washed with brine and dried over sodium sulfate. After filtration and removal of the solvent the crude product was purified by chromatography on silica gel to give 2 in 77-98% yield.

### (RS)-2-(4-Bromo-benzyloxy)-tetrahydro-pyran

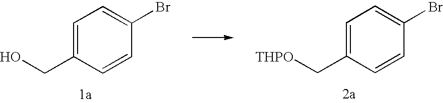

¹H-NMR (CDCl₃): δ=7.47 (2H), 7.24 (2H), 4.73 (1H), 4.69 (1H), 4.46 (1H), 3.89 (1H), 3.54 (1H), 1.86 (1H), 1.74 (1H), 1.69-1.50 (4H) ppm.

### (RS)-2-[2-(4-Bromo-phenyl)-ethoxy]-tetrahydro-pyran

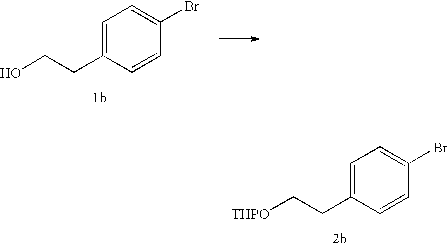

¹H-NMR (CDCl₃): δ=7.51 (2H), 7.29 (2H), 5.20-4.50 (2H), 3.65 (2H), 1.20 (3H), 1.04 (6H), 0.96 (6H) ppm.

### (RS)-2-[3-(4-Bromo-phenyl)-propoxy]-tetrahydro-pyran

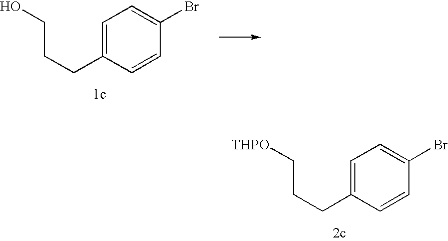

¹H-NMR (CDCl₃): δ=7.39 (2H), 7.07 (2H), 4.56 (1H), 3.86 (1H), 3.75 (1H), 3.50 (1H), 3.39 (1H), 2.67 (1H), 1.94-1.48 (9H) ppm.

### (RS)-2-[2-(3-Bromo-phenyl)-ethoxy]-tetrahydro-pyran

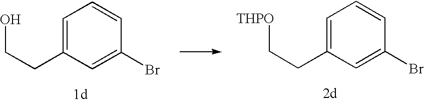

¹H-NMR (CDCl₃): δ=7.41 (1H), 7.34 (1H), 7.19-7.11 (2H), 4.59 (1H), 3.93 (1H), 3.72 (1H), 3.60 (1H), 3.45 (1H), 2.88 (2H), 1.87-1.44 (6H) ppm.

### (RS)-2-[3-(3-Bromo-phenyl)-propoxy]-tetrahydro-pyran

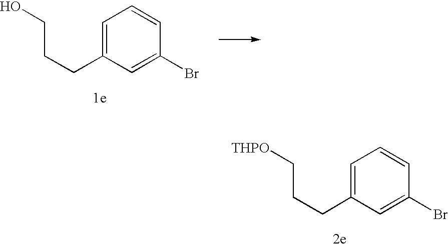

¹H-NMR (CDCl₃): δ=7.36 (1H), 7.31 (1H), 7.17-7.11 (2H), 4.57 (1H), 3.86 (1H), 3.76 (1H), 3.50 (1H), 3.39 (1H), 2.68 (2H), 1.91 (2H), 1.86-1.49 (6H) ppm.

### 2-(3-(4-Bromo-3-methylphenoxy)propoxy)tetrahydro-2H-pyran

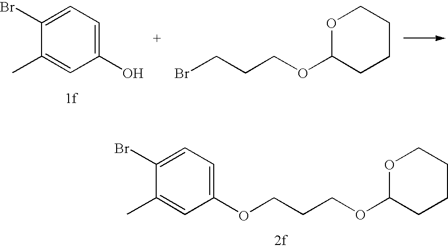

4-Bromo-3-methylphenol (28.83 mmol, 5.392 g) was dissolved in DMSO (70 ml). Potassium hydroxide (129.7 mmol, 7.279 g) was added. After 5 min stirring 2-(3-bromopropoxy)-tetrahydro-2H-pyran (44.68 mmol, 9.969 g) was added. The reaction mixture was stirred at room temperature overnight and then partioned between water (250 ml) and dichloromethane (250 ml). The aqueous phase was extracted with dichloromethane (2×150 ml). The combined organic layers were washed with brine (3×250 ml), dried (MgSO₄), and the solvent was evaporated. The residue was purified by column chromatography (pentane/ethyl acetate 19:1) to yield 2-(3-(4-bromo-3-methylphenoxy)propoxy)tetrahydro-2H-pyran (9.391 g, 99%) as a colorless oil.

¹H-NMR (CDCl₃, 400 MHz): δ=1.49-1.85 (m, 6H, CH₂ [THP]), 2.06 (quint., 2H, CH₂), 2.35 (s, 3H, CH₃), 3.45-3.52 (m, 1H, O—CH₂ [THP]), 3.53-3.59 (m, 1H, O—CH₂), 3.81-3.87 (m, 1H, O—CH₂ [THP]), 3.88-3.94 (m, 1H, O—CH₂), 4.04 (t, 2H, O—CH₂), 4.59 (m_{c}, 1H, O—CH—O [THP]), 6.60-6.63 (m, 1H, Ar—H), 6.79 (d, 1H, Ar—H), 7.38 (d, 1H, Ar—H). ¹³C-NMR (CDCl₃, 100 MHz): δ=19.8 (CH₂ [THP]), 23.3 (CH₃), 25.6 (CH₂ [THP]), 29.9 (CH₂), 30.9 (CH₂ [THP]), 62.6 (O—CH₂ [THP]), 64.1 (O—CH₂), 65.3 (O—CH₂), 99.1 (O—CH—O [THP]), 113.7 (Ar—CH), 115.4 (Ar—C), 117.3 (Ar—CH), 132.9 (Ar—CH), 138.9 (Ar—C), 158.4 (Ar—C).

### 2-(3-(4-Bromo-3,5-dimethylphenoxy)propoxy)tetrahydro-2H-pyran

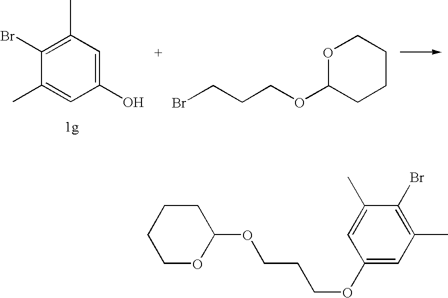

4-Bromo-3,5-dimethylphenol (20.02 mmol, 4.026 g) was dissolved in DMSO (50 ml). Potassium hydroxide (90.01 mmol, 5.056 g) was added. After 5 min stirring 2-(3-bromopropoxy)-tetrahydro-2H-pyran (31.04 mmol, 6.924 g) was added. The reaction mixture was stirred at room temperature overnight and then partioned between water (250 ml) and dichloromethane (250 ml). The aqueous phase was extracted with dichloromethane (3×100 ml). The combined organic layers were washed with brine (3×250 ml), dried (MgSO₄), and the solvent was evaporated. The residue was purified by column chromatography (pentane/ethyl acetate 39:1) to yield 2-(3-(4-bromo-3,5-dimethylphenoxy)propoxy)tetrahydro-2H-pyran (6.875 g, quant.) as a colorless oil.

¹H-NMR (CDCl₃, 400 MHz): δ=1.49-1.86 (m, 6H, CH₂ [THP]), 2.05 (quint., 2H, CH₂), 2.37 (s, 6H, 2×CH₃), 3.47-3.53 (m, 1H, O—CH₂ [THP]), 3.53-3.59 (m, 1H, O—CH₂), 3.82-3.88 (m, 1H, O—CH₂ [THP]), 3.88-3.94 (m, 1H, O—CH₂), 4.03 (t, 2H, O—CH₂), 4.59 (m_{c}, 1H, O—CH—O [THP]), 6.65 (s, 2H, Ar—H). ¹³C-NMR (CDCl₃, 100 MHz): δ=19.8 (CH₂ [THP]), 24.2 (CH₃), 25.6 (CH₂ [THP]), 29.8 (CH₂), 30.8 (CH₂ [THP]), 62.5 (O—CH₂ [THP]), 64.1 (O—CH₂), 65.3 (O—CH₂), 99.1 (O—CH—O [THP]), 114.6 (Ar—CH), 118.3 (Ar—C), 139.2 (Ar—CH), 157.7 (Ar—C).

### Part B

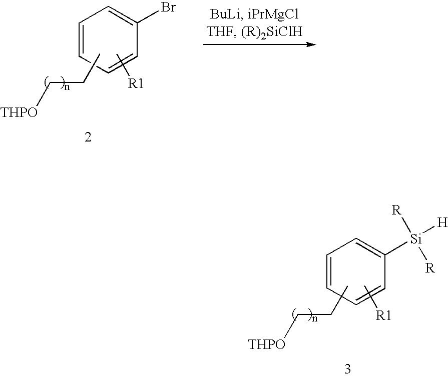

### General Method

14.2 ml of a 2M isopropylmagnesium bromide solution in tetrahydrofuran (THF) was diluted with 140 ml THF and cooled to 5° C. 22.6 ml of a 2.5M solution of butyl-lithium in n-hexane was added followed by the solution of 14.2 mmol 2 in 12 ml THF. After 2 hours at 5-10° C. 17.6 g chloro-diisopropyl-silane was added, the cooling bath removed and stirring continued for 2 hours. The reaction mixture was added to a solution of sodium bicarbonate and extracted with ethyl acetate. The combined organic extracts were washed with brine and dried over sodium sulfate. After filtration and removal of the solvent the crude product was purified by chromatography on silica gel to give 93-99% of 3.

### (RS)-Diisopropyl-[4-(tetrahydro-pyran-2-yloxymethyl)-phenyl]-silane

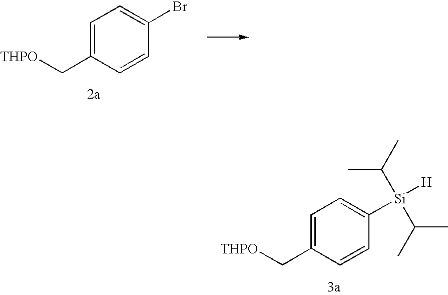

¹H-NMR (CDCl₃): δ=7.43 (2H), 7.28 (2H), 4.73 (1H), 4.66 (1H), 4.42 (1H), 3.86 (2H), 3.49 (1H), 1.89-1.43 (6H), 1.14 (2H), 0.99 (6H), 0.92 (6H) ppm.

### (RS)-Diisopropyl-[4-[2-(tetrahydro-pyran-2-yloxy)-ethyl]-phenyl]-silane

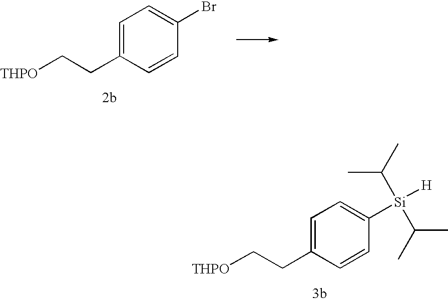

¹H-NMR (CDCl₃): δ=7.43 (2H), 7.22 (2H), 4.60 (1H), 3.98-3.87 (2H), 3.71-3.60 (2H), 3.42 (1H), 2.91 (2H), 1.79 (1H), 1.69 (1H), 1.63-1.43 (4H), 1.21 (2H), 1.05 (6H), 0.97 (6H) ppm.

### (RS)-Diisopropyl-{4-[3-(tetrahydro-pyran-2-yloxy)-propyl]-phenyl}-silane

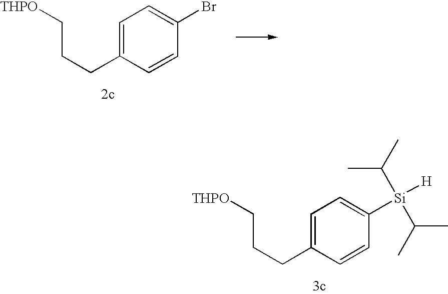

¹H-NMR (CDCl₃): δ=7.42 (2H), 7.19 (2H), 4.59 (1H), 3.92 (1H), 3.87 (1H), 3.78 (1H), 3.50 (1H), 3.42 (1H), 2.71 (2H), 1.94 (2H), 1.84 (1H), 1.72 (1H), 1.63-1.49 (4H), 1.22 (2H), 1.06 (6H), 0.99 (6H) ppm.

### (RS)-Diisopropyl-{3-[2-(tetrahydro-pyran-2-yloxy)-ethyl]-phenyl}-silane

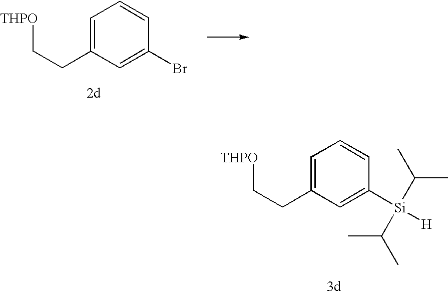

¹H-NMR (CDCl₃): δ=7.39 (1H), 7.34 (1H), 7.29-7.24 (2H), 4.60 (1H), 3.95 (1H), 3.92 (1H), 3.72 (1H), 3.62 (1H), 3.44 (1H), 2.91 (2H), 1.80 (1H), 1.68 (1H), 1.62-1.43 (4H), 1.22 (2H), 1.06 (6H), 0.99 (6H) ppm.

### (RS)-Diisopropyl-{3-[3-(tetrahydro-pyran-2-yloxy)-propyl]-phenyl}-silane

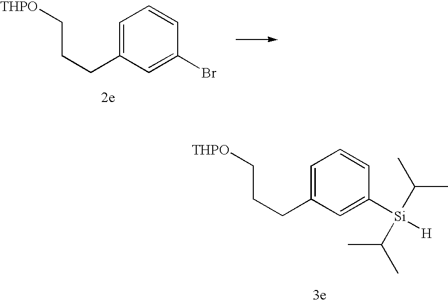

¹H-NMR (CDCl₃): δ=7.83 (2H), 7.26 (1H), 7.21 (1H), 4.58 (1H), 3.92 (1H), 3.88 (1H), 3.78 (1H), 3.50 (1H), 3.40 (1H), 2.70 (2H), 1.93 (2H), 1.85 (1H), 1.73 (1H), 1.64-1.59 (4H), 1.23 (2H), 1.06 (6H), 0.99 (6H) ppm.

### Di-iso-propyl(2-methyl-4-(3-(tetrahydro-2H-pyran-2-yloxy)propoxy)phenyl)silane

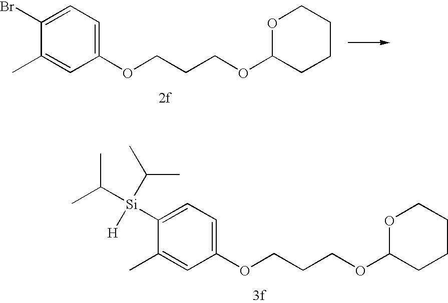

In a flame-dried flask isopropylmagnesium chloride (2.0M in THF, 30.0 mmol, 15.0 ml) was diluted with THF (150 ml) and cooled to 0° C. N-butyl lithium (1.6M in hexane, 60.0 mmol, 37.5 ml) was added. After stirring for 30 min at 0° C. 2-(3-(4-bromo-3-methylphenoxy)propoxy)tetrahydro-2H-pyran (15.00 mmol, 4.939 g) in THF (12 ml) was added dropwise. After stirring for 2 h at 0° C. chlorodiisopropylsilane (90.0 mmol, 15.4 ml) was added dropwise. The ice bath was removed and the mixture was stirred for another 2 h. Then it was poured into diluted NaHCO₃ solution and extracted with etyl acetate. The combined organic layers were washed with brine, dried (MgSO₄), and the solvent was evaporated. The residue was purified by column chromatography (pentane/ethyl acetate 39:1) to yield di-iso-propyl(2-methyl-4-(3-(tetrahydro-2H-pyran-2-yloxy)propoxy)phenyl)silane (5.167 g, 95%).

¹H-NMR (CDCl₃, 400 MHz): δ=0.95 (d, 6H, 2×CH₃), 1.07 (d, 6H, 2×CH₃), 1.18-1.28 (m, 2H, 2×CH), 1.49-1.86 (m, 6H, CH₂ [THP]), 2.07 (quint., 2H, CH₂), 2.40 (s, 3H, CH₃), 3.47-3.52 (m, 1H, O—CH₂ [THP]), 3.55-3.61 (m, 1H, O—CH₂), 3.80-3.88 (m, 1H, O—CH₂ [THP]), 3.89-3.95 (m, 1H, O—CH₂), 4.03 (t, 1H, Si—H), 4.05-4.10 (m, 2H, O—CH₂), 4.60 (m_{c}, 1H, O—CH—O [THP]), 6.70-6.75 (m, 2H, Ar—H), 7.32 (d, 1H, Ar—H). ¹³C-NMR (CDCl₃, 100 MHz): δ=11.4 (CH), 19.1 (CH₃), 19.3 (CH₃), 19.8 (CH₂ [THP]), 23.6 (CH₃), 25.6 (CH₂ [THP]), 29.9 (CH₂), 30.9 (CH₂ [THP]), 62.5 (O—CH₂ [THP]), 64.3 (O—CH₂), 64.6 (O—CH₂), 99.1 (O—C—O [THP]), 110.9 (Ar—CH), 116.3 (Ar—CH), 124.4 (Ar—C), 137.6 (Ar—CH), 146.0 (Ar—C), 160.1 (Ar—C).

### (2,6-Dimethyl-4-(3-(tetrahydro-2H-pyran-2-yloxy)propoxy)phenyl)di-iso-propylsilane

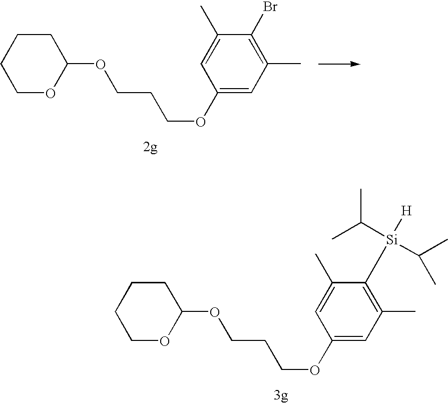

In a flame dried flask 2-(3-(4-bromo-3,5-dimethylphenoxy)propoxy)tetrahydro-2H-pyran (20.02 mmol, 6.873 g) was dissolved in dry THF (150 ml) and cooled to −78° C. (acetone-dry ice bath). n-Butyl lithium solution (1.6M in hexane, 22.03 mmol, 13.8 ml) was added dropwise. After stirring for 1 h at −78° C. iso-Pr₂SiHCl (22.03 mmol, 3.76 ml) was added dropwise. The reaction mixture was allowed to warm slowly to room temperature and stirred for 40 h. Then it was poured into diluted NaHCO₃ solution and the organic phase was separated. The aqueous phase was extracted with ethyl acetate. The combined organic extracts were washed with brine, dried over MgSO₄ and solvents were removed. The residue was purified by column chromatography (pentane/ethyl acetate 19:1) to give (2,6-dimethyl-4-(3-(tetrahydro-2H-pyran-2-yloxy)propoxy)phenyl)di-iso-propylsilane as a colorless oil in 97% yield (7.342 g).

¹H-NMR (CDCl₃, 400 MHz): δ=0.92 (d, 6H, 2×CH₃), 1.14 (d, 6H, 2×CH₃), 1.25-1.33 (m, 2H, 2×CH), 1.49-1.86 (m, 6H, CH₂ [THP]), 2.06 (quint., 2H, CH₂), 2.41 (s, 6H, 2×CH₃), 3.47-3.52 (m, 1H, O—CH₂ [THP]), 3.55-3.61 (m, 1H, O—CH₂), 3.83-3.87 (m, 1H, O—CH₂ [THP]), 3.88-3.94 (m, 1H, O—CH₂), 4.02-4.09 (m, 2H, O—CH₂), 4.11 (t, 1H, Si—H), 4.60 (m_{c}, 1H, O—CH—O [THP]), 6.56 (s, 2H, Ar—H). ¹³C-NMR (CDCl₃, 100 MHz): δ=13.7 (CH), 19.8 (CH₂ [THP]), 19.9 (CH₃), 20.4 (CH₃), 24.9 (CH₃), 25.6 (CH₂ [THP]), 29.9 (CH₂), 30.9 (CH₂ [THP]), 62.5 (O—CH₂ [THP]), 64.3 (O—CH₂), 64.6 (O—CH₂), 99.1 (O—CH—O [THP]), 113.8 (Ar—CH), 124.4 (Ar—C), 146.3 (Ar—C), 159.6 (Ar—C).

### Di-tert-butyl(4-(2-(tetrahydro-2H-pyran-2-yloxy)ethyl)phenyl)silane

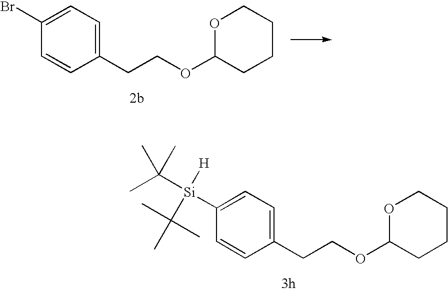

In a flame dried flask 2-(4-bromophenethoxy)tetrahydro-2H-pyran (8.81 mmol, 2.51 g) was dissolved in dry THF (35 ml) and cooled to −78° C. (acetone dry ice bath). n-Butyl lithium solution (1.6M in hexane, 1.0 eq, 8.81 mmol, 5.51 ml) was added dropwise. After stirring for 1 h at −78° C. (tert-Bu)₂SiHCl (3.4 eq, 30.0 mmol, 6.09 ml) was added dropwise. The reaction mixture was allowed to warm slowly to room temperature and stirred for 48 h. Then it was poured into diluted NaHCO₃ solution and the organic phase was separated. The aqueous phase was extracted with pentane. The combined organic extracts were washed with brine, dried over MgSO₄ and solvents were removed. The residue was purified by column chromatography (pentane/ethyl acetate 39:1) to give di-tert-butyl(4-(2-(tetrahydro-2H-pyran-2-yloxy)ethyl)phenyl)silane as a white solid in 74% yield (2.27 g).

¹H-NMR (CDCl₃, 400 MHz): δ=1.03 (s, 18H, Si(tBu)₂), 1.40-1.84 (m, 6H, CH₂ [THP]), 2.91 (t, 2H, ³J_{H—H}=7.4 Hz, CH₂), 3.37-3.43 (m, 1H, O—CH₂ [THP]), 3.61-3.67 (m, 2H, O—CH₂), 3.84 (s, 1H, Si—H), 3.91-3.97 (m, 1H, O—CH₂ [THP]), 4.59 (m_{c}, 1H, O—CH—O [THP]), 7.21 (d, 2H, ³J_{H—H}=8.1 Hz, Ar—H), 7.49 (d, 2H, ³J_{H—H}=8.1 Hz, Ar—H). ¹³C-NMR (CDCl₃, 100 MHz): δ=19.2 (Si—C), 19.5 (CH₂ [THP]), 25.6 (CH₂ [THP]), 29.1 (CH₃), 30.8 (CH₂ [THP]), 36.5 (CH₂), 62.1 (O—CH₂ [THP]), 68.2 (O—CH₂), 98.7 (O—CH—O [THP]), 128.4 (Ar—CH), 132.9 (Ar—C), 135.9 (Ar—CH), 140.0 (Ar—C). ²⁹Si-NMR (CDCl₃, 79 MHz): δ=12.9 (¹J_{Si—H}=187 MHz). MS (ESI positive): 349.01 [M+H]⁺. HR-ESI-MS: 371.2372 [M+Na]⁺ (calc. for C₂₁H₃₆NaO₂Si: 371.2377).

### (RS)-Diphenyl-{4-[2-(tetrahydro-pyran-2-yloxy)-ethyl]-phenyl}-silane

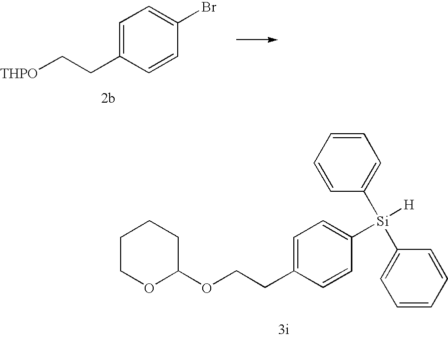

¹H-NMR (CDCl₃): δ=7.62 (4H), 7.55 (2H), 7.50-7.37 (6H), 7.31 (2H), 5.50 (1H), 4.64 (1H), 3.99 (1H), 3.77 (1H), 3.67 (1H), 3.49 (1H), 2.97 (2H), 1.90-1.48 (6H) ppm.

### (RS)-Diisobutyl-[4-(tetrahydro-pyran-2-yloxymethyl)-phenyl]-silane

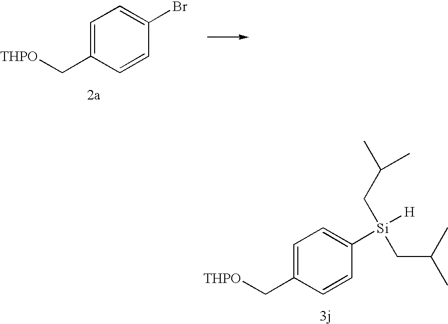

¹H-NMR (CDCl₃): δ=7.52 (2H), 7.35 (2H), 4.80 (1H), 4.73 (1H), 4.49 (1H), 4.38 (1H), 3.93 (1H), 3.56 (1H), 1.95-1.49 (8H), 0.94 (6H), 0.92 (6H), 0.82 (4H) ppm.

### (RS)-Diisobutyl-{4-[2-(tetrahydro-pyran-2-yloxy)-ethyl]-phenyl}-silane

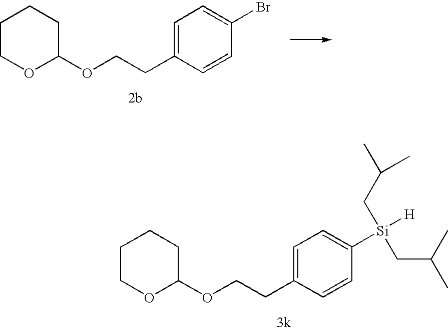

¹H-NMR (CDCl₃): δ=7.45 (2H), 7.23 (2H), 4.60 (1H), 4.36 (1H), 3.95 (1H), 3.71 (1H), 3.63 (1H), 3.43 (1H), 2.91 (2H), 1.87-1.43 (8H), 0.93 (6H), 0.91 (6H), 0.81 (4H) ppm.

### Part C

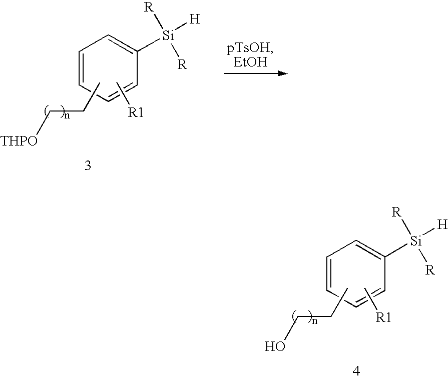

### General Method

To a solution of 9.36 mmol 3 in 120 ml ethanol were added 1.61 g p-toluenesulfonic acid monohydrate and the mixture was stirred for 2 hours at 23° C. The reaction mixture was added to a solution of sodium bicarbonate extracted with dichloromethane. The combined organic extracts were washed with brine and dried over sodium sulfate. After filtration and removal of the solvent the crude product was purified by chromatography on silica gel to give 70-93% of 4.

### (4-Diisopropylsilanyl-phenyl)-methanol

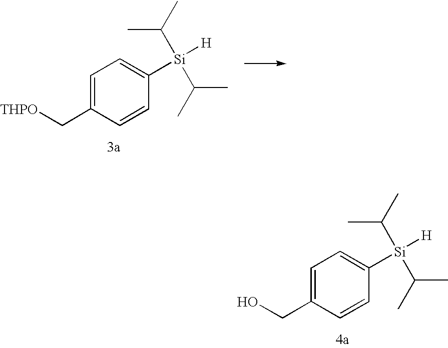

¹H-NMR (CDCl₃): δ=7.51 (2H), 7.35 (2H), 4.70 (2H), 3.94 (1H), 1.64 (1H), 1.23 (2H), 1.06 (6H), 0.98 (6H) ppm.

### 2-(4-Diisopropylsilanyl-phenyl)-ethanol

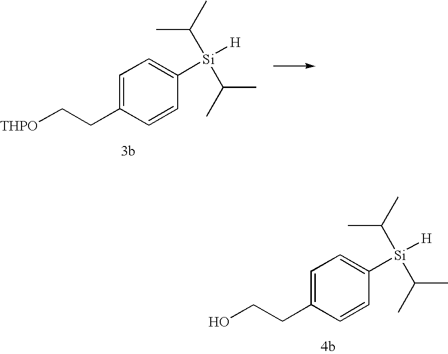

¹H-NMR (CDCl₃): δ=7.46 (2H), 7.22 (2H), 3.93 (1H), 3.88 (2H), 2.87 (2H), 1.53 (1H), 1.22 (2H), 1.06 (6H), 0.99 (6H) ppm.

### 3-(4-Diisopropylsilanyl-phenyl)-propan-1-ol

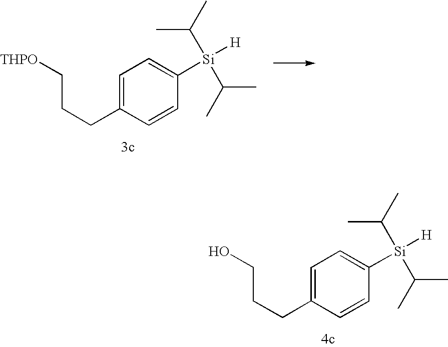

¹H-NMR (CDCl₃): δ=7.44 (2H), 7.19 (2H), 3.92 (1H), 3.69 (2H), 2.71 (2H), 1.91 (2H), 1.30 (1H), 1.21 (2H), 1.06 (6H), 0.99 (6H) ppm.

### 2-(3-Diisopropylsilanyl-phenyl)-ethanol

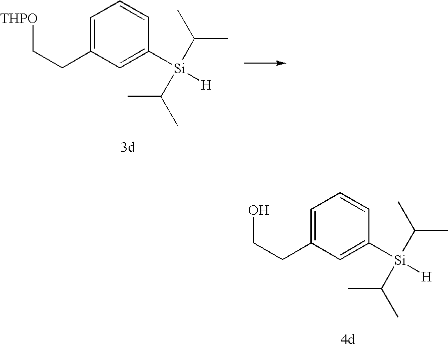

¹H-NMR (CDCl₃): δ=7.39 (1H), 7.37 (1H), 7.30 (1H), 7.24 (1H), 3.93 (1H), 3.87 (2H), 2.87 (2H), 1.39 (1H), 1.23 (2H), 1.06 (6H), 0.99 (6H) ppm.

### 3-(3-Diisopropylsilanyl-phenyl)-propan-1-ol

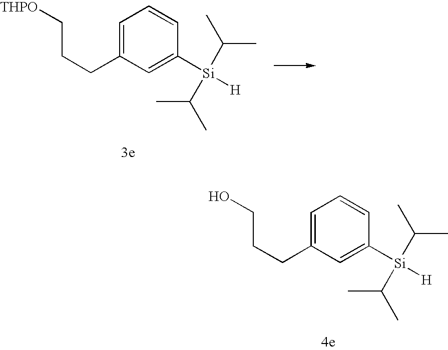

¹H-NMR (CDCl₃): δ=7.33 (2H), 7.26 (1H), 7.21 (1H), 3.92 (1H), 3.69 (2H), 2.71 (2H), 1.90 (2H), 1.30 (1H), 1.22 (2H), 1.06 (6H), 0.99 (6H) ppm.

### 3-(4-(Di-iso-propylsilyl)-3,5-dimethylphenoxy)propan-1-ol

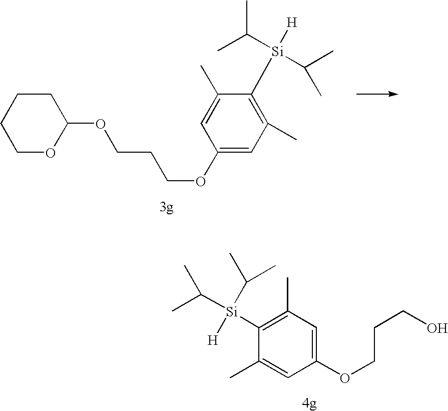

(2,6-Dimethyl-4-(3-(tetrahydro-2H-pyran-2-yloxy)propoxy)phenyl)di-iso-propylsilane (5.00 mmol, 1.893 g) was dissolved in ethanol (60 ml). Pyridinium p-toluenesulfonate (PPTS, 10 mol %, 0.50 mmol, 126 mg) was added and the resulting solution was stirred for 3 h at 55° C. Then the reaction mixture was poured into diluted NaHCO₃ solution and extracted with dichloromethane. The combined organic extracts were washed with water, dried over Na₂SO₄ and solvents were removed. The residue was purified by column chromatography (pentane/ethyl acetate 4:1) to give 3-(4-(di-iso-propylsilyl)-3,5-dimethylphenoxy)propan-1-ol in 72% yield (1.062 g).

¹H-NMR (CDCl₃, 400 MHz): δ=0.91 (d, 6H, 2×CH₃), 1.14 (d, 6H, 2×CH₃), 1.19-1.33 (m, 2H, 2×CH), 1.80 (br s, 1H, OH), 2.03 (quint., 2H, CH₂), 2.41 (s, 6H, 2×CH₃), 3.86 (t, 2H, O—CH₂), 4.11 (t, 2H, O—CH₂), 4.12 (t, 1H, Si—H), 6.56 (s, 2H, Ar—H). ¹³C-NMR (CDCl₃, 100 MHz): δ=12.7 (CH), 19.9 (CH₃), 20.4 (CH₃), 25.0 (CH₃), 32.2 (CH₂), 60.9 (O—CH₂), 65.5 (O—CH₂), 113.7 (Ar—CH), 125.0 (Ar—C), 146.4 (Ar—C), 159.3 (Ar—C).

### (4-Diisobutylsilanyl-phenyl)-methanol

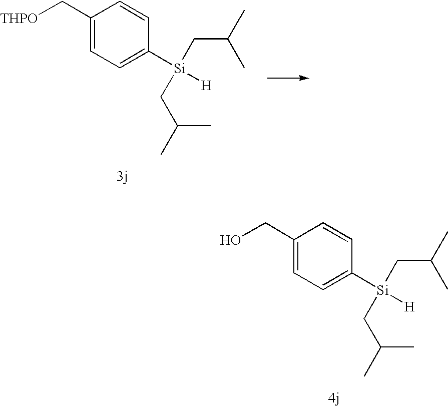

¹H-NMR (CDCl₃): δ=7.59 (2H), 7.39 (2H), 4.74 (2H), 4.43 (1H), 1.79 (2H), 1.68 (1H), 0.99 (6H), 0.97 (6H), 0.87 (4H) ppm.

### 2-(4-Diisobutylsilanyl-phenyl)-ethanol

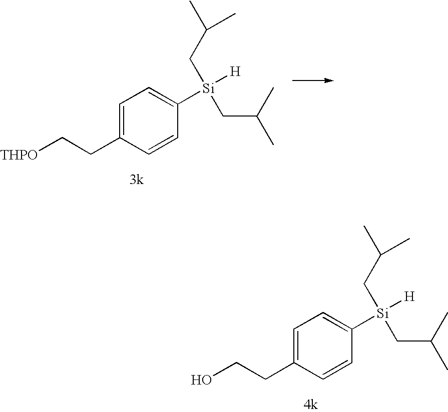

¹H-NMR (CDCl₃): δ=7.53 (2H), 7.26 (2H), 4.42 (1H), 3.92 (2H), 2.92 (2H), 1.80 (2H), 1.51 (1H), 0.97 (6H), 0.99 (6H), 0.87 (4H) ppm.

### Part D

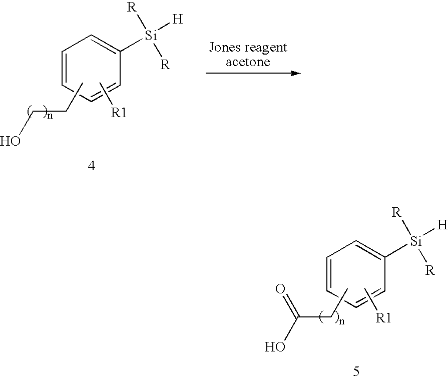

### General Method

To a solution of 2 mmol 4 in 14 ml acetone was added at 0° C. 2.25 ml of Jones reagent. After 15 minutes water was added and the mixture extracted with ethyl acetate. The combined organic extracts were washed with brine and dried over sodium sulfate. After filtration and removal of the solvent the crude product was purified by chromatography on silica gel to give 52-70% of 5.

### 4-Diisopropylsilanyl-benzoic acid

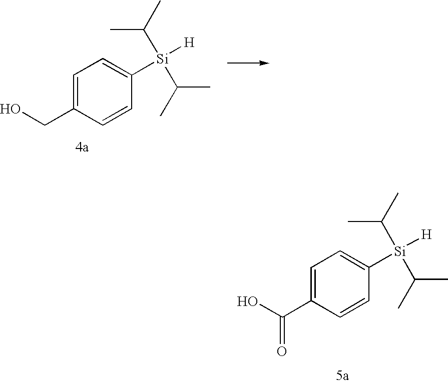

¹H-NMR (CDCl₃): δ=8.07 (2H), 7.64 (2H), 3.99 (1H), 1.27 (2H), 1.08 (6H), 0.99 (6H) ppm.

### (4-Diisopropylsilanyl-phenyl)-acetic acid

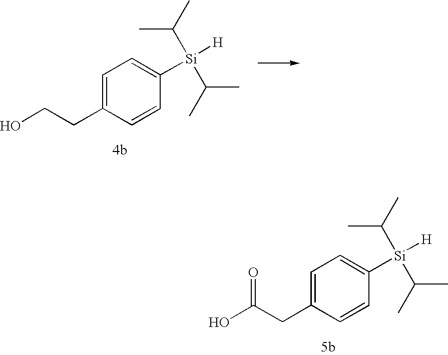

¹H-NMR (CDCl₃): δ=7.48 (2H), 7.28 (2H), 3.93 (1H), 3.65 (2H), 1.22 (2H), 1.06 (6H), 0.99 (6H) ppm.

### 3-(4-Diisopropylsilanyl-phenyl)-propionic acid

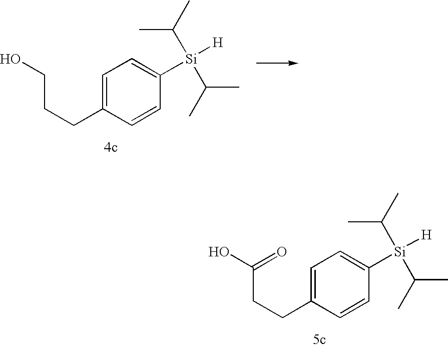

¹H-NMR (CDCl₃): δ=7.45 (2H), 7.20 (2H), 3.93 (1H), 2.97 (2H), 2.70 (2H), 1.22 (2H), 1.06 (6H), 0.99 (6H) ppm.

### (3-Diisopropylsilanyl-phenyl)-acetic acid

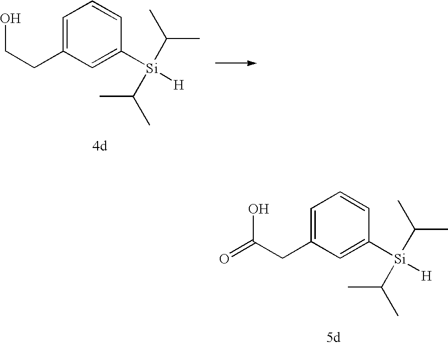

¹H-NMR (CDCl₃): δ=7.45-7.39 (2H), 7.34-7.30 (2H), 3.93 (1H), 3.65 (2H), 1.22 (2H), 1.06 (6H), 0.99 (6H) ppm.

### 3-(3-Diisopropylsilanyl-phenyl)-propionic acid

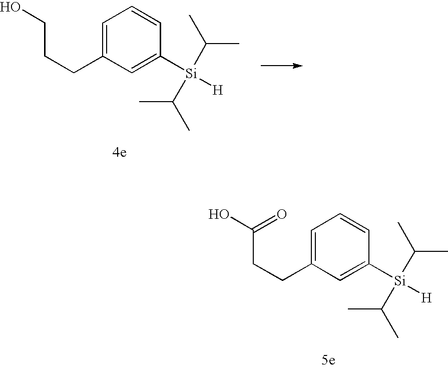

¹H-NMR (CDCl₃): δ=7.37 (1H), 7.35 (1H), 7.28 (1H), 7.22 (1H), 3.92 (1H), 2.96 (2H), 2.69 (2H), 1.22 (2H), 1.06 (6H), 0.98 (6H) ppm.

### 3-(4-(Di-iso-propylsilyl)-3-methylphenoxy)propanoic acid

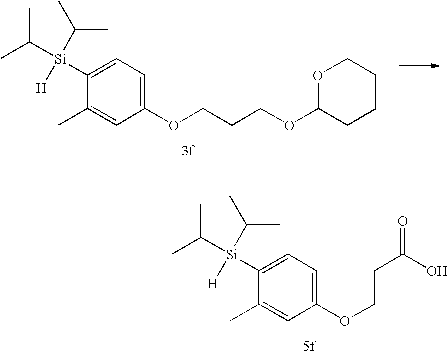

Di-iso-propyl(2-methyl-4-(3-(tetrahydro-2H-pyran-2-yloxy)propoxy)phenyl)silane (5.00 mmol, 1.821 g) was dissolved in ethanol (60 ml). p-Toluenesulfonic acid (1.0 eq, 5.00 mmol, 861 mg) was added and the reaction mixture stirred for 4 h at room temperature. Then it was poured into diluted NaHCO₃ solution and the aqueous phase extracted with ethyl acetate. The combined organic extracts were washed with water and brine, dried over Na₂SO₄ and solvents were removed. The residue was dissolved in acetone (30 ml) and cooled to 0° C. (ice bath). Jones reagent (8M, 6.0 eq., 30.0 mmol, 3.75 ml) was added slowly drop by drop. The reaction mixture was stirred for 15 min at 0° C., then quenched with water and extracted with ethyl acetate. The combined organic extracts were washed with water (3×) and brine, dried over Na₂SO₄ and solvents were removed. The residue was purified by column chromatography (pentane/ethyl acetate/acetic acid 90:9:1) to give 3-(4-(di-iso-propylsilyl)-3-methylphenoxy)propanoic acid as a white solid in 61% yield (895 mg).

¹H-NMR (CDCl₃, 400 MHz): δ=0.95 (d, 6H, 2×CH₃), 1.07 (d, 6H, 2×CH₃), 1.18-1.30 (m, 2H, 2×CH), 2.41 (s, 3H, CH₃), 2.85 (t, 2H, CH₂), 4.04 (t, 1H, Si—H), 4.25 (t, 2H, O—CH₂), 6.70-6.75 (m, 2H, Ar—H), 7.33 (d, 1H, Ar—H). ¹³C-NMR (CDCl₃, 100 MHz): δ=11.4 (CH), 19.0 (CH₃), 19.3 (CH₃), 23.6 (CH₃), 34.5 (CH₂), 62.8 (O—CH₂), 110.9 (Ar—CH), 116.3 (Ar—CH), 125.2 (Ar—C), 137.7 (Ar—CH), 146.2 (Ar—C), 159.4 (Ar—C), 177.1 (C═O).

### 3-(4-(Di-iso-propylsilyl)-3,5-dimethylphenoxy)propanoic acid

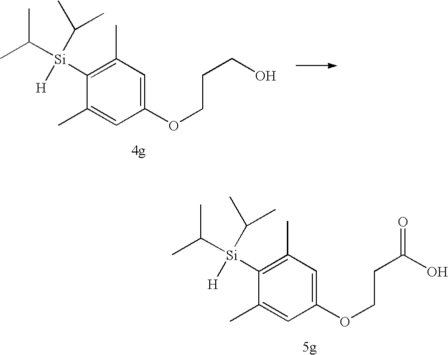

3-(4-(Di-iso-propylsilyl)-3,5-dimethylphenoxy)propan-1-ol (2.00 mmol, 589 mg) was dissolved in acetone (12 ml) and cooled to 0° C. (ice bath). Jones reagent (8M, 6.0 eq., 12.0 mmol, 1.50 ml) was added slowly drop by drop. The reaction mixture was stirred for 15 min at 0° C., then quenched with water and extracted with ethyl acetate. The combined organic extracts were washed with water (3×) and brine, dried over Na₂SO₄ and solvents were removed. The residue was purified by column chromatography (pentane/ethyl acetate/acetic acid 90:9:1) to give 3-(4-(di-iso-propylsilyl)-3,5-dimethylphenoxy)propanoic acid as a white solid in 72% yield (447 mg).

¹H-NMR (CDCl₃, 400 MHz): δ=0.93 (d, 6H, 2×CH₃), 1.16 (d, 6H, 2×CH₃), 1.25-1.37 (m, 2H, 2×CH), 2.43 (s, 6H, 2×CH₃), 2.84 (t, 2H, CH₂), 4.14 (t, 1H, Si—H), 4.24 (t, 2H, O—CH₂), 6.58 (s, 2H, Ar—H), 9.72 (br s, 1H, CO₂H). ¹³C-NMR (CDCl₃, 100 MHz): δ=12.7 (CH), 19.9 (CH₃), 20.4 (CH₃), 24.9 (CH₃), 34.6 (CH₂), 62.5 (O—CH₂), 113.8 (Ar—CH), 125.2 (Ar—C), 146.4 (Ar—C), 158.9 (Ar—C), 177.5 (C═O).

### 2-(4-(Di-tert-butylsilyl)phenyl)acetic acid

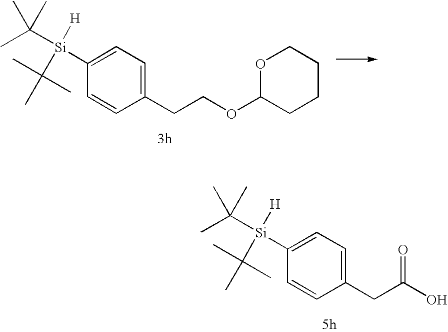

Di-tert-butyl(4-(2-(tetrahydro-2H-pyran-2-yloxy)ethyl)phenyl)silane (4.00 mmol, 1.39 g) was dissolved in ethanol (50 ml). p-Toluenesulfonic acid (1.0 eq, 4.00 mmol, 689 mg) was added and the reaction mixture stirred for 4 h at room temperature. Then it was poured into diluted NaHCO₃ solution and the aqueous phase extracted with ethyl acetate. The combined organic extracts were washed with water and brine, dried over Na₂SO₄ and solvents were removed. The residue was dissolved in acetone (25 ml) and cooled to 0° C. (ice bath). Jones reagent (8M, 6.0 eq., 24.0 mmol, 3.0 ml) was added slowly drop by drop. The reaction mixture was stirred for 15 min at 0° C., then quenched with water and extracted with ethyl acetate. The combined organic extracts were washed with water (3×) and brine, dried over Na₂SO₄ and solvents were removed. The residue was purified by column chromatography (pentane/ethyl acetate/acetic acid 90:9:1) to give 2-(4-(di-tert-butylsilyl)phenyl)acetic acid as a white solid in 80% yield (894 mg).

¹H-NMR (CDCl₃, 400 MHz): δ=1.04 (s, 18H, Si(tBu)₂), 3.65 (s, 2H, CH₂), 3.85 (s, 1H, Si—H), 7.27 (d, 2H, ³J_{H—H}=7.8 Hz, Ar—H), 7.54 (d, ³J_{H—H}=8.1 Hz, 2H, Ar—H). ¹³C-NMR (CDCl₃, 100 MHz): δ=19.2 (Si—C), 29.1 (CH₃), 41.2 (CH₂), 128.6 (Ar—CH), 134.0 (Ar—C), 134.7 (Ar—C), 136.2 (Ar—CH), 177.7 (CO₂H). ²⁹Si-NMR (CDCl₃, 79 MHz): δ=12.9 (¹J_{Si—H}=185 MHz). MS (ESI negative): 277.03 [M−H]⁻. HR-ESI-MS: 233.1735 [M−H]⁻-CO₂ (calc. for C₁₅H₂₅Si: 233.1726).

### 4-Diisobutylsilanyl-benzoic acid

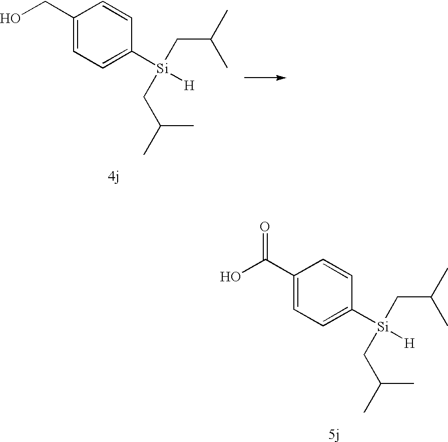

¹H-NMR (CDCl₃): δ=8.07 (2H), 7.67 (2H), 4.43 (1H), 1.75 (2H), 0.94 (6H), 0.93 (6H), 0.86 (4H) ppm.

### (4-Diisobutylsilanyl-phenyl)-acetic acid

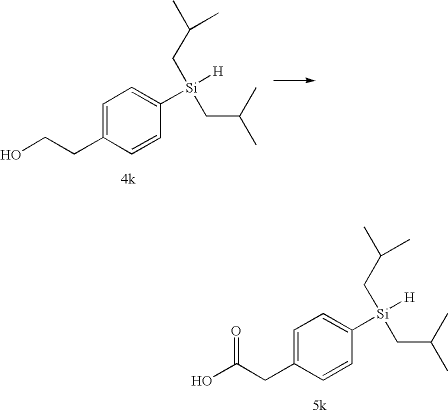

¹H-NMR (CDCl₃): δ=7.50 (2H), 7.27 (2H), 4.37 (1H), 3.65 (2H), 1.74 (2H), 0.94 (6H), 0.93 (6H), 0.82 (4H) ppm.

### Part E

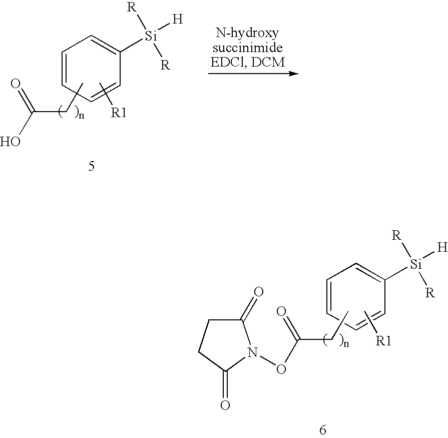

### General Method

To a solution of 600 μmol 5 in 6 ml dichloro-methane were added 76 mg N-hydroxy-succinimide, 126 mg (3-Dimethylamino-propyl)-ethyl-carbodiimide hydrochloride and the mixture was stirred for 16 hours at 23° C. After addition of water and extraction with dichloro-methane the combined organic extracts were dried over sodium sulfate. After filtration and solvent evaporation the crude product was purified by chromatography on silica gel to give 73-99% of 6.

### 4-Diisopropylsilanyl-benzoic acid 2,5-dioxo-pyrrolidin-1-yl ester

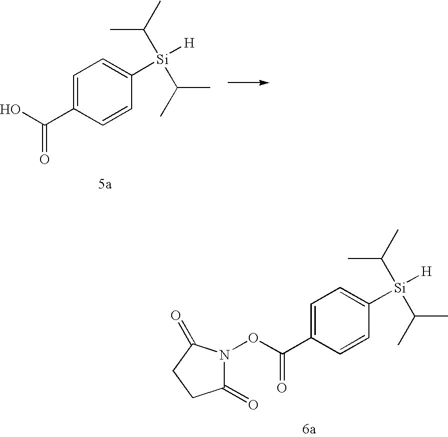

¹H-NMR (CDCl₃): δ=8.09 (2H), 7.67 (2H), 3.99 (1H), 2.91 (4H), 1.27 (2H), 1.07 (6H), 0.98 (6H) ppm.

### (4-Diisopropylsilanyl-phenyl)-acetic acid 2,5-dioxo-pyrrolidin-1-yl ester

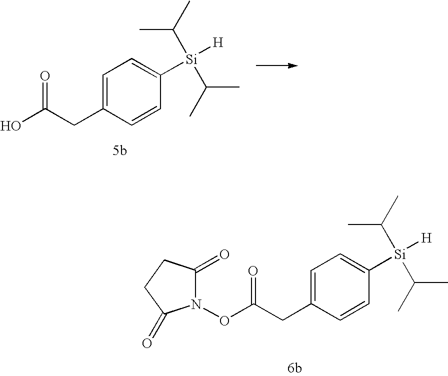

¹H-NMR (CDCl₃): δ=7.51 (2H), 7.32 (2H), 3.93 (3H), 2.83 (4H), 1.22 (2H), 1.06 (6H), 0.98 (6H) ppm.

### 3-(4-Diisopropylsilanyl-phenyl)-propionic acid 2,5-dioxo-pyrrolidin-1-yl ester

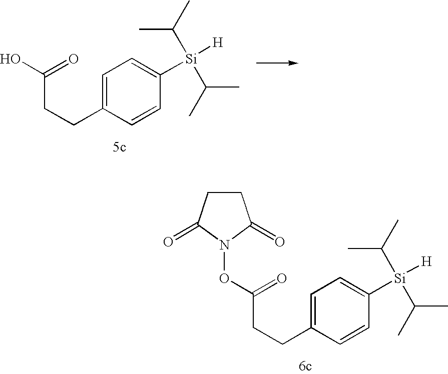

¹H-NMR (CDCl₃): δ=7.46 (2H), 7.21 (2H), 3.92 (1H), 3.06 (2H), 2.93 (2H), 2.85 (4H), 1.21 (2H), 1.06 (6H), 0.98 (6H) ppm.

### (3-Diisopropylsilanyl-phenyl)-acetic acid 2,5-dioxo-pyrrolidin-1-yl ester

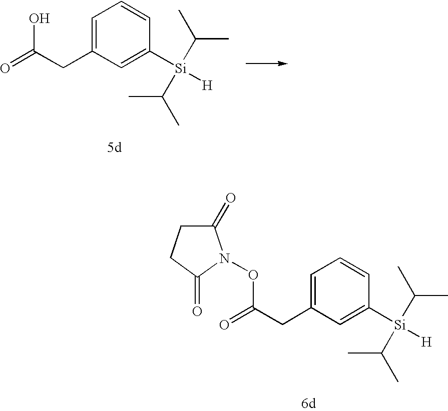

¹H-NMR (CDCl₃): δ=7.46 (2H), 7.35 (2H), 3.94 (3H), 2.83 (4H), 1.22 (2H), 1.06 (6H), 0.98 (6H) ppm.

### 3-(3-Diisopropylsilanyl-phenyl)-propionic acid 2,5-dioxo-pyrrolidin-1-yl ester

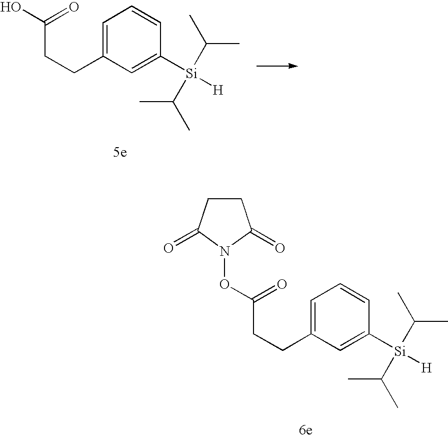

¹H-NMR (CDCl₃): δ=7.38 (1H), 7.35 (1H), 7.30 (1H), 7.23 (1H), 3.92 (1H), 3.05 (2H), 2.92 (2H), 2.85 (4H), 1.22 (2H), 1.06 (6H), 0.98 (6H) ppm.

### 4-Diisobutylsilanyl-benzoic acid 2,5-dioxo-pyrrolidin-1-yl ester

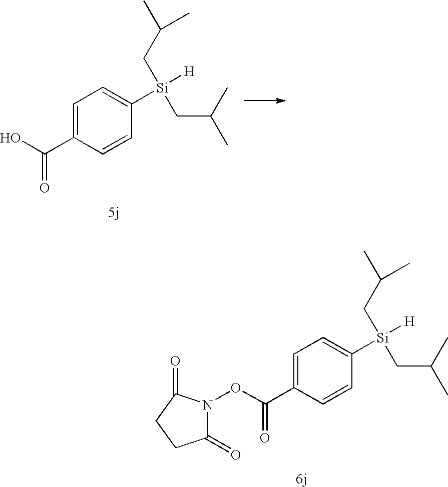

¹H-NMR (CDCl₃): δ=8.08 (2H), 7.69 (2H), 4.41 (1H), 2.91 (4H), 1.73 (2H), 0.93 (6H), 0.91 (6H), 0.86 (4H) ppm.

### (4-Diisobutylsilanyl-phenyl)-acetic acid 2,5-dioxo-pyrrolidin-1-yl ester

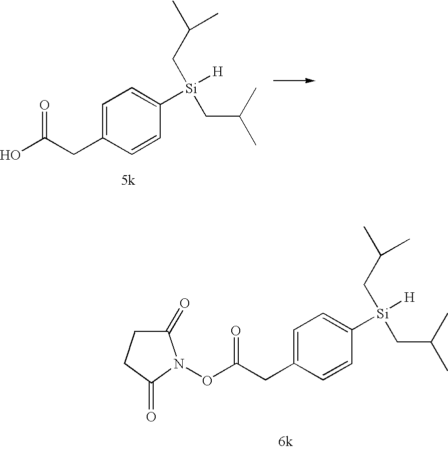

¹H-NMR (CDCl₃): δ=7.53 (2H), 7.32 (2H), 4.38 (1H), 3.93 (2H), 2.83 (4H), 1.74 (2H), 0.93 (6H), 0.92 (6H), 0.82 (4H) ppm.

### Part F1

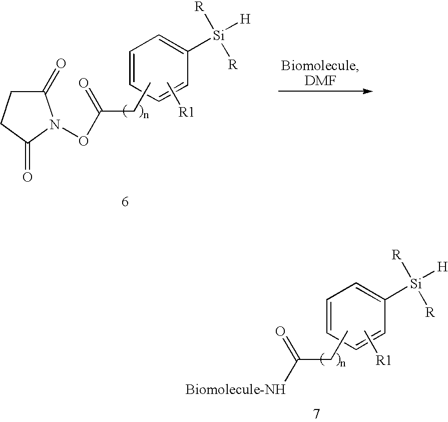

### General Method

0.05 mmol biomolecule or resin bound protected peptide suspended in 2 ml DMF was treated with 0.15 mmol silane active ester 6 for 12 h. The reaction mixture was filtered and in case of the resin bound product, the resin was washed with DMF and dichloromethane. After cleavage from the resin with 1 ml of a mixture of 85% TFA, 5% water, 5% phenol, and 5% triisopropylsilane, the product was precipitated with MTBE and purified by HPLC to give 12% of 7. Reaction mixtures with soluble biomolecules were evaporated and the residue was purified by column chromatography.

### 4-(Diisopropylsilanyl)-phenyl)-acetyl-Val-βAla-Phe-Gly-NH₂

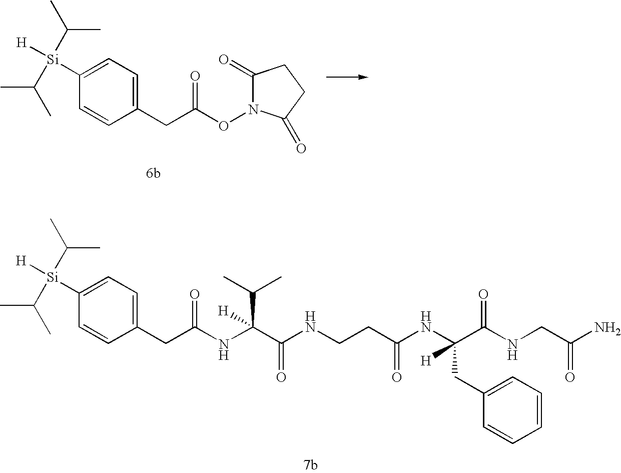

MS (ES+): 624.27

### Part F2

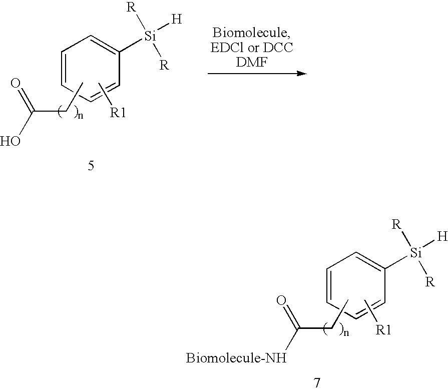

### General Method

0.05 mmol biomolecule or resin bound protected peptide suspended in 2 ml DMF was treated with 0.15 mmol silane acid 5, 57 mg (0.15 mmol) HBTU, 23 mg (0.15 mmol) HOBT and 26 μl (0.15 mmol) diisopropyl ethyl amine for 12 h. The reaction mixture was filtered and the resin was washed with DMF and dichloromethane. After cleavage from the resin with 1 ml of a mixture of 85% TFA, 5% water, 5% phenol, and 5% triisopropylsilane, the product was precipitated with MTBE and purified by HPLC to give 6-61% of 7.

### 4-(Diisopropylsilanyl)-phenyl)-acetyl-Val-βAla-Phe-Gly-NH₂

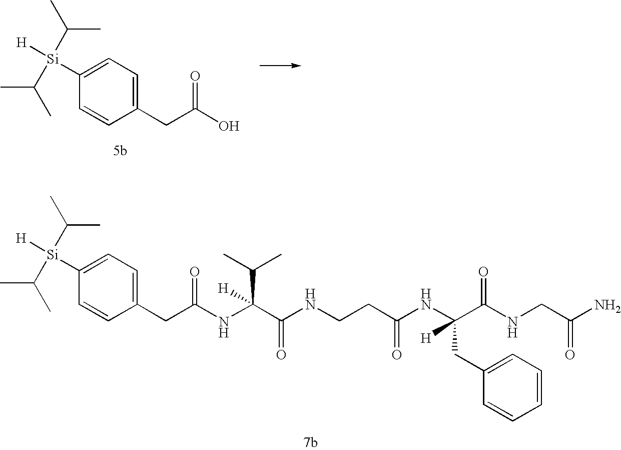

MS (ES+): 624.27

### 4-(Di-tert-butylsilanyl)-phenylacetyl-Val-βAla-Phe-Gly-NH₂

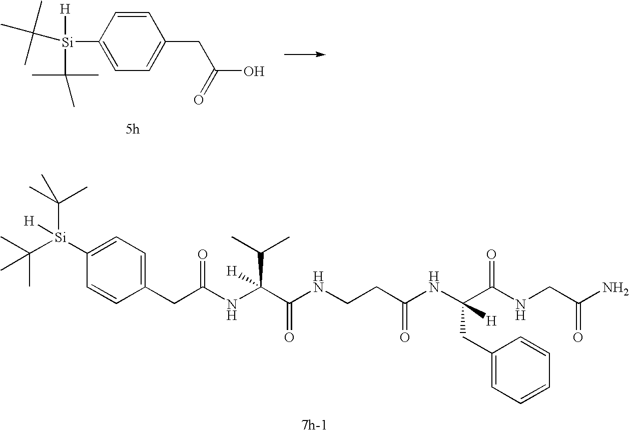

MS (ES+): 652.00

### 4-(Di-tert-Butylsilanyl)-phenylacetyl-Ava-Gln-Trp-Ala-Val-NMeGly-His(3Me)-[4-(R)-amino-5-(S)-methylheptanoyl]-Cpa-NH₂

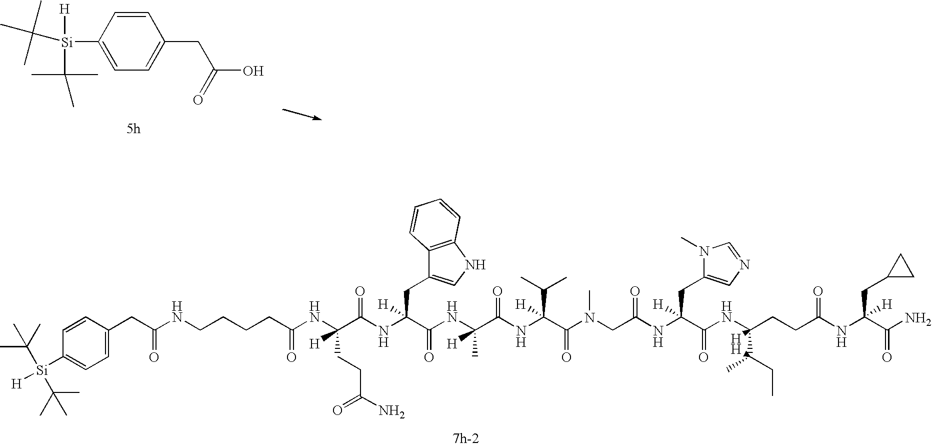

MS (ES+): 1335.89

### 4-(Di-tert-butylsilanyl)-phenylacetyl-Arg-Ava-Gln-Trp-Ala-Val-NMeGly-His-Sta-Leu-NH₂

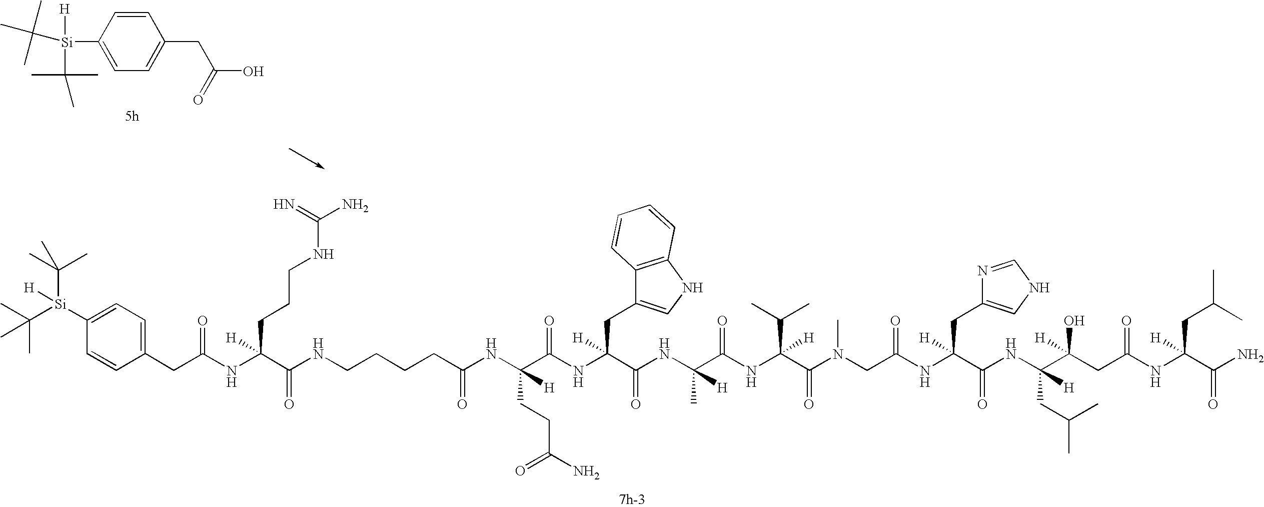

MS (ES+): 1495.94

### 4-(Di-tert-butylsilanyl)-phenylacetyl-Arg-Ava-Gln-Trp-Ala-Val-Gly-His(3Me)-Sta-Leu-NH₂

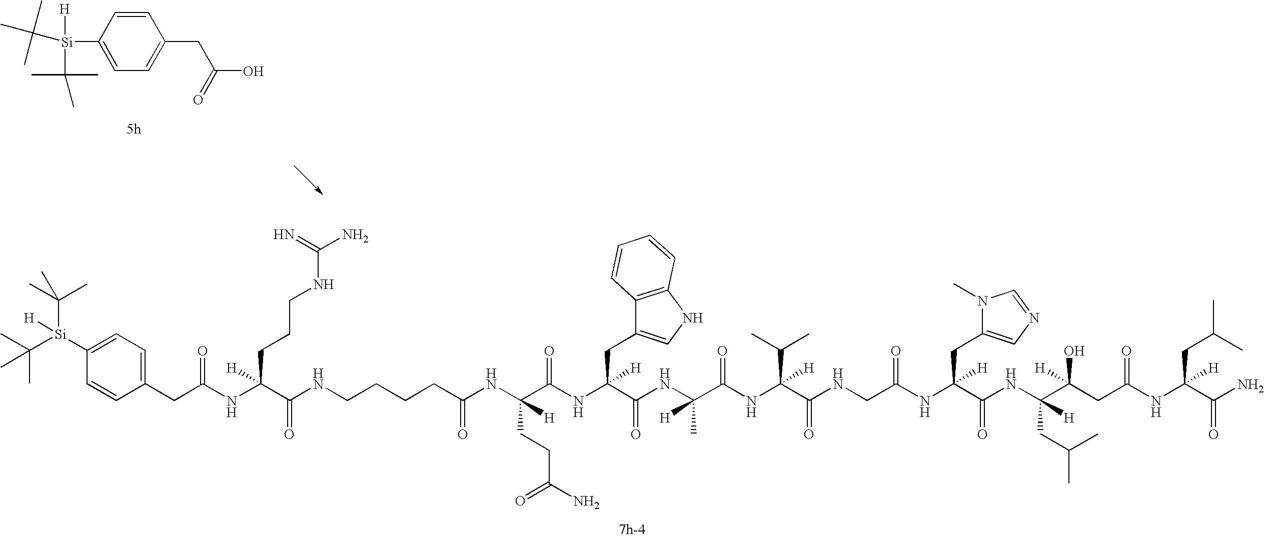

MS (ES+): 1495.94

### 2-(4-(di-tert-butylsilyl)phenyl)-N-(3-(3-(4-hydroxy-5-(hydroxymethyl)tetrahydrofuran-2-yl)-5-methyl-2,6-dioxo-2,3-dihydropyrimidin-1(6H)-yl)propyl)acetamide

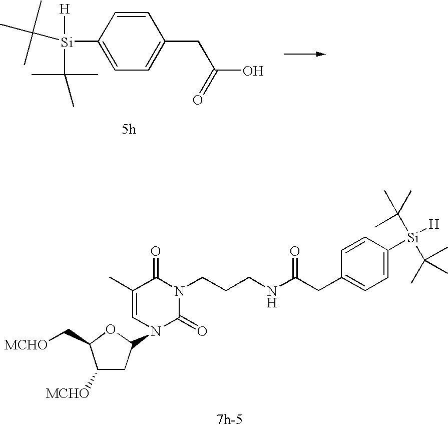

¹H-NMR (CDCl₃): δ=7.57 (2H), 7.50 (1H), 7.33 (2H), 6.68 (1H), 6.25 (1H), 4.52 (1H), 3.88 (3H), 3.60 (2H), 3.48 (2H), 3.18 (3H), 3.17 (3H), 3.13 (1H), 2.27 (1H), 2.09 (1H), 1.89 (3H), 1.81-1.42 (20H), 1.06 (18H) ppm.

### N-benzyl-2-(4-(di-tert-butylsilyl)phenyl)acetamide

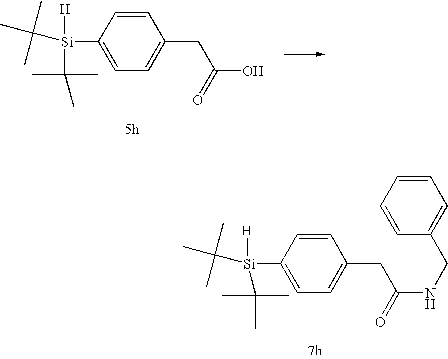

(Di-tert-butylsilyl)phenyl)acetic acid (0.800 mmol, 223 mg), was dissolved in dry dichloromethane (8.0 ml). Benzyl amine (1.3 eq, 1.04 mmol, 0.11 ml) and N-(3-dimethylaminopropyl)-N′-ethylcarbodiimide hydrochloride (1.4 eq, 1.12 mmol, 215 mg) were added. The reaction mixture was stirred overnight at room temperature, then quenched with water and extracted with dichloromethane. The combined organic extracts were washed with water and brine, dried over Na₂SO₄ and solvents were removed. The residue was purified by column chromatography (pentane/ethyl acetate 4:1) to give 194 mg of N-benzyl-2-(4-(di-tert-butylsilyl)phenyl)acetamide (66%) as a white solid.

¹H-NMR (CDCl₃, 400 MHz): δ 1.03 (s, 18H, Si(tBu)₂), 3.63 (s, 2H, CH₂), 3.85 (s, 1H, Si—H), 4.43 (d, 2H, ³J_{H—H}=5.8 Hz, N—CH₂), 5.66 (br s, 1H, NH), 7.15 (d, 2H, ³J_{H—H}=7.8 Hz, Ar—H), 7.22-7.30 (m, 5H, Ar—H), 7.55 (d, 2H, ³J_{H—H}=7.8 Hz, Ar—H). ¹³C-NMR (CDCl₃, 100 MHz): δ=19.1 (Si—C), 29.0 (CH₃), 43.8 (CH₂), 44.1 (CH₂), 127.5 (Ar—CH), 127.6 (Ar—CH), 128.7 (Ar—CH), 128.8 (Ar—CH), 134.8 (Ar—C), 135.6 (Ar—C), 136.6 (Ar—CH), 138.2 (Ar—C), 170.9 (CONH). ²⁹Si-NMR (CDCl₃, 79 MHz): δ=12.9 (¹J_{Si—H}=186 MHz). MS (ESI positive): 368.2 [M+H]⁺. HR-EI-MS: 367.2325 [M]⁺ (calc. for C₂₃H₃₃NOSi: 367.2331).

### N-benzyl-3-(4-(di-iso-propylsilyl)-3-methylphenoxy)propanamide

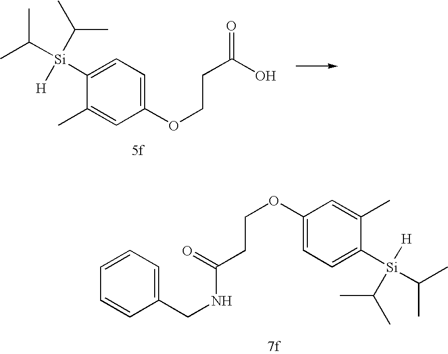

3-(4-(Di-iso-propylsilyl)-3-methylphenoxy)propanoic acid (0.80 mol, 236 mg) was dissolved in dry dichloromethane (8 ml). N-(3-dimethylaminopropyl)-N′-ethylcarbodiimide hydrochloride (1.4 eq, 1.12 mmol, 215 mg) and benzyl amine (1.3 eq, 1.04 mmol, 0.11 ml) were added. The reaction mixture was stirred overnight at room temperature, then quenched with water and extracted with dichloromethane. The combined organic extracts were washed with water and brine, dried over Na₂SO₄ and solvents were removed. The residue was purified by column chromatography (pentane/ethyl acetate 3:2) to give 245 mg of N-benzyl-3-(4-(di-iso-propylsilyl)-3-methylphenoxy)propanamide (80%) as a white solid.

¹H-NMR (CDCl₃, 400 MHz): δ=0.96 (d, 6H, 2×CH₃), 1.07 (d, 6H, 2×CH₃), 1.18-1.30 (m, 2H, 2×CH), 2.40 (s, 3H, CH₃), 2.70 (t, 2H, CH₂), 4.04 (t, 1H, Si—H), 4.29 (t, 2H, O—CH₂), 4.49 (d, 2H, N—CH₂), 6.20 (br s, 1H, NH), 6.67-6.71 (m, 2H, Ar—H), 7.25-7.35 (m, 6H, Ar—H).

¹³C-NMR (CDCl₃, 100 MHz): δ=11.8 (CH), 19.5 (CH₃), 19.7 (CH₃), 24.0 (CH₃), 37.4 (CH₂), 44.2 (N—CH₂), 64.4 (O—CH₂), 111.3 (Ar—CH), 116.8 (Ar—CH), 125.8 (Ar—C), 128.1 (Ar—CH), 128.3 (Ar—CH), 129.3 (Ar—CH), 138.1 (Ar—CH), 138.7 (Ar—C), 146.7 (Ar—C), 159.7 (Ar—C), 171.1 (C═O).

### N-benzyl-3-(4-(di-iso-propylsilyl)-3,5-dimethylphenoxy)propanamide

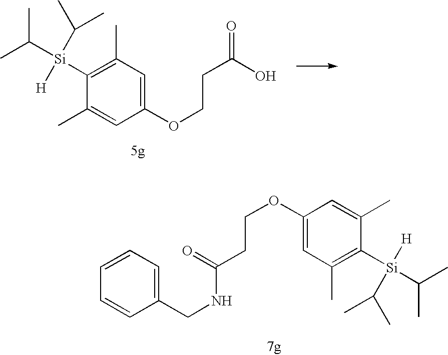

3-(4-(Di-iso-propylsilyl)-3,5-dimethylphenoxy)propanoic acid (0.80 mol, 247 mg) was dissolved in dry dichloromethane (8 ml). N-(3-dimethylaminopropyl)-N′-ethylcarbodiimide hydrochloride (1.4 eq, 1.12 mmol, 215 mg) and benzyl amine (1.3 eq, 1.04 mmol, 0.11 ml) were added. The reaction mixture was stirred overnight at room temperature, then quenched with water and extracted with dichloromethane. The combined organic extracts were washed with water and brine, dried over Na₂SO₄ and solvents were removed. The residue was purified by column chromatography (pentane/ethyl acetate 2:1) to give 212 mg of N-benzyl-3-(4-(di-iso-propylsilyl)-3,5-dimethylphenoxy)propanamide (67%) as a white solid.

¹H-NMR (CDCl₃, 400 MHz): δ=0.91 (d, 6H, 2×CH₃), 1.14 (d, 6H, 2×CH₃), 1.23-1.33 (m, 2H, 2×CH), 2.40 (s, 6H, 2×CH₃), 2.69 (t, 2H, CH₂), 4.11 (t, 1H, Si—H), 4.27 (t, 2H, O—CH₂), 4.48 (d, 2H, N—CH₂), 6.21 (br s, 1H, NH), 6.52 (s, 2H, Ar—H), 7.25-7.35 (m, 5H, Ar—H). ¹³C-NMR (CDCl₃, 100 MHz): δ=12.7 (CH), 19.9 (CH₃), 20.4 (CH₃), 25.0 (CH₃), 37.0 (CH₂), 43.8 (N—CH₂), 63.8 (O—CH₂), 113.8 (Ar—CH), 125.5 (Ar—C), 127.6 (Ar—CH), 127.8 (Ar—CH), 128.8 (Ar—CH), 138.3 (Ar—C), 146.5 (Ar—C), 158.7 (Ar—C), 170.7 (C═O).

### Part G

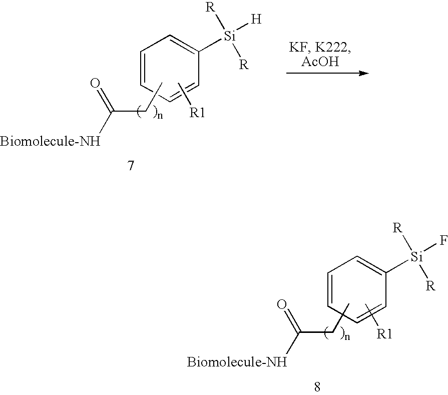

### General Method

2 mg (3.21 μM) silane 7 were solved in 320 μl THF and treated with 0.19 mg (3.21 μM) KF, 1.21 mg (3.21 μM) K222, 0.44 mg (3.21 μM) K₂CO₃ and 0.55 μl acetic acid. The reaction mixture was stirred at 50° 70° C. for 30-60 min and followed by HPLC to give 50-90% conversion to 8.

### [4-(fluoro-diisopropyl-silanyl)-phenyl]-acetic acid-Val-βAla-Phe-Gly-amide

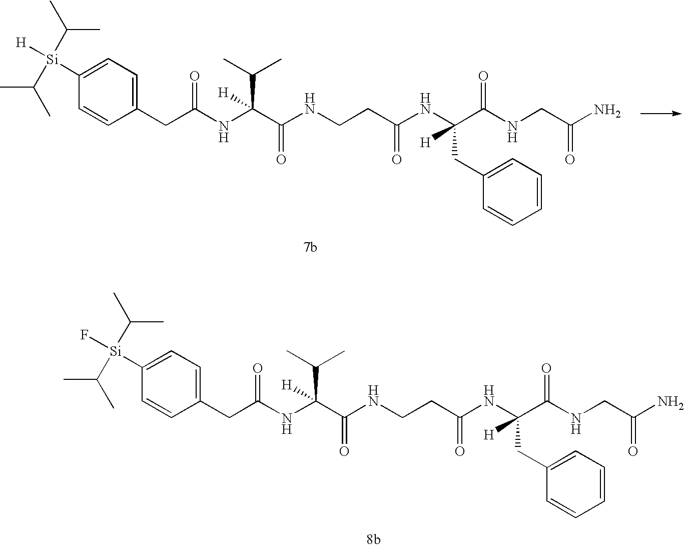

MS (ES+): 642.12

### 4-(Fluordi-tert-butylsilyl)-phenylacetyl-Ava-Gln-Trp-Ala-Val-NMeGly-His(3Me)-[4-(R)-amino-5-(S)-methylheptanoyl]-Cpa-NH₂

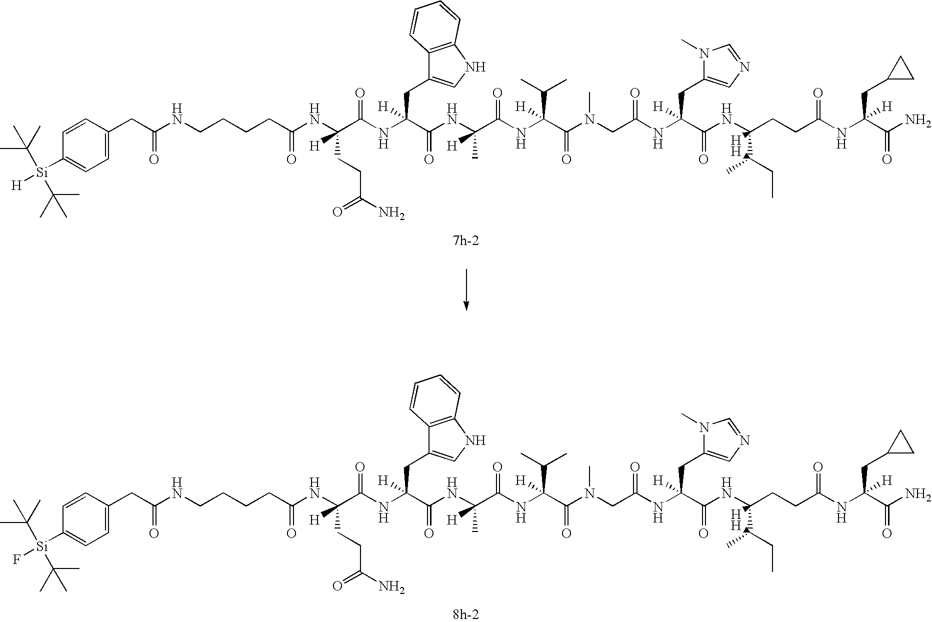

MS (ES+): 1353.78

### 4-(Fluor-di-tert-butylsilanyl)-phenylacetyl-Arg-Ava-Gln-Trp-Ala-Val-NMeGly-His-Sta-Leu-NH₂

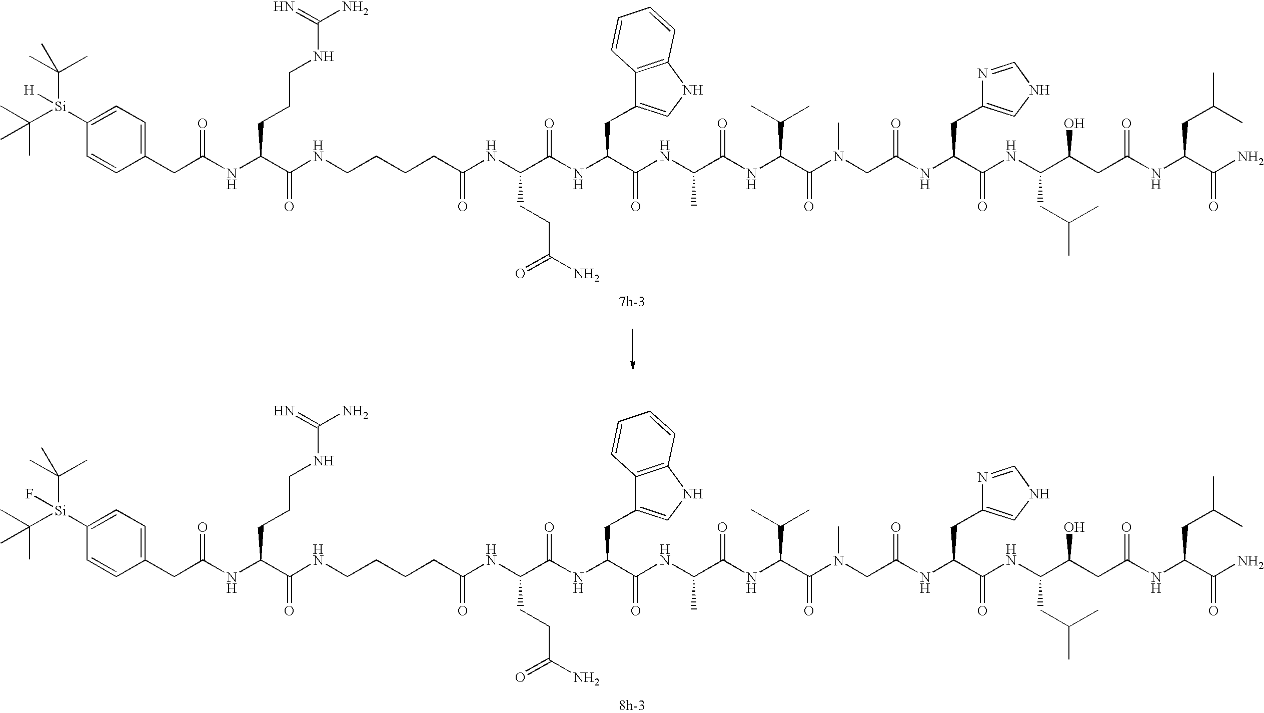

MS (ES+): 1513.92

### 4-(Fluor-di-tert-butylsilanyl)-phenylacetyl-Arg-Ava-Gln-Trp-Ala-Val-Gly-His(3Me)-Sta-Leu-NH₂

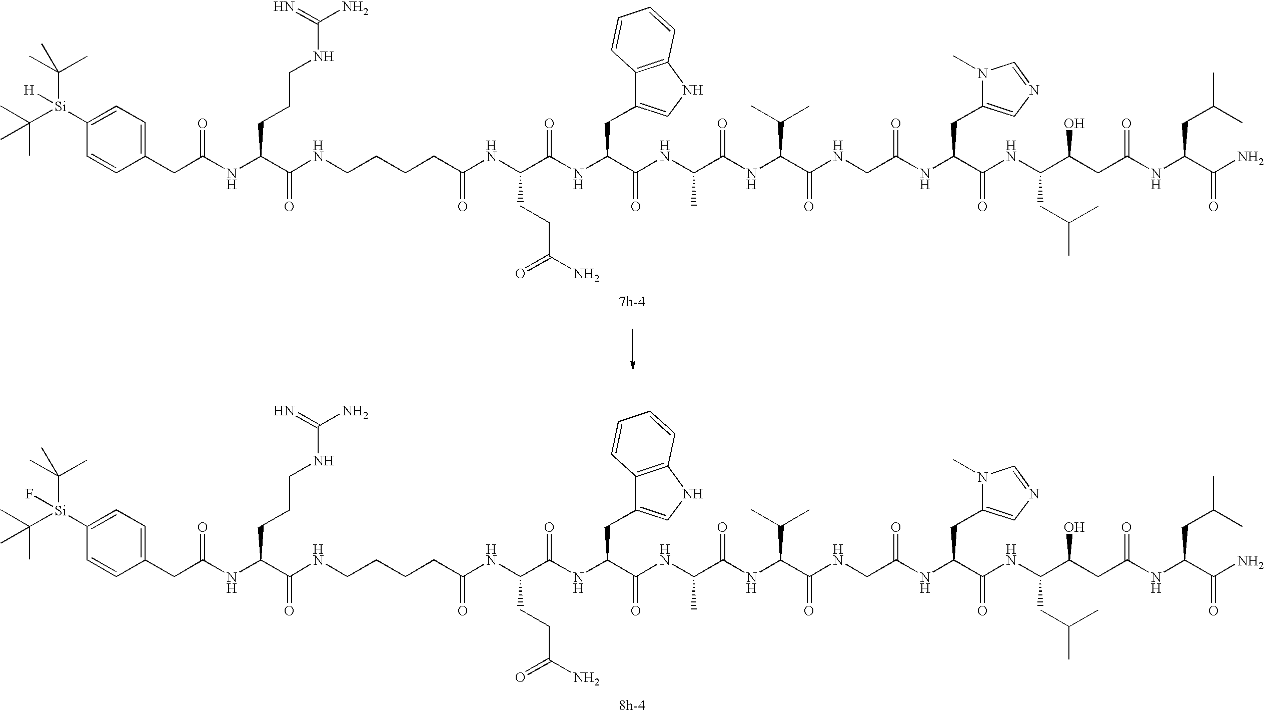

MS (ES+): 1513.92

### 2-[4-(Di-tert-butyl-fluoro-silanyl)-phenyl]-N-{3-[(R)-3-((4S,5R)-4-hydroxy-5-hydroxy-methyl-tetrahydro-furan-2-yl)-5-methyl-2,6-dioxo-3,6-dihydro-2H-pyrimidin-1-yl]-propyl}-acetamide

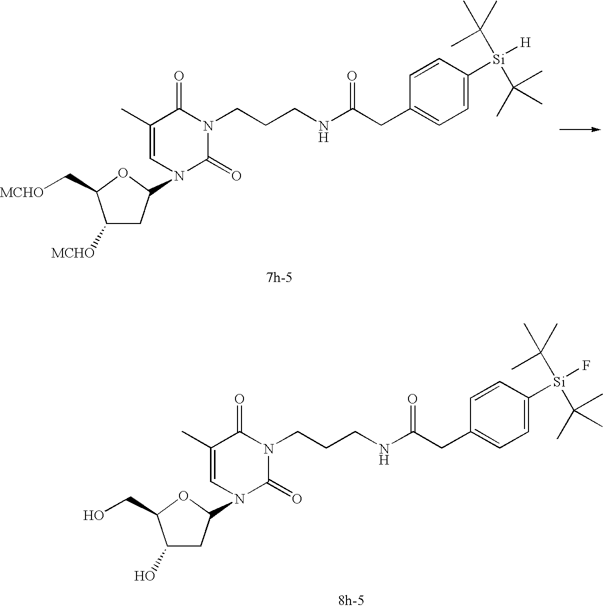

¹H-NMR (CDCl₃): δ=7.58 (3H), 7.34 (2H), 6.67 (1H), 6.21 (1H), 4.35 (1H), 3.84 (3H), 3.69 (2H), 3.47 (2H), 3.31 (1H), 3.10 (3H), 2.15 (4H), 1.84 (3H), 1.70 (2H), 1.04 (18H) ppm.

¹⁹F-NMR (CDCl₃): δ=−187.27

### N-Benzyl-2-(4-(di-tert-butylfluorosilyl)phenyl)acetamide

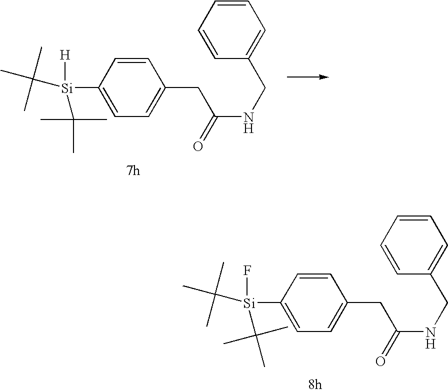

N-Benzyl-2-(4-(di-tert-butylsilyl)phenyl)acetamide (0.300 mmol, 110 mg) was dissolved in dry THF (3.0 ml). Acetic acid (3.0 eq, 0.900 mmol, 52 μl), Kryptofix® 2.2.2 (1.5 eq, 0.450 mmol, 169 mg) and then spray-dried potassium fluoride (1.5 eq, 0.450 mmol, 26.1 mg) were added. The reaction mixture was heated under reflux for 4 h. Solvents were removed and the residue was purified by column chromatography (dichloromethane/methanol 99:1) to give 113 mg of N-benzyl-2-(4-(di-tert-butylfluorosilyl)phenyl)acetamide (98%) as a white solid.

¹H-NMR (CDCl₃, 400 MHz): δ=1.05 (d, 18H, ⁴J_{H—F}=1.0 Hz, S (tBu)₂), 3.63 (s, 2H, CH₂), 4.43 (d, 2H, ³J_{H—H}=5.6 Hz, N—CH₂), 5.64 (br s, 1H, NH), 7.16 (d, 2H, ³J_{H—H}=8.1 Hz, Ar—H), 7.24-7.31 (m, 5H, Ar—H), 7.59 (d, 2H, ³J_{H—H}=7.8 Hz, Ar—H). ¹³C-NMR (CDCl₃, 100 MHz): δ=20.4 (d, ²J_{C—F}=12.4 Hz, Si—C), 27.5 (CH₃), 43.8 (CH₂), 44.1 (CH₂), 127.6 (Ar—CH), 127.6 (Ar—CH), 128.8 (Ar—CH), 128.8 (Ar—CH), 132.9 (d, ²J_{C—F}=13.9 Hz, Ar—C), 134.8 (Ar—CH), 136.4 (Ar—C), 138.2 (Ar—C), 170.7 (CONH). ¹⁹F-NMR (CDCl₃, 376 MHz): δ=−188.8. ²⁹Si-NMR (CDCl₃, 79 MHz): δ 13.9 (¹J_{Si—F}=298 MHz). MS (ESI positive): 386.2 [M+H]. HR-EI-MS: 385.2228 [M]⁺ (calc. for C₂₃H₃₂FNOSi: 385.2237).

### N-benzyl-3-(4-(fluorodi-iso-propylsilyl)-3-methylphenoxy)propanamide

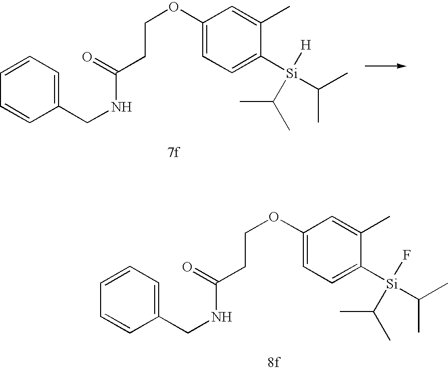

N-benzyl-3-(4-(di-iso-propylsilyl)-3-methylphenoxy)propanamide (0.300 mmol, 115 mg) was dissolved in dry THF (3.0 ml). Acetic acid (3.0 eq, 0.900 mmol, 52 μl), Kryptofix® 2.2.2 (1.5 eq, 0.450 mmol, 169 mg) and then spray-dried potassium fluoride (1.5 eq, 0.450 mmol, 26.1 mg) were added. The reaction mixture was heated under reflux for 4 h. Solvents were removed and the residue was purified by column chromatography (pentane/ethyl acetate 3:2) to give 56.1 mg of N-benzyl-3-(4-(fluorodi-iso-propylsilyl)-3-methylphenoxy)propanamide (47%) as a white solid.

¹H-NMR (CDCl₃, 400 MHz): δ=0.98 (d, 6H, 2×CH₃), 1.09 (d, 6H, 2×CH₃), 1.22-1.33 (m, 2H, 2×CH), 2.40 (d, 3H, CH₃), 2.70 (t, 2H, CH₂), 4.30 (t, 2H, O—CH₂), 4.49 (d, 2H, N—CH₂), 6.14 (br s, 1H, NH), 6.70-6.73 (m, 2H, Ar—H), 7.27-7.38 (m, 6H, Ar—H). ¹³C-NMR (CDCl₃, 100 MHz): δ=13.3 (d, CH), 17.0 (CH₃), 17.2 (d, CH₃), 23.3 (d, CH₃), 37.0 (CH₂), 43.8 (N—CH₂), 64.0 (O—CH₂), 110.9 (Ar—CH), 116.7 (Ar—CH), 123.0 (d, Ar—C), 127.7 (Ar—CH), 127.8 (Ar—CH), 128.9 (Ar—CH), 138.4 (d, Ar—CH), 138.3 (Ar—C), 145.9 (Ar—C), 159.8 (Ar—C), 170.5 (C═O). ¹⁹F-NMR (CDCl₃, 376 MHz): δ=−183.6.

### N-benzyl-3-(4-(fluorodi-iso-propylsilyl)-3,5-dimethylphenoxy)propanamide

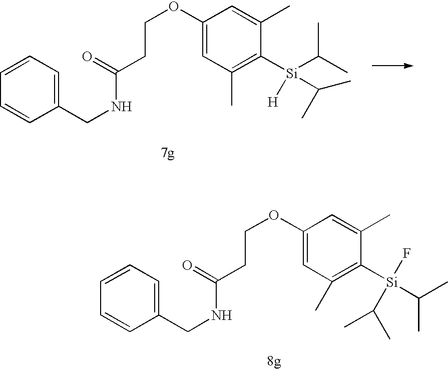

N-benzyl-3-(4-(di-iso-propylsilyl)-3,5-dimethylphenoxy)propanamide (0.20 mmol, 79.5 mg) was dissolved in dry THF (2.0 ml). Acetic acid (5.0 eq, 1.00 mmol, 57 μl), Kryptofix® 2.2.2 (2.5 eq, 0.50 mmol, 188 mg) and then spray-dried potassium fluoride (2.5 eq, 0.50 mmol, 29.1 mg) were added. The reaction mixture was heated under reflux for 16 h. Solvents were removed and the residue was purified by column chromatography (pentane/ethyl acetate 2:1) to give 67.5 mg of N-benzyl-3-(4-(fluorodi-iso-propylsilyl)-3,5-dimethylphenoxy) propanamide (81%) as a white solid.

¹H-NMR (CDCl₃, 400 MHz): δ=0.96 (d, 6H, 2×CH₃), 1.15 (d, 6H, 2×CH₃), 1.23-1.35 (m, 2H, 2×CH), 2.38 (d, 3H, CH₃), 2.69 (t, 2H, CH₂), 4.28 (t, 2H, O—CH₂), 4.49 (d, 2H, N—CH₂), 6.16 (br s, 1H, NH), 6.52 (s, 2H, Ar—H), 7.27-7.34 (m, 5H, Ar—H). ¹³C-NMR (CDCl₃, 100 MHz): δ=15.2 (d, CH), 17.5 (d, CH₃), 17.8 (CH₃), 24.1 (d, CH₃), 37.0 (CH₂), 43.8 (N—CH₂), 63.8 (O—CH₂), 114.3 (Ar—CH), 123.7 (d, Ar—C), 127.6 (Ar—CH), 127.8 (Ar—CH), 128.8 (Ar—CH), 138.3 (Ar—C), 146.4 (Ar—C), 159.2 (Ar—C), 170.6 (C═O). ¹⁹F-NMR (CDCl₃, 376 MHz): δ−176.9.

### BRIEF DESCRIPTION OF DRAWINGS

FIGS. 1A, 1B, 1C, 2A, 2B, 2C, 3A, 3B AND 3C SHOW INTENSITY/RETENTION TIME CURVES FOR VARIOUS PRODUCTS.

### Example II

### Part A

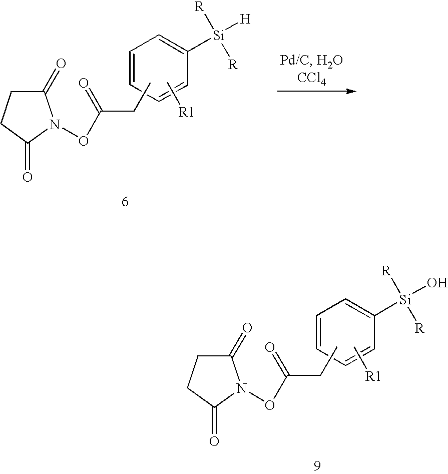

### General Method

To a solution of 399 μmol 6 in 1.68 ml tetrachloromethane was added 7.27 mg palladium (10% on charcoal) and the mixture stirred for 16 hours at 23° C. 72 μl water were added and stirring was continued for additional 75 hours at 23° C. Sodium sulfate was added and after filtration the solvent was evaporated. The crude product was purified by chromatography on silica gel to give 64-84% of 9.

### 4-(Hydroxy-diisopropyl-silanyl)-benzoic acid 2,5-dioxo-pyrrolidin-1-yl ester

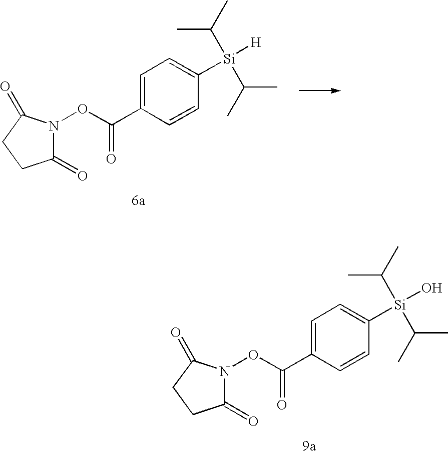

¹H-NMR (CDCl₃): δ=8.11 (2H), 7.71 (2H), 2.92 (4H), 1.87 (1H), 1.24 (2H), 1.05 (6H), 0.96 (6H) ppm.

### [4-(Hydroxy-diisopropyl-silanyl)-phenyl]-acetic acid 2,5-dioxo-pyrrolidin-1-yl ester

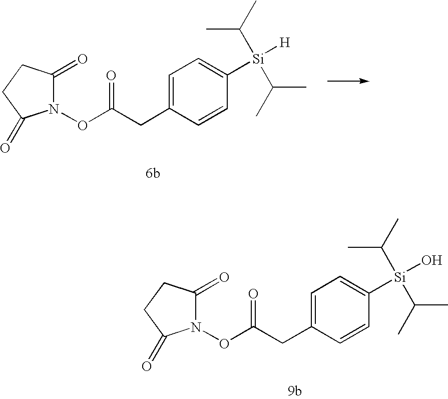

¹H-NMR (CDCl₃): δ=7.55 (2H), 7.35 (2H), 3.94 (2H), 2.83 (4H), 1.80 (1H), 1.21 (2H), 1.05 (6H), 0.97 (6H) ppm.

### 3-[3-(Hydroxy-diisopropyl-silanyl)-phenyl]-propionic acid 2,5-dioxo-pyrrolidin-1-yl ester

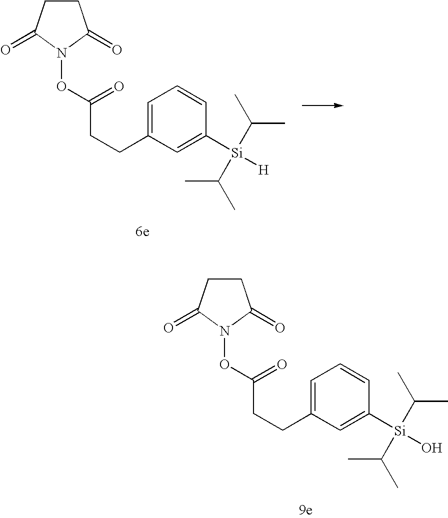

¹H-NMR (CDCl₃): δ=7.45 (1H), 7.41 (1H), 7.31 (1H), 7.25 (1H), 3.09 (2H), 2.93 (2H), 2.84 (4H), 1.21 (2H), 1.06 (6H), 0.98 (6H) ppm.

### 4-(Hydroxy-diisobutyl-silanyl)-benzoic acid 2,5-dioxo-pyrrolidin-1-yl ester

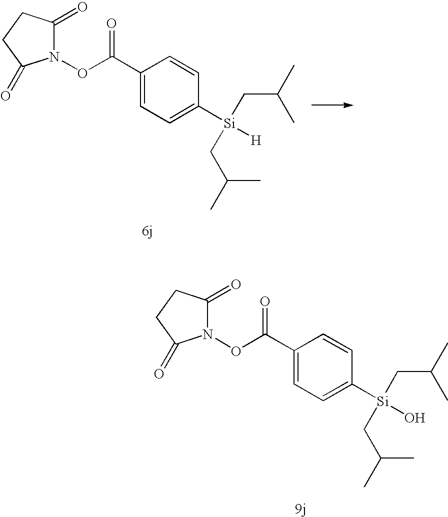

¹H-NMR (CDCl₃): δ=8.11 (2H), 7.73 (2H), 2.91 (4H), 1.79 (2H), 1.60 (1H), 0.92 (6H), 0.89 (6H), 0.86 (4H) ppm.

### [4-(Hydroxy-diisobutyl-silanyl)-phenyl]-acetic acid 2,5-dioxo-pyrrolidin-1-yl ester

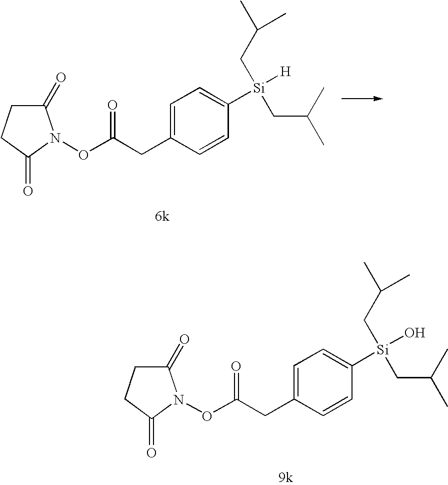

¹H-NMR (CDCl₃): δ=7.57 (2H), 7.34 (2H), 3.94 (2H), 2.83 (4H), 1.82 (2H), 0.93 (6H), 0.91 (6H), 0.84 (4H) ppm.

### 2-(4-(Di-tert-butyl(hydroxy)silyl)phenyl)acetic acid

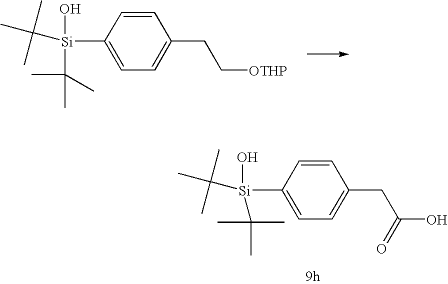

Di-tert-butyl(4-(2-(tetrahydro-2H-pyran-2-yloxy)ethyl)phenyl)silanol (1.00 mmol, 365 mg) was dissolved in ethanol (12 ml). p-Toluenesulfonic acid (1.0 eq, 1.00 mmol, 172.2 mg) was added and the reaction mixture stirred for 4 h at room temperature. Then it was poured into diluted NaHCO₃ solution and the aqueous phase extracted with dichloromethane. The combined organic extracts were washed with water, dried over Na₂SO₄ and solvents were removed. The residue was dissolved in acetone (6 ml) and cooled to 0° C. (ice bath). Jones reagent (8M, 6.0 eq, 6.00 mmol, 0.75 ml) was added slowly drop by drop. The reaction mixture was stirred for 15 min at 0° C., then quenched with water and extracted with ethyl acetate. The combined organic extracts were washed with water (2×) and brine, dried over Na₂SO₄ and solvents were removed. The residue was purified by column chromatography (pentane/ethyl acetate/acetic acid 85:14:1) to give 2-(4-(di-tert-butyl(hydroxy)silyl)phenyl)acetic acid as a white solid in 77% yield (227 mg).

¹H-NMR (CDCl₃, 400 MHz): δ=1.03 (s, 18H, Si(tBu)₂), 3.66 (s, 2H, CH₂), 7.29 (d, 2H, ³J_{H—H}=8.1 Hz, Ar—H), 7.61 (d, 2H, ³J_{H—H}=8.1 Hz, Ar—H). ¹³C-NMR (CDCl₃, 100 MHz): δ=20.5 (Si—C), 28.1 (CH₃), 41.1 (CH₂), 128.5 (Ar—CH), 134.2 (Ar—C), 134.9 (Ar—CH), 135.0 (Ar—C), 177.0 (CO₂H). ²⁹Si-NMR (CDCl₃, 79 MHz): δ=4.0. MS (ESI positive): 294.97 [M+H]⁺. MS (ESI negative): 293.02 [M−H]⁻. HR-ESI-MS: 249.1679 [M−H]—CO₂ (calc. for C₁₅H₂₅OSi: 249.1680).

### Part B

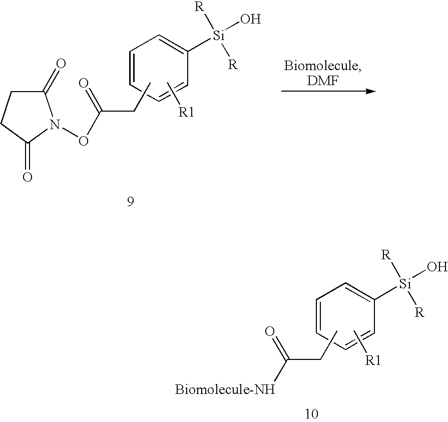

### General Method

0.05 mmol biomolecule or resin bound protected peptide suspended in 2 ml DMF was treated with 0.15 mmol silanol active ester 9 for 12 h. The reaction mixture was filtered and in case of the resin bound product, the resin was washed with DMF and dichloromethane. After cleavage from the resin with 1 ml of a mixture of 85% TFA, 5% water, 5% phenol, and 5% triisopropylsilane, the product was precipitated with MTBE and purified by HPLC to give 12-45% of 10. Reaction mixtures with soluble biomolecules were evaporated and the residue was purified by column chromatography to give 68-80% of 10.

### 4-(Hydroxy-diisopropyl-silanyl)-phenylacetyl-Val-βAla-Phe-Gly-NH₂

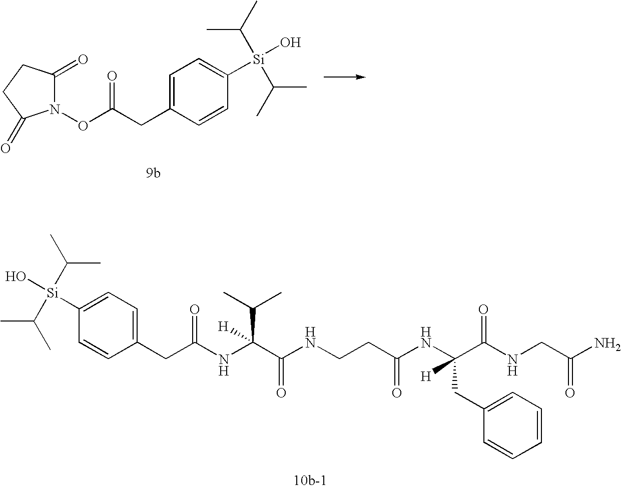

MS (ES+): 640.29

### N-Benzyl-2-[4-(hydroxy-diisopropyl-silanyl)-phenyl]-acetamide

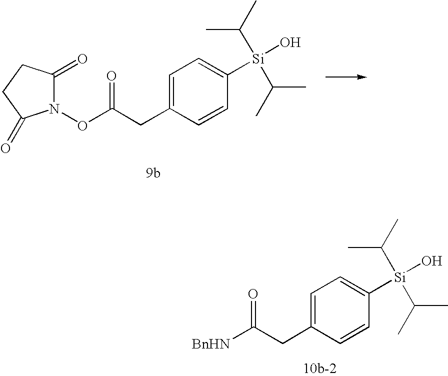

¹H-NMR (400 MHz, CDCl₃): δ=7.55 (d, ³J=8.0, 2H, aryl), 7.30-7.24 (m, 5H, aryl), 7.16 (d, ³J=8.0, 2H, aryl), 5.66 (bs, 1H, NH), 4.43 (d, ³J=5.8, 2H, CH₂—N), 3.63 (s, 2H, CH₂), 1.21 (sept, ³J=7.3 Hz, 2H, Si—CH), 1.05 (d, ³J=7.3 Hz, CH₃—Si, 6H), 0.97 (d, ³J=7.3 Hz, CH₃—Si, 6H) ppm. ¹³C-NMR (100.6 MHz, CDCl₃): δ [ppm]=170.7, 138.0, 135.8, 134.8, 134.6, 128.7, 128.65, 127.4, 43.9, 43.6, 17.2, 16.9, 12.4. ²⁹Si-NMR (79.4 MHz, CDCl₃): δ [ppm]=7.41. IR (KBr)=3280, 2943, 2863, 1648, 1556. MS (ESI+): m/z (%)=356 (M+1, 80%), 151 (34%), 126 (100%), 101 (58%). HRMS calcd for C₂₁H₂₉O₂NSi) 355.1967, found 355.1973.

### 2-[4-(Hydroxy-diisopropyl-silanyl)-phenyl]-N-(3-{(R)-3-[(4S,5R)-4-(1-methoxy-cyclohexyloxy)-5-(1-methoxy-cyclohexyloxymethyl)-tetrahydro-furan-2-yl]-5-methyl-2,6 dioxo-3,6-dihydro-2H-pyrimidin-1-yl}-propyl)-acetamide

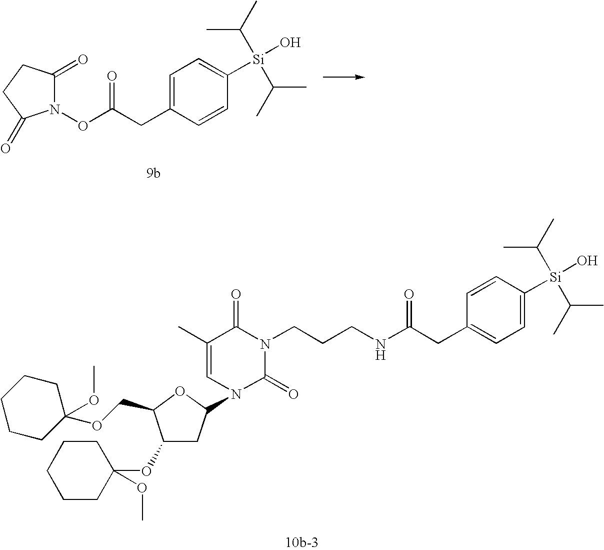

¹H-NMR (400 MHz, MeOD): δ=7.62 (1H), 7.40 (2H), 7.29 (2H), 6.26 (1H), 4.54 (1H), 4.13 (1H), 3.90 (2H), 3.61 (2H), 3.50 (2H), 3.18 (6H), 3.16 (1H), 2.30 (1H), 2.19 (1H), 1.90 (3H), 1.80-1.34 (20H), 1.14 (2H), 1.00 (6H), 0.92 (6H) ppm.

### 3-(Hydroxy-diisopropyl-silanyl)-phenylpropionyl-Val-βAla-Phe-Gly-NH₂

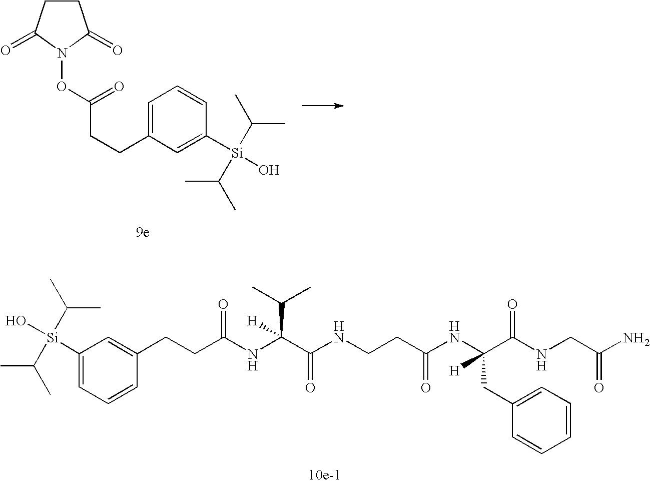

MS (ES+): 654.11

### N-Benzyl-3-[3-(hydroxy-diisopropyl-silanyl)-phenyl]-propionamide

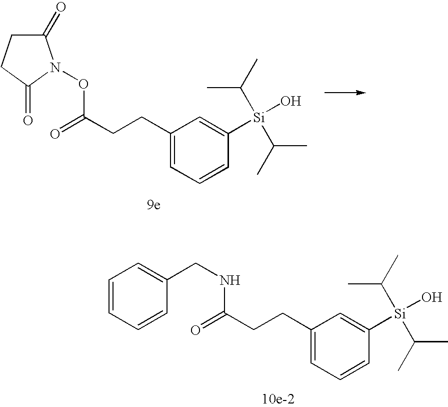

¹H-NMR (400 MHz, CDCl₃): δ=7.45 (m, 2H, aryl), 7.40-7.26 (m, 5H, aryl), 7.22 (m, 2H, aryl), 5.62 (bs, 1H, NH), 4.44 (d, ³J=5.6 Hz, 2H, CH₂N), 3.06 (t, ³J=8 Hz, 2H, CH₂), 2.57 (t, ³J=8 Hz, 2H, CH₂), 1.33-1.21 (m, 2H, Si—CH), 1.09 (d, ³J=7.4 Hz, 6H, CH₃), 1.00 (d, ³J=7.1 Hz, 6H, CH₃) ppm. ¹³C-NMR (100.6 MHz, CDCl₃): δ [ppm]=171.9, 139.8, 135.8, 134.0, 132.0, 129.5, 128.7, 127.8, 127.5, 43.6, 38.6, 31.8, 17.2, 16.9, 12.4. ²⁹Si-NMR (79.4 MHz, CDCl₃): δ [ppm]=7.31. IR (KBr)=3327, 2941, 2860, 1653, 1564, 1458. MS (ESI+): m/z (%)=369 (M+, 4%), 326 (84%), 107 (100%), 91 (90%). HRMS calcd for C₂₂H₃₁O₂NSi 369.2124, found 369.2100.

### N-benzyl-2-(4-(di-tert-butyl(hydroxy)silyl)phenyl)acetamide

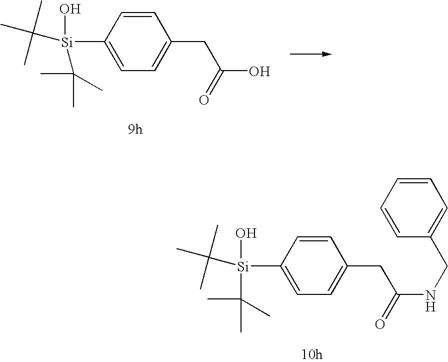

2-(4-(Di-tert-butyl(hydroxy)silyl)phenyl)acetic acid (0.20 mol, 58.9 mg), was dissolved in dry dichloromethane (3 ml). N-(3-dimethylaminopropyl)-N′-ethylcarbodiimide hydrochloride (1.4 eq, 0.28 mmol, 53.7 mg) and benzyl amine (1.3 eq, 0.26 mmol, 28.4 μl) were added. The reaction mixture was stirred overnight at room temperature, then quenched with water and extracted with dichloromethane. The combined organic extracts were washed with water and brine, dried over Na₂SO₄ and solvents were removed. The residue was purified by column chromatography (pentane/ethyl acetate 3:2) to give 55.8 mg of N-benzyl-2-(4-(di-tert-butyl(hydroxy)silyl)phenyl)acetamide (76%) as a white solid.

¹H-NMR (CDCl₃, 400 MHz): δ=1.03 (s, 18H, Si(tBu)₂), 3.63 (s, 2H, CH₂), 4.42 (d, 2H, ³J_{H—H}=5.6 Hz, N—CH₂), 5.65 (br s, 1H, NH), 7.14 (d, 2H, ³J_{H—H}=8.1 Hz, Ar—H), 7.21-7.30 (m, 5H, Ar—H), 7.63 (d, 2H, ³J_{H—H}=8.1 Hz, Ar—H). ¹³C-NMR (CDCl₃, 100 MHz): δ=20.5 (Si—C), 28.1 (CH₃), 43.8 (CH₂), 44.0 (CH₂), 127.5 (Ar—CH), 127.6 (Ar—CH), 128.6 (Ar—CH), 128.8 (Ar—CH), 128.8 (Ar—C), 135.3 (Ar—CH), 135.7 (Ar—C), 138.2 (Ar—C), 170.9 (CONH). ²⁹Si-NMR (CDCl₃, 79 MHz): δ=3.6. MS (ESI positive): 384.2 [M+H].

### 4-(di-tert-Butyl-hydroxysilanyl)-phenylacetyl-Val-βAla-Phe-Gly-NH₂

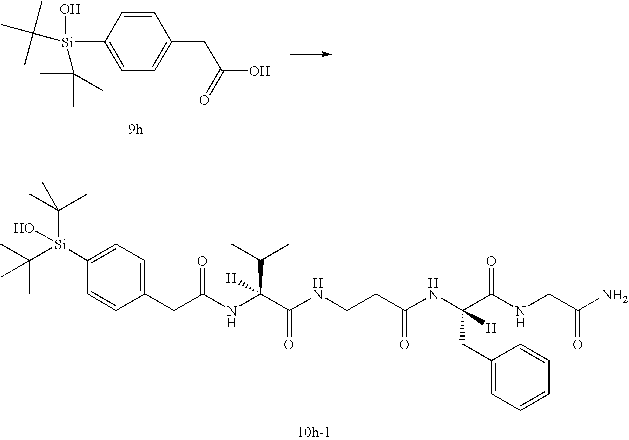

MS (ES+): 668.39

### Part C

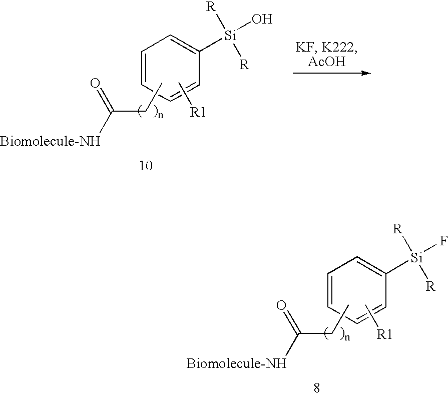

### General Method

6.12 μM silanol 10 were solved in 600 μl THF and treated with 1.42 mg (24.47 μM) KF, 9.21 mg (24.47 μM) K222, 1.69 mg (12.23 μM) K₂CO₃ and 4.2 μl acetic acid. The reaction mixture was stirred at 50° for 30 min and followed by HPLC purification to give 30-41% of 8.

### 4-(Fluoro-diisopropyl-silanyl)-phenylacetyl-Val-βAla-Phe-Gly-NH₂

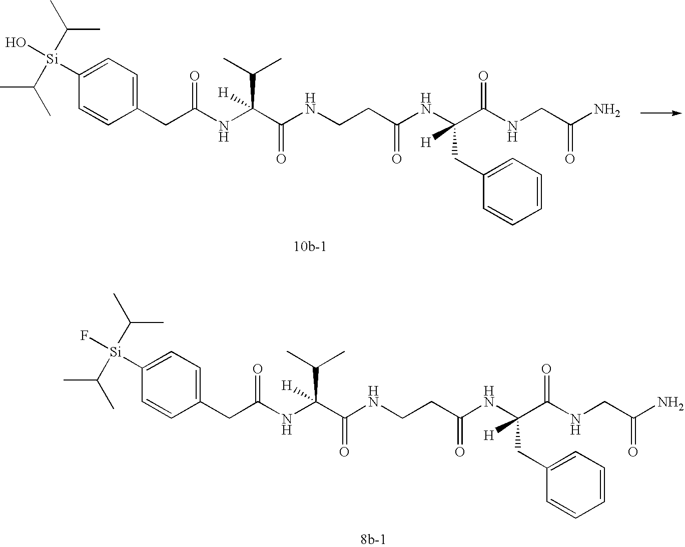

MS (ES+): 641.99

N-Benzyl-2-[4-(fluoro-diisopropyl-silanyl)-phenyl]-acetamide

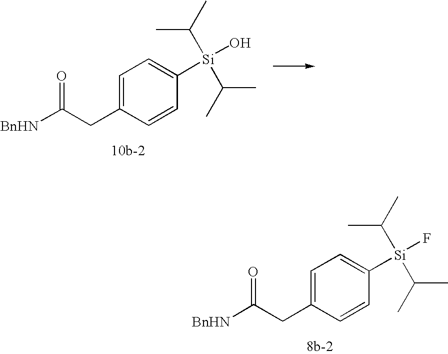

¹H-NMR (400 MHz, CDCl₃): δ=7.54 (d, ³J=8.0, 2H, aryl), 7.32-7.24 (m, 5H, aryl), 7.17 (d, ³J=8.0, 2H, aryl), 5.68 (bs, 1H, NH), 4.44 (d, ³J=5.8, 2H, CH₂—N), 3.64 (s, 2H, CH₂), 1.21 (sept, ³J=7.3 Hz, 2H, Si—CH), 1.07 (d, ³J=7.3 Hz, CH₃—Si, 6H), 1.00 (d, ³J=7.3 Hz, CH₃—Si, 6H) ppm. ¹³C-NMR (100.6 MHz, CDCl₃): δ [ppm]=170.5, 138.0, 136.5, 134.5, 134.5, 132.0, 128.8, 128.6, 127.5, 43.9, 34.6, 16.66, 16.65, 16.5, 12.3, 12.1. ²⁹Si-NMR (79.4 MHz, CDCl₃): δ [ppm]=19.7, 15.9. ¹⁹F-NMR (376 MHz, CDCl₃): δ [ppm]=−187. IR (KBr)=3299, 2949, 2867, 1643, 1561. MS (ESI+): m/z (%)=358 (M+1, 52%), 243 (12%), 102 (100%). HRMS calcd for C₂₁H₂₈ONSiF 357.1924, found 357.1913.

### 2-(4-fluorodi-iso-propylsilyl)phenyl)-N-(3-(3-(4-hydroxy-5-(hydroxymethyl)tetrahydrofuran-2-yl)-5-methyl-2,6-dioxo-2,3-dihydropyrimidin-1(6H)-yl)propyl)acetamide

After fluorination, deprotection was achieved by treatment with 2 eq. TFA in dichloromethane at ambient temperature. The reaction mixture was neutralized with bicarbonate. The phases were separated and the aqueous phase was extracted with dichloromethane. The combined organic phases were washed with brine, dried over sodium sulfate, filtrated and concenrated. The residue was purified by chromatography.

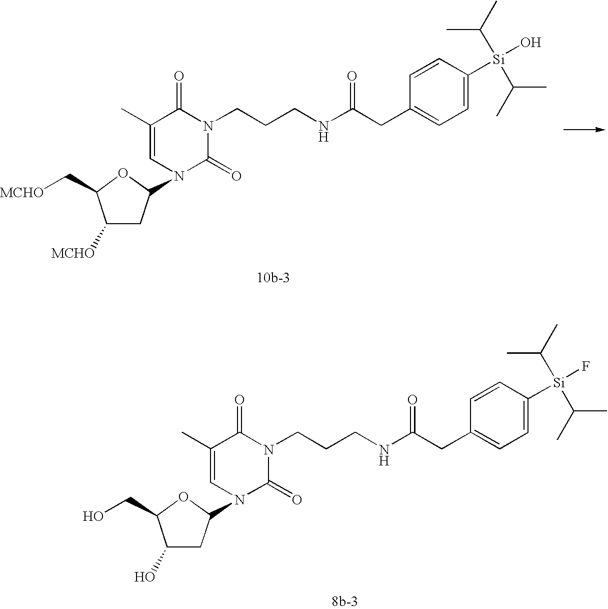

¹H-NMR (400 MHz, MeOH): δ=7.83 (1H), 7.51 (2H), 7.37 (2H), 6.29 (1H), 4.39 (1H), 3.91 (2H), 3.79 (2H), 3.54 (2H), 3.21 (2H), 2.28-2.15 (2H), 1.90 (3H), 1.82 (2H), 1.26 (2H), 1.06 (6H), 0.99 (6H) ppm. ¹⁹F-NMR (376 MHz, MeOH): δ=−188.79 ppm.

### 3-(Fluoro-diisopropyl-silanyl)-phenylpropionyl-Val-βAla-Phe-Gly-NH₂

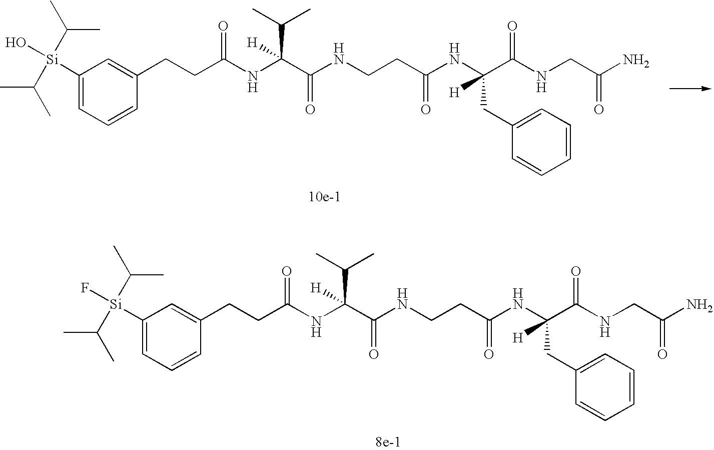

MS (ES+): 655.89

N-Benzyl-3-[3-(fluoro-diisopropyl-silanyl)-phenyl]-propionamide

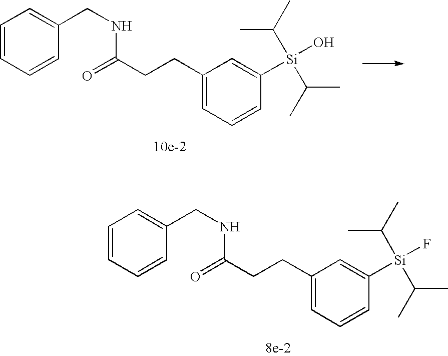

¹H-NMR (400 MHz, CDCl₃): δ=7.39 (m, 2H, aryl), 7.30-7.24 (m, 5H, aryl), 7.17 (m, 2H, aryl), 5.58 (bs, 1H, NH), 4.41 (d, ³J=5.6 Hz, 2H, CH₂N), 3.02 (d, ³J=8 Hz, 2H, CH₂), 2.52 (d, ³J=8 Hz, 2H, CH₂), 1.26 (sept, ³J=7.3 Hz, 2H, Si—CH), 1.07 (d, ³J=7.4 Hz, 6H, CH₃), 1.00 (d, ³J=7.4 Hz, 6H, CH₃) ppm. ¹³C-NMR (100.6 MHz, CDCl₃): δ [ppm]=171.7, 138.0, 133.7, 131.7, 130.1, 128.7, 128.0, 127.8, 127.5, 43.6, 38.6, 31.8, 16.7, 16.5, 12.2, 12.1. ¹⁹F-NMR (376 MHz, CDCl₃): δ [ppm]=−188. IR (KBr)=3290, 2947, 2868, 1647, 1548. MS (ESI+): m/z (%)=372 (M+1, 50%), 321 (19%), 305 (27%), 237 (100%). HRMS calcd for C₂₂H₃₀ONSiF 371.2080, found 371.2078.

### 4-(Fluor-di-tert-butylsilyl)-phenylacetyl-Val-βAla-Phe-Gly-NH₂

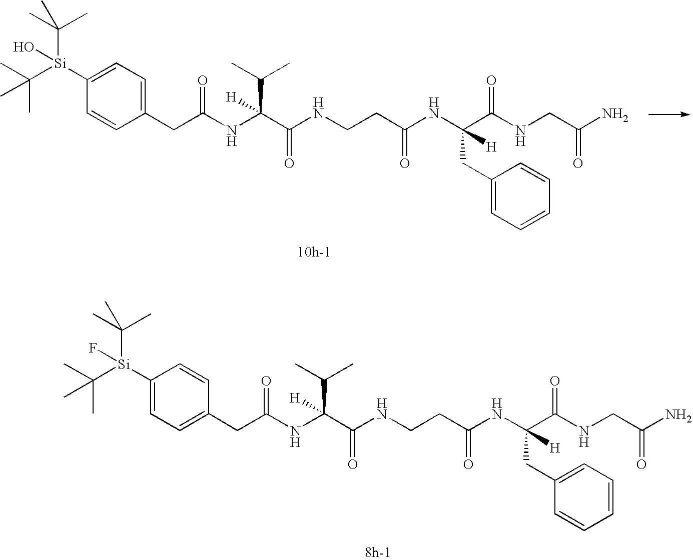

MS (ES+): 670.34

### Fluorination of Model Compounds

### Silanes

### General Method

To a solution of 120 μmol silane in 0.49 ml THF were added 31.7 mg 18-crown-6, 8.3 mg potassium carbonate, 20.6 μl acetic acid and 6.96 mg potassium fluoride. The mixture was stirred 2.5 hours at 23° C. and 1.5 hours at 50° C. After purification by chromatography on silica gel 49-87% of the title compounds were obtained as an oil.

### [4-(Fluoro-diisopropyl-silanyl)-phenyl]-acetic acid

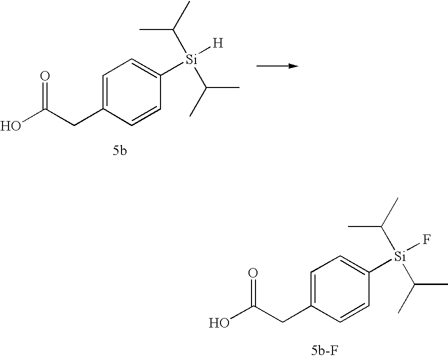

¹H-NMR (400 MHz, CDCl₃): δ=7.52 (2H), 7.32 (2H), 3.67 (2H), 1.26 (2H), 1.07 (6H), 1.01 (6H) ppm. ¹⁹F-NMR (376 MHz, CDCl₃): δ=−187.0 ppm.

### 2-[4-(Fluoro-diisopropyl-silanyl)-phenyl]-ethanol

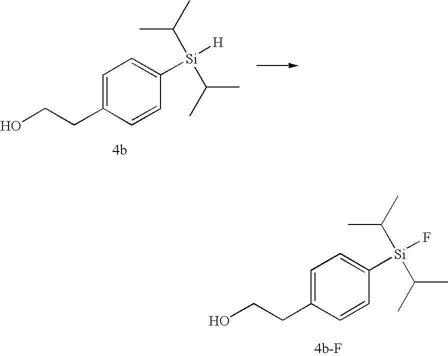

¹H-NMR (400 MHz, CDCl₃): δ=7.50 (2H), 7.27 (2H), 3.39 (2H), 2.89 (2H), 1.48-1.36 (1H), 1.27 (2H), 1.08 (6H), 1.02 (6H) ppm. ¹⁹F-NMR (376 MHz, CDCl₃): δ=−187.0 ppm.

### (RS)-Fluoro-diisopropyl-[4-[2-(tetrahydro-pyran-2-yloxy)-ethyl]-phenyl]-silane

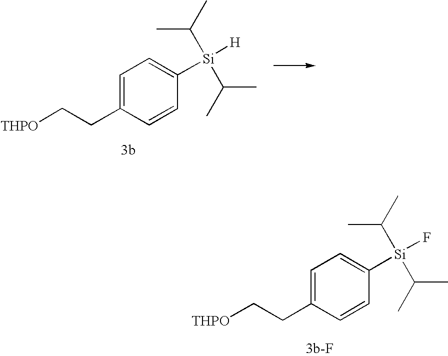

¹H-NMR (400 MHz, CDCl₃): δ=7.47 (2H), 7.28 (2H), 4.60 (1H), 3.95 (1H), 3.70-3.61 (2H), 3.42 (1H), 2.93 (2H), 1.98-1.40 (6H), 1.26 (2H), 1.07 (6H), 1.00 (6H) ppm. ¹⁹F-NMR (376 MHz, CDCl₃): δ=−187.2 ppm.

### (RS)-Fluoro-diisopropyl-[4-(tetrahydro-pyran-2-yloxymethyl)-phenyl]-silane

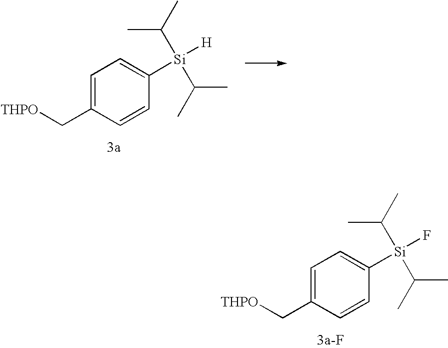

¹H-NMR (400 MHz, CDCl₃): δ=7.53 (2H), 7.40 (2H), 4.82 (1H), 4.74 (1H), 4.51 (1H), 3.93 (1H), 3.57 (1H), 1.93-1.52 (6H), 1.27 (2H), 1.08 (6H), 1.01 (6H) ppm. ¹⁹F-NMR (376 MHz, CDCl₃): δ=−187.2 ppm.

### 4-(Fluoro-diisopropyl-silanyl)-benzoic acid

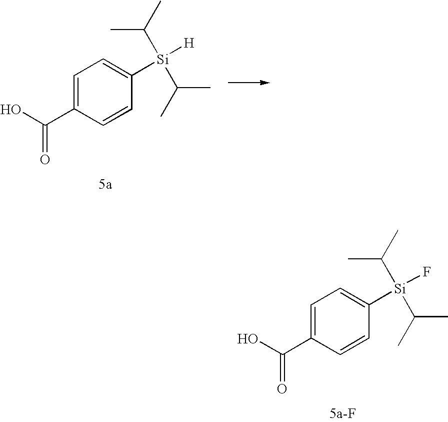

¹H-NMR (400 MHz, CDCl₃): δ=8.11 (2H), 7.68 (2H), 1.31 (2H), 1.09 (6H), 1.02 (6H) ppm. ¹⁹F-NMR (376 MHz, CDCl₃): δ=−187.5 ppm.

### [4-(Fluoro-diisopropyl-silanyl)-phenyl]-methanol

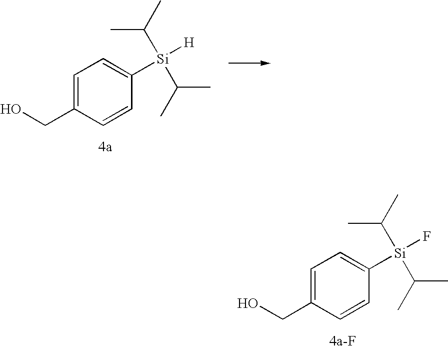

¹H-NMR (400 MHz, CDCl₃): δ=7.55 (2H), 7.40 (2H), 4.72 (2H), 1.65 (1H), 1.27 (2H), 1.08 (6H), 1.01 (6H) ppm. ¹⁹F-NMR (376 MHz, CDCl₃): δ=−187.3 ppm.

### (RS)-Fluoro-diisopropyl-{4-[3-(tetrahydro-pyran-2-yloxy)-propyl]-phenyl}-silane

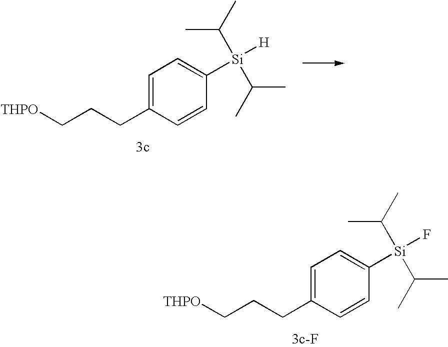

¹H-NMR (400 MHz, CDCl₃): δ=7.46 (2H), 7.23 (2H), 4.59 (1H), 3.87 (1H), 3.78 (1H), 3.50 (1H), 3.42 (1H), 2.72 (2H), 1.95 (2H), 1.84 (1H), 1.72 (1H), 1.64-1.48 (4H), 1.26 (2H), 1.08 (6H), 1.01 (6H) ppm. ¹⁹F-NMR (376 MHz, CDCl₃): δ=−187.0 ppm.

### 3-[4-(Fluoro-diisopropyl-silanyl)-phenyl]-propan-1-ol

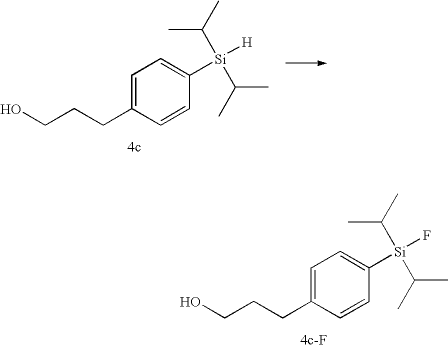

¹H-NMR (400 MHz, CDCl₃): δ=7.47 (2H), 7.23 (2H), 3.69 (2H), 2.73 (2H), 1.92 (2H), 1.59 (1H), 1.26 (2H), 1.08 (6H), 1.02 (6H) ppm. ¹⁹F-NMR (376 MHz, CDCl₃): δ=−187.0 ppm.

### 3-[4-(Fluoro-diisopropyl-silanyl)-phenyl]-propionic acid

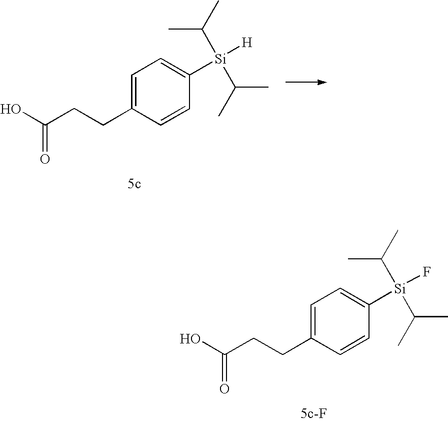

¹H-NMR (400 MHz, CDCl₃): δ=7.48 (2H), 7.24 (2H), 2.98 (2H), 2.71 (2H), 1.24 (2H), 1.08 (6H), 1.01 (6H) ppm. ¹⁹F-NMR (376 MHz, CDCl₃): δ=−187.0 ppm.

### (RS)-Fluoro-diisopropyl-[3-[3-(tetrahydro-pyran-2-yloxy)-propyl]-phenyl]-silane

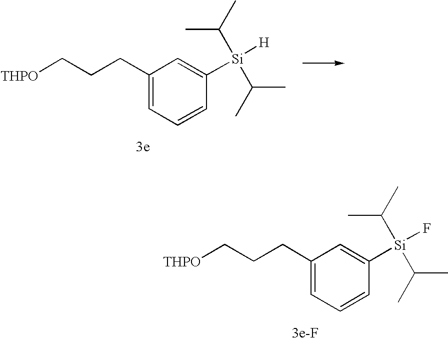

¹H-NMR (400 MHz, CDCl₃): δ=7.37-7.25 (4H), 4.57 (1H), 3.87 (1H), 3.78 (1H), 3.50 (1H), 3.40 (1H), 2.72 (2H), 1.93 (2H), 1.83 (1H), 1.72 (1H), 1.63-1.49 (4H), 1.26 (2H), 1.08 (6H), 1.01 (6H) ppm. ¹⁹F-NMR (376 MHz, CDCl₃): δ=−187.3 ppm.

### 3-[3-(Fluoro-diisopropyl-silanyl)-phenyl]-propan-1-ol

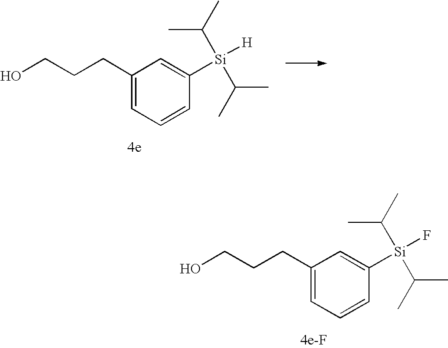

¹H-NMR (400 MHz, CDCl₃): δ=7.39-7.26 (4H), 3.70 (2H), 2.73 (2H), 1.91 (2H), 1.28 (2H), 1.09 (6H), 1.02 (6H) ppm. ¹⁹F-NMR (376 MHz, CDCl₃): δ=−187.3 ppm.

### 3-[3-(Fluoro-diisopropyl-silanyl)-phenyl]-propionic acid

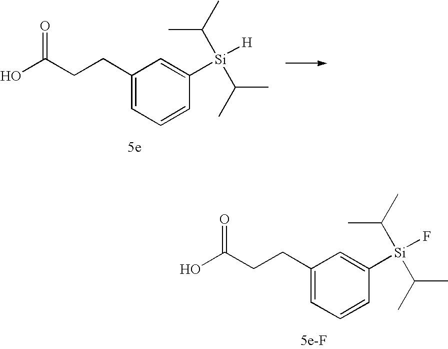

¹H-NMR (400 MHz, CDCl₃): δ=7.42-7.25 (4H), 2.98 (2H), 2.69 (2H), 1.27 (2H), 1.07 (6H), 1.01 (6H) ppm. ¹⁹F-NMR (376 MHz, CDCl₃): δ=−187.2 ppm.

### (RS)-Fluoro-diisopropyl-[3-[2-(tetrahydro-pyran-2-yloxy)-ethyl]-phenyl]-silane

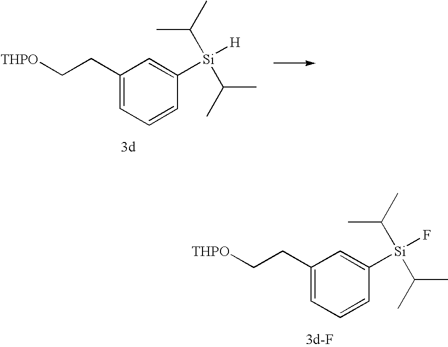

¹H-NMR (400 MHz, CDCl₃): δ=7.42 (1H), 7.38 (1H), 7.31 (2H), 4.59 (1H), 3.95 (1H), 3.71 (1H), 3.61 (1H), 3.44 (1H), 2.92 (2H), 1.80 (1H), 1.68 (1H), 1.62-1.42 (4H), 1.26 (2H), 1.08 (6H), 1.01 (6H) ppm. ¹⁹F-NMR (376 MHz, CDCl₃): δ=−187.4 ppm.

### 2-[3-(Fluoro-diisopropyl-silanyl)-phenyl]-ethanol

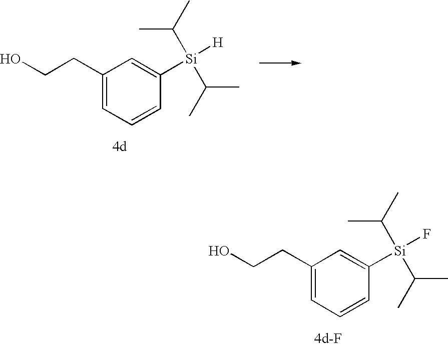

¹H-NMR (400 MHz, CDCl₃): δ=7.41 (2H), 7.35 (1H), 7.30 (1H), 3.87 (2H), 2.89 (2H), 1.39 (1H), 1.27 (2H), 1.08 (6H), 1.02 (6H) ppm. ¹⁹F-NMR (376 MHz, CDCl₃): δ=−187.1 ppm.

### [3-(Fluoro-diisopropyl-silanyl)-phenyl]-acetic acid

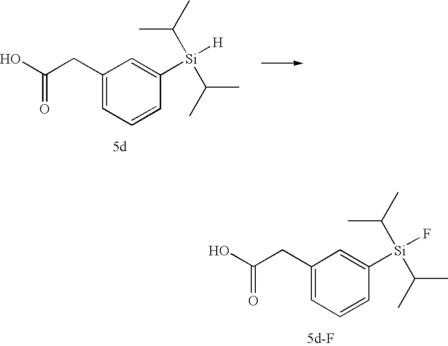

¹H-NMR (400 MHz, CDCl₃): δ=7.48-7.31 (4H), 3.38 (2H), 1.28 (2H), 1.08 (6H), 1.02 (6H) ppm. ¹⁹F-NMR (376 MHz, CDCl₃): δ=−187.1 ppm.

### [4-(Fluoro-diisobutyl-silanyl)-phenyl]-acetic acid

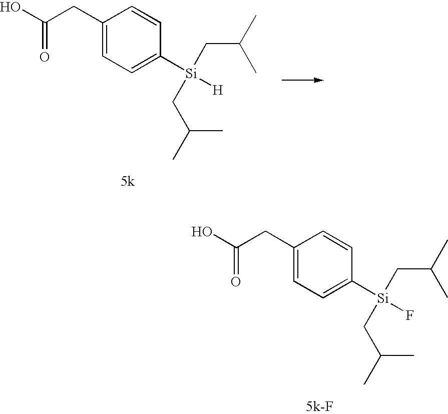

¹H-NMR (400 MHz, CDCl₃): δ=7.53 (2H), 7.32 (2H), 3.67 (2H), 1.85 (2H), 0.96-0.86 (16H) ppm. ¹⁹F-NMR (376 MHz, CDCl₃): δ=−169.6 ppm.

### (RS)-Fluoro-diisobutyl-{4-[2-(tetrahydro-pyran-2-yloxy)-ethyl]-phenyl}-silane

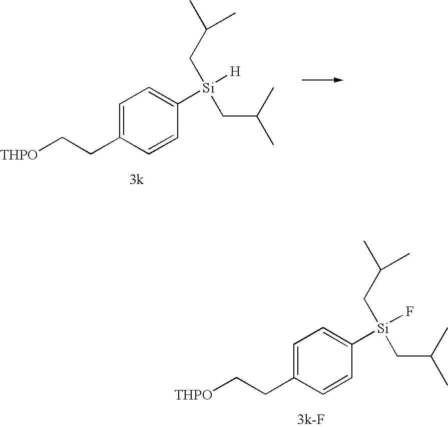

¹H-NMR (400 MHz, CDCl₃): δ=7.48 (2H), 7.27 (2H), 4.59 (1H), 3.95 (1H), 3.69 (1H), 3.63 (1H), 3.43 (1H), 2.92 (2H), 1.84 (2H), 1.80 (1H), 1.69 (1H), 1.63-1.43 (4H), 0.98-0.85 (16H) ppm. ¹⁹F-NMR (376 MHz, CDCl₃): δ=−169.6 ppm.

### (RS)-Fluoro-diisobutyl-[4-(tetrahydro-pyran-2-yloxymethyl)-phenyl]-silane

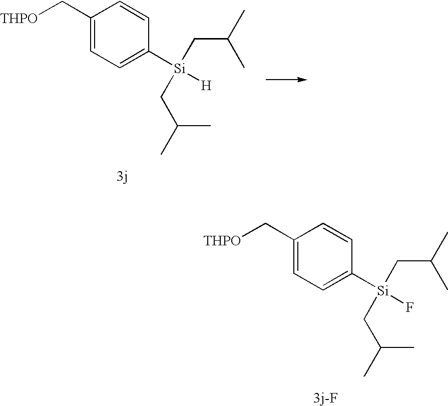

¹H-NMR (400 MHz, CDCl₃): δ=7.55 (2H), 7.40 (2H), 4.81 (1H), 4.73 (1H), 4.51 (1H), 3.93 (1H), 3.56 (1H), 1.92-1.51 (8H), 0.98-0.85 (16H) ppm. ¹⁹F-NMR (376 MHz, CDCl₃): δ=−169.7 ppm.

### 4-(Fluoro-diisobutyl-silanyl)-benzoic acid

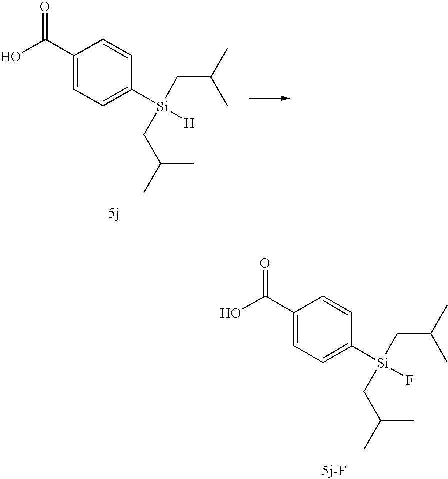

¹H-NMR (400 MHz, CDCl₃): δ=8.12 (2H), 7.69 (2H), 1.85 (2H), 0.98-0.83 (16H) ppm. ¹⁹F-NMR (376 MHz, CDCl₃): δ=−170.4 ppm.

### (RS)-Fluoro-diphenyl-{4-[2-(tetrahydro-pyran-2-yloxy)-ethyl]-phenyl}-silane

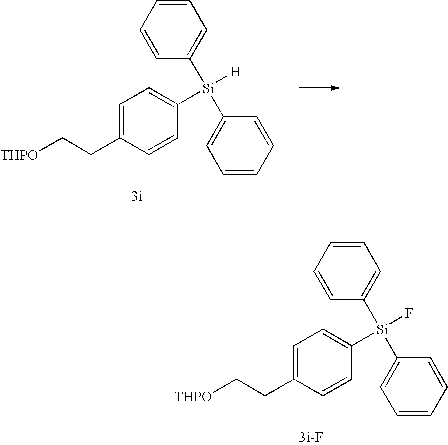

¹H-NMR (400 MHz, CDCl₃): δ=7.68-7.30 (14H), 4.64 (1H), 3.99 (1H), 3.77 (1H), 3.66 (1H), 3.48 (1H), 2.97 (2H), 1.88-1.60 (6H) ppm. ¹⁹F-NMR (376 MHz, CDCl₃): δ=−169.4 ppm.

### Silanols, Preparation of Model Compounds

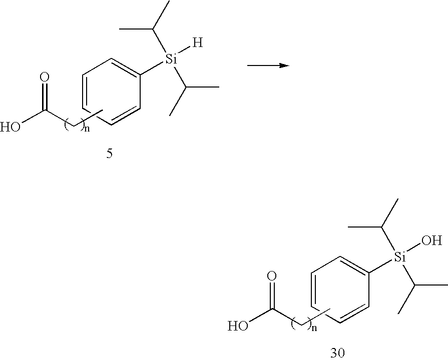

### General Method

To a solution of 399 μmol 5 (Example I, Part D) in 1.68 ml tetrachloro-methane was added 7.27 mg Palladium (10% on charcoal) and the mixture stirred for 16 hours at 23° C. 72 μl water were added and stirring was continued for additional 75 hours at 23° C. Sodium sulfate was added and after filtration the solvent was evaporated. The crude product was purified by chromatography on silica gel to give 68-75% of the title compound 30 as an oil.

### 4-(Hydroxy-diisopropyl-silanyl)-benzoic acid

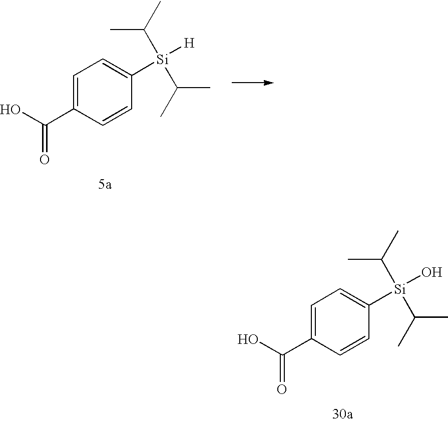

¹H-NMR (400 MHz, CDCl₃): δ=8.09 (2H), 7.69 (2H), 1.25 (2H), 1.06 (6H), 0.98 (6H) ppm.

### 4-(Hydroxy-diisopropyl-silanyl)-phenyl]-acetic acid

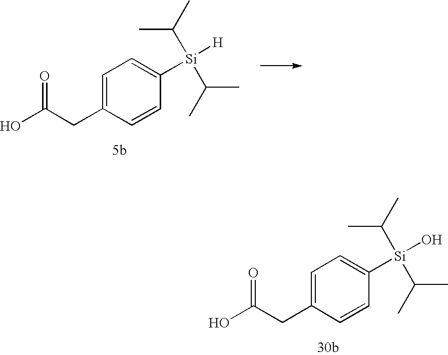

¹H-NMR (400 MHz, CDCl₃): δ=7.51 (2H), 7.29 (2H), 5.20-4.50 (2H), 3.65 (2H), 1.20 (3H), 1.04 (6H), 0.96 (6H) ppm.

### 4-(Hydroxy-diisobutyl-silanyl)-benzoic acid

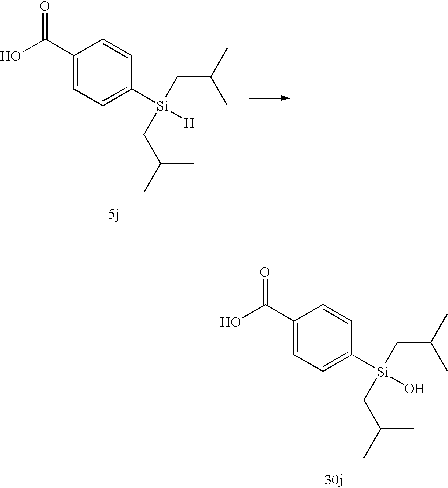

¹H-NMR (400 MHz, CDCl₃): δ=8.08 (2H), 7.70 (2H), 1.82 (2H), 0.93 (6H), 0.91 (6H), 0.88 (4H) ppm.

### [4-(Hydroxy-diisobutyl-silanyl)-phenyl]-acetic acid

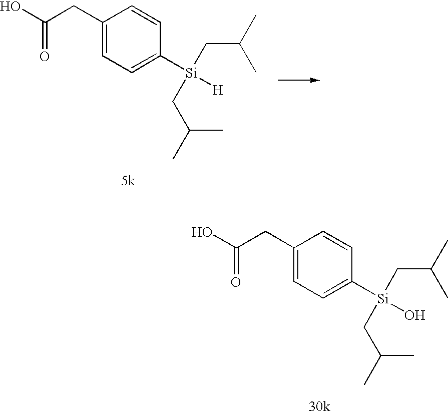

¹H-NMR (CDCl₃): δ=7.54 (2H), 7.29 (2H), 3.65 (2H), 1.82 (2H), 0.93 (6H), 0.91 (6H), 0.84 (4H) ppm.

Silanols, fluorination

To a solution of 139 μmol silanol 30 in 0.57 ml THF were added 36.7 mg 18-crown-6, 9.6 mg potassium carbonate, 23.8 μl acetic acid and 8.07 mg potassium fluoride. The mixture was stirred at 50° C. for 1.5 hours and purified by chromatography on silica gel to yield 17 mg 46-60% of the title compounds 30-F as an oil.

### 4-(Fluoro-diisopropyl-silanyl)-benzoic acid

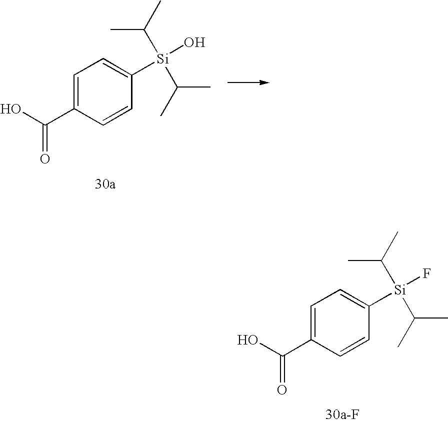

¹H-NMR (400 MHz, CDCl₃): δ=8.11 (2H), 7.68 (2H), 1.31 (2H), 1.09 (6H), 1.02 (6H) ppm. ¹⁹F-NMR (376 MHz, CDCl₃): δ=−187.5 ppm.

### [4-(Fluoro-diisopropyl-silanyl)-phenyl]-acetic acid

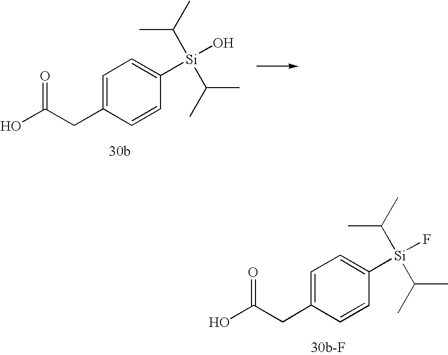

¹H-NMR (400 MHz, CDCl₃): δ=7.52 (2H), 7.32 (2H), 3.67 (2H), 1.26 (2H), 1.07 (6H), 1.01 (6H) ppm. ¹⁹F-NMR (376 MHz, CDCl₃): δ=−187.0 ppm.

### 4-(Fluoro-diisobutyl-silanyl)-benzoic acid

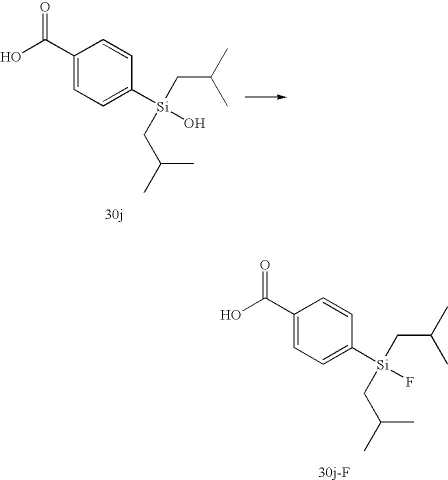

¹H-NMR (400 MHz, CDCl₃): δ=8.12 (2H), 7.69 (2H), 1.85 (2H), 0.98-0.83 (16H) ppm. ¹⁹F-NMR (376 MHz, CDCl₃): δ=−170.4 ppm.

### [4-(Fluoro-diisobutyl-silanyl)-phenyl]-acetic acid

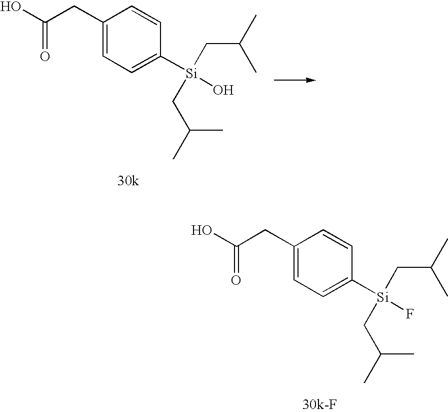

¹H-NMR (400 MHz, CDCl₃): δ=7.53 (2H), 7.32 (2H), 3.67 (2H), 1.85 (2H), 0.96-0.86 (16H) ppm.

¹⁹F-NMR (376 MHz, CDCl₃): δ=−169.6 ppm.

### Example III

### Part A

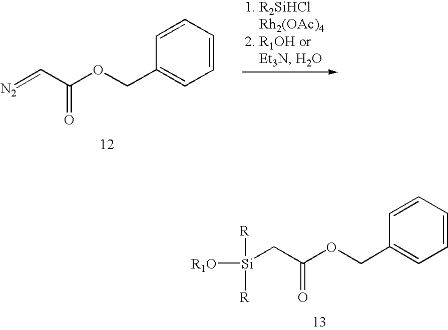

### (Di-tert-butyl-hydroxy-silanyl)-acetic acid benzyl ester

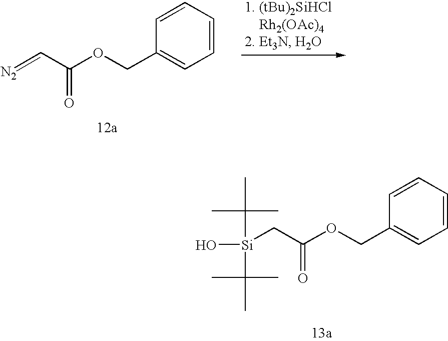

A solution of benzyl diazoacetate 12a (4.00 mmol, 705 mg) in anhydrous dichloromethane (1 ml) was added very slowly at room temperature, using a syringe pump (2 mmol/h), to a solution of di-tert-butylchlorosilane (4.00 mmol, 0.82 ml) and Rh₂(OAc)₄ (0.012 mmol, 5.7 mg) in anhydrous dichloromethane (1.5 ml). The mixture was cooled to 0° C. Then a solution of triethylamine (5.00 mmol, 0.70 ml) in anhydrous dichloromethane (1 ml) and water (5.00 mmol, 0.09 ml) were added successively dropwise. The suspension was stirred for 3 h and then treated at 0° C. with a saturated solution of NaHCO₃ and the organic layer was separated. The aqueous layer was extracted with dichloromethane and the combined extracts were washed with brine, dried (MgSO₄) and evaporated in vacuo. The residue was purified by column chromatography (pentane-EtOAc 19:1) to give 835 mg benzyl 2-(di-tert-butyl(hydroxy)silyl)acetate 13a (68%) as a white solid.

¹H-NMR (CDCl₃, 400 MHz): δ=1.01 (s, 18H, Si(tBu)₂), 2.05 (s, 2H, SiCH₂), 5.09 (s, 2H, COCH₂), 7.30-7.39 (m, 5H, Ar—H). ¹³C-NMR (CDCl₃, 100 MHz): δ=20.7 (SiCH₂), 20.8 (SiC), 27.4 (CH₃), 66.7 (OCH₂), 128.5 (Ar—CH), 128.6 (Ar—CH), 128.8 (Ar—CH), 136.0 (Ar—C), 173.8 (C═O). ²⁹Si-NMR (CDCl₃, 79 MHz): δ=10.1. MS (ESI positive): 309.2 [M+H]⁺. HR-ESI-MS: 331.1700 [M+Na]⁺ (calc. for C₁₇H₂₈NaO₃Si: 331.1700).

### Benzyl 2-(benzyloxydi-tert-butylsilyl)acetate

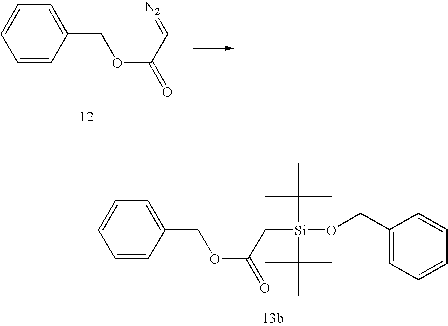

A solution of benzyl diazoacetate 12 (2.80 mmol, 493 mg) in anhydrous dichloromethane (2 ml) was added very slowly, using a syringe pump (2 mmol/h), at room temperature to a solution of di-tert-butylchlorosilane (2.93 mmol, 0.59 ml) and Rh₂(OAc)₄ (0.030 mmol, 14 mg) in anhydrous dichloromethane (3 ml). The crude product, obtained as an oil after evaporation of the solvent, was diluted with anhydrous DMF (2 ml) and then added dropwise to a solution of imidazole (8.40 mmol, 572 mg), 4-DMAP (0.040 mmol, 5 mg) and anhydrous benzyl alcohol (14.0 mmol, 1.45 ml) in anhydrous DMF (4 ml). The solution was stirred overnight at room temperature then treated at 0° C. with a saturated solution of NaHCO₃. The aqueous layer was extracted with dichloromethane and the combined extracts were washed with brine, dried (MgSO₄) and evaporated in vacuo. The residue was purified by column chromatography (pentane/ethyl acetate 19:1) to give 122 mg benzyl 2-(benzyloxydi-tert-butylsilyl)acetate 13b (11%) as a colorless oil and 289 mg benzyl 2-(di-tert-butyl(hydroxy)silyl)acetate (33%) 13a as a white solid. Benzyl 2-(benzyloxydi-tert-butylsilyl)acetate: ¹H-NMR (CDCl₃, 400 MHz): δ=1.06 (s, 18H, Si(tBu)₂), 2.19 (s, 2H, SiCH₂), 4.95 (s, 2H, SiOCH₂), 5.02 (s, 2H, COCH₂), 7.20-7.35 (m, 10H, Ar—H). ¹³C-NMR (CDCl₃, 100 MHz): δ=20.5 (SiCH₂), 21.8 (SiC), 28.0 (CH₃), 66.4 (OCH₂), 66.4 (OCH₂), 125.9 (Ar—CH), 126.9 (Ar—CH), 128.2 (Ar—CH), 128.3 (Ar—CH), 128.6 (Ar—CH), 128.6 (Ar—CH), 136.0 (Ar—C), 141.2 (Ar—C), 173.0 (C═O). ²⁹Si-NMR (CDCl₃, 79 MHz): δ=8.4. MS (ESI positive): 399.2 [M+H]⁺. HR-ESI-MS: 421.2161 [M+Na]⁺ (calc. for C₂₄H₃₄NaO₃Si: 421.2169).

### Part B

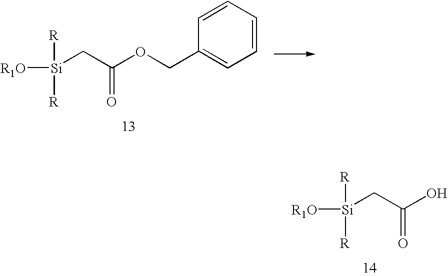

### (Di-tert-butyl-hydroxy-silanyl)-acetic acid

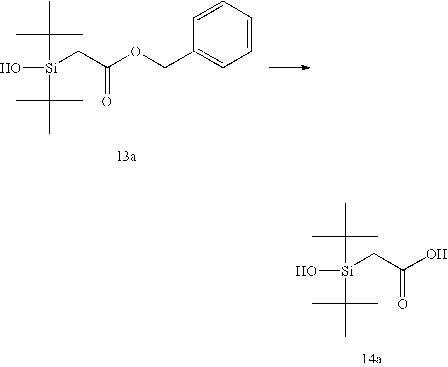

Into an argon flushed flask 10% Pd/C (30 mg) was added. Benzyl 2-(di-tert-butyl(hydroxy)silyl)acetate 13a (2.70 mmol, 834 mg) in EtOAc (10 ml) was then added. The argon atmosphere was then replaced by hydrogen. The reaction mixture was stirred overnight at room temperature and then filtered over Celite (eluent: EtOAc). Evaporation of the solvent gave 552 mg 2-(di-tert-butyl(hydroxy)silyl)acetic acid 14a (94%). ¹H-NMR 6=1.05 (s, 18H, Si(tBu)₂), 2.04 (s, 2H, SiCH₂). ¹³C-NMR (CDCl₃, 100 MHz): δ=20.7 (SiCH₂), 21.0 (SiC), 27.4 (CH₃), 178.5 (C═O). MS (ESI positive): 219.2 [M+H].

### Part C

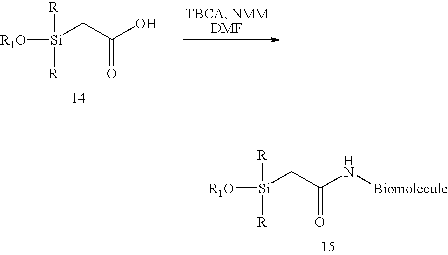

### (Di-tert-butyl-hydroxy-silanyl)-acetyl-Val-βAla-Phe-Gly-NH₂

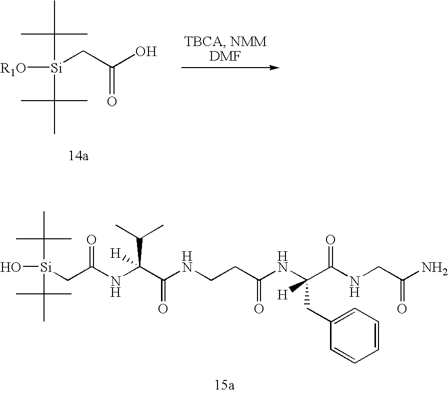

30 mg Di-tert-butyl-hydroxy-silanyl)-acetic acid 14a (137 μM), 45 mg (137 μM) TBCA and 14 mg (137 μM) NMM were solved in 1 ml DMF and stirred for 30 min. The reaction mixture was added to 69 μM of the resin bound protected peptide suspended in 2 ml DMF for 14 h. The resin was filtered, washed with DMF and dichloromethane. After cleavage from the resin with 1 ml of a mixture of 85% TFA, 5% water, 5% phenol, and 5% triisopropylsilane, the product was precipitated with MTBE and purified by HPLC to give 33% of 15a.

MS (ES+): 592.20

### Part D

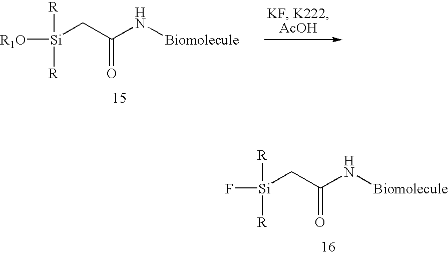

### (Di-tert-butyl-fluoro-silanyl)-acetyl-Val-βAla-Phe-Gly-NH₂

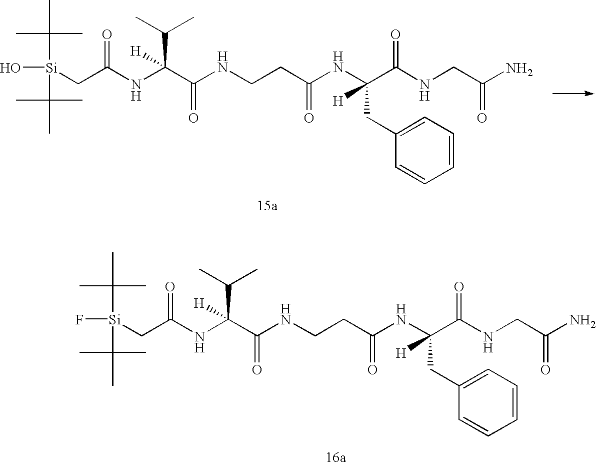

### Fluorination Model Compound

### Benzyl 2-(di-tert-butylfluorosilyl)acetate

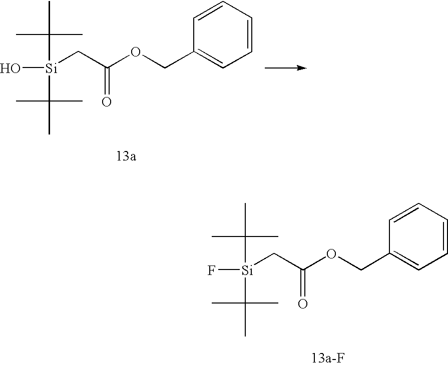

To benzyl 2-(di-tert-butyl(hydroxy)silyl)acetate 13a (2.70 mmol, 833 mg) and 4-methoxysalicylaldehyde (2.70 mmol, 411 mg) in anhydrous dichloromethane (30 ml) was added at room temperature boron trifluoride diethyl etherate (5.40 mmol, 0.68 ml). After stirring the reaction mixture at room temperature for 4 h, the solution was hydrolyzed with water (5 ml) and vigourously stirred for 10 min. The organic layer was washed with brine, dried over MgSO₄, filtered and solvents removed by evaporation under vacuum. The crude product was purified by column chromatography (pentane-EtOAc 99:1) to give 740 mg benzyl 2-(di-tert-butylfluorosilyl)acetate 13a-F (88%) as a colorless oil. ¹H-NMR δ=1.07 (s, 18H, Si(tBu)₂), 2.18 (d, 2H, SiCH₂), 5.10 (s, 2H, COCH₂), 7.29-7.38 (m, 5H, Ar—H). ¹³C-NMR (CDCl₃, 100 MHz): δ=20.8 (d, SiC), 20.9 (d, SiC), 27.0 (CH₃), 66.5 (OCH₂), 128.2 (Ar—CH), 128.5 (Ar—CH), 128.6 (Ar—CH), 136.0 (Ar—C), 171.5 (d, C═O). ¹⁹F-NMR (CDCl₃, 376 MHz): δ=−181.0 (m). MS (ESI positive): 311.1 [M+H].

### Example IV

### 4-[3-(Ethoxy-diisopropyl-silanyl)-propylcarbamoyl]-butyric acid

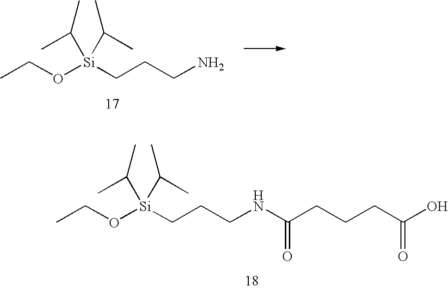

The solution of 500 mg (2.3 mmol) 3-(ethoxy-diisopropyl-silanyl)-propylamine 17 in 24 ml dichloro-methane was cooled to 3° C., 276 mg dihydro-pyran-2,6-dione was added and the mixture was stirred for 30 minutes. The solvent was removed and 776 mg of the title compound 18 were isolated without further purification. ¹H-NMR (CDCl₃): δ=6.84-6.27 (1H), 6.01 (1H), 3.77 (2H), 3.28 (2H), 2.46 (2H), 2.32 (2H), 2.01 (2H), 1.64 (2H), 1.24 (3H), 1.06 (14H), 0.66 (2H) ppm.

### 4-[3-(Ethoxy-diisopropyl-silanyl)-propylcarbamoyl]-butyric acid 2,5-dioxo-pyrrolidin-1-yl ester

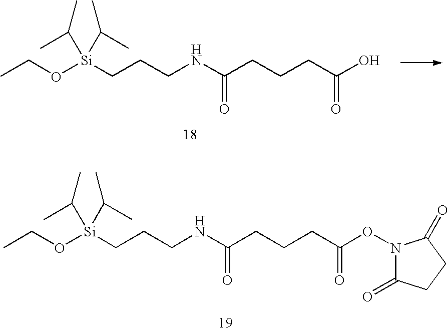

To 300 mg (0.9 mmol) 4-[3-(ethoxy-diisopropyl-silanyl)-propylcarbamoyl]-butyric acid 18, solved in 9 ml dichloro-methane were added 114 mg N-hydroxy-succinimide, 189 mg (3-Dimethylamino-propyl)-ethyl-carbodiimide hydrochloride and the mixture was stirred for 16 hours at 23° C. After addition of water and extraction with dichloromethane the combined organic extracts were dried over sodium sulfate. After filtration and solvent evaporation the crude product was purified by chromatography on silica gel to give 172 mg (44%) of 19.

¹H-NMR (CDCl₃): δ=6.10 (1H), 3.76 (2H), 3.28 (2H), 2.88 (4H), 2.72 (2H), 2.33 (2H), 2.15 (2H), 1.62 (2H), 1.22 (3H), 1.05 (14H), 0.66 (2H) ppm.

### 4-[3-(Ethoxy-diisopropyl-silanyl)-propylcarbamoyl]-butyric-Val-βAla-Phe-Gly-NH₂

0.05 mmol resin bound protected peptide suspended in 2 ml DMF was treated with 0.15 mmol active ester 19 for 12 h. The reaction mixture was filtered and the resin was washed with DMF and dichloromethane. After cleavage from the resin with 1 ml of a mixture of 85% TFA, 5% water, 5% phenol, and 5% triisopropylsilane, the product was precipitated with MTBE and purified by HPLC to give 12% of 20.

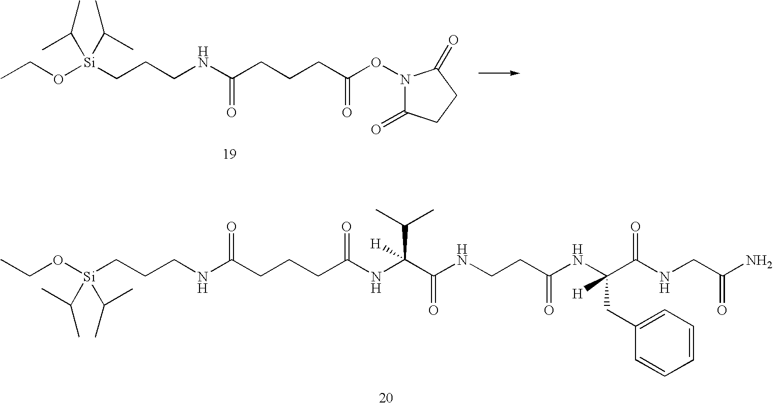

MS (ES+): 704.99

### Pentanedioic acid ((S)-1-{2-[(S)-1-(carbamoylmethyl-carbamoyl)-2-phenyl-ethylcarbamoyl]-ethylcarbamoyl}-2-methyl-propyl)-amide[3-(fluoro-diisopropyl-silanyl)-propyl]-amide

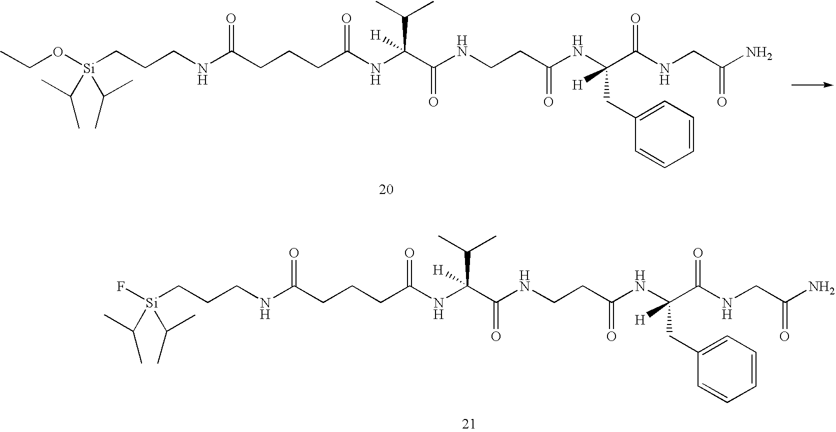

MS (ES+): 679.20

### Model Compounds of Example 4

### N-[3-(Ethoxy-diisopropyl-silanyl)-propyl]-benzamide

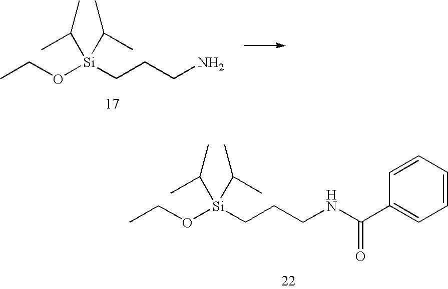

To the solution of 1.0 g 3-(ethoxy-diisopropyl-silanyl)-propylamine 17 in 10 ml dichloro-methane were added at 3° C. 535 μl benzoyl chloride, 638 μl triethyl-amine and the mixture was stirred for 1.5 hours. The mixture was poured into water, extracted with dichloro-methane and the combined organic extracts were dried over sodium sulfate. After filtration und evaporation of the solvent 1.44 g (97%) of the title compound 22 were isolated as an oil.

¹H-NMR (400 MHz, CDCl₃): δ=7.78 (2H), 7.50 (1H), 7.43 (2H), 6.40 (1H), 3.74 (2H), 3.46 (2H), 1.74 (2H), 1.19 (3H), 1.04 (14H), 0.71 (2H) ppm.

### N-(3-(ethoxydi-iso-propylsilyl)propyl)biphenyl-4-carboxamide

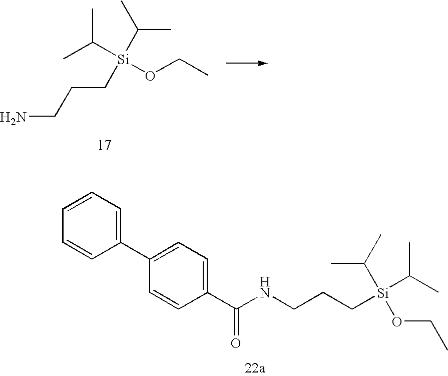

To an ice-cooled solution of 3-aminopropyldi-iso-propylethoxysilane (5.00 mmol, 1.09 g) and triethylamine (6.00 mmol, 0.84 ml) in anhydrous dioxane (3 ml) a solution of 4-biphenylcarbonylchloride (6.00 mmol, 1.30 g) in anhydrous dioxane (12 ml) was added dropwise. The reaction mixture was stirred for 1.5 h at room temperature. Water (30 ml) was added and the aqueous layer extracted with dichloromethane. The combined organic layers were dried over Na₂SO₄ and concentrated under reduced pressure. Purification of the crude product by column chromatography (n-hexane/ethyl acetate 4:1) afforded 1.09 g N-(3-(ethoxydi-iso-propylsilyl)propyl)biphenyl-4-carboxamide (55%) as a white solid.

¹H-NMR (CD₃CN, 300 MHz): δ=0.68-0.73 (m, 2H, SiCH₂), 1.01 (s, 14H, Si(iPr)₂), 1.14 (t, 3H, ³J_{H—H}=7.0 Hz, CH₃), 1.62-1.72 (m, 2H, CH₂), 3.31-3.38 (q, 2H, ³J_{H—H}=7.0 Hz, NCH₂), 3.68-3.75 (q, 2H¹³J_{H—H}=6.4 Hz, OCH₂), 7.20 (br s, 1H, NH), 7.37-7.50 (m, 3H, Ar—H), 7.67-7.72 (m, 4H, Ar—H), 7.85-7.88 (m, 2H, Ar—H). ¹³C-NMR (CD₃CN, 75 MHz): δ=8.5 (SiCH₂), 13.3 (SiCH), 18.0 (CH₃), 19.1 (CH₃), 24.4 (CH₂), 43.8 (NCH₂), 59.6 (OCH₂), 118.2 (br, Ar—CH), 127.8 (Ar—CH), 127.9 (Ar—CH), 128.5 (Ar—CH), 128.9 (Ar—CH), 129.9 (Ar—CH), 134.9 (Ar—C), 140.8 (Ar—C), 144.3 (Ar—C), 167.3 (C═O). ²⁹Si-NMR (CD₃CN, 79 MHz): δ=14.2.

MS (ESI positive): 370.2 [M-OEt+OH+H]⁺. HR-ESI-MS: 370.2029 [M-OEt+OH+H]⁺ (calc. for C₂₂H₃₂NO₂Si: 370.2202), 392.1754 [M-OEt+OH+Na]⁺(calc. for C₂₂H₃₁NNaO₂Si: 392.2022).

### Fluorination of Model Compounds

### N-[3-(Fluoro-diisopropyl-silanyl)-propyl]-benzamide

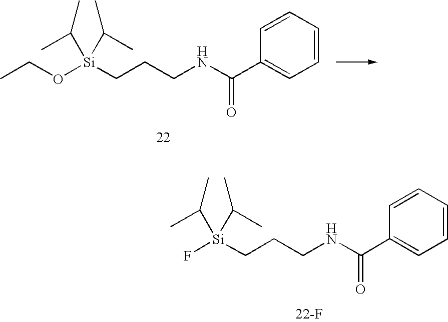

50 mg (156 μmol) N-[3-(ethoxy-diisopropyl-silanyl)-propyl]-benzamide 22 was transformed in analogy to silanes to yield after isolation and purification 28 mg (61%) of the title compound 22-F as an oil.

¹H-NMR (400 MHz, CDCl₃): δ=7.80 (2H), 7.54 (1H), 7.47 (2H), 3.50 (2H), 1.79 (2H), 1.68 (1H), 1.12-1.03 (14H), 0.81 (2H) ppm. ¹⁹F-NMR (376 MHz, CDCl₃): δ=−181.3 ppm.

### N-(3-(fluorodi-iso-propylsilyl)propyl)biphenyl-4-carboxamide

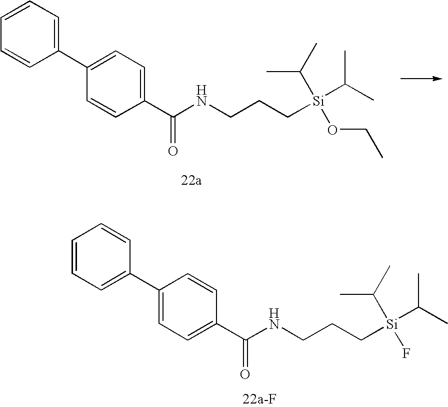

N-(3-(ethoxydi-iso-propylsilyl)propyl)biphenyl-4-carboxamide (100 mg, 0.251 mmol) was dissolved in anhydrous diethylether (5 ml). Boron trifluoride diethyl etherate (0.126 mmol, 16 μl) was added. The reaction mixture was heated under reflux for 30 min. The solvent was removed and the crude product was purified by column chromatography (n-hexane/ethyl acetate 4:1) to give 90.8 mg N-(3-(fluorodiisopropylsilyl)propyl)biphenyl-4-carboxamide (97%) as a white solid.

¹H-NMR (CD₃CN, 300 MHz): δ=0.75-0.83 (m, 2H, SiCH₂), 1.05 (m, 14H, Si(iPr)₂), 1.65-1.75 (m, 2H, CH₂), 3.33-3.39 (q, 2H¹³J_{H—H}=7.0 Hz, NCH₂), 7.16 (br s, 1H, NH), 7.37-7.51 (m, 3H, Ar—H), 7.67-7.73 (m, 4H, Ar—H), 7.85-7.89 (m, 2H, Ar—H). ¹³C-NMR (CD₃CN, 75 MHz): δ=7.4 (d, ²J_{C—F}=13.2 Hz, SiCH₂), 12.1 (d, ²J_{C—F}=13.2 Hz, SiCH), 16.2 (d, ³J_{C—F}=1.7 Hz, CH₃), 22.7 (d, ³J_{C—F}=1.7 Hz, CH₂), 42.5 (NCH₂), 117.3 (br, Ar—CH), 126.8 (Ar—CH), 127.0 (Ar—CH), 127.5 (Ar—CH), 128.0 (Ar—CH), 128.9 (Ar—CH), 133.8 (Ar—C), 139.8 (Ar—C), 143.4 (Ar—C), 166.4 (C═O). ¹⁹F-NMR (CD₃CN, 282 MHz): δ=−181.7. ²⁹Si-NMR (CD₃CN, 79 MHz): δ 29.8 (¹J_{Si—F}=298 Hz). MS (ESI positive): 372.2. HR-ESI-MS: 372.2010 [M+H]⁺ (calc. for C₂₂H₃₁FNOSi: 372.2159).

### Example V

### Pent-4-enoic acid 2,5-dioxo-pyrrolidin-1-yl ester

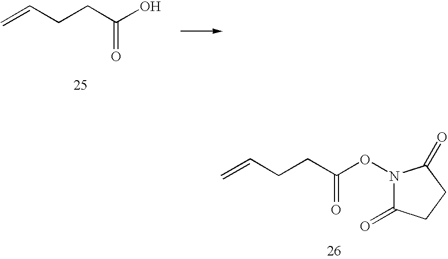

To a solution of 5 g (49.9 mmol) pent-4-enoic acid 25 in 250 ml dichloromethane were added 6.32 g N-hydroxy-succinimide, 10.48 g mg (3-Dimethylamino-propyl)-ethyl-carbodiimide hydrochloride and the mixture was stirred for 16 hours at 23° C. After addition of water and extraction with dichloro-methane the combined organic extracts were dried over sodium sulfate. After filtration and solvent evaporation the crude product was purified by chromatography on silica gel to give 8.8 g (89%) of the title compound 26 as an oil.

¹H-NMR (CDCl₃): δ=5.89 (1H), 5.17 (1H), 5.12 (1H), 2.87 (4H), 2.75 (2H), 2.53 (2H) ppm.

### Part A

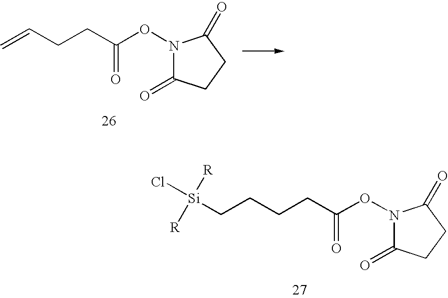

### 5-(Chloro-diisobutyl-silanyl)-pentanoic acid 2,5-dioxo-pyrrolidin-1-yl ester

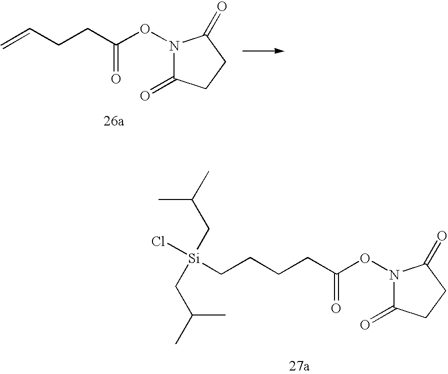

500 mg (2.54 mmol) pent-4-enoic acid 2,5-dioxo-pyrrolidin-1-yl ester 26a were solved in 2 ml THF and 907 mg chloro-diisobutyl-silane followed by a catalytic amount of Karstedts catalyst were added. After 2 hours of stirring the mixture containing title compound 27a was used directly for the next reaction.

### Part B

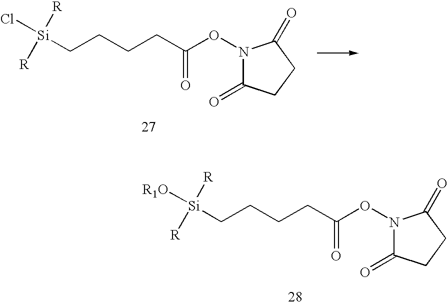

### 5-[Diisobutyl-(4-phenyl-butoxy)-silanyl]-pentanoic acid 2,5-dioxo-pyrrolidin-1-yl ester

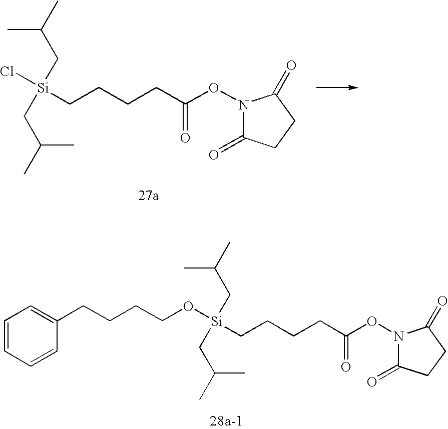

To the reaction mixture containing 5-(chloro-diisobutyl-silanyl)-pentanoic acid 2,5-dioxo-pyrrolidin-1-yl ester 27a (2.54 mmol at maximum) were added at 3° C. 2 ml THF, 781 μl 4-phenyl-butan-1-ol, 763 μl 1,5-diaza-bicyclo[3.3.3]undecane. After 2 hours the mixture was pured into water and extracted with ethyl acetate. The combined organic extracts were washed with brine and dried over sodium sulfate. After filtration and removal of the solvent the crude product was purified by chromatography on silica gel to give 762 mg (61%) of the title compound 28a as an oil.

¹H-NMR (CDCl₃): δ=7.35-7.28 (3H), 7.24-7.19 (2H), 3.63 (2H), 2.85 (4H), 2.66 (4H), 1.89-1.46 (12H), 0.99 (14H), 0.67 (2H) ppm.

### 5-[(4-Polystyrene-methoxy-benzyloxy)-diisobutyl-silanyl]-pentanoic acid 2,5-dioxo-pyrrolidin-1-yl ester

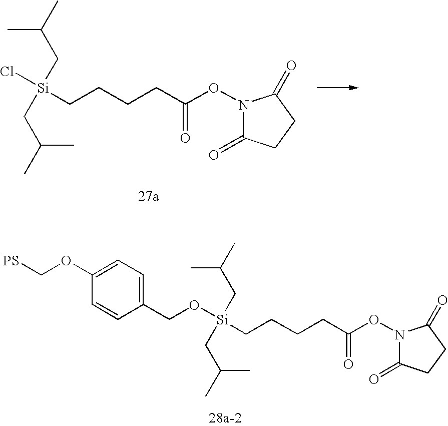

To a suspension of 566 mg Wang resin in 10 ml dichloro-methane was added the reaction mixture containing 5-(chloro-diisobutyl-silanyl)-pentanoic acid 2,5-dioxo-pyrrolidin-1-yl ester 27a (2.54 mmol at maximum) 398 μl 1,5-diaza-bicyclo[3.3.3]undecane. The mixture was stirred for 2.5 hours at 23° C. and after filtration the residue was washed with dichloro-methane and dried. 713 mg (68%) of the title compound 28a-2 were isolated as solid.

### 5-(Polystyrene-methoxy-diisobutyl-silanyl)-pentanoic acid 2,5-dioxo-pyrrolidin-1-yl ester

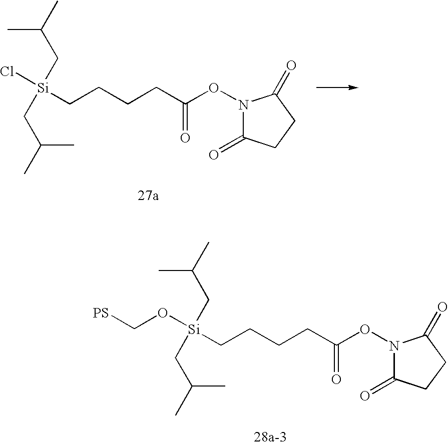

To a suspension of 576 mg hydroxymethyl-polystyrene resin in 5 ml dichloro-methane was added the reaction mixture containing 5-(chloro-diisobutyl-silanyl)-pentanoic acid 2,5-dioxo-pyrrolidin-1-yl ester 27a (2.54 mmol at maximum) and 398 μl 1,5-diaza-bicyclo[3.3.3]undecane. The mixture was stirred for 2.5 hours at 23° C. and after filtration the residue was washed with dichloro-methane and dried. 762 mg (62%) of the title compound 28a-3 were isolated as solid.

### 5-(Polystyrene-ethoxy-diisobutyl-silanyl)-pentanoic acid 2,5-dioxo-pyrrolidin-1-yl ester

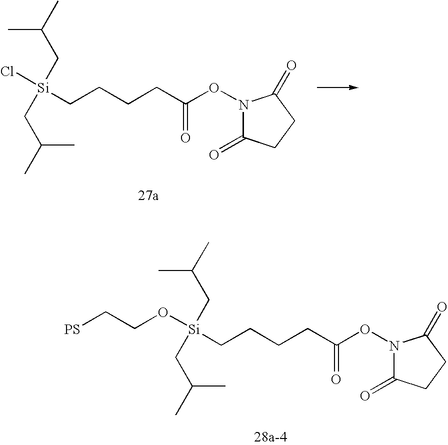

To a suspension of 576 mg 2-hydroxyethyl-polystyrene resin in 10 ml dichloro-methane was added the reaction mixture containing 5-(chloro-diisobutyl-silanyl)-pentanoic acid 2,5-dioxo-pyrrolidin-1-yl ester 27a (2.54 mmol at maximum) and 398 μl 1,5-diaza-bicyclo[3.3.3]undecane. The mixture was stirred for 2.5 hours at 23° C. and after filtration the residue was washed with dichloro-methane and dried. 741 mg (76%) of the title compound 28a-4 were isolated as solid.

### Part C

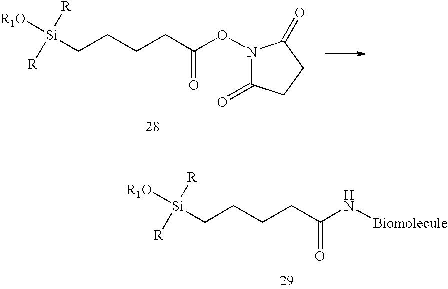

### 5-[Diisobutyl-(4-phenyl-butoxy)-silanyl]-pentanoic acid phenylamide

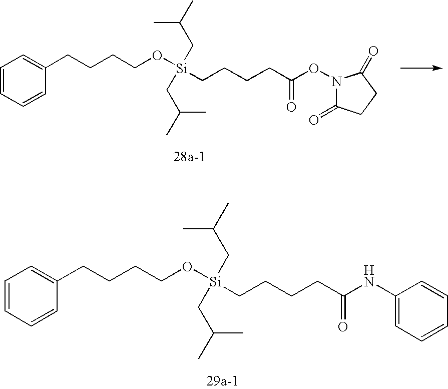

To a solution of 100 mg (204 μmol) 5-[Diisobutyl-(4-phenyl-butoxy)-silanyl]-pentanoic acid 2,5-dioxo-pyrrolidin-1-yl ester 28a-1 in 2 ml dichloro-methane were added 18.5 μl aniline, 28.3 μl triethylamine and a catalytic amount of dimethyl-pyridin-4-yl-amine. The mixture was stirred at 23° C. for 2 days and purified by chromatography to yield 49 mg (51%) of the title compound 29a-1 were isolated as an oil.

¹H-NMR (CDCl₃): δ=7.54 (2H), 7.40-7.10 (9H), 3.63 (2H), 2.65 (2H), 2.38 (2H), 1.91-1.29 (8H), 0.98 (14H), 0.69 (2H) 0.63 (4H) ppm.

### Part D

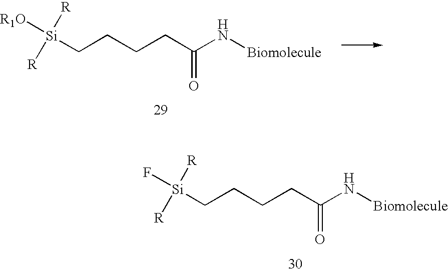

### 5-(Fluoro-diisobutyl-silanyl)-pentanoic acid phenylamide

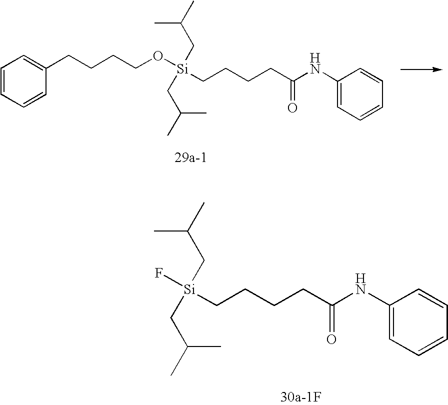

49 mg (105 μmol) 5-[Diisobutyl-(4-phenyl-butoxy)-silanyl]-pentanoic acid phenylamide 29a-1 was transformed in analogy to silanols or silanes to yield after isolation and purification 22 mg (62%) of the title compound 30a-1F as an oil.

¹H-NMR (400 MHz, CDCl₃): δ=7.54 (2H), 7.35 (2H), 7.17 (1H), 7.13 (1H), 2.40 (2H), 1.90 (2H), 1.81 (2H), 1.53 (2H), 0.99+1.00 (12H), 0.77 (2H), 0.71 (4H) ppm. ¹⁹F-NMR (376 MHz, CDCl₃): δ=−166.9 ppm.

### Radiochemistry

¹⁸F-Fluoride was azeotropically dried in the presence of Kryptofix 222 (5 mg), potassium carbonate (1 mg) in 1-2 ml CH₃CN/H₂O (3:1) by heating under nitrogen at 100-130° C. for 15-30 min. During this time 2−3×1 ml CH₃CN were added and evaporated to give the dried Kryptofix 222/K₂CO₃ complex (up to 43 GBq). After drying, a solution of the precursor (150-300 μl of 5-90 mM in DMSO) without or with AcOH (3-5 μl) was added. The reaction mixture was incubated in the range of ambient temperature (AT)-110° C. for 15-20 min to effect labeling. The crude reaction mixture was analyzed by analytical HPLC (columns: Hamilton PRP-1, 250×4.1 mm, 7μ or ACE C18, 50×4.6 mm, 3μ). The peak of the [¹⁸F]-labeled product was confirmed by coninjection or by comparison with the HPLC retention time of its non-radioactive reference molecule.

### [¹⁸F]Fluoro-diisopropyl-{4-[2-(tetrahydro-pyran-2-yloxy)-ethyl]-phenyl}-silane

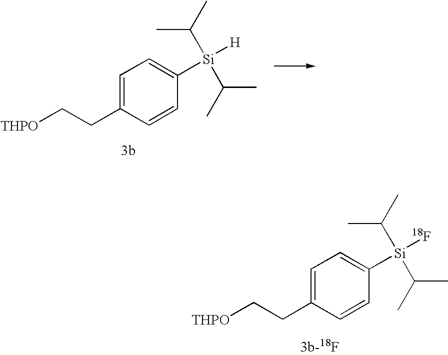

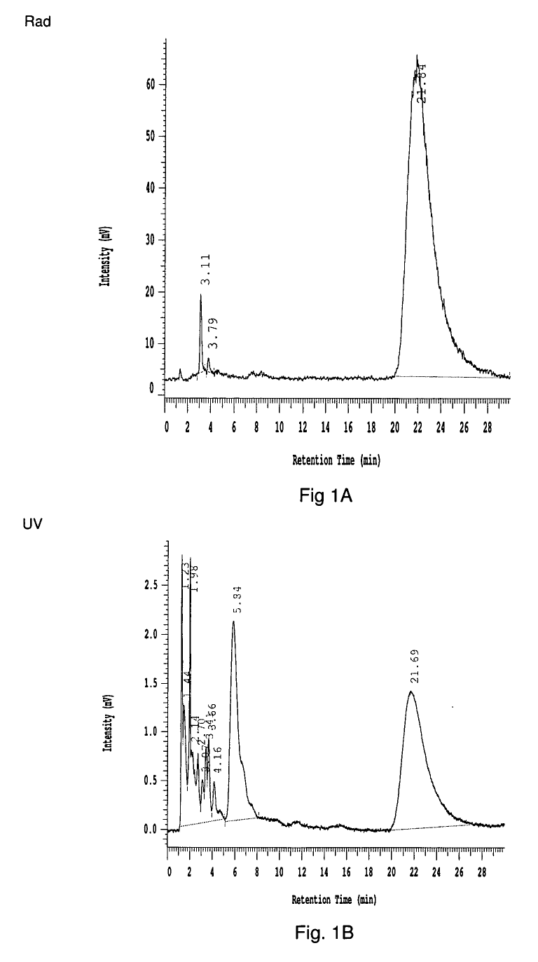

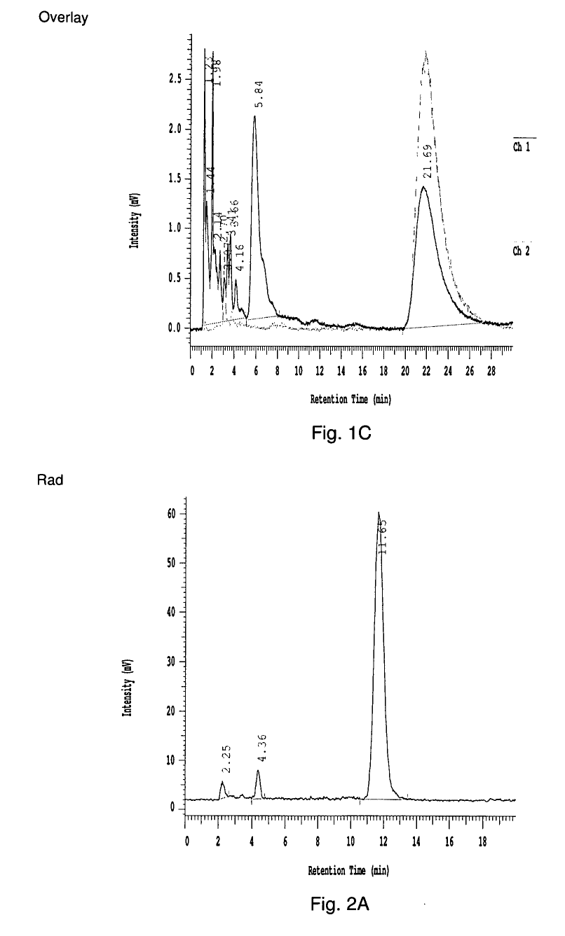

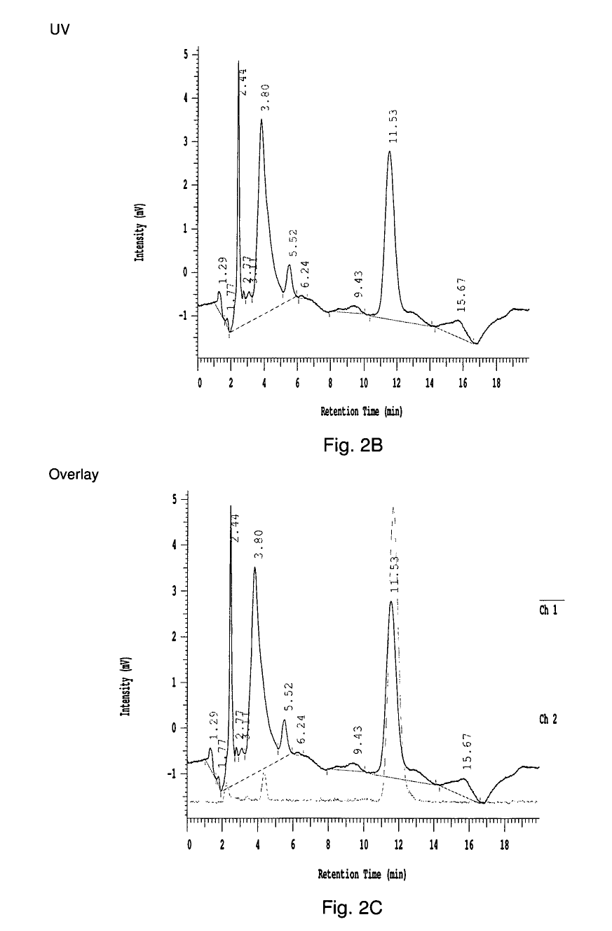

A solution of di-iso-propyl-{4-[2-(tetrahydro-pyran-2-yloxy)-ethyl]-phenyl}-silane (5.0 mg) in anhydrous DMSO (300 μl) was added to the dried Kryptofix 222/K₂CO₃ complex (4.3 GBq). After heating at 90° C. for 30 min an aliquot of the reaction mixture was diluted with CH₃CN (1 ml). The crude reaction mixture was analyzed using an analytical HPLC (PRP-1, CH₃CN/H₂O 90:10 isocratic, 1.0 ml/min). The yield determined by HPLC was 97%. The F-18 labeled product was confirmed by co-injection of an aliquot of the reaction mixture with the F-1 g cold standard on analytical HPLC (refer to FIGS. 1A, 1B, 1C).

### 2-[4-([¹⁸F]Fluoro-diisopropyl-silanyl)-phenyl]-ethanol

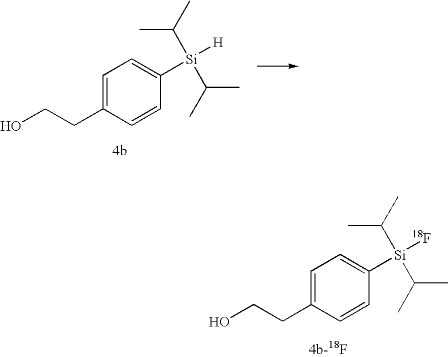

A solution of 2-(4-di-iso-propylsilanyl-phenyl)-ethanol (6.3 mg) in anhydrous DMSO (300 μl) and AcOH (3 μl) was added to the dried Kryptofix 222/K₂CO₃ complex (589 MBq). After heating at 65° C. for 15 min an aliquot of the reaction mixture was diluted with CH₃CN (1 ml). The crude reaction mixture was analyzed using an analytical HPLC (PRP-1, CH₃CN/H₂O 85:15 isocratic, 1.2 ml/min). The yield determined by HPLC was 70%.

### 2-[4-([¹⁸F]fluoro-di-iso-propyl-silanyl)-phenyl]-ethanol from the silanol

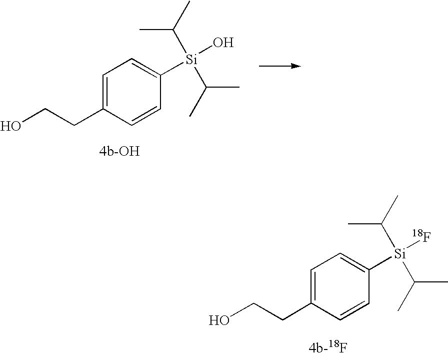

A solution of 2-[4-(hydroxy-di-iso-propyl-silanyl)-phenyl]-ethanol (5.1 mg) in anhydrous DMSO (300 μl) and AcOH (3 μl) was added to the dried Kryptofix 222/K₂CO₃ complex (364 MBq). After heating at 65° C. for 15 min an aliquot of the reaction mixture was diluted with CH₃CN (1 ml). The crude reaction mixture was analyzed using an analytical HPLC (PRP-1, CH₃CN/H₂O 85:15 isocratic, 1.2 ml/min). The yield determined by HPLC was 86%. Radiosynthesis of N-benzyl-2-(4-([¹⁸F]fluoro-di-iso-propylsilyl)phenyl)acetamide

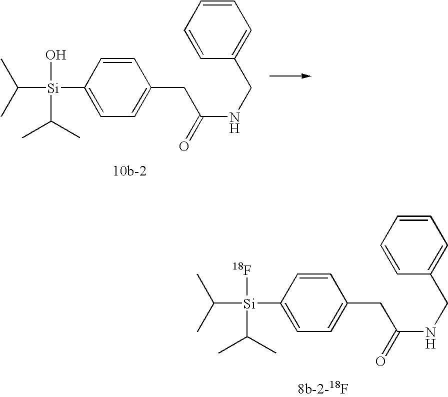

A solution of N-benzyl-2-(4-(hydroxyl-di-iso-propylsilyl)phenyl)acetamide (5.0 mg) in anhydrous DMSO (300 μl) and AcOH (3 μl) was added to the dried Kryptofix 222/K₂CO₃ complex (1.51 GBq). After heating at 65° C. for 15 min an aliquot of the reaction mixture was diluted with CH₃CN (1 ml). The crude reaction mixture was analyzed using an analytical HPLC (PRP-1, CH₃CN/H₂O 75:25 isocratic, 1.0 ml/min). The yield determined by HPLC was 90%.

### Radiosynthesis of 2-(4-fluorodi-iso-propylsilyl)phenyl)-N-(3-(3-(4-hydroxy-5-(hydroxymethyl)tetrahydrofuran-2-yl)-5-methyl-2,6-dioxo-2,3-dihydropyrimidin-1(6H)-yl)propyl)acetamide

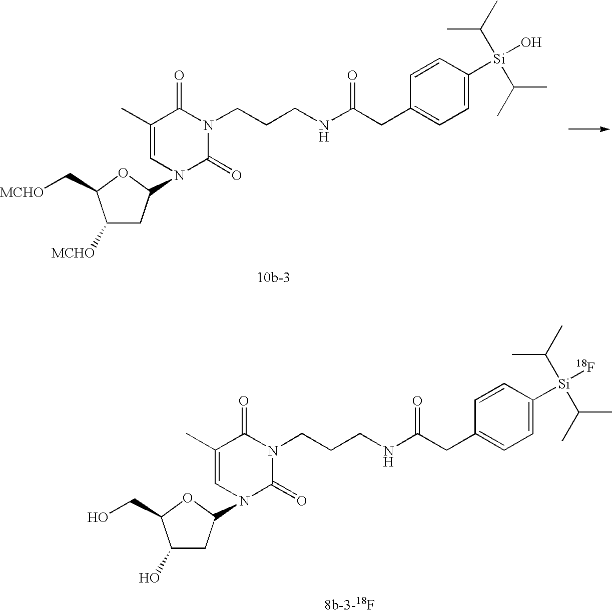

A solution of 2-(4-(hydroxyl-di-iso-propylsilyl)phenyl)-N-(3-(3-(4-(1-methoxycyclo-hexyloxy)-5-((1-methoxycyclohexyloxy)methyl)tetrahydrofuran-2-yl)-5-methyl-2,6-dioxo-2,3-dihydropyrimidin-1(6H)-yl)propyl)acetamide (2.0 mg) in anhydrous DMSO (150 μl) and AcOH (5 μl) was added to the dried Kryptofix 222/K₂CO₃ complex (4.1 GBq). After 20 min at 90° C. an aliquot of the reaction mixture was diluted with CH₃CN (1 ml). The crude reaction mixture was analyzed using an analytical HPLC (PRP-1, gradient CH₃CN/H₂O 30:70-100:0 in 10 min, then CH₃CN/H₂O 100:0 for 15 min, 1.0 ml/min). The yield of the protected product determined by HPLC was 72%. For the deprotection step, the reaction mixture was diluted with 9 ml H₂O, the passed through a Waters tC18 Sep-Pak light cartridge. The cartridge as washed with 2×5 ml H₂O and then eluted with 1.0 ml CH₃CN. To the eluate 0.5 ml of 1 N HCl was added. After incubation at AT for 5 min an aliquot of the reaction mixture was diluted with CH₃CN (1 ml). The crude reaction mixture was analyzed using an analytical HPLC (PRP-1, gradient CH₃CN/H₂O 30:70-100:0 in 10 min, then CH₃CN/H₂O 100:0 for 15 min, 1.0 ml/min). The radiochemical purity of the product determined by HPLC was 72%.

### 2-[4-([¹⁸F]Fluoro-diisopropyl-silanyl)-phenyl]-acetyl-Val-βAla-Phe-Gly-NH₂

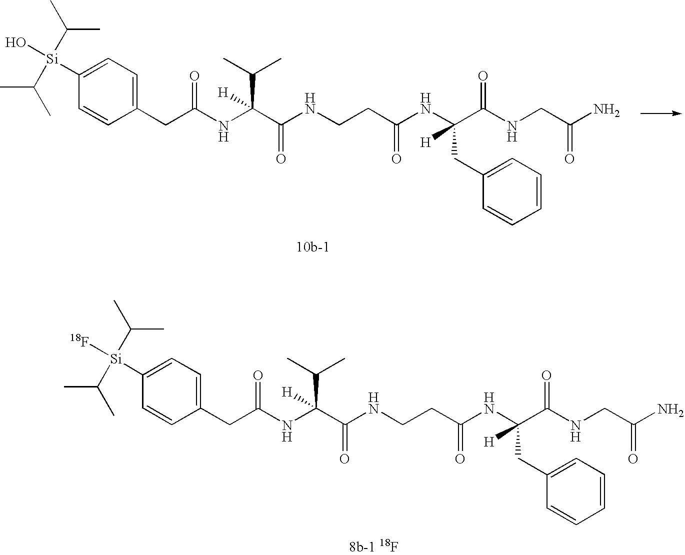

[¹⁸F]Fluoride was eluted from the QMA Light cartridge (Waters) into a Reactivial (10 ml) with a solution of Kryptofix 222 (5 mg), potassium carbonate (1 mg) in water (500 μl) and MeCN (1.5 ml). The solvent was removed by heating at 100° C. under vacuum with a stream of nitrogen. Anhydrous MeCN (1 ml) was added and evaporated as before. This step was repeated again to give the dried Kryptofix 222/K₂CO₃ complex (2.0 GBq). A solution of 2-[4-(hydroxy-diisopropyl-silanyl)-phenyl]-acetyl-Val-βAla-Phe-Gly-NH₂ 10b-1 (1.0 mg) in anhydrous DMSO (300 μl) and AcOH (3 μl) was added. After heating at 90° C. for 15 mins an aliquot of the reaction mixture was diluted with MeCN (1 ml). The crude reaction mixture was analyzed using an analytical HPLC (Column Hamilton PRP-1, 250×4.1 mm, 7μ, 1.0 ml/min, solvent MeCN/H₂O 45:55 isocratic 15 mins). The yield determined by HPLC was 53%. The peak of the [¹⁸F]-labeled product was confirmed by coninjection.

### Radiosynthesis of N-benzyl-3-(3-([¹⁸F]fluoro-di-iso-propylsilyl)phenyl)propanamide

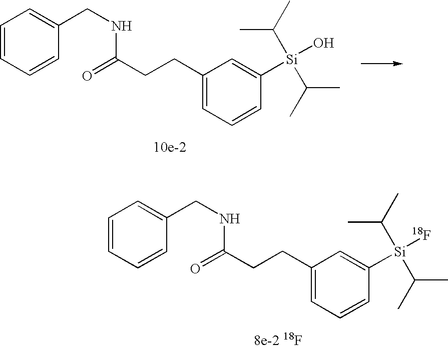

A solution of N-benzyl-3-(3-(hydroxyl-di-iso-propylsilyl)phenyl)propanamide (5.0 mg) in anhydrous DMSO (300 μl) and AcOH (3 μl) was added to the dried Kryptofix 222/K₂CO₃ complex (1.61 GBq). After heating at 65° C. for 15 min an aliquot of the reaction mixture was diluted with CH₃CN (1 ml). The crude reaction mixture was analyzed using an analytical HPLC (PRP-1, CH₃CN/H₂O 80:20 isocratic, 1.0 ml/min). The yield determined by HPLC was 89%.

### 3-[3-([13F]Fluoro-diisopropyl-silanyl)-phenyl]-propanyl-Val-βAla-Phe-Gly-NH₂

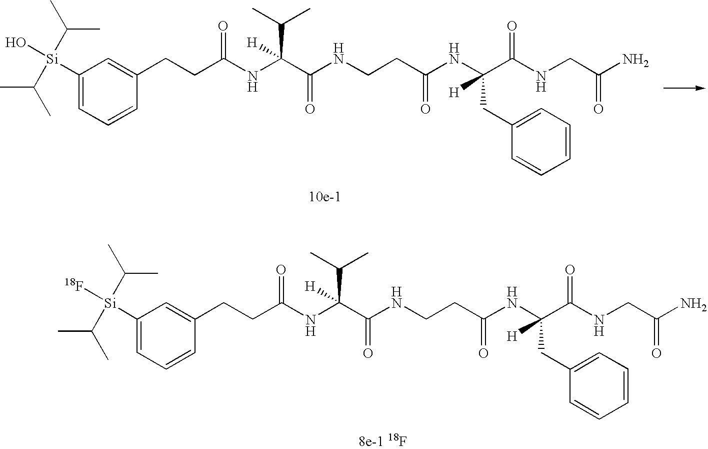

A solution of 3-[3-(hydroxy-di-iso-propyl-silanyl)-phenyl]-propyl-Val-βAla-Phe-Gly-NH₂ (2.0 mg) in anhydrous DMSO (300 μl) and AcOH (3 μl) was added to the dried Kryptofix 222/K₂CO₃ complex (4.36 GBq). After heating at 90° C. for 30 min an aliquot of the reaction mixture was diluted with CH₃CN (1 ml). The crude reaction mixture was analyzed using an analytical HPLC (PRP-1, 10 mM K₂HPO₄ in CH₃CN/H₂O (7:3)/10 mM K₂HPO₄ in H₂O 70:30 isocratic, 0.8 ml/min). The yield determined by HPLC was 46%. The F-18 labeled product was confirmed by co-injection of an aliquot of the reaction mixture with the F-19 cold standard on analytical HPLC (refer to FIGS. 2A, 2B, 2C).

### Radiosynthesis of N-benzyl-3-(4-([¹⁸F]fluoro-di-iso-propylsilyl)-methylphenoxy)propanamide from the hydrosilane

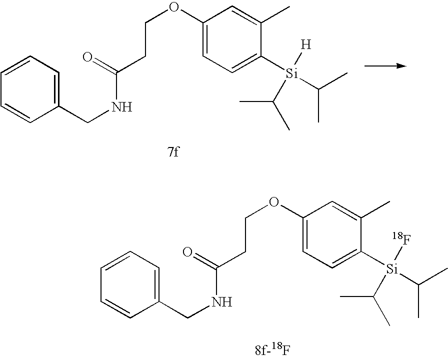

A solution of N-benzyl-3-(4-(di-iso-propylsilyl)-3-methylphenoxy)propanamide (5.0 mg) in anhydrous DMSO (300 μl) was added to the dried Kryptofix 222/K₂CO₃ complex (2.1 GBq). After heating at 65° C. for 15 min an aliquot of the reaction mixture was diluted with CH₃CN (1 ml). The crude reaction mixture was analyzed using an analytical HPLC (PRP-1, CH₃CN/H₂O 75:25 isocratic, 1.0 ml/min). The yield determined by HPLC was 73%.

### Radiosynthesis of N-benzyl-3-(4-([¹⁸F]fluoro-di-iso-propylsilyl)-3,5-dimethylphenoxy) propanamide

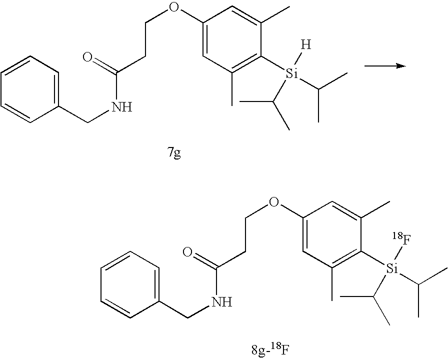

A solution of N-benzyl-3-(4-(di-iso-propylsilyl)-3,5-dimethylphenoxy)propanamide (5.0 mg) in anhydrous DMSO (300 μl) was added to the dried Kryptofix 222/K₂CO₃ complex (3.3 GBq). After heating at 90° C. for 15 min an aliquot of the reaction mixture was diluted with CH₃CN (1 ml). The crude reaction mixture was analyzed using an analytical HPLC (PRP-1, CH₃CN/H₂O 80:20 isocratic, 1.0 ml/min). The yield determined by HPLC was 48%. BENZYL 2-(di-tert-butyl-[¹⁸F]fluorosilyl)acetate

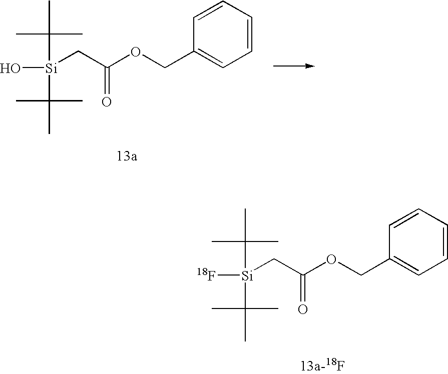

A solution of benzyl 2-(di-tert-butyl(hydroxy)silyl)acetate (5.0 mg) in anhydrous DMSO (300 μl) and AcOH (3 μl) was added to the dried Kryptofix 222/K₂CO₃ complex. After 15 min at RT an aliquot of the reaction mixture was diluted with CH₃CN (1 ml). The crude reaction mixture was analyzed using an analytical HPLC (PRP-1, CH₃CN/H₂O 85:15 isocratic, 1.2 ml/min). The yield determined by HPLC was 79%.

### Radiosynthesis of 1-(di-tert-butylfluorosilyl)acetyl-Val-βAla-Phe-Gly-NH₂

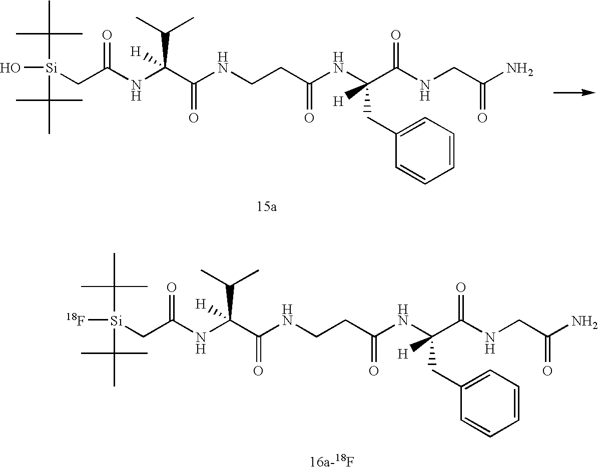

A solution of 2-(di-tert-butyl(hydroxy)silyl)acetyl-Val-βAla-Phe-Gly-NH₂ (2 mg) in anhydrous DMSO (300 μl) and AcOH (3 μl) was added to the dried Kryptofix 222/K₂CO₃ complex. After heating at 50° C. for 15 min an aliquot of the reaction mixture was diluted with CH₃CN (1 ml). The crude reaction mixture was analyzed using an analytical HPLC (PRP-1, 10 mM K₂HPO₄ in CH₃CN/H₂O (7:3)/10 mM K₂HPO₄ in H₂O 60:40 isocratic, 0.8 ml/min). The yield determined by HPLC was 82%.

### Radiosynthesis of N-benzyl-2-(4-(di-tert-butyl[¹⁸F]fluorosilyl)phenyl)acetamide from the silane

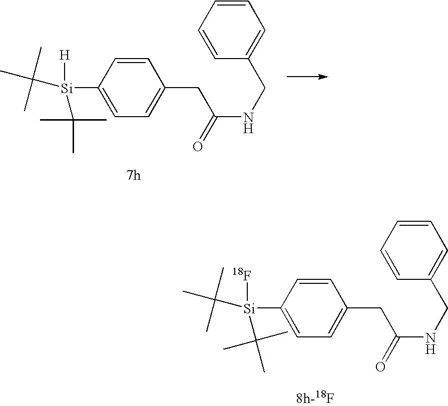

A solution of N-benzyl-2-(4-(di-tert-butylsilyl)phenyl)acetamide (5 mg) in anhydrous DMSO (300 μl) was added to the dried Kryptofix 222/K₂CO₃ complex (2.3 GBq). After 15 min at 65° C. an aliquot of the reaction mixture was diluted with CH₃CN (1 ml). The crude reaction mixture was analyzed using an analytical HPLC (PRP-1, CH₃CN/H₂O 80:20 isocratic, 0.8 ml/min). The yield determined by HPLC was 73%.

### Radiosynthesis of N-benzyl-2-(4-(di-tert-butyl[¹⁸F]fluorosilyl)phenyl)acetamide from the silanol

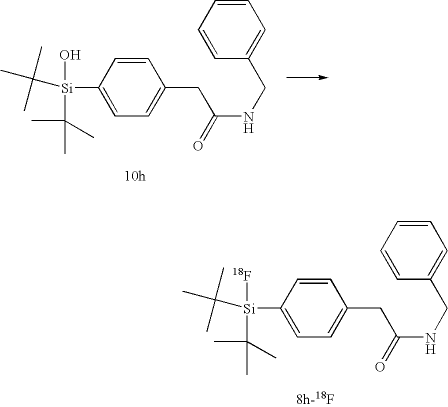

A solution of N-benzyl-2-(4-(di-tert-butyl(hydroxy)silyl)phenyl)acetamide (5 mg) in anhydrous DMSO (300 μl) and AcOH (3 μl) was added to the dried Kryptofix 222/K₂CO₃ complex (1.9 GBq). After 15 min at 65° C. an aliquot of the reaction mixture was diluted with CH₃CN (1 ml). The crude reaction mixture was analyzed using an analytical HPLC (PRP-1, CH₃CN/H₂O 80:20 isocratic, 0.8 ml/min). The yield determined by HPLC was 68%.

### Radiosynthesis of 4-(Fluordi-tert-butylsilyl)-phenylacetyl-Ava-Gln-Trp-Ala-Val-NMeGly-His(3Me)-[4-(R)-amino-5-(S)-methylheptanoyl]-Cpa-NH₂

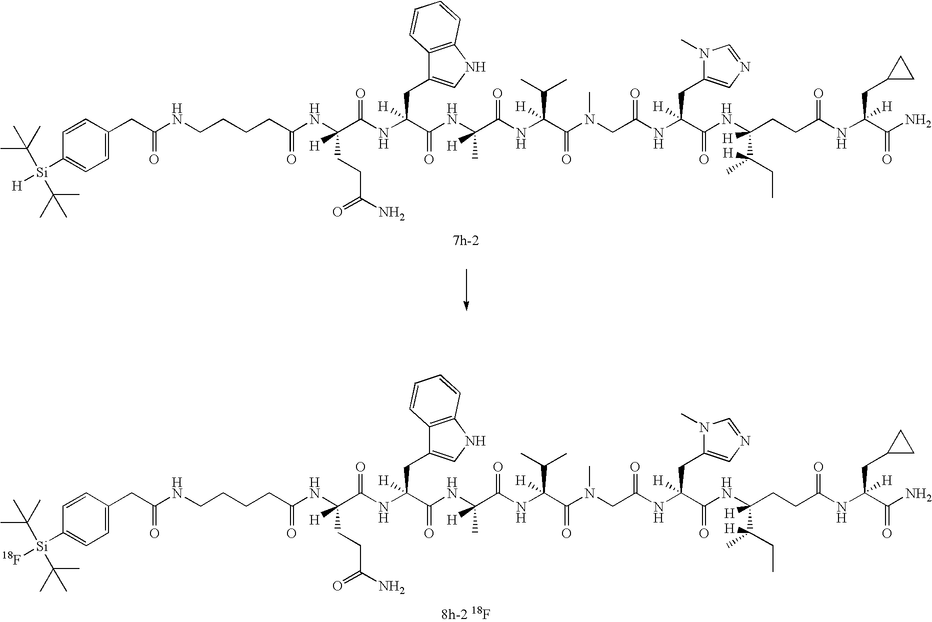

A solution of 2-(4-(di-tert-butylsilyl)phenyl)acetyl-Ava-Gln-Trp-Ala-Val-NMeGly-His(3Me)-[4-(R)-amino-5-(S)-methylheptanoyl]-Cpa-NH₂ (1.5 mg) in anhydrous DMSO (150 μl) was added to the dried Kryptofix 222/K₂CO₃ complex (43 GBq). After heating at 70° C. for 30 min an aliquot of the reaction mixture was diluted with CH₃CN (1 ml). The crude reaction mixture was analyzed using an analytical HPLC (PRP-1, 10 mM K₂HPO₄ in CH₃CN/H₂O (7:3)/10 mM K₂HPO₄ in H₂O 70:30 isocratic, 0.8 ml/min). The yield determined by HPLC was 19%.

### Radiosynthesis of 2-(4-(di-tert-butyl[¹⁸F]fluorosilyl)phenyl)acetyl-Val-βAla-Phe-Gly-NH₂ from the silane

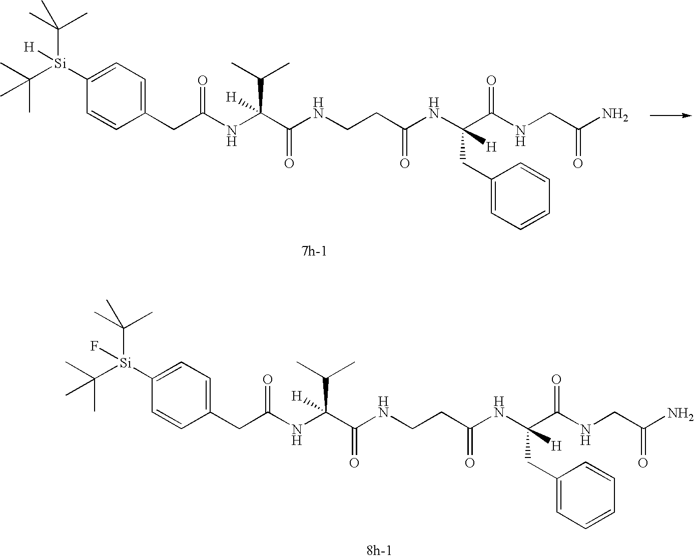

A solution of 2-(4-(di-tert-butylsilyl)phenyl)acetyl-Val-βAla-Phe-Gly-NH₂ (2.0 mg) in anhydrous DMSO (300 μl) was added to the dried Kryptofix 222/K₂CO₃ complex (25.4 GBq). After heating at 90° C. for 20 min the reaction mixture was diluted with 4.0 ml HPLC eluent (CH₃CN/H₂O (55:45)+0.1% TFA) and an aliquot of this solution was diluted with CH₃CN (1 ml). The crude reaction mixture was analyzed using an analytical HPLC (PRP-1, 10 mM K₂HPO₄ in CH₃CN/H₂O (7:3)/10 mM K₂HPO₄ in H₂O 75:25 isocratic, 0.8 ml/min). The yield determined by HPLC was 35%. This solution was injected into a semi-prep HPLC (column: ACE C18, 250×10 mm, 5μ; CH₃CN/H₂O (55:45)+0.1% TFA, isocratic, 3.0 ml/min) and the desired product peak was collected (2.6 GBq, 15% d.c.).

### Radiosynthesis of 2-(4-(di-tert-butyl[¹⁸F]fluorosilyl)phenyl)acetyl-Arg-Ava-Gln-Trp-Ala-Val-NMeGly-His-Sta-Leu-NH₂

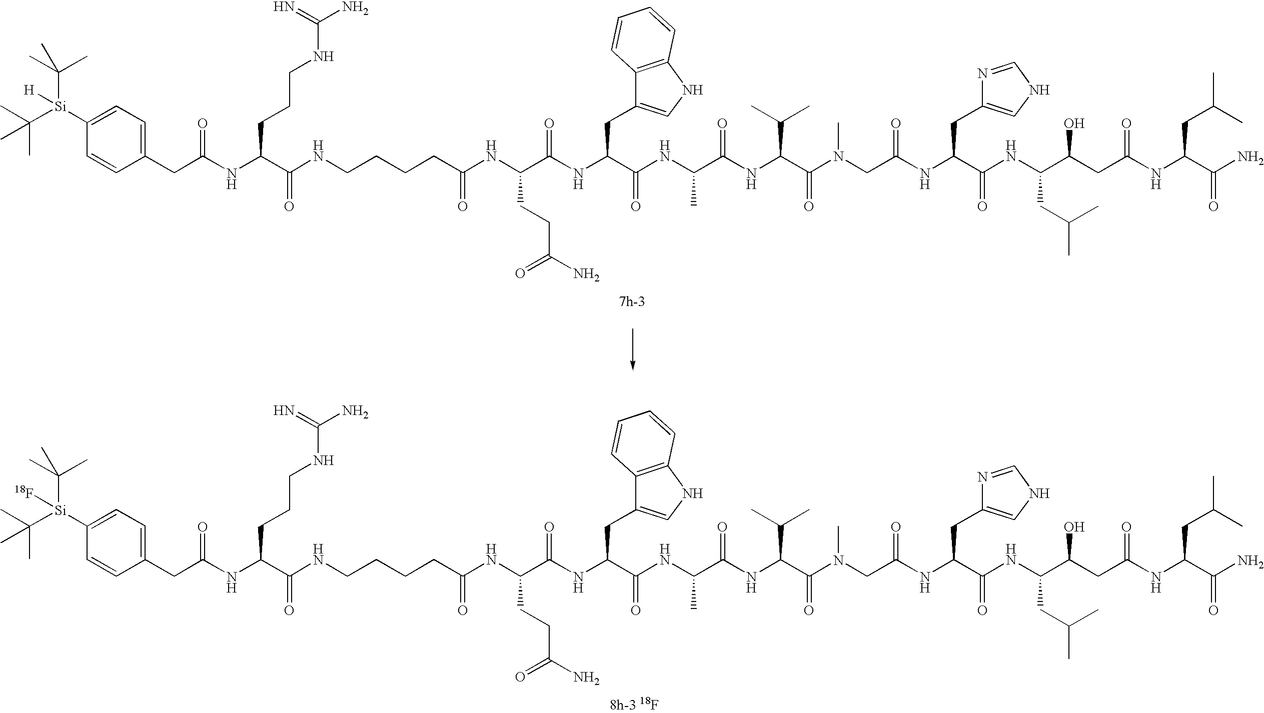

A solution of 2-(4-(di-tert-butylsilyl)phenyl)acetyl-Arg-Ava-Gln-Trp-Ala-Val-NMeGly-His-Sta-Leu-NH₂ (2.0 mg) in anhydrous DMSO (150 μl) was added to the dried Kryptofix 222/K₂CO₃ complex (2.30 GBq). After heating at 110° C. for 20 min an aliquot of the reaction mixture was diluted with CH₃CN (1 ml). The crude reaction mixture was analyzed using an analytical HPLC (ACE C18, gradient 10 mM K₂HPO₄ in CH₃CN/H₂O (7:3)/10 mM K₂HPO₄ in H₂O 5:95-95:5 in 7 min, 2.0 ml/min). The yield determined by HPLC was 75%. The reaction mixture was diluted with 4.0 ml HPLC eluent (CH₃CN/H₂O (45:55)+0.1% TFA). This solution was injected into a semi-prep HPLC (column: ACE C18, 250×10 mm, 5μ; CH₃CN/H₂O (45:55)+0.1% TFA, isocratic, 3.0 ml/min) and the desired product peak was collected (455 MBq, 25% d.c.).

### Radiosynthesis of 2-(4-(di-tert-butyl[¹⁸F]fluorosilyl)phenyl)acetyl-Arg-Ava-Gln-Trp-Ala-Val-Gly-His (3Me)-Sta-Leu-NH₂

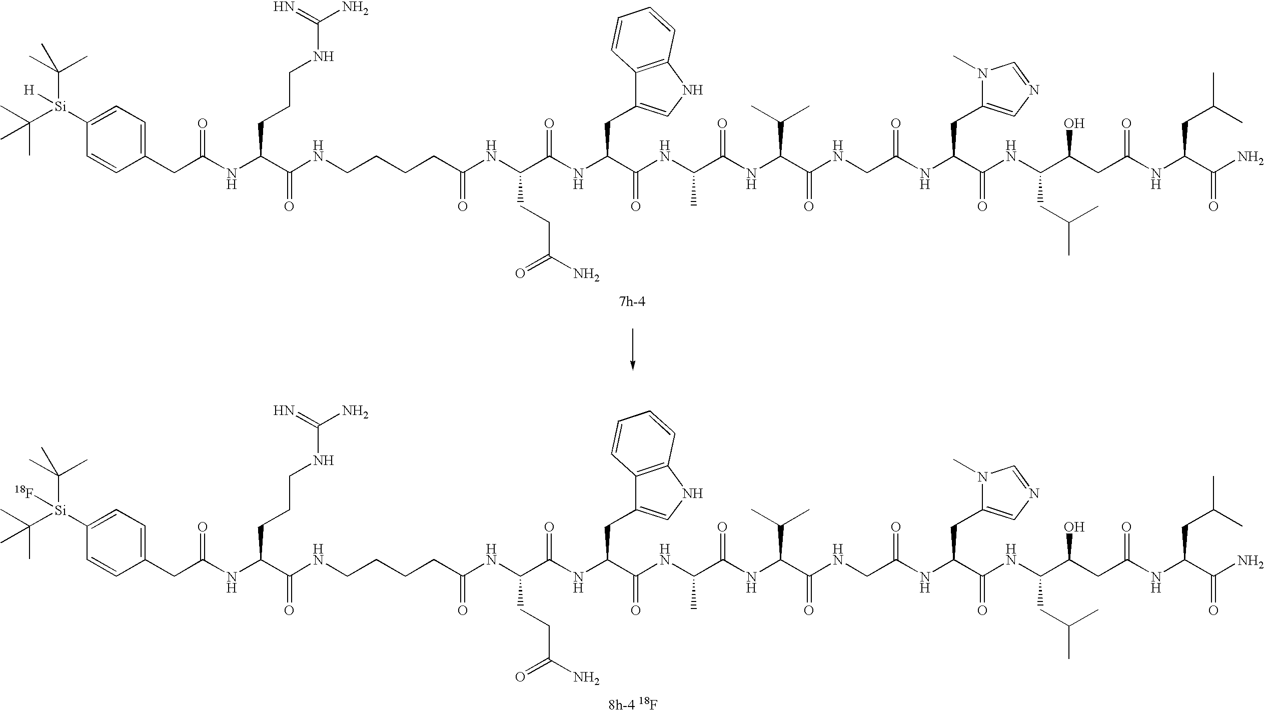

A solution of 2-(4-(di-tert-butylsilyl)phenyl)acetyl-Arg-Ava-Gln-Trp-Ala-Val-Gly-His(3Me)-Sta-Leu-NH₂ (2.0 mg) in anhydrous DMSO (150 μl) was added to the dried Kryptofix 222/K₂CO₃ complex (257 MBq). After heating at 110° C. for 20 min an aliquot of the reaction mixture was diluted with CH₃CN (1 ml). The crude reaction mixture was analyzed using an analytical HPLC (ACE C18, gradient 10 mM K₂HPO₄ in CH₃CN/H₂O (7:3)/10 mM K₂HPO₄ in H₂O 5:95-95:5 in 7 min, 2.0 ml/min). The yield determined by HPLC was 85%. The reaction mixture was diluted with 4.0 ml HPLC eluent (CH₃CN/H₂O (45:55)+0.1% TFA). This solution was injected into a semi-prep HPLC (column: ACE C18, 250×10 mm, 5p; CH₃CN/H₂O (45:55)+0.1% TFA, isocratic, 3.0 ml/min) and the desired product peak was collected (55 MBq, 30% d.c.).

### Radiosynthesis of 2-(4-(di-tert-butyl-[¹⁸F]fluorosilyl)phenyl)-N-(3-(3-(4-hydroxy-5-(hydroxymethyl)tetrahydrofuran-2-yl)-5-methyl-2,6-dioxo-2,3-dihydropyrimidin-1(6H)-yl)propyl)acetamide

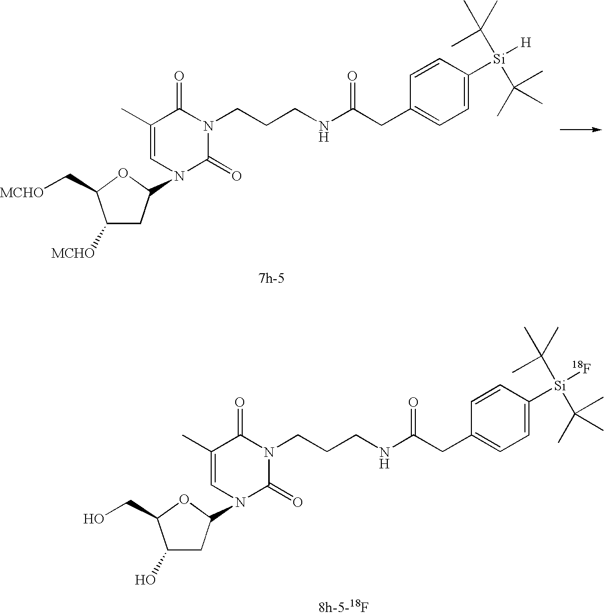

A solution of 2-(4-(di-tert-butylsilyl)phenyl)-N-(3-(3-(4-(1-methoxycyclohexyloxy)-5-((1-methoxycyclohexyloxy)methyl)tetrahydrofuran-2-yl)-5-methyl-2,6-dioxo-2,3-dihydro-pyrimidin-1(6H)-yl)propyl)acetamide (2.0 mg) in anhydrous DMSO (150 μl) was added to the dried Kryptofix 222/K₂CO₃ complex (4.03 GBq). After 20 min at 90° C. an aliquot of the reaction mixture was diluted with CH₃CN (1 ml). The crude reaction mixture was analyzed using an analytical HPLC (PRP-1, gradient CH₃CN/H₂O 30:70-100:0 in 10 min, then CH₃CN/H₂O 100:0 for 15 min, 1.0 ml/min). The sum of yields of the protected, monoprotected and deprotected product(s) determined by HPLC was 68%. For the deprotection step, the reaction mixture was diluted with 9 ml H₂O, the passed through a Waters tC18 Sep-Pak light cartridge. The cartridge as washed with 2×5 ml H₂O and then eluted with 1.0 ml CH₃CN. To the eluate 0.5 ml of 1 N HCl was added. After incubation at AT for 5 min an aliquot of the reaction mixture was diluted with CH₃CN (1 ml). The crude reaction mixture was analyzed using an analytical HPLC (PRP-1, gradient CH₃CN/H₂O 30:70-100:0 in 10 min, then CH₃CN/H₂O 100:0 for 15 min, 1.0 ml/min). The radiochemical purity of the product determined by HPLC was 95%.

### Radiosynthesis of N-(3-([¹⁸F]fluorodiisopropylsilyl)propyl)biphenyl-4-carboxamide

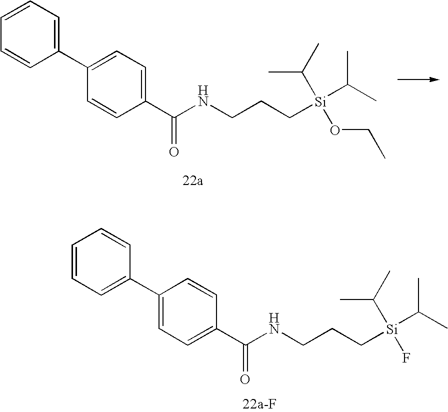

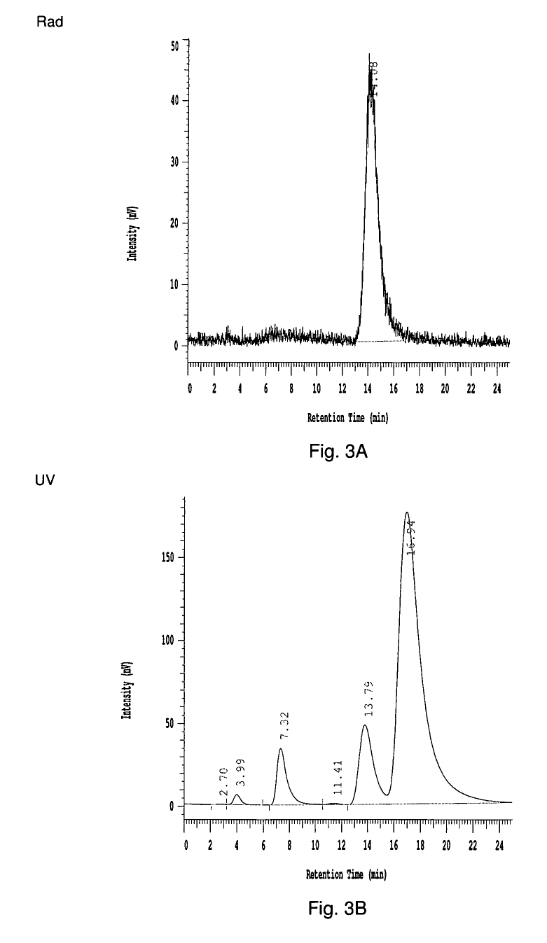

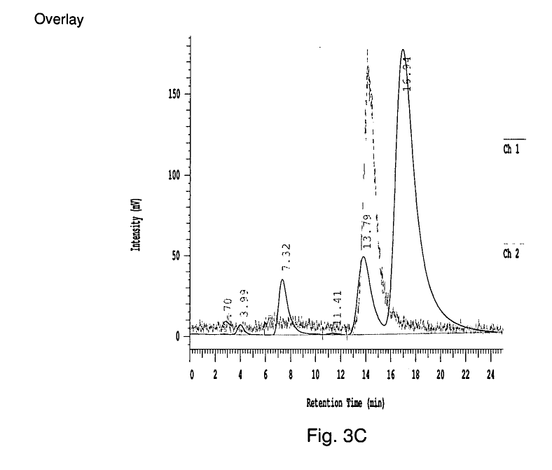

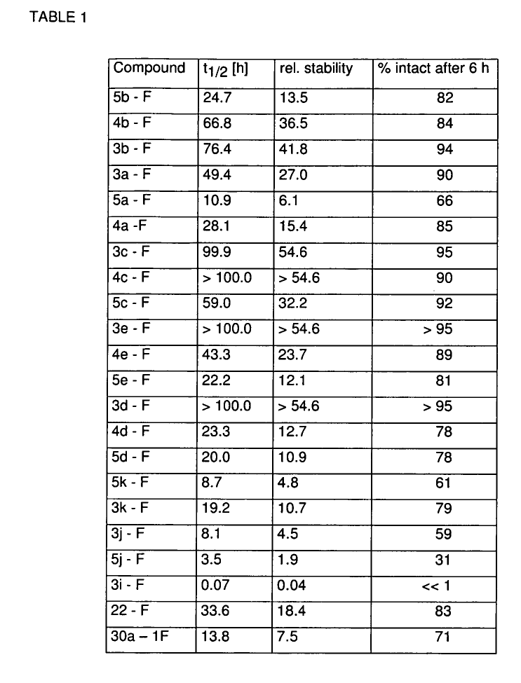

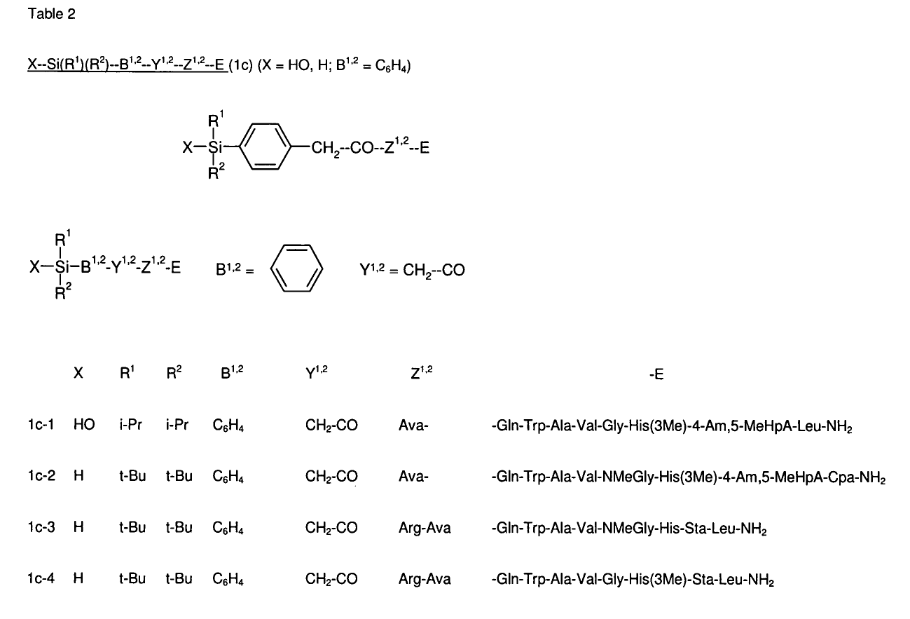

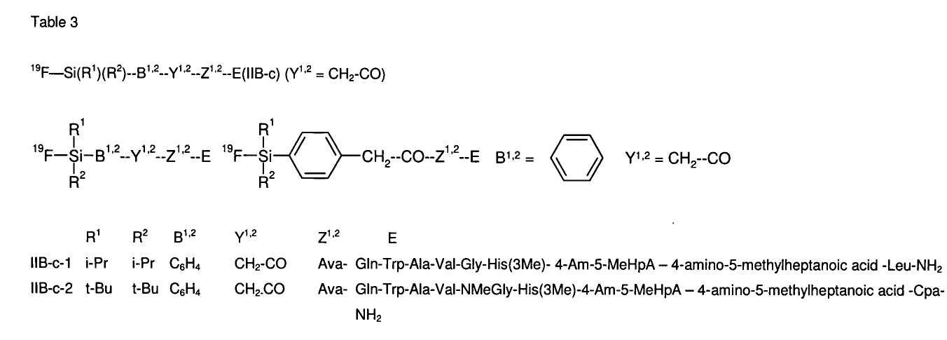

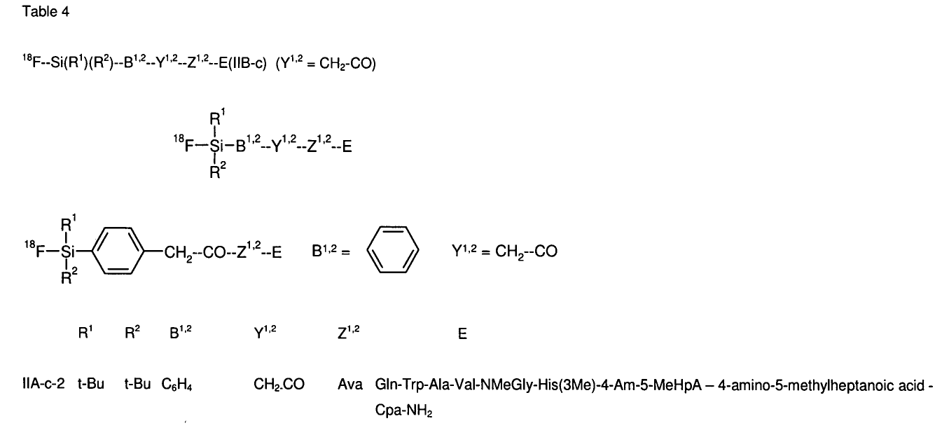

A solution of N-(3-(ethoxydiisopropylsilyl)propyl)biphenyl-4-carboxamide (5.0 mg) in anhydrous DMSO (300 μl) and AcOH (3 μl) was added to the dried Kryptofix 222/K₂CO₃ complex. After heating at 65° C. for 15 min an aliquot of the reaction mixture was diluted with CH₃CN (1 ml). The crude reaction mixture was analyzed using an analytical HPLC (PRP-1, CH₃CN/H₂O 85:15 isocratic, 1.0 ml/min). The yield determined by HPLC was 96%. The F-18 labeled product was confirmed by co-injection of an aliquot of the reaction mixture with the F-1 g cold standard on analytical HPLC (refer to FIGS. 3A, 3B, 3C).

### Benzyl 2-(di-tert-butyl-[¹⁸F]fluorosilyl)acetate from the benzyloxysilane

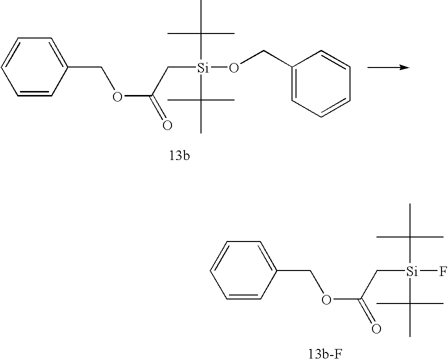

A solution of benzyl 2-(benzyloxydi-tert-butylsilyl)acetate 13b (6.0 mg) in anhydrous DMSO (300 μl) and AcOH (3 μl) was added to the dried Kryptofix 222/K₂CO₃ complex (3.4 GBq). After 15 min at RT an aliquot of the reaction mixture was diluted with CH₃CN (1 ml). The crude reaction mixture was analyzed using an analytical HPLC (CH₃CN/H₂O 85:15 isocratic, 1.2 ml/min, t_{R}(product)=8.7 min). The yield determined by HPLC was 28%. The peak of the [¹⁸F]-labeled product was confirmed by coinjection.

### Examples for Hydrolytic Stability

The time dependent degree of hydrolysis of compounds having general chemical Formula II in which F is a fluorine atom of isotope 19 to compounds having general chemical Formula I in which X is a hydroxy group was determined at physiological pH (7.0). From the kinetics the hydrolytic halflife (t_{1/2}) measured in hours [h] of compounds having general chemical Formula II in which F is a fluorine atom of isotope 19 were calculated (Table 1, Column 2).

The ratio of the hydrolytic halflifes of the compounds having general chemical Formula II in which F is a fluorine atom of isotope 19 and the radioactive halflife of the 18-F isotope (1.83 hours) is defined as relative stability (Table 1, Column 3).

After 6 hours 90% of the 18-F isotope have been decayed. The fraction of intact compounds having general chemical Formula II in which F is a fluorine atom of isotope 19 in % is given in Table 1, Column 4.

Compounds having general chemical Formula II in which F is a fluorine atom of isotope 18 are considered as useful for PET applications if the relative stability of their analogs bearing a fluorine atom of isotope 19 exceed a factor of four and if at least 50% of these compounds remain intact after 6 hours under hydrolytic conditions at pH 7.

### Data are Listed in Table 1

In vitro binding affinity In vitro binding affinity and specificity of Bombesin analogs for the human bombesin 2 receptor (GRPR) were assessed via a competitive receptor-binding assay using ¹²⁵I-[Tyr⁴]-Bombesin (Perkin Elmer; specific activity 81.4 TBq/mmol) as GRPR-specific radioligand. The assay was performed based on the scintillation proximity assay (SPA) technology (J. W. Carpenter et al., Meth. Mol. Biol., 2002; 190:31-49) using GRPR-containing cell membranes (Perkin Elmer) and wheat germ agglutinin (WGA)-coated PVT beads (Amersham Bioscience).

Briefly, GRPR-containing membranes and WGA-PVT beads were mixed in assay buffer (50 mM Tris/HCl pH 7.2, 5 mM MgCl₂, 1 mM EGTA, Complete protease inhibitor (Roche Diagnostics GmbH) and 0.3% PEI) to give final concentrations of approximately 100 μg/ml protein and 40 mg/ml PVT-SPA beads. The ligand ¹²⁵I-[Tyr⁴]-Bombesin was diluted to 0.5 nM in assay buffer. The test compounds were dissolved in DMSO to give 1 mM stock solutions. Later on, they were diluted in assay buffer to 8 μM-1.5 μM.

Synthesis of H—Y-E: Solid-phase peptide synthesis (SPPS) involves the addition of amino acid residues to a growing peptide chain that is linked to an insoluble support or matrix, such as polystyrene. The C-terminal residue of the peptide is first anchored to a commercially available support (e.g., Rink amide resin) with its amino group protected with an N-protecting agent, fluorenylmethoxycarbonyl (FMOC) group. The amino protecting group is removed with suitable deprotecting agent such as piperidine for FMOC and the next amino acid residue (in N-protected form) is added with a coupling agents such as dicyclohexylcarbodiimide (DCC), di-isopropyl-cyclohexylcarbodiimide (DCCl), hydroxybenzotriazole (HOBt). Upon formation of a peptide bond, the reagents are washed from the support. After addition of the final residue of (Y), the peptide is attached to the solid support is ready for the coupling of RG-L₁-B₁—OH.

The assay was then performed as follows: First, 10 μl of compound solution to be tested for binding were placed in white 384 well plates (Optiplate-384, Perkin-Elmer). At next, 20 μl GRPR/WGA-PVT bead mixture and 20 μl of the ligand solution were added. After 90 minutes incubation at room temperature, another 50 μl of assay buffer were added, the plate sealed and centrifuged for 10 min at 520×g at room temperature. Signals were measured in a TopCount (Perkin Elmer) for 1 min integration time per well. The IC₅₀ was calculated by nonlinear regression using the GraFit data analysis software (Erithacus Software Ltd.). Furthermore, the Kᵢ was calculated based on the IC₅₀ for test compound as well as the K_{D} and the concentration of the ligand ¹²⁵I-[Tyr⁴]-Bombesin. Experiments were done with quadruple samples.

Aminoacid Abbreviations

All natural amino acids are represented by 3-letter codes. Unless otherwise stated all the aminoacids have L-configurations.

### Sta—Statine

His(3Me)—3-methylhisitidine

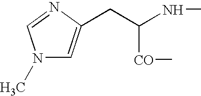

Ava—5-aminovaleric acid
AOC—8-aminooctanoic acid
tBuGly—t-butylglycine
tBuAla—t-butylalanine
βhLeu—β-homoleucine
βhlle—β-homoisoleucine

### Lys(Me)₂—εF-N,N-dimethyllysine

DOA—3,6-dioxa-8-aminooctanoic acid
4-Am-5-MeHpA—4-amino-5-methylheptanoic acid
4-Am-5-MeHxA—4-amino-5-methylhexanoic acid

1,4-cis-ACHC—1,4-cis-aminocyclohexamecarboxylic acid

AHMHxA—(3R,4S)-4-amino-3-hydroxy-5-methylhexanoic acid

1. A compound having general chemical Formula I 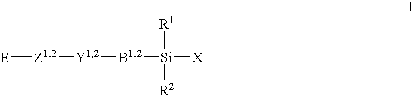
wherein
X represents a leaving group suitable for fluorination selected from the group comprising hydrogen and OR³,
wherein R³ represents hydrogen, C₁-C₁₀ alkyl, C₂-C₁₀ alkenyl, C₂-C₁₀ alkynyl, aryl, heteroaryl or aralkyl,
R¹ and R², independently, are selected from the group comprising hydrogen, linear and branched C₁-C₁₀ alkyl, aryl, heteroaryl and aralkyl,
—B¹— is selected from the group comprising —[CH₂]ₘ-D-[CH₂]ₙ-A-,
wherein n and m, independently, are any integer from 0 to 5,
-D- represents a bond, —S—, —O— or —NR⁴—,
wherein R⁴ represents hydrogen, C₁-C₁₀ alkyl, aryl, heteroaryl or aralkyl,
A represents alkyl, unsubstituted or substituted aryl or heteroaryl,
E-Z¹-Y¹— represents a moiety selected from the group comprising EO-C(═O)—, ENR⁵—C(═O)—, EC(═O)—O—, EC(═O)—NR⁵—, ENR⁵—SO₂—, ESO₂—NR⁵—, E-O—, E-(S)ₚ—, E-NR⁵—, ENR⁶C(═O)—NR⁷—, ENR⁶—C(═O)NR⁷—, ENR⁶C(═S)—NR⁷—, ENR⁶—C(═S)NR⁷—, EO-C(═O)O—, EOC(═O)—O—, EOC(═S)—O—, EO-C(═S)O—,
wherein the long single bond explicitly shown in the Formulae herein above is the bond between Z¹ and Y¹, and 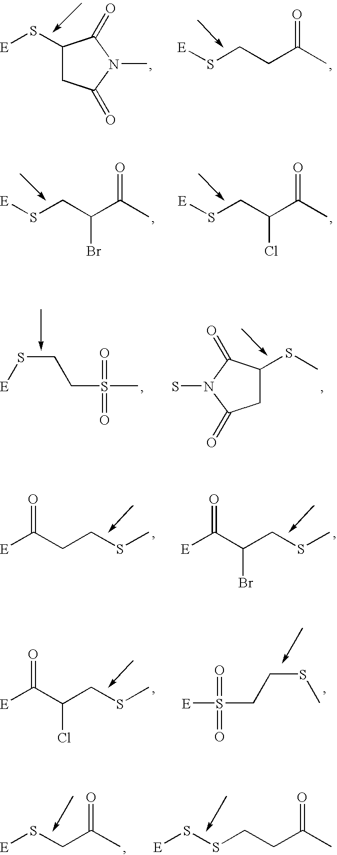
wherein the arrows shown in the Formulae herein above indicate the bond between Z¹ and Y¹,
R⁵, R⁶ and R⁷, independently, represent hydrogen, linear or branched C₁-C₁₀ alkyl, aryl, heteroaryl or aralkyl,
p is any integer from 1 to 3, and
wherein E-Z¹- is a targeting agent radical and E- is a biomolecule,
or
—B²— represents a C₁-C₁₀ alkyl-, unsubstituted or substituted -aryl- or unsubstituted or substituted -heteroaryl-,
—Y²— is selected from the group comprising a bond, —C(═O)—, —SO₂—, —C(═O)—(CH₂)_{d}—, —S(═O)—, —C(═O)—C≡C—, —C(═O)—[CH₂]ₘ-D-[CH₂]ₙ—, —SO₂—[CH₂]ₘ-D-[CH₂]ₙ—, —O—C(═O)—, —NR¹⁰—, —O—, —(S)ₚ—, —NR¹²—C(═O)—,
—NR¹²—C(═S)—, —O—C(═S)—, —C₁-C₆-cycloalkyl-, -alkenyl-,
-heterocycloalkyl-, unsubstituted or substituted -aryl-, unsubstituted or substituted -heteroaryl-, -aralkyl-, -heteroaralkyl-, -alkyloxy-, -aryloxy-, aralkoxy —NR¹³—SO₂—, —SO₂—NR¹³—, —O—C(═O)—NR¹³—, —NR¹²—C(═O)—NR¹³—, NH—NH— or —O—NH—,
wherein d is an integer from 1 to 6,
m and n, independently, are any integer from 0 to 5,
-D- represents a bond, —S—, —O— or —NR⁹—,
wherein R⁹ represents hydrogen, C₁-C₁₀ alkyl, aryl, heteroaryl or aralkyl,
p is an integer from 1 to 3,
R¹⁰ and R¹², independently, are selected from the group comprising hydrogen, unsubstituted or substituted linear or branched C₁-C₁₀ alkyl, aryl, heteroaryl and aralkyl and
R¹³ represents hydrogen, unsubstituted or substituted linear or branched C₁-C₁₀ alkyl, aryl, cycloalkyl, heterocycloalkyl, heteroaryl, aralkyl or heteroaralkyl,
wherein E-Z²- is a targeting agent radical, wherein E is a biomolecule and Z² represents a moiety selected from the group comprising a bond and a spacer,
wherein the spacer is a natural or un-natural amino acid sequence or a non-amino acid group.
and a pharmaceutically acceptable salt of an inorganic or organic acid thereof, a hydrate, complex, ester, amide, solvate and prodrug thereof.

2. The compound according to claim 1, wherein 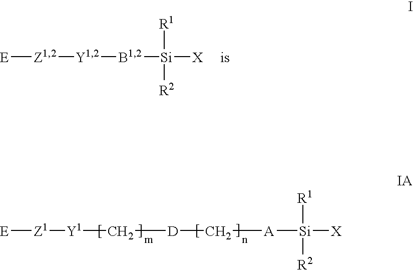
wherein:
X, R¹, R², A, n, m, D and E-Z¹-Y¹ have same meanings as in Formula I according to claim 1, and
E-Z¹ is a targeting agent radical and E- is a biomolecule.

3. The compound according to claim 1, wherein 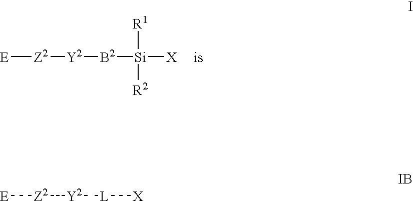
wherein
X, E, Z², Y², A, n, m, and D have the same meanings as in Formula I according to claim 1,
-L- is 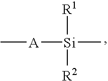
wherein R¹ and R², independently, are selected from the group comprising hydrogen, branched or linear C₁-C₁₀ alkyl, aryl, heteroaryl or aralkyl,
A represents a C₁-C₁₀ alkyl, unsubstituted or substituted aryl or unsubstituted or substituted heteroaryl, and
Y²— is a functional group or a chain containing functional group connecting -L- to Z²- and which is selected from the group comprising a bond, —C(═O)—, —SO₂—, —C(═O)—(CH₂)_{d}—,
—SO—, —C(═O)—C≡C—, —C(═O)-[CH₂]ₘ-D-[CH₂]ₙ—, —SO₂—[CH₂]ₘ-D-[CH₂]ₙ—)—O—C(═O)—, —NR¹⁰—, —O—, —(S)ₚ—, —NR¹²—C(═O)—, —NR¹²—C(═S)—, —O—C(═S)—, —C₁-C₆ cycloalkyl-, —NR¹³SO₂—, —SO₂NR¹³—, OC(═O)—NR¹³—, —NR¹²C(═O)NR¹³—, —NH—NH—, and —O—NH—,
wherein d is an integer from 1 to 6,
m and n, independently, are any integer from 0 to 5,
-D- represents a bond, —S—, —O— or —NR⁹—,
wherein R⁹ represents hydrogen, C₁-C₁₀ alkyl, aryl, heteroaryl or aralkyl,
p is any integer from 1 to 3,
R¹⁰ and R¹², independently, are selected from the group comprising hydrogen, unsubstituted or substituted or branched or linear C₁-C₁₀ alkyl, aryl, heteroaryl and aralkyl, and
R¹³ represents hydrogen, unsubstituted or substituted linear or branched C₁-C₆ alkyl, aryl, cycloalkyl, heterocycloalkyl, heteroaryl, aralkyl or heteroaralkyl.

4. The compound according to claim 1 wherein the leaving group, X is selected from the group consisting of hydrogen or OR³ wherein R³ is hydrogen, (C₁-C₁₀)alkyl, C₁-C₁₀ alkenyl or C₁-C₁₀ alkynyl.

5. The compound according to claim 1 wherein R³ is hydrogen, C₁-C₆ alkyl, C₁-C₆ alkenyl or C₁-C₆ alkynyl.

6. The compound according to claim 1 wherein R³ is hydrogen or C₁-C₆ alkyl.

7. The compound according to claim 1 wherein R¹ and R², independently, are branched C₂-C₅ alkyl.

8. The compound according to claim 1 wherein R¹ and R² are selected from the group comprising iso-propyl, tert-butyl and iso-butyl.

9. The compound according to claim 1 wherein —Y^{1,2}— is selected from the group comprising —C(═O)— and —SO₂—.

10. The compound according to claim 1, wherein -Z²- is an amino acid sequence comprising two (2) to twenty (20) amino acid residues.

11. The compound according to claim 1, wherein -Z²- is Arg-Ser, Arg-Ava, Lys(Me)2-β-ala, Lys(Me)-2-ser, Arg-β-ala, Ser-Ser, Ser-Thr, Arg-Thr, S-alkylcysteine, Cysteic acid, thioalkylcysteine (S—S-Alkyl) or 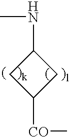
wherein k and l is 0-4.

12. A compound according to claim 1, wherein -Z²- is a non-amino acid moiety selected from the group comprising
—C(═O)—(CH₂)ₚ—NH—, with p being an integer from 2 to 0, and —C(═O)—(CH₂—CH₂—O)_{q}—CH₂—CH₂—NH—,
wherein q being an integer from 0 to 5,
—NH-cycloalkyl-CO— wherein cycloalkyl is selected from C₅-C₈ cycloalkyl, more preferably C₆ atom cycloalkyl, and
—NH-heterocycloalkyl-(CH₂)ₙ—CO—
wherein heterocycloalkyl is selected from C₅-C₈ heterocycloalkyl containing carbon atoms and 1, 2, 3 or 4 oxygen, nitrogen or sulfur heteroatoms and v is an integer of from 1 to 4.

13. The compound according to claim 1, wherein the biomolecule E is selected from the group comprising peptides, peptidomimetics, small molecules and oligonucleotides.

14. The compound according to claim 1 wherein the targeting agent radical -Z¹-E is —NR′-peptide, —NR′—(CH₂)ₙ-peptide, —NR′— small molecules, —NR′—(CH₂)ₙ— small molecules, —NR′-oligonucleotide or —NR′—(CH₂)ₙ— oligonucleotide wherein R′ is selected from the group comprising hydrogen and alkyl, n is from 1 to 6.

15. The compound according to claim 1, wherein the biomolecule E is a peptide comprising from 4 to 100 amino acids.

16. The compound according to claim 1, wherein the biomolecule E is selected from the group comprising somatostatin and derivatives thereof and related peptides, somatostatin receptor specific peptides, neuropeptide Y and derivatives thereof and related peptides, neuropeptide Y₁ and the analogs thereof, bombesin and derivatives thereof and related peptides, gastrin, gastrin releasing peptide and the derivatives thereof and related peptides, epidermal growth factor (EGF of various origin), insulin growth factor (IGF) and IGF-1, integrins (α₃β₁, αᵥβ₃, αᵥβ₅, αllb₃), LHRH agonists and antagonists, transforming growth factors, particularly TGF-α; angiotensin; cholecystokinin receptor peptides, cholecystokinin (CCK) and the analogs thereof; neurotensin and the analogs thereof, thyrotropin releasing hormone, pituitary adenylate cyclase activating peptide (PACAP) and the related peptides thereof, chemokines, substrates and inhibitors for cell surface matrix metalloproteinase, prolactin and the analogs thereof, tumor necrosis factor, interleukins (IL-1, IL-2, IL-4 or IL-6), interferons, vasoactive intestinal peptide (VIP) and the related peptides thereof.

17. The compound according to claim 1, wherein the biomolecule E is bombesin, somatostatin or neuropeptide Y₁ and analogs thereof.

18. The compound according to claim 1, wherein the biomolecule E comprises bombesin analogs having sequence III or IV:
AA₁-AA₂-AA₃-AA₄-AA₅-AA₆-AA₇-AA₈-NT₁T₂ (type A) III, with
T₁=T₂=H, T₁=H, T₂=OH, T₁=CH₃, T₂=OH
AA₁=Gln, Asn, Phe(4-CO—NH₂)
AA₂=Trp, D-Trp
AA₃=Ala, Ser, Val
AA₄=Val, Ser. Thr
AA₅=Gly, (N-Me)Gly
AA₆=His, His(3-Me), (N-Me)His, (N-Me)His(3-Me)
AA₇=Sta, Statine analogs and isomers, 4-Am,5-MeHpA, 4-Am,5-MeHxA and γ-substituted aminoacids
AA₈=Leu, Cpa, Cba, CpnA, Cha, t-buGly, tBuAla, Met, Nle, iso-Bu-Gly
AA₁-AA₂-AA₃-AA₄-AA₅-AA₆-AA₇-AA₈-NT₁T₂ (type B) IV, with:
T₁=T₂=H, T₁=H, T₂=OH, T₁=CH₃, T₂=OH
AA₁=Gln, Asn, Phe(4-CO—NH₂)
AA₂=Trp, D-Trp
AA₃=Ala, Ser, Val
AA₄=Val, Ser. Thr
AA₅=βAla, β²- and β³-amino acids as shown herein after 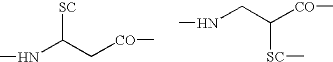
wherein SC represents side chain found in proteinogenic amino acids and homologs of proteinogenic amino acids,
AA₆=His, His(3-Me), (N-Me)His, (N-Me)His(3-Me)
AA₇=Phe, Tha, NaI,
AA₈=Leu, Cpa, Cba, CpnA, Cha, t-buGly, tBuAla, Met, Nle, iso-Bu-Gly.

19. The compound according to claim 1 selected from (SEQ ID NOS 5-6, 9-11 and 13, respectively, in order of appearance)

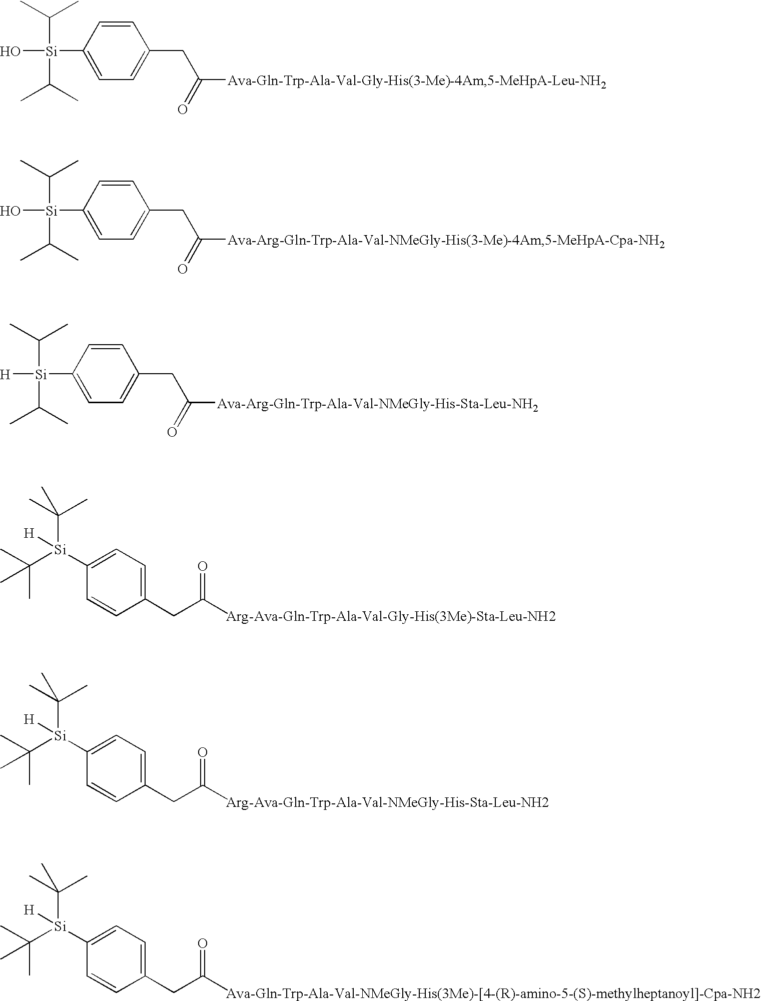

20. A compound having the general chemical Formula II 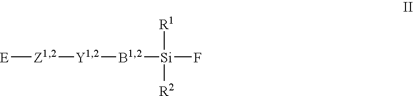
wherein B^{1,2}, Y^{1,2}, E, R¹ and R² have the same meanings as in Formula I, F is fluorine isotope wherein F is selected from radioactive or non-radioactive isotope, and a pharmaceutically acceptable salt of an inorganic or organic acid thereof, a hydrate, complex, ester amide, solvate and prodrug thereof.

21. The compound according to claim 20, wherein 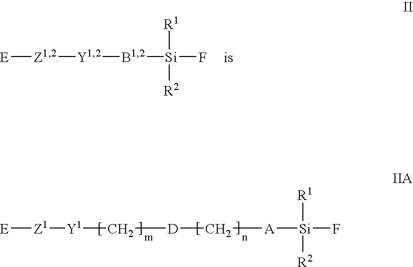
F is fluorine isotope wherein F is selected from radioactive or non-radioactive isotope,
R¹, R², A, n, m, D and E-Z¹-Y¹ have the same meanings as in Formula I above, and
E-Z¹- is a targeting agent radical, wherein E is a biomolecule.

22. The compound according to claim 20, wherein 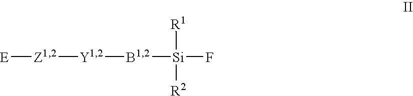
has the following meaning
E-Z²-Y²-L-F IIB
wherein
F is fluorine isotope wherein F is selected from radioactive or non-radioactive isotope, 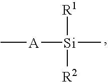
-L- is
wherein R¹ and R², independently, are selected from the group comprising hydrogen, linear or branched C₁-C₁₀ alkyl, aryl, heteroaryl and aralkyl and A represents alkyl, unsubstituted or substituted aryl or unsubstituted or substituted heteroaryl,
—Y²— is a functional group or a chain containing functional group connecting -L- to -Z²- and which is selected from the group comprising a bond, —C(═O)—, —SO₂—,
—C(═O)—(CH₂)_{d}—, —SO—, —C(═O)—C≡C—, —C(═O)—[CH₂]ₘ-D-[CH₂]ₙ—, —SO₂—[CH₂]ₘ-D-[CH₂]ₙ—, —O—C(═O)—, —NR¹⁰—, —O—, —(═S)ₚ—, —NR¹²—C(═O)—, —NR¹²—C(═S)—, —O—C(═S)—, —C₁-C₆ cycloalkyl-, -alkenyl-, -heterocycloalkyl-, unsubstituted or substituted aryl-, unsubstituted or substituted -heteroaryl, -aralkyl-, -heteroaralkyl, -alkyloxy-, aryloxy-, -aralkyloxy-, -aryl-, —NR³SO₂—, —SO₂NR¹³—, OC(═O)—NR¹³—, —NR¹²C(═O)NR¹³—, —NH—NH—, and —O—NH—,
wherein d is an integer from 1 to 6,
m and n, independently, are any integer from 0 to 5,
-D- represents a bond, —S—, —O— or —NR⁹—,
Wherein R⁹ represents hydrogen, C₁-C₁₀ alkyl, aryl, heteroaryl or aralkyl,
p is any integer from 1 to 3,
R¹⁰ and R¹², independently, are selected from the group comprising hydrogen, unsubstituted or substituted or linear or branched C₁-C₁₀ alkyl, aryl, heteroaryl and aralkyl, and
R¹³ represents hydrogen, substituted or unsubstituted, linear or branched C₁-C₁₀ alkyl, aryl, cycloalkyl, heterocycloalkyl, heteroaryl, aralkyl or heteroaralkyl.
E-Z²- is a targeting agent radical, wherein E is a targeting agent and Z² represents a moiety selected from the group comprising a bond and a spacer, wherein the spacer is a natural or un-natural amino acid sequence or a non-amino acid group and
E is a biomolecule.

23. The compound according to claim 20 wherein F=¹⁸F.

24. The compound according to claim 20 selected from (Structures disclose SEQ ID NOS 10, 10, 11 and 11, respectively, in order of appearance)
IIA-c-2:
(SEQ ID NO: 14)
¹⁸F-Si(tBu)₂-C₆H₄-CH₂-CO-Ava--Gln-Trp-Ala-Val-
NMeGly-His(3Me)-4-Am,5-MeHpA-Cpa-NH₂,
IIB-c-1:
(SEQ ID NO: 15)
¹⁹F-Si(iPr)₂-C₆H₄-CH₂-CO-Ava--Gln-Trp-Ala-Val-Gly-
His(3Me)-4-Am,5-MeHpA-Leu-NH₂,
IIB-c-2:
(SEQ ID NO: 16)
¹⁹F-Si(tBu)₂-C₆H₄-CH₂-CO-Ava--Gln-Trp-Ala-Val-
NMeGly-His(3Me)-4-Am,5-MeHpA-Cpa-NH₂. 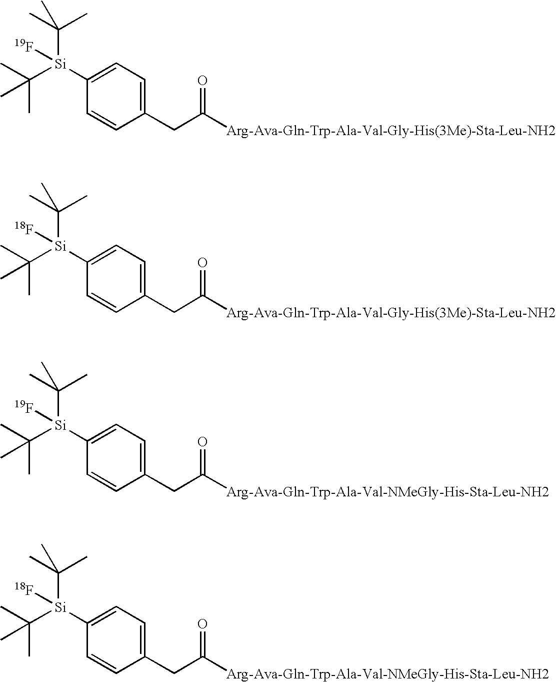
[4-(Fluoro-di-iso-propyl-silanyl)-phenyl]-acetic acid
2-[4-(Fluoro-di-iso-propyl-silanyl)-phenyl]-ethanol
4-(Fluoro-di-iso-propyl-silanyl)-benzoic acid
[4-(Fluoro-di-iso-propyl-silanyl)-phenyl]-methanol
3-[4-(Fluoro-di-iso-propyl-silanyl)-phenyl]-propan-1-ol
3-[4-(Fluoro-di-iso-propyl-silanyl)-phenyl]-propionic acid
3-[3-(Fluoro-di-iso-propyl-silanyl)-phenyl]-propan-1-ol
3-[3-(Fluoro-di-iso-propyl-silanyl)-phenyl]-propionic acid
2-[3-(Fluoro-di-iso-propyl-silanyl)-phenyl]-ethanol
[3-(Fluoro-di-iso-propyl-silanyl)-phenyl]-acetic acid
[4-(Fluoro-di-iso-propyl-silanyl)-phenyl]-acetic acid and
4-(Fluoro-di-iso-propyl-silanyl)-benzoic acid
wherein fluoro means ¹⁸F or ¹⁹F.

25. A compound having general chemical Formula III 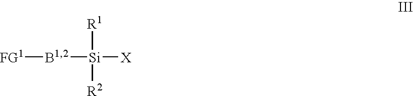
wherein
FG¹- represents —OH, -Hal, —N₃, —CO₂R⁸, —NHR⁵, —N═C═O, —O═C═N, —S═C═N, —N═C═S, —O—SO₂-Aryl, —O—SO₂-Alkyl, —SO₂-Hal, —S₃H, —SH, —O—C(═O)—Hal, —O—C(═S)-Hal, 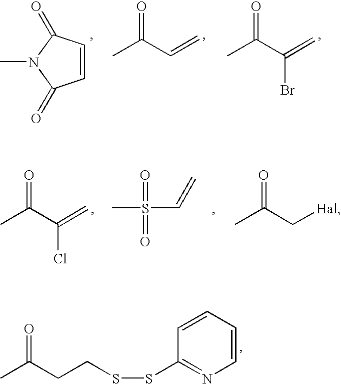
wherein Hal represents a halogen atom, and
R⁸ represents hydrogen, C₁-C₁₀ alkyl, C₂-C₁₀ alkenyl, aralkyl or 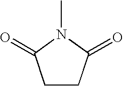
and wherein X, R¹, R² and B^{1,2} have the same meanings as in Formula I.

26. The compound according to claim 25, wherein 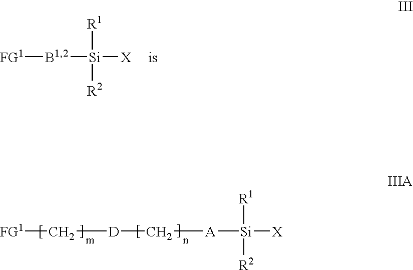
wherein X, R¹, R², A, D, m and m have the same meanings as in Formula IA and FG¹ has the same meaning as the Formula III.

27. The compound selected from
(Di-tert-butyl-hydroxy-silanyl)-acetic acid
[4-(Hydroxy-diisopropyl-silanyl)-phenyl]-acetic acid
[4-(Hydroxy-diisopropyl-silanyl)-phenyl]-acetic acid
(4-Diisopropylsilanyl-phenyl)-acetic acid
(4-Diisopropylsilanyl-phenyl)-acetic acid 2,5-dioxo-pyrrolidin-1-yl ester
[4-(Hydroxy-diisopropyl-silanyl)-phenyl]-acetic acid 2,5-dioxo-pyrrolidin-1-yl ester
4-Diisopropylsilanyl-benzoic acid
4-(Hydroxy-diisopropyl-silanyl)-benzoic acid
4-Diisopropylsilanyl-benzoic acid 2,5-dioxo-pyrrolidin-1-yl ester
4-(Hydroxy-diisopropyl-silanyl)-benzoic acid 2,5-dioxo-pyrrolidin-1-yl ester
3-(4-Diisopropylsilanyl-phenyl)-propionic acid
3-(4-Diisopropylsilanyl-phenyl)-propionic acid 2,5-dioxo-pyrrolidin-1-yl ester
3-(3-Diisopropylsilanyl-phenyl)-propionic acid
3-(3-Diisopropylsilanyl-phenyl)-propionic acid 2,5-dioxo-pyrrolidin-1-yl ester
(3-Diisopropylsilanyl-phenyl)-acetic acid
(3-Diisopropylsilanyl-phenyl)-acetic acid 2,5-dioxo-pyrrolidin-1-yl ester
(4-Diisobutylsilanyl-phenyl)-acetic acid
(4-Diisobutylsilanyl-phenyl)-acetic acid 2,5-dioxo-pyrrolidin-1-yl ester
[4-(Hydroxy-diisobutyl-silanyl)-phenyl]-acetic acid 2,5-dioxo-pyrrolidin-1-yl ester
[4-(Hydroxy-diisobutyl-silanyl)-phenyl]-acetic acid
4-Diisobutylsilanyl-benzoic acid
4-(Hydroxy-diisobutyl-silanyl)-benzoic acid
4-Diisobutylsilanyl-benzoic acid 2,5-dioxo-pyrrolidin-1-yl ester
4-(Hydroxy-diisobutyl-silanyl)-benzoic acid 2,5-dioxo-pyrrolidin-1-yl ester
4-[3-(Ethoxy-diisopropyl-silanyl)-propylcarbamoyl]-butyric acid
4-[3-(Ethoxy-diisopropyl-silanyl)-propylcarbamoyl]-butyric acid 2,5-dioxo-pyrrolidin-1-yl ester
5-[Diisobutyl-(4-phenyl-butoxy)-silanyl]-pentanoic acid 2,5-dioxo-pyrrolidin-1-yl ester
5-[(4-Polystyrene-methoxy-benzyloxy)-diisobutyl-silanyl]-pentanoic acid 2,5-dioxo-pyrrolidin-1-yl ester
5-(Polystyrene-methoxy-diisobutyl-silanyl)-pentanoic acid 2,5-dioxo-pyrrolidin-1-yl ester
5-(Polystyrene-ethoxy-diisobutyl-silanyl)-pentanoic acid 2,5-dioxo-pyrrolidin-1-yl ester.

28. A method for producing a compound having general chemical Formula I as defined in claim 1, wherein a compound of general Formula III, is reacted with a compound of general Formula IV:
E-FG² IV
wherein
FG² has the meaning as given herein above for FG¹, and E has the same meaning as defined in claim 1.

29. A method for producing a compound having general chemical Formula II, as defined in claim 20 wherein X is F, wherein F is a fluorine isotope, said method comprising the step of reacting a compound having general chemical Formula I with a fluorinating agent.

30. The method according to claim 29 wherein the step of reacting is at a reaction temperature of 80° C. or less.

31. The method according to claim 30 wherein the reaction temperature is 50° C. or less.

32. A compound selected from
[4-([¹⁸F]Fluoro-di-iso-propyl-silanyl)-phenyl]-acetic acid
2-[4-([¹⁸F]Fluoro-di-iso-propyl-silanyl)-phenyl]-ethanol
4-([¹⁸F]Fluoro-di-iso-propyl-silanyl)-benzoic acid
[4-([¹⁸F]Fluoro-di-iso-propyl-silanyl)-phenyl]-methanol
3-[4-([¹⁸F]Fluoro-di-iso-propyl-silanyl)-phenyl]-propan-1-ol
3-[4-([¹⁸F]Fluoro-di-iso-propyl-silanyl)-phenyl]-propionic acid
3-[3-([¹⁸F]Fluoro-di-iso-propyl-silanyl)-phenyl]-propan-1-ol
3-[3-([¹⁸F]Fluoro-di-iso-propyl-silanyl)-phenyl]-propionic acid
2-[3-([¹⁸F]Fluoro-di-iso-propyl-silanyl)-phenyl]-ethanol
[3-([¹⁸F]Fluoro-di-iso-propyl-silanyl)-phenyl]-acetic acid
[4-([¹⁸F]Fluoro-di-iso-butyl-silanyl)-phenyl]-acetic acid
4-([¹⁸F]Fluoro-di-iso-butyl-silanyl)-benzoic acid
N-(3-([¹⁸F]Fluoro-dimethylsilyl)propyl)biphenyl-4-carboxamide
N-(3-([¹⁸F]Fluoro-di-iso-propylsilyl)propyl)biphenyl-4-carboxamide
[¹⁸F]Fluoro-di-iso-propyl-{4-[2-(tetrahydro-pyran-2-yloxy)-ethyl]-phenyl}-silane
[¹⁸F]Fluoro-di-iso-propyl-[4-[2-(tetrahydro-pyran-2-yloxy)-ethyl]-phenyl]-si lane
(SEQ ID NO: 76)
2-[4-([¹⁸F]Fluoro-di-iso-propyl-silanyl)-phenyl]-
acetyl-Val-βAla-Phe-Gly-NH₂;
(SEQ ID NO: 78)
3-[3-([^{18F}]Fluoro-di-iso-propyl-silanyl)-phenyl]-
propanyl-Val-βAla-Phe-Gly-NH₂;
(SEQ ID NO: 92)
2-[4-([¹⁸F]Fluoro-di-iso-propyl-silanyl)-phenyl]-
acetyl-Ala-Gln-Trp-Gly-His(3-Me)-FA02010-Leu-NH₂
Benzyl 2-(di-tert-butyl-[¹⁸F]fluorosilyl)acetate
Benzyl 2-(di-tert-butyl-[¹⁸F]fluorosilyl)acetate
(SEQ ID NO: 80)
1-(Di-tert-butylfluorosilyl)acetyl-Val-βAla-Phe-
Gly-NH₂
N-Benzyl-2-(4-(di-tert-butyl[¹⁸F]fluorosilyl)phenyl)acetamide
N-Benzyl-2-(4-(di-tert-butyl[¹⁸F]fluorosilyl)phenyl)acetamide
(SEQ ID NO: 93)
2-(4-(Di-tert-butyl[¹⁸F]fluorosilyl)phenyl)acetyl-
Ala-Gln-Trp-Gly-His(3-Me)-FA02010-Leu-NH₂

33. A composition comprising a compound having general chemical Formula I or II a pharmaceutically acceptable salt of an inorganic or organic acid thereof, a hydrate, complex, ester, amide, solvate and prodrug thereof.

34. The composition according to claim 33, further comprising a pharmaceutically acceptable carrier, diluent, adjuvant or excipient.

35. A method for imaging wherein the method comprising the step of introducing into a patient a detectable quantity of a labelled compound having general chemical Formula II according to claim 20, or a pharmaceutically acceptable salt of an inorganic or organic acid thereof, a hydrate, complex, ester, amide, solvate and prodrug thereof and imaging said patient.

36. A kit comprising a vial containing a predetermined quantity of the compound having general chemical Formula I according to claim 1, or a pharmaceutically acceptable salt of an inorganic or organic acid thereof, a hydrate, complex, ester, amide, solvate and prodrug.

37. A compound having general chemical Formula I or II according to claim 1, or a pharmaceutically acceptable salt of an inorganic or organic acid thereof, a hydrate, complex, ester, amide, solvate and prodrug thereof for use as medicament.

38. A compound having general chemical Formula II according to claim 20, or a pharmaceutically acceptable salt of an inorganic or organic acid thereof, a hydrate, complex, ester, amide, solvate and prodrug thereof for use as diagnostic imaging agent.

39. A compound having general chemical Formula II according to claim 20, or a pharmaceutically acceptable salt of an inorganic or organic acid thereof, a hydrate, complex, ester, amide, solvate and prodrug thereof for use as imaging agent for positron emission tomography (PET).

40. A use of the compound having any one of general chemical Formulae I or II according to claim 1, or of a pharmaceutically acceptable salt of an inorganic or organic acid thereof, a hydrate, complex, ester, amide, solvate and prodrug thereof, for the manufacture of a medicament.

41. A use of the compound having any one of general chemical Formulae I or II according to claim 1, or of a pharmaceutically acceptable salt of an inorganic or organic acid thereof, a hydrate, complex, ester, amide, solvate and prodrug thereof for the manufacture of a diagnostic imaging agent.

42. The use according to claim 41 wherein the diagnostic imaging agent is for positron emission tomography.

43. The use according to claim 41 for the manufacture of a diagnostic imaging agent for imaging tissue at a target site using the imaging agent.

44. The use according to claim 41 for imaging of tumors, imaging of inflammatory and/or neurodegenerative diseases, such as multiple sclerosis or Alzheimer's disease, or imaging of angiogenesis-associated diseases, such as growth of solid tumors, and rheumatoid arthritis.


SEQUENCE LISTING (100 sequences)

SEQ ID NO: 1 - protein, 8 aa, Artificial Sequence
  Description of Artificial Sequence Synthetic peptide
QWAVGHXL

SEQ ID NO: 2 - protein, 8 aa, Artificial Sequence
  Description of Artificial Sequence Synthetic peptide
QWAVGHXL

SEQ ID NO: 3 - protein, 8 aa, Artificial Sequence
  Description of Artificial Sequence Synthetic peptide
QWAVGHXL

SEQ ID NO: 4 - protein, 8 aa, Artificial Sequence
  Description of Artificial Sequence Synthetic peptide
QWAVGHXL

SEQ ID NO: 5 - protein, 9 aa, Artificial Sequence
  Description of Artificial Sequence Synthetic peptide
XQWAVGHXL

SEQ ID NO: 6 - protein, 10 aa, Artificial Sequence
  Description of Artificial Sequence Synthetic peptide
XRQWAVGHXX

SEQ ID NO: 7 - protein, 8 aa, Artificial Sequence
  Description of Artificial Sequence Synthetic peptide
QWAVGHXX

SEQ ID NO: 8 - protein, 8 aa, Artificial Sequence
  Description of Artificial Sequence Synthetic peptide
QWAVGHXL

SEQ ID NO: 9 - protein, 10 aa, Artificial Sequence
  Description of Artificial Sequence Synthetic peptide
XRQWAVGHXL

SEQ ID NO: 10 - protein, 10 aa, Artificial Sequence
  Description of Artificial Sequence Synthetic peptide
RXQWAVGHXL

SEQ ID NO: 11 - protein, 10 aa, Artificial Sequence
  Description of Artificial Sequence Synthetic peptide
RXQWAVGHXL

SEQ ID NO: 12 - protein, 8 aa, Artificial Sequence
  Description of Artificial Sequence Synthetic peptide
QWAVGHXL

SEQ ID NO: 13 - protein, 9 aa, Artificial Sequence
  Description of Artificial Sequence Synthetic peptide
XQWAVGHXX

SEQ ID NO: 14 - protein, 9 aa, Artificial Sequence
  Description of Artificial Sequence Synthetic peptide
XQWAVGHXX

SEQ ID NO: 15 - protein, 9 aa, Artificial Sequence
  Description of Artificial Sequence Synthetic peptide
XQWAVGHXL

SEQ ID NO: 16 - protein, 9 aa, Artificial Sequence
  Description of Artificial Sequence Synthetic peptide
XQWAVGHXX

SEQ ID NO: 17 - protein, 8 aa, Artificial Sequence
  Description of Artificial Sequence Synthetic peptide
QWAVGHXL

SEQ ID NO: 18 - protein, 4 aa, Artificial Sequence
  Description of Artificial Sequence Synthetic peptide
VAFG

SEQ ID NO: 19 - protein, 4 aa, Artificial Sequence
  Description of Artificial Sequence Synthetic peptide
VAFG

SEQ ID NO: 20 - protein, 9 aa, Artificial Sequence
  Description of Artificial Sequence Synthetic peptide
XQWAVGHXX

SEQ ID NO: 21 - protein, 10 aa, Artificial Sequence
  Description of Artificial Sequence Synthetic peptide
RXQWAVGHXL

SEQ ID NO: 22 - protein, 10 aa, Artificial Sequence
  Description of Artificial Sequence Synthetic peptide
RXQWAVGHXL

SEQ ID NO: 23 - protein, 8 aa, Artificial Sequence
  Description of Artificial Sequence Synthetic peptide
QWAVGHXX

SEQ ID NO: 24 - protein, 4 aa, Artificial Sequence
  Description of Artificial Sequence Synthetic peptide
VAFG

SEQ ID NO: 25 - protein, 9 aa, Artificial Sequence
  Description of Artificial Sequence Synthetic peptide
XQWAVGHXX

SEQ ID NO: 26 - protein, 10 aa, Artificial Sequence
  Description of Artificial Sequence Synthetic peptide
RXQWAVGHXL

SEQ ID NO: 27 - protein, 8 aa, Artificial Sequence
  Description of Artificial Sequence Synthetic peptide
QWAVGHXX

SEQ ID NO: 28 - protein, 8 aa, Artificial Sequence
  Description of Artificial Sequence Synthetic peptide
QWAVGHXG

SEQ ID NO: 29 - protein, 10 aa, Artificial Sequence
  Description of Artificial Sequence Synthetic peptide
RXQWAVGHXL

SEQ ID NO: 30 - protein, 8 aa, Artificial Sequence
  Description of Artificial Sequence Synthetic peptide
QWAVGHXG

SEQ ID NO: 31 - protein, 4 aa, Artificial Sequence
  Description of Artificial Sequence Synthetic peptide
VAFG

SEQ ID NO: 32 - protein, 8 aa, Artificial Sequence
  Description of Artificial Sequence Synthetic peptide
QWAVGHXL

SEQ ID NO: 33 - protein, 8 aa, Artificial Sequence
  Description of Artificial Sequence Synthetic peptide
QWAVGHXG

SEQ ID NO: 34 - protein, 8 aa, Artificial Sequence
  Description of Artificial Sequence Synthetic peptide
QWAVGHXX

SEQ ID NO: 35 - protein, 8 aa, Artificial Sequence
  Description of Artificial Sequence Synthetic peptide
QWAVGHXX

SEQ ID NO: 36 - protein, 8 aa, Artificial Sequence
  Description of Artificial Sequence Synthetic peptide
QWAVGHXA

SEQ ID NO: 37 - protein, 4 aa, Artificial Sequence
  MOD_RES (1)..(1): 3-(Hydroxy-diisopropyl-silanyl)- phenylpropionyl-Val
VAFG

SEQ ID NO: 38 - protein, 4 aa, Artificial Sequence
  Description of Artificial Sequence Synthetic peptide
VAFG

SEQ ID NO: 39 - protein, 4 aa, Artificial Sequence
  Description of Artificial Sequence Synthetic peptide
VAFG

SEQ ID NO: 40 - protein, 4 aa, Artificial Sequence
  Description of Artificial Sequence Synthetic peptide
VAFG

SEQ ID NO: 41 - protein, 4 aa, Artificial Sequence
  Description of Artificial Sequence Synthetic peptide
VAFG

SEQ ID NO: 42 - protein, 8 aa, Artificial Sequence
  Description of Artificial Sequence Synthetic peptide
QWAVGHXX

SEQ ID NO: 43 - protein, 8 aa, Artificial Sequence
  Description of Artificial Sequence Synthetic peptide
QWAVGHXG

SEQ ID NO: 44 - protein, 4 aa, Artificial Sequence
  Description of Artificial Sequence Synthetic peptide
VAFG

SEQ ID NO: 45 - protein, 4 aa, Artificial Sequence
  Description of Artificial Sequence Synthetic peptide
VAFG

SEQ ID NO: 46 - protein, 8 aa, Artificial Sequence
  Description of Artificial Sequence Synthetic peptide
QWAVGHXL

SEQ ID NO: 47 - protein, 4 aa, Artificial Sequence
  Description of Artificial Sequence Synthetic peptide
VAFG

SEQ ID NO: 48 - protein, 8 aa, Artificial Sequence
  Description of Artificial Sequence Synthetic peptide
QWAVGHXL

SEQ ID NO: 49 - protein, 8 aa, Artificial Sequence
  Description of Artificial Sequence Synthetic peptide
QWAVGHXX

SEQ ID NO: 50 - protein, 8 aa, Artificial Sequence
  Description of Artificial Sequence Synthetic peptide
QWAVGHXL

SEQ ID NO: 51 - protein, 8 aa, Artificial Sequence
  Description of Artificial Sequence Synthetic peptide
QWAVGHXL

SEQ ID NO: 52 - protein, 8 aa, Artificial Sequence
  Description of Artificial Sequence Synthetic peptide
QWAVAHXX

SEQ ID NO: 53 - protein, 8 aa, Artificial Sequence
  Description of Artificial Sequence Synthetic peptide
QWAVAHFX

SEQ ID NO: 54 - protein, 8 aa, Artificial Sequence
  Description of Artificial Sequence Synthetic peptide
QWAVAHFL

SEQ ID NO: 55 - protein, 8 aa, Artificial Sequence
  Description of Artificial Sequence Synthetic peptide
QWAVAHFL

SEQ ID NO: 56 - protein, 8 aa, Artificial Sequence
  Description of Artificial Sequence Synthetic peptide
QWAVAHLL

SEQ ID NO: 57 - protein, 8 aa, Artificial Sequence
  Description of Artificial Sequence Synthetic peptide
QWAVAHIL

SEQ ID NO: 58 - protein, 8 aa, Artificial Sequence
  Description of Artificial Sequence Synthetic peptide
QWAVAHLG

SEQ ID NO: 59 - protein, 8 aa, Artificial Sequence
  Description of Artificial Sequence Synthetic peptide
QWAVAHFX

SEQ ID NO: 60 - protein, 8 aa, Artificial Sequence
  Description of Artificial Sequence Synthetic peptide
QWAVAHFX

SEQ ID NO: 61 - protein, 8 aa, Artificial Sequence
  Description of Artificial Sequence Synthetic peptide
QWAVAHFG

SEQ ID NO: 62 - protein, 8 aa, Artificial Sequence
  Description of Artificial Sequence Synthetic peptide
QWAVAHXG

SEQ ID NO: 63 - protein, 8 aa, Artificial Sequence
  Description of Artificial Sequence Synthetic peptide
QWAVAHXG

SEQ ID NO: 64 - protein, 8 aa, Artificial Sequence
  Description of Artificial Sequence Synthetic peptide
QWAVAHFX

SEQ ID NO: 65 - protein, 8 aa, Artificial Sequence
  Description of Artificial Sequence Synthetic peptide
QWAVAHFL

SEQ ID NO: 66 - protein, 8 aa, Artificial Sequence
  Description of Artificial Sequence Synthetic peptide
QWAVAHFL

SEQ ID NO: 67 - protein, 8 aa, Artificial Sequence
  Description of Artificial Sequence Synthetic peptide
QWAVAHFL

SEQ ID NO: 68 - protein, 8 aa, Artificial Sequence
  Description of Artificial Sequence Synthetic peptide
QWAVAHFL

SEQ ID NO: 69 - protein, 8 aa, Artificial Sequence
  Description of Artificial Sequence Synthetic peptide
QWAVAHFL

SEQ ID NO: 70 - protein, 8 aa, Artificial Sequence
  Description of Artificial Sequence Synthetic peptide
QWAVAHFL

SEQ ID NO: 71 - protein, 8 aa, Artificial Sequence
  Description of Artificial Sequence Synthetic peptide
QWAVAHIG

SEQ ID NO: 72 - protein, 8 aa, Artificial Sequence
  Description of Artificial Sequence Synthetic peptide
QWAVGHXX

SEQ ID NO: 73 - protein, 8 aa, Artificial Sequence
  Description of Artificial Sequence Synthetic peptide
QWAVGHXX

SEQ ID NO: 74 - protein, 8 aa, Artificial Sequence
  Description of Artificial Sequence Synthetic peptide
QWAVGHXA

SEQ ID NO: 75 - protein, 8 aa, Artificial Sequence
  Description of Artificial Sequence Synthetic peptide
QWAVGHXA

SEQ ID NO: 76 - protein, 4 aa, Artificial Sequence
  Description of Artificial Sequence Synthetic peptide
VAFG

SEQ ID NO: 77 - protein, 4 aa, Artificial Sequence
  Description of Artificial Sequence Synthetic peptide
VAFG

SEQ ID NO: 78 - protein, 4 aa, Artificial Sequence
  Description of Artificial Sequence Synthetic peptide
VAFG

SEQ ID NO: 79 - protein, 4 aa, Artificial Sequence
  Description of Artificial Sequence Synthetic peptide
VAFG

SEQ ID NO: 80 - protein, 4 aa, Artificial Sequence
  Description of Artificial Sequence Synthetic peptide
VAFG

SEQ ID NO: 81 - protein, 4 aa, Artificial Sequence
  Description of Artificial Sequence Synthetic peptide
VAFG

SEQ ID NO: 82 - protein, 8 aa, Artificial Sequence
  Description of Artificial Sequence Synthetic peptide
QWAVGHXL

SEQ ID NO: 83 - protein, 9 aa, Artificial Sequence
  Description of Artificial Sequence Synthetic peptide
XQWAVGHXX

SEQ ID NO: 84 - protein, 4 aa, Artificial Sequence
  Description of Artificial Sequence Synthetic peptide
VAFG

SEQ ID NO: 85 - protein, 4 aa, Artificial Sequence
  Description of Artificial Sequence Synthetic peptide
VAFG

SEQ ID NO: 86 - protein, 10 aa, Artificial Sequence
  Description of Artificial Sequence Synthetic peptide
RXQWAVGHXL

SEQ ID NO: 87 - protein, 10 aa, Artificial Sequence
  Description of Artificial Sequence Synthetic peptide
RXQWAVGHXL

SEQ ID NO: 88 - protein, 10 aa, Artificial Sequence
  Description of Artificial Sequence Synthetic peptide
RXQWAVGHXL

SEQ ID NO: 89 - protein, 10 aa, Artificial Sequence
  Description of Artificial Sequence Synthetic peptide
RXQWAVGHXL

SEQ ID NO: 90 - protein, 8 aa, Artificial Sequence
  Description of Artificial Sequence Synthetic peptide
QWAVGHXL

SEQ ID NO: 91 - protein, 8 aa, Artificial Sequence
  Description of Artificial Sequence Synthetic peptide
QWAVGHXL

SEQ ID NO: 92 - protein, 7 aa, Artificial Sequence
  Description of Artificial Sequence Synthetic peptide
AQWGHXL

SEQ ID NO: 93 - protein, 7 aa, Artificial Sequence
  Description of Artificial Sequence Synthetic peptide
AQWGHXL

SEQ ID NO: 94 - protein, 9 aa, Artificial Sequence
  Description of Artificial Sequence Synthetic peptide
QWAVGHXXL

SEQ ID NO: 95 - protein, 9 aa, Artificial Sequence
  Description of Artificial Sequence Synthetic peptide
QWAVGHXXX

SEQ ID NO: 96 - protein, 6 aa, Artificial Sequence
  Description of Artificial Sequence Synthetic peptide
KFXWKT

SEQ ID NO: 97 - protein, 6 aa, Artificial Sequence
  Description of Artificial Sequence Synthetic peptide
XMFYWK

SEQ ID NO: 98 - protein, 8 aa, Artificial Sequence
  Description of Artificial Sequence Synthetic peptide
CLITRCRY

SEQ ID NO: 99 - protein, 8 aa, Artificial Sequence
  Description of Artificial Sequence Synthetic peptide
CLIVRCRY

SEQ ID NO: 100 - protein, 8 aa, Artificial Sequence
  Description of Artificial Sequence Synthetic peptide
QWAVGHXL